SEQUENCE LISTING (6 sequences)

SEQ ID NO: 1 - DNA, 21 bp, Artificial sequence
  DAZL primer
ccaccacagtttcagaatgtc

SEQ ID NO: 2 - DNA, 23 bp, Artificial sequence
  DAZL primer
caaagtttgagtgtgatttacca

SEQ ID NO: 3 - DNA, 21 bp, Artificial sequence
  Oct-4 Primer
gaggaagctgacaacaatgaa

SEQ ID NO: 4 - DNA, 21 bp, Artificial sequence
  Oct-4 Primer
ggttttctttccctagctcct

SEQ ID NO: 5 - DNA, 21 bp, Artificial sequence
  Oct-4 Primer
caggagatatgcaaagcagaa

SEQ ID NO: 6 - DNA, 20 bp, Artificial sequence
  Oct-4 Primer
agcctcaaaatcctctcgtt

(19) United States
(12) Patent Application Publication
(10) Pub. No.: US 2008/0159999 A1
(43) Pub. Date: Jul. 3, 2008

(54) Compositions and methods for identifying, isolating and enriching germline-like stem cells from amniotic fluid

(76) Inventor: Konstantinos Stefanidis, Athens (GR)

Correspondence Address: THE NATH LAW GROUP, 112 South West Street, Alexandria, VA, 22314, US

(21) Appl. No.: 11/976,321

(22) Filed: Oct. 23, 2007

Related U.S. Application Data
(60) Provisional application No. 60/853,420, filed on Oct. 23, 2006.

(51) Int. Cl. A61K 35/12; C12N 5/06; C12Q 1/68; A61P 1/00; A61P 25/00; A61P 43/00; A61P 17/00; G01N 33/53; C12Q 1/02

(52) U.S. Cl. 424/93.21; 435/366; 435/378; 435/6; 435/7.21; 435/29; 435/405

(57) ABSTRACT

The present invention is directed to pluripotent embryonic stem cells derived from amniotic fluid and the methods for isolating, expanding and differentiating these cells, and their therapeutic uses such as manipulating the cells by gene transfection and other means for therapeutic applications.

### FIELD OF THE INVENTION

The present invention relates generally to the field of stem cells. More specifically, this invention relates to isolated amniotic fluid pluripotent stem cell populations, and methods for identifying, isolating and enriching for such stem cells.

### BACKGROUND OF THE INVENTION

Stem cells are undifferentiated cells with the ability to undergo both renewal and differentiation. Stem cells derived from the embryo are termed embryonic stem (ES) cells. ES cells are pluripotent and thus posses the capability of developing into any organ or tissue type or, at least potentially, into a complete embryo.

Pluripotent embryonic stem cells have been traditionally derived from embryonic sources. One type can be isolated from cells of the inner cell mass (ICM) at the blastula stage of a pre-implantation embryo (Evans and Kaufman, Nature 292,154-156, 1981; U.S. Pat. No. 6,200,806). A second type can be isolated from primordial germ cells (PGCs) in the mesenteric or genital ridges of embryos and has been termed the embryonic germ cell (EG) (U.S. Pat. No. 5,453,357, U.S. Pat. No. 6,245,566).

Human embryonic stem (hES) cells display a distinct group of cell surface antigens such as SSEA-3, SSEA-4, TRA-2-54 (alkaline phosphatase), TRA-1-60 and TRA-1-81, in addition to expressing specific transcription factors such as OCT-4, NANOG, SOX-2, FGF-4 and REX-1 (Henderson, et al., (2002) Stem Cells 20:329-337; Draper, et al., (2002). J. Anat. 200:249-258; Mitsui et al., (2003) Cell 113:631-642; Chambers et al., (2003) Cell 113:643-655), the disclosures of which are incorporated by reference herein in their entirety).

Despite tremendous interest in ES cell research, the destruction of embryos in order to harvest and experiment on ES cells is controversial and thus the use human embryos or human fetal tissues for ES research is prohibited or strictly regulated in various jurisdictions. Therefore, there is a need for a method of obtaining stem cells from an alternative source that does not raise ethical concerns.

Amniotic fluid cells (AFC) have been suggested to be an attractive alternative to the traditional methods for obtaining ES cells. United States patent application 20050042595 discloses a method for isolating and growing multipotent amniotic fetal stem cells from amniotic fluid cells. These cells, however, are considered to be fetal mesenchymal stem cells and are considered to be only multipotent, thus have the differentiation potential for adipogenic, osteogenic and neurogenic cell lineages (Bossolasco et al. Cell Research. 2006; 16:329-336).

Accordingly, there exists a need for improving methods for identifying, isolating and growing pluripotent ES cells found in the amniotic fluid. Therefore, it is desirable to develop new culture media and new methods for identifying, isolating and propagating pluripotent embryonic stem cells from amniotic fluid cells.

### SUMMARY OF THE INVENTION

The present invention is directed to pluripotent embryonic stem cells derived from amniotic fluid and the methods for isolating, expanding and differentiating these cells, and their therapeutic uses such as manipulating the fetal stem cells by gene transfection and other means for therapeutic applications. The embryonic stem cells are pluripotent and express DAZL. These cells are also characterized as germline-like stem cells (GLSC) due to the fact that they express DAZL.

The present invention is therefore directed to DAZL expressing amniotic fluid stem cells that are pluripotent and characterized as germline-like; methods for isolating, expanding and differentiating these cells from amniotic fluid; and culture medium that is useful for enriching for DAZL expressing amniotic fluid embryonic stem cells.

In aspects of the invention is an amniotic fluid cell composition comprising:
pluripotent embryonic stem cells expressing DAZL; and
amniotic fluid.

In further aspects of the invention are isolated pluripotent embryonic stem cells isolated from amniotic fluid that express DAZL.

In aspects of the invention, there is provided a novel method for the isolation, identification, culture, and characterization of pluripotent embryonic stem cells from amniotic fluid, these stem cells being characterized asembryonic germ cells (EG) (germline-like).

According to another aspect of the invention is a method for isolation of pluripotent embryonic stems cells expressing DAZL from amniotic fluid, the method comprising culturing amniotic fluid cells in a medium comprising amniotic fluid and at least one growth agent for a sufficient time for at least a portion of said amniotic fluid cells to adhere to a substrate, and further culturing non-adherent amniotic fluid cells, identifying the amniotic fluid cells expressing at least DAZL and isolating said amniotic fluid cells expressing at least DAZL.

According to an aspect of the invention is a method for enriching DAZL positive stem cells from amniotic fluid.

According to another aspect of the invention is a method for generating a population of cells enriched for pluripotent amniotic fluid stem cells comprising isolating DAZL positive cells from amniotic fluid and proliferating the DAZL positive cells in a culture medium.

According to another aspect of the invention, the GLSC expresses at least one cell surface antigen, said at least one cell surface antigen being DAZL.

According to another aspect of the invention, the GLSC expresses C-kit and SSEA-4.

According to another aspect, the GLSC express cell surface antigens that bind with antibodies having the binding specificity of monoclonal antibodies Oct-4 and TRA-1-81.

According to an aspect of the invention is a composition and method to provide a germline like stem cell line characterized by expression of one or more of the following markers: DAZL(+); alkaline phosphatase(+); SSEA-1(−); SSEA-3(+); SSEA-4(+); TRA-1-60(+); and TRA-1-81(+).

According to another aspect of the invention is a composition and method to provide a GLSC cell line having the characteristics of human embryonic germ cells.

According to another aspect of the invention is a composition and method to provide an embryonic germ-like stem cell line capable of proliferation in an undifferentiated state after continuous culture for at least 5-10 generations.

According to another aspect of the present invention is a method to provide an amniotic fluid embryonic stem cell line expressing DAZL, wherein the stem cells differentiate into cells derived from mesoderm, endoderm, and ectoderm germ layers when the stem cells are injected into an immunocompromised mouse.

According to another aspect of the present invention, is a cell culture media that provides for long term cell culture of GLSCs expressing DAZL.

According to another aspect of the present invention, the GLSCs of the present invention may be used for gene therapy and tissue engineering.

According to another aspect of the present invention is the use of a substantially enriched population of pluripotent DAZL positive germline-like stem cells harvested from amniotic fluid to treat a disease in a human.

According to another aspect of the present invention is the use of a substantially enriched population of pluripotent DAZL positive germline-like stem cells harvested from amniotic fluid in the preparation of a medicament to treat a disease in a human.

A pluripotent amniotic fluid cell composition comprising amniotic fluid cells and a culture medium comprising amniotic fluid and at least one growth agent.

According to another aspect of the present invention is a method for culturing germline-like stem cells from amniotic fluid, said method comprising:

(a) culturing amniotic fluid cells on a substrate for a sufficient time to permit a portion of said amniotic fluid cells to adhere to said substrate;

(b) isolating a non-adherent portion of said amniotic fluid cells;

(b) identifying from said non-adherent portion of amniotic fluid cells cells expressing at least one germline-like stem marker, said at least one germline-like stem marker being DAZL.

According to another aspect of the present invention is a method of proliferating a population of cells enriched for pluripotent germline-like stem cells comprising:

(a) growing in a first vessel, said population of cells in a culture medium;

(b) selecting and separating at least one DAZL positive cell from said population of cells;

(c) introducing the separated said at least one DAZL positive cell to a second vessel in said culture medium; and

(d) proliferating said at least one DAZL positive cell in said second vessel.

According to another aspect of the present invention is a method of differentiating DAZL positive germline-like stem cell comprising providing an amniotic fluid sample and inducing differentiation of DAZL positive cells within said sample by exposing said sample to one or more differentiation-inducing agents.

According to another aspect of the present invention is a method of differentiating DAZL positive pluripotent fetal stem cells comprising:

(a) providing an amniotic fluid sample;

(b) obtaining cells from said sample; and

(c) inducing differentiation of DAZL positive cells from step (b) within said sample by exposing said cells to one or more differentiation-inducing agents.

According to another aspect of the present invention is a method for storing pluripotent fetal stem cells comprising the steps of:

(a) obtaining an amniotic fluid sample from a human subject;

(b) isolating a substantially enriched population the DAZL positive germline-like stem cell from the sample; and

(c) cryopreserving the isolated substantially enriched population of DAZL positive pluripotent fetal stem cells.

According to another aspect of the present invention is a method of treating a disease in a human comprising administering a substantially enriched population of pluripotent DAZL positive germline-like stem cells into an individual in need thereof.

According to another aspect of the present invention is a use of a substantially enriched population of pluripotent DAZL positive germline-like stem cells harvested from amniotic fluid to treat a disease in a human.

According to another aspect of the present invention is a use of a substantially enriched population of pluripotent DAZL positive germline-like stem cells harvested from amniotic fluid in the preparation of a medicament to treat a disease in a human.

According to another aspect of the present invention is a composition comprising culture medium for the growth of germline-like stem cells from amniotic fluid, said culture medium comprising about 20% aminotic fluid and about 80% basal medium, said basal medium comprising: about 80% Dulbeco's modified Eagle's medium; about 20% serum replacement; and wherein said culture medium is supplemented with 1 mM L-glutamine, 1% nonessential amino acids, antibiotics and a growth agent.

Other features and advantages of the present invention will be come apparent from the following detailed description. It should be understood, however, that the detailed description and the specific examples while indicating embodiments of the invention are given by way of illustration only, since changes and various modifications within the spirit and scope of the invention will become apparent to those skilled in the art.

### BRIEF DESCRIPTION OF THE DRAWINGS

The present invention will become more fully understood from the detailed description given herein and from the accompanying drawings, which are given by way of illustration only and are not intended to limit the scope of the invention wherein:

FIGS. 1A-I show photomicrographs of human amniotic fluid cells (AFC) culturing in Stefanidis medium. After 2 days of culture (A); after 3 days of culture (B); after 4 days of culture (C); after 6 days of culture (D); after 8 days of culture (E); after 9 days of culture (F); after 12 days of culture (G); after 13 days of culture with the attached cells under investigation (H); after 13 days of culture with the floating cells under investigation (I).

FIGS. 2A-C show photomicrographs of human embryonic germ cell colonies. After 15 days of culture (A); after 20 days of culture (B); after 28 days of culture (C).

FIG. 3 shows photomicrographs of human amniotic fluid cells (AFC) cultured in Stefanidis medium that were differentiated into human fibroblasts.

FIGS. 4A-B show electron photomicrographs of human germ-like stem cell (A) and a human embryoid body formed from a germline-like stem cell (B).

FIG. 5 shows the flow cytometric detection of 7AAD, DAZL and c-kit. Amniotic fluid cells were identified by two scatter regions, R1 and R2, on a forward scatter (FSC) vs. side scatter (SSC) dot plot (A) and were analysed separately for marker expression, here 7AAD⁺ cells and the negative unstained control is shown in overlay histograms (C and D). Because of high degree of autofluorescence in R2 gated population (D), only R1 gated cells were used for further analysis. Cells negative for 7AAD (R3) were then selected for the assessment of DAZL and c-kit positive cells (B); their expression frequencies were assessed as percentages of the viable amniotic fluid cells by subtracting their appropriate isotype controls (E and F).

FIG. 6 shows photomicrographs of human embryonic germ cell (EG) colonies showing positive immunohistochemical staining for: Human embryonic germ cell colonies (A1); A1 colony showing immunoreactivity to Oct-3/4 (A2); A1 colony shows immunoreactivity for stage specific embryonic antigen-4 (SSEA-4) (A3); Human embryonic germ cell colonies (B1); B1 colonies shows immunoreactivity to a cell surface antigen that binds with the antibody having the binding specificity of the monoclonal antibody designated TRA-1-81 (B2); Human embryonic germ cell colonies (C1); C1 colonies show immunoreactivity to cell surface antigen DAZL (C2).

FIGS. 7A-B shows the expression of DAZL (A) and Oct-4 (B) mRNA expression by RT-PCR.

FIG. 8 shows that GLSCs may be differentiated to neurogenic cells and express the s-100 neurogenic cell marker.

FIG. 9 shows a Cy5/Cy3 false colour image of the microarray analysis of microarray # 4800038 comparing amniotic fluid-derived cells cultured according to the present invention and human embryonic germ cells.

FIG. 10 shows the microarray data visualized in a doublelog scatter plot.

### DETAILED DESCRIPTION OF THE EMBODIMENTS

The present invention is based on using amniotic fluid as a source to obtain a population of stem cells which have a pluripotent differentiation capacity and therefore are a viable source of stem cells that can be used therapeutically. These cells express DAZL. The present invention discloses compositions of these cells for therapeutic use; methods of isolating, enriching isolating and maintaining the cells in culture; and therapeutic uses of the cells. An advantage of the invention is that the cells can be efficiently isolated and propagated from a source that is less controversial such that the method overcomes the ethical considerations associated with traditional known methods used to harvest embryonic stem cells. The ability of maintaining the cells of the invention as cell lines permits clinical investigation of these cells and the dynamics of interaction in their cellular and chemical environment.

Various terms are used herein in this application which are generally defined as follows and are well known by one of skill in the art:

“Amniotic fluid or amniotic fluid samples” means samples of fluid obtained from within the amnion. The amniotic fluid may or may not be filtered from cellular material, such as cells.

“Amniotic Fluid-Derived Cells or Amniotic Fluid Cells (AFC)” are cells that are contained in amniotic fluid samples obtained during amniocentesis at, for example, about 17-22 weeks of gestation.

“Amniocentesis” means puncture of the amnion, the thin-walled sac of fluid in which a developing fetus is suspended during pregnancy.

“Anlagen” is the rudiment or the primordia of an organ, tissue or part thereof.

“Antibody” as used in this invention includes intact molecules as well as fragments thereof, such as Fab, Fab′, F(ab′)2, and Fv that can bind the epitopic determinant as disclosed by Ladner et al., in U.S. Pat. No. 4,946,788. If required, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See, e.g., Coligan, et al., Current Protocols in Immunology, Wiley Interscience, current edition). “Purified antibody” means an antibody that is at least 60%, by weight, free from proteins and naturally-occurring organic molecules with which it is naturally associated. In aspects of the invention, the preparation is at least 75%, more preferably 90%, and most preferably at least 99%, by weight, antibody, e.g., an anti-SSEA-1 specific antibody. The purified antibody may be obtained, for example, by affinity chromatography using recombinantly-produced protein or conserved motif peptides and standard techniques.

“Blastocyst” is a preimplantation embryo that develops froms a morula. The blastocyst has an out layer called the trophoblast that is required for implantation into the uterine epithelium and an inner cell mass that contains the embryonic stem cells and will give rise to the embryo proper. The blastocyst contains a blastocoel or a blastocoelic cavity.

“Cell” as used herein also refers to individual cells, cell lines, or cultures derived from such cells. The term “cell line” as used herein refers to human AFC or cells derived therefrom and maintained in in vitro culture.

“Cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

“Conditioned medium” refers to a growth medium that is further supplemented by factors derived from media obtained from cultures of feeder cells on which human AFC can be cultured.

“DAZL” (deletion in azoospermia like) is a marker expressed in embryonic germ cells. The gene encodes RNA binding proteins. DAZL gene expression is unique as it is expressed before meiosis in male and female gonads. This pattern of expression suggests that these genes participate in the early proliferation, differentiation and maintenance of male and female embryonic germ cells.

“Embryonic germ cells” or “EG cells” are cells derived from primordial germ cells (PGCs). The term “embryonic germ cell” is used to describe cells of the present invention that exhibit an embryonic pluripotent cell phenotype. The terms “human embryonic germ cell (EG)” or “embryonic germ cell” can be used interchangeably herein to describe human cells, or cell lines thereof, of the present invention that exhibit a pluripotent embryonic stem cell phenotype as defined herein. Thus, EG cells are cells capable of differentiation into cells of ectodermal, endodermal, and mesodermal germ layers. EG cells can also be characterized by the presence or absence of markers associated with specific epitope sites identified by the binding of particular antibodies and the absence of certain markers as identified by the lack of binding of certain antibodies.

“Embryoid body” (EB) is a three dimensional structure that forms from differentiated embryonic stem cells. Cellular derivatives of all three germ layers have been generated from embryoid bodies, such as hematopoietic, endothelial, muscle and neuronal cells.

“Epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

“Feeder cells” as used herein can refer to cells that are maintained in culture and are co-cultured with target cells. Target cells can be embryonic germline-like stem cells and cultured cells for example. Feeder cells (e.g. fibroblasts) can provide, for example, peptides, polypeptides, electrical signals, organic molecules (e.g., steroids), nucleic acid molecules, growth factors (e.g., bFGF), other factors (e.g., cytokines such as LIF and steel factor), and metabolic nutrients to target cells. Feeder cells, in aspects of the invention, grow in a mono-layer.

“Germline-like stem cell or embryonic germline-like stem cell (GLSC) are pluripotent or multipotent stem cells. These cells possess characteristics of pluripotent embryonic stem (ES) cells and embryonic germ cells (EG).

“Long term” refers to a cell culture of more than 30 days.

“Multipotent” refers to cells that are capable, through its progeny, of giving rise to several different cell types.

“Non-essential Amino acids” refers to the amino acids L-alanine, L-asparagine, L-aspartic acid, L-glutamic acid, L-glycine, L-proline, and L-serine.

“Primordial germ cells” (PGCs) is used to describe undifferentiated embryonic germ cells isolated over a period of time post-fertilization from anlagen or from yolk sac, mesenteries, or gonadal ridges of human embryos/fetus. PGCs are the source from which EG cells are derived. Gonocytes of later testicular stages also can be useful sources of PGCs.

“Pluripotent” refers to cells that retain the developmental potential to differentiate into a wide range of cell lineages including the germline. The terms “embryonic stem cell phenotype” and “embryonic stem-like cell” also are used interchangeably herein to describe cells that are undifferentiated and thus are pluripotent cells and that are capable of being visually distinguished from other adult cells of the same animal.

“Plated” or “plating” as used herein in reference to cells can refer to establishing cell cultures in vitro. For example, cells can be diluted in cell culture media and then added to a cell culture plate, dish, or flask. Cells may be plated at a variety of concentrations and/or cell densities.

“Proliferation” as used herein in reference to cells can refer to a group of cells that can increase in number over a period of time.

“Recombinant product” as used herein can refer to the product produced from a DNA sequence that comprises at least a portion of the modified nuclear DNA. This product can be a peptide, a polypeptide, a protein, an enzyme, an antibody, an antibody fragment, a polypeptide that binds to a regulatory element (a term described hereafter), a structural protein, an RNA molecule, and/or a ribozyme, for example.

“Stefanidis medium” means a novel stem cell culture medium capable of supporting growth of human AFCs and GLSCs expressing DAZL as well as other markers. According to an aspect of the invention, Stefanidis medium is prepared using about 20% amniotic fluid with 80% basal medium, itself comprising 80% Dulbeco's modified Eagle's medium (DMEM) (Gibco BRL, Rockville, Md.) supplemented with 20% KnockOut SR, a serum-free replacement originally optimized for human ES cells (Gibco BRL, Rockville, Md.)], 1 mM L-Glutamine, 1% nonessential amino acids stock (Gibco BRL, Rockville, Md.), penicillin and streptomycin and 4 ng/ml bFGF.

### DAZL-Expressing Pluripotent Stem Cells, Compositions Thereof and Stem Cell Culture Medium

The invention provides pluripotent embryonic stem cells isolated from amniotic fluid wherein the cells express one or more markers for pluripotent embryonic germ cells including DAZL. In another aspect of the invention, the cells also express SSEA-4, TRA-1-60 and Oct-4 markers as demonstrated by a variety of methods known to those of skill in the art such as but not limited to reverse-transcription (RT-PCR), immunofluorescence (IF), or methods of analysis of differential gene expression, microarray analysis and related techniques. According to aspects of the invention, the cells maintain a normal karyotype during prolonged cultivation in vitro.

The pluripotent embryonic stem cells of the invention in addition to at least expressing DAZL (and other markers) may also express the c-kit receptor. The c-kit receptor protein, also known as c-Kit receptor, Steel factor receptor, stem cell factor receptor and CD 117 in standardized terminology of leukocyte antigens, is constitutively expressed in hematopoietic stem cells and germ cells. The c-kit receptor plays a fundamental role during the establishment, maintenance and function of germ cells. In the embryonal gonad, the c-kit receptor and its ligand SCF are required for the survival and proliferation of primordial germ cells.

In accordance with the present invention, the embryonic stem cells are obtained from human amniotic fluid. Large quantities of amniotic fluid cells can be obtained from subjects during pregnancy and/or at birth depending on which cell source is used. The stem cells obtained from these sources may be cultured in various media, such as DMEM, F-12, M199, RPMI and combinations thereof, supplemented with fetal bovine serum (FBS), whole human serum (WHS), or supplemented with growth factors, cytokines, hormones, vitamins, antibiotics, or any combination thereof. A novel Stefanidis medium as herein described is preferred either alone or in combination with any of the elements recited supra.

The embryonic stem cells of the invention may also be expanded in the presence of an agent which suppresses cellular differentiation. Such agents are well-known in the art (Dushnik-Levinson, M. et al., “Embryogenesis in vitro: Study of Differentiation of Embryonic Stem Cells,” Biol. Neonate, Vol. 67, 77-83, 1995, the disclosure of which is incorporated herein by reference). Examples of agents which suppress cellular differentiation include leukemia inhibitory factor (LIF) and stem cell factor. On the other hand, agents such as hydrocortisone, Ca²⁺, keratinocyte growth factor (KGF), TGF-β, retinoic acid, insulin, prolactin, sodium butyrate, TPA, DIVISO, NMF, DMF, collagen, laminin, heparan SO₄, androgen, estrogen, and combinations thereof may be used to induce differentiation (Culture of Epithelial Cells, (R. Ian Freshney ed., Wiley-Liss 1992)).

Furthermore, the cells of the invention may also be cultured in the presence of one or more of the following at the stated final concentration: forskolin ([3R-(3α,4αβ, 5B, 6B, 6aα, 10α, 10αβ, 10bα)]-5-(acetyloxy)-3-ethenyldodecahydro-6,10,10b-trihydroxy-3,4a,7,7,10a-pentamethyl-1H-naphtho[2,1-b]pyran-1-one) at 10 μM, cholera toxin at 10 μM, isobutylmethylxanthine (IBMX) at 0.1 mM, dibutyrladenosine cyclic monophosphate (dbcAMP) at 1 mM. Other suitable agents for use in the invention are described in International Patent Application, WO 2005/017117, and herein incorporated by reference in its entirety.

The cells may be assessed for viability, proliferation potential, and longevity using standard techniques in the art. For example, a trypanblue exclusion assay, a fluorescein diacetate uptake assay, a propidium iodide uptake assay, or other techniques known in the art may be used to assess viability. A thymidine uptake assay, an MTT cell proliferation assay, or other techniques known in the art may be used to assess proliferation. Longevity may be determined by the maximum number of population doublings in extended cultures or other techniques known in the art. Additionally, cells of different lineages may be derived by inducing differentiation of fetal stem cells and as evidenced by changes in cellular antigens. Various differentiation-inducing agents are used to accomplish such differentiation, such as growth factors (for example EGF, AFGF, bFGF, PIDGF, TGFβ), hormones (including but not limited to insulin, triiodothyronine, hydrocortisone, and dexamethasone), cytokines (for example IL-1α or P, IFN-γ, TFN), matrix elements (for example collagen, laminin, heparan sulfate, Matrigel), retinoic acid, transferrin, TPA, and DMSO. Such differentiation-inducing agents are known to those of ordinary skill in the art (Culture of Epithelial Cells, (R. Ian Freshney ed., Wiley-Liss 1992)). Identification of differentiated cells may be accomplished by staining the cells with tissue-specific antibodies according to techniques known in the art.

The present invention is also directed to compositions comprising the embryonic stem cells expressing DAZL. In aspects, the composition comprises embryonic stem cells expressing DAZL, amniotic fluid cells and a novel stem cell culture medium. The stem cell culture medium (hereinafter referred to as Stefanidis™ medium) of the present invention comprises amniotic fluid and one or more growth factors, cytokines, hormones, vitamins, antibiotics, cellular agents, chemicals or any combination thereof.

The present invention is also directed to a novel cell culture medium that is particularly advantageous for the isolation and propagation of the cells of the invention. The medium, Stefanidis medium, comprises amniotic fluid; at least one or more growth factors, cytokines, hormones, vitamins, antibiotics, cellular agents, chemicals or any combination thereof; basal growth medium; and optionally a serum replacement medium. The source of the amniotic fluid may be from any type of animal such as, but not limited to mammals. In another aspect of the invention, the source may be from a primate. In one aspect, the source of the amniotic fluid is human. The amniotic fluid may be obtained at any time of the gestational period or at birth as desired. In aspects, there is provided about 5% to about 50% amniotic fluid in the medium.

The basal growth medium can be selected from any suitable commercially available medium such as but not limited to Dulbecco's Modified Eagle Medium (“DMEM”), Basal Media Eagle (BME), DMEM/F-12 (1:1 DMEM and F-12 vol:vol); Medium 199; F-12 (Ham) Nutrient Mixture; F-10 (Ham) Nutrient Mixture; Minimal Essential Media (MEM), Williams' Media E; and RPMI 1640, all of which are available from Gibco BRL/Life Technologies, Inc., (Gaithersburg, Md.).

In the methods of the present invention, the isolation and propagation of pluripotent embryonic stem cells expressing DAZL may be done without adding serum to the culture medium. Therefore, according to a further aspect of the invention, the basal growth medium may comprise about 50% to about 90% serum replacement medium. In aspects of the invention, Knockout Serum™ replacement (Gibco) is used.

In aspects the Stefanidis medium comprises about 80% DMEM and about 20% serum replacement medium of the basal growth medium. The use of other basal growth media suitable that would be suitable for growth of the amniotic fluid cells of the present invention will be readily apparent to those skilled in the art. A variety of agents such as, but not limited to IGF-1, IGFBP-2, inhibin B, T4, taurine, cortisol, MCP-1 may also be included in the Stefanidis medium. The Stefanidis medium may also contain at least one of; non-essential and essential amino acids; a pyruvate salt; a reducing agent and combinations thereof. In aspects the amino acid is L-glutamine.

It will be apparent to those in the art that certain changes in the specific chemical components employed in the preparation of Stefanidis medium can be tolerated without affecting the function or altering the effectiveness of the medium. Also, it will be appreciated that numerous non-nutrient materials, e.g., antibiotics, can be added to a growth medium without affecting the basic functionality of the medium. It will also be understood that certain components of the medium or the serum-free supplements can be substituted by equivalent substances or by preparations from different sources or with minor deviations of purity without affecting the functionality of the medium. Any such substitutions and additions are contemplated to be encompassed herein. Also, it will be understood that the medium of the instant invention can be prepared in a number of different ways known to those of ordinary skill in the art. For example, it can be prepared as one or more concentrated stock mixtures or solutions and then combined and diluted out as desired. Further, it is contemplated that the medium can be subjected to different physical treatments, for example, autoclaving, filtration, lyophilization, etc., and may be used as such with complete equivalence.

### Amniotic Fluid Cells and Germline-Like Stem Cells and Methods of Cell Culture

In one embodiment, the invention provides for a method of growing amniotic fluid cells (AFC) and isolating embryonic stem cells expressing DAZL from the amniotic fluid. As disclosed, the pluripotent embryonic stem cells of the invention feature many of the characteristics of pluripotent embryonic germ cells. The present invention provides an alternative source of human ES cells, thus eliminating the requirement to produce or disaggregate a normal, competent embryo.

Samples of amniotic fluid (5-15 ml) were obtained after ultrasonography-guided amniocentesis performed on pregnant women with a gestational age ranging from 17 to 22 weeks. The samples were centrifuged at 1800 rpm for 5 minutes twice, and the pellets removed and resuspended in 10 ml of Stefanidis medium as described in the examples section, in a 75 cm² flask and incubated at 37° C. with 5% humidified CO₂. After about 96 hours to about 128 hours, the non-adhering portion of amniotic fluid cells in the supernatant was collected. The non-adherent portion of amniotic cells were centrifuged and plated in a) 5 ml of Stefanidis medium (flask-A) or b) 5 ml of DMEM-high glucose supplemented with 20% fetal bovine serum and glutamine and basic fibroblast growth factor (4 ng/ml) (flask-B) in 25 cm² flask and incubated at 37° C. with 5% humidified CO₂.

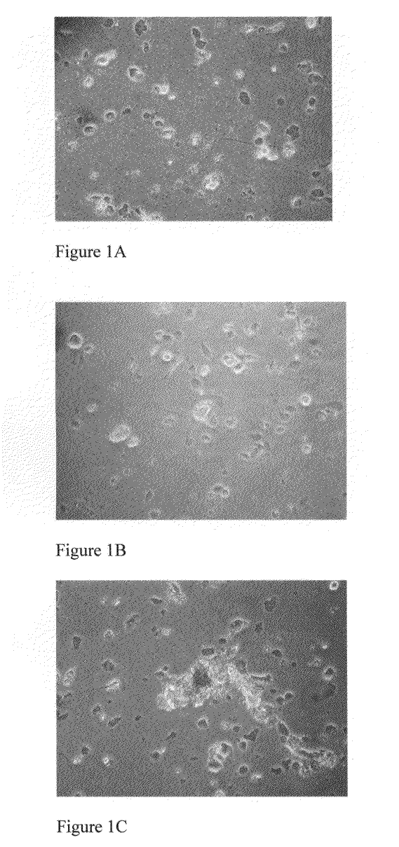

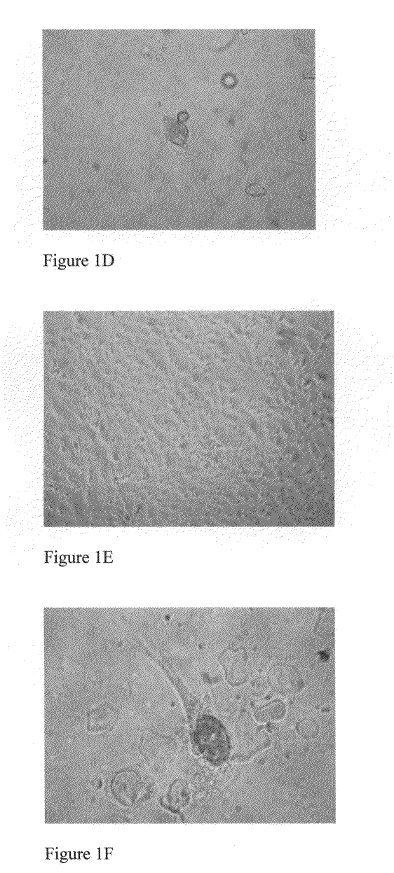

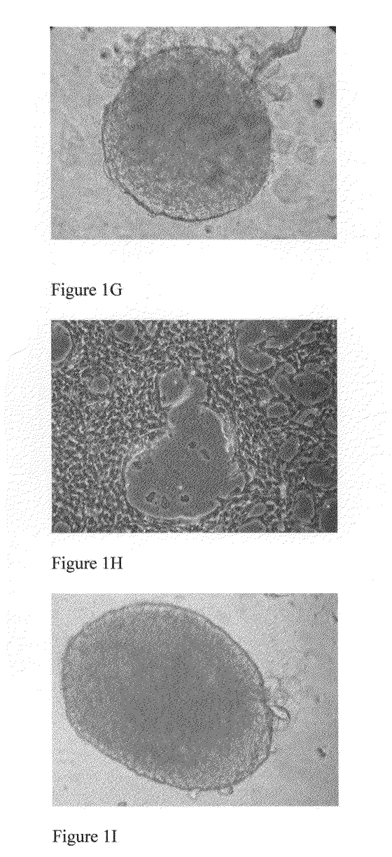

As can be seen in FIGS. 1A-I, the colonies of GLSC began to appear 10-20 days after plating the non-adhering amniotic fluid cells in the culture flask-A containing Stefanidis medium. Human fibroblasts began to appear in the culture flask-B.

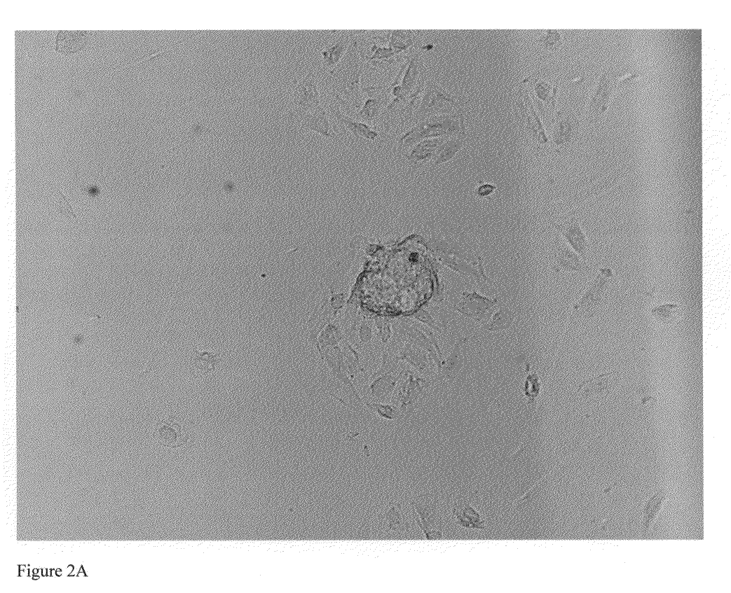

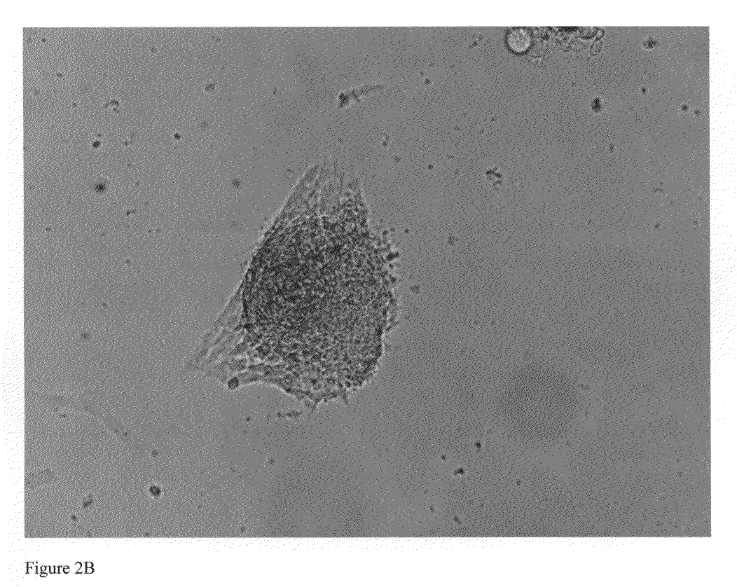

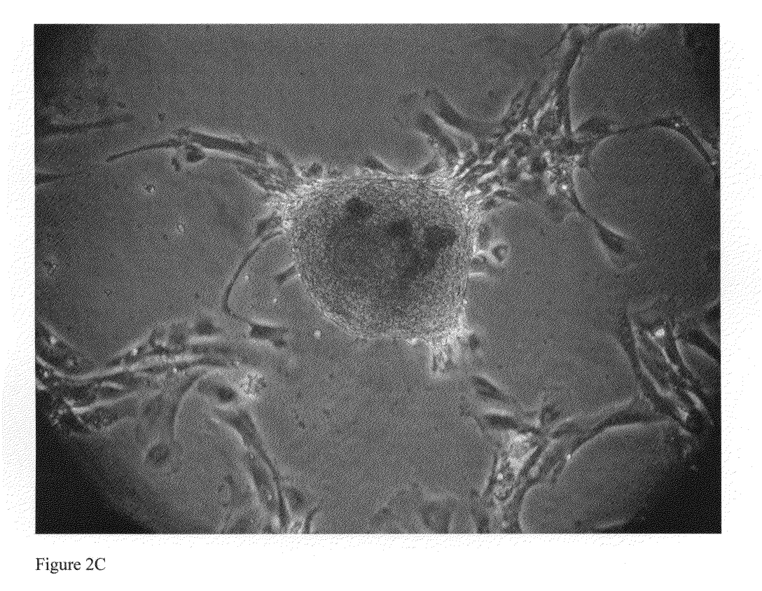

Morphologically, the GLSCs formed 4-6 well defined colonies and resembled ES cells, with a small cytoplasm-to-nuclear ration and multiple nucleoli and cytoplasmic lipid bodies (FIGS. 2A-C).

Alpha-fetoprotein and the beta-subunit of human chorionic gonadotrophin were readily detected by immunoassay in the supernatants of the GLSC cultures grown to high density. Alpha-fetoprotein is a characteristic product of endoderm cells and human chorionic gonadotrophin secretion is characteristic of trophoblastic differentiation.

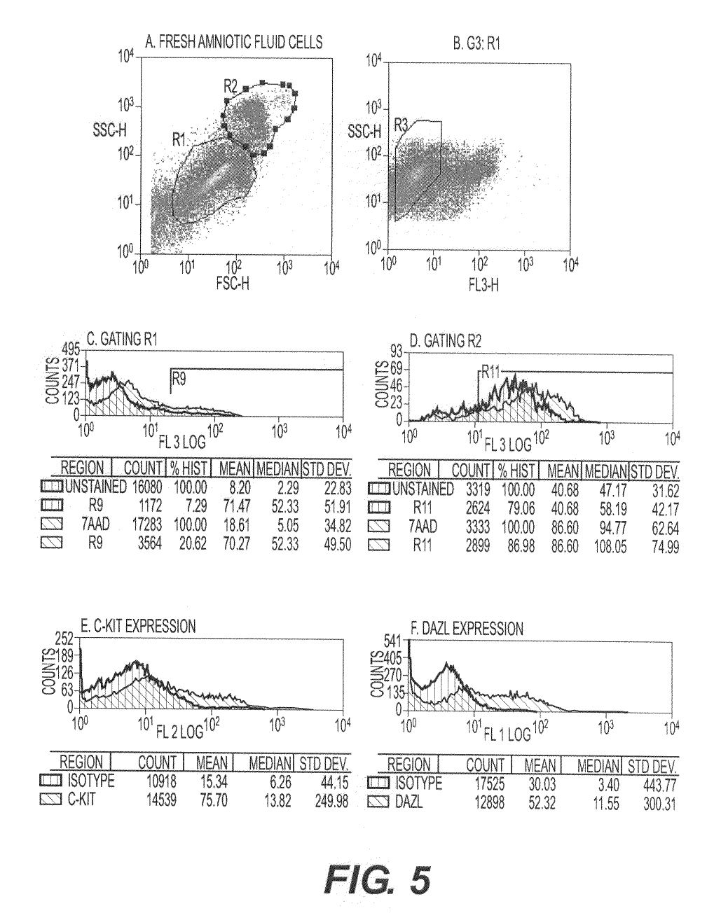

By flow cytometry analysis it was found that about of 15-30% of fresh amniotic fluid cells express DAZL (FIG. 5). It was also found that these GLSC have medium-large volume and express Oct-4. Furthermore, in the study it was observed with flow cytometry analysis that a subpopulation within amniotic fluid cell samples can be found to be Oct-4 and SSEA-4 positive. The fact that only ˜0.2% of the cells expresses the two molecular markers of Oct-4 and SSEA-4 suggests that only a distinct subpopulation of amniotic fluid cells is embryonic-like stem cells at 17-22 weeks of gestation.

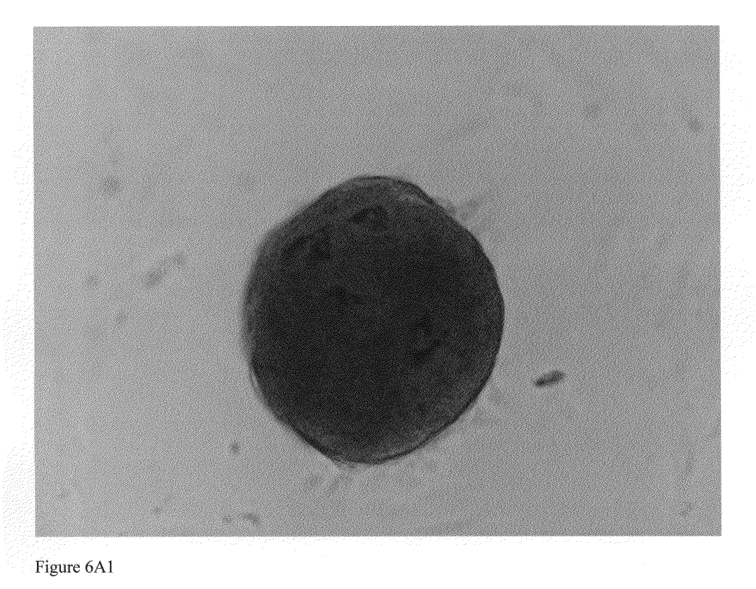

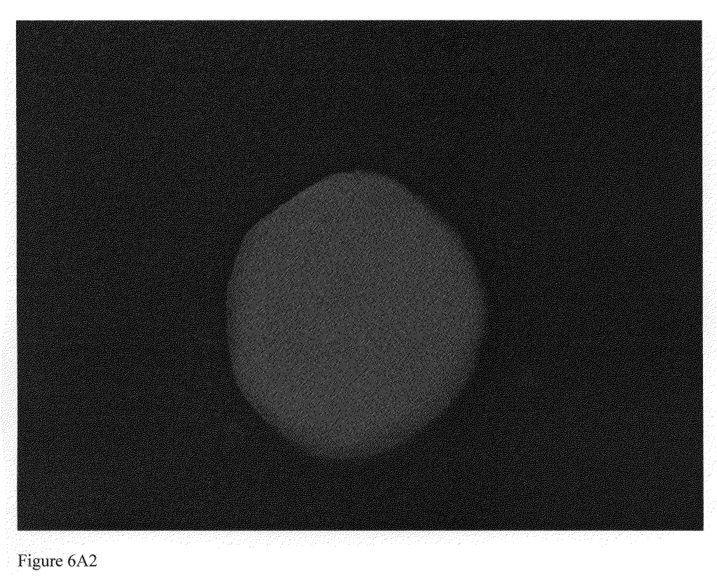

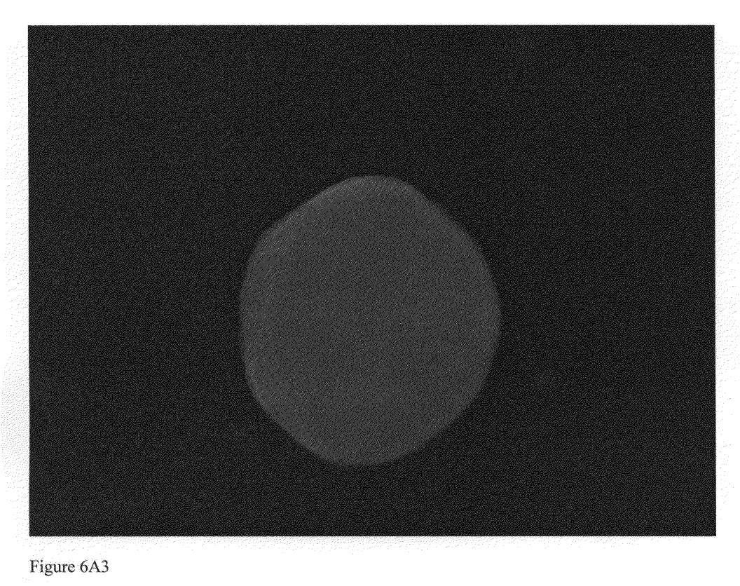

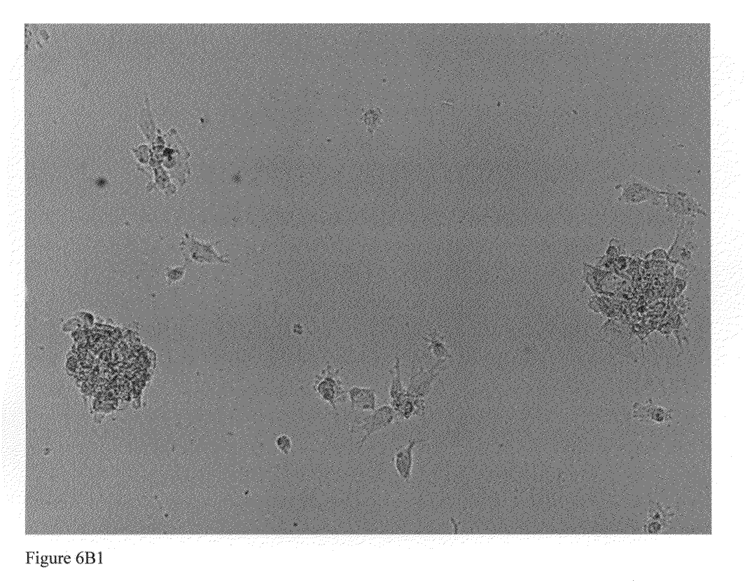

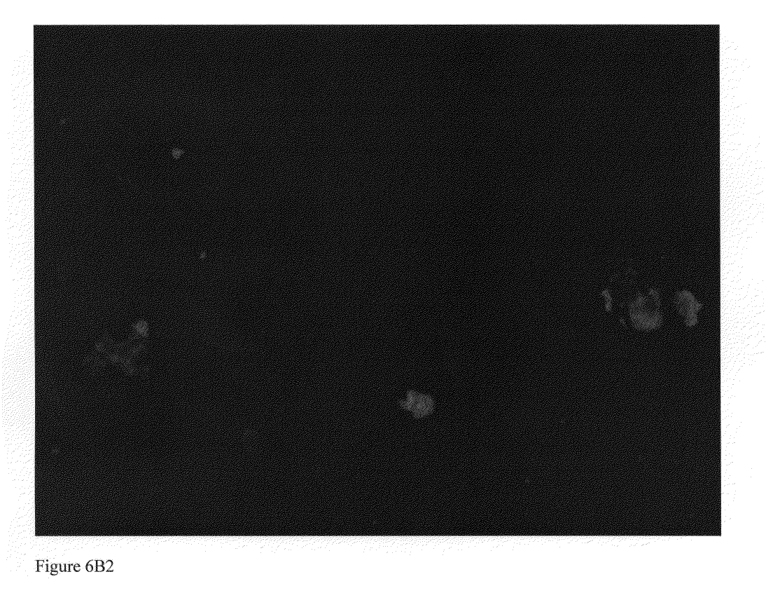

The GLSCs have strong expression of molecular markers of DAZL, and Oct-4 and SSEA-4, and TRA-1-60, and also express Oct-4 mRNA. In contrast human fibroblast cells did not express molecular markers of Oct-4 and SSEA-4 and also did not express Oct-4 and mRNA (FIGS. 6A1-C2).

The resulting GLSCs can be maintained in an undifferentiated state for at least two months in culture and may be cultured for at least about 5-10 generations.

The amniotic fluid-derived cells can be pluripotent stem cells or multipotent stem cells. For example, the amniotic fluid-derived cells can be multipotent stem cells characterized by a) the ability to grow in continuous culture and b) the presence of at least one, or two, or three, or four, or five, or all of the markers selected from the group consisting of: SSEA-3, SSEA-4, Tra1-60, Tra1-81, Tra2-54, and Oct-4. These stem cells can further express at least one marker selected from the group consisting of: HLA Class I, CD13, CD44, CD49b, and CD105. The amniotic fluid-derived cells can be pluripotent stem cells characterized by a) the ability to grow in continuous culture, and b) the presence of at least one, or two, or three, or four, or five, or all of the markers selected from the group consisting of: SSEA3, SSEA4, Tra1-60, Tra1-81, Tra2-54, and Oct-4. The stem cells can further express at least one marker selected from the group consisting of: HLA Class I, CD13, CD44, CD49b, and CD105 as disclosed in U.S. Pat. No. 5,677,136, herein incorporated by reference in its entirety.

Importantly it was also shown that at least one germ cell specific gene DAZL, was expressed by human ES cells but not by human ICM. The existing gene expression data are consistent with the idea that the closest in vivo equivalent to ES cells clearly is not the ICM or primitive ectoderm but an early germ cell. The present results are in agreement with a review article from James Thomson titled “a germ cell origin of embryonic stem cells” (Development 2005; 132:227-233). Human ES cells in a population express the early germ cell markers related (STELLA) and deleted in azoospermia like (DAZL), indicating that a minor subset of randomly differentiating cells in a minor subset of randomly differentiating cells in a mixed population is mot responsible for the expression of germ cell markers in ES cell cultures.

The DAZL gene, known also as DAZL1, DAZLA or DAZH, is an autosomal homolog of the DAZ (Deletion in Azoospermia) gene present on the Y chromosome (Saxena, R. et al. Nature Genet. 14, 292-299, 1996). These genes encode RNA binding proteins, found to be expressed specifically in germ cells in the testis. Later studies have demonstrated that the DAZL gene expression is unique as it is expressed before meiosis in male and female gonads (Seligman and Page, Biochem. Biophys. Res. Corn. 245, 878-82, 1998). This pattern of expression suggests that these genes participate in the early proliferation, differentiation and maintenance of male and female germ cells. Expression studies of a DAZL homolog in the mouse, denoted Dazl, suggest that this gene is expressed as early as when primordial germ cells appear in the developing embryonic gonads. The similarity between the DazI and DAZL expression in male and female gonads suggests that DAZL gene is expressed in early human gonad development as well, presumably in primordial germ cells.

Numerous genes are known to be expressed exclusively in male or female germ cells, mainly in meiotic or postmeiotic cells, but not in the earliest stages of gametogenesis. The expression of the human DAZL gene in both male and female germ cells so early during embryonic development is unusual. In the mouse, only a very few genes are known to be expressed exclusively in male and female germ cells early during gametogenesis, but no human homologous genes were studied. The mouse germ cell nuclear antigen (GCNA1) is expressed in primordial germ cells, and later in oogonia and prospermatogonia, as is the DAZL gene, but no DNA sequences of GCNA1 are available (Endres and May, Dev. Biol. 163, 331-340, 1994). The TIAR gene, which is also an RNA-binding protein such as DazI, was found to be expressed in primordial germ cells (Beck, A. R. P. et al. Proc. Natl. Acad. Sci. USA 95, 2331-2336, 1998).

According to another aspect of the invention, the cells of the present invention do not require feeder layers to grow and also do not require the presence of serum. Furthermore, by modifying culture conditions, the cells of the invention or fibroblasts could be generated in vitro, from AFCs. Throughout the process and at its end, the human ES cells retain normal karyotypes. While not wishing to be bound to any particular theory, it may be hypothesized that the pluripotent embryonic stem cells of the invention expressing DAZL most closely represent early germ cells.

The conclusions that could be drawn from these findings are that amniotic fluid samples contain pluripotent stem cells such as embryonic-like stem cells and differentiated cells. In an aspect of the invention the source of amniotic fluid may be mammalian. In another aspect of the invention, the source may be from a primate. In yet another aspect, the source is human.

In another aspect, the invention provides a method for screening agents that induce the pluripotent embryonic stem cells expressing DAZL to differentiate. In one aspect of the method, components including the compound and at least one cell of the invention are incubated under conditions sufficient to allow the components to interact. The effect of the compound on the cells is determined before and after incubating in the presence of the compound. The appearance in culture of a restricted developmental lineage cell indicates differentiation of the cells by the compound.

Another aspect of the present invention provides methods for selection of pluripotent or multipotent amniotic fluid stem cells using the DAZL mRNA as a marker. Labeled oligonucleotide or polynucleotide probes or antibodies, or other agents which selectivity bind said mRNA may be used for labelling DAZL positive cells and separating them using methods known in the art.

The DAZL specific antibodies, in aspects of the invention are monoclonal antibodies and can be used to separate germ stem cells by separation methods known in the art.

In another aspect, a selectable marker such as DAZL is expressed in a restricted developmental lineage cell. The restricted developmental lineage cell contains a recombinant polynucleotide that encodes the selectable marker such that the marker is expressed from a restricted developmental lineage cell specific promoter. The DAZL positive GLSCs of the present invention may serve as tools to identify new developmental lineage specific cells and their associated promoters such as but not limited to lines of spermatogonia and oogonia.

In aspects of the invention, the pluripotent embryonic stem cells of the invention line will constitute a purified preparation of an undifferentiated stem cell line. In another aspect of the invention, the stem cell line is a permanent cell line, distinguished by the characteristics identified above. They have normal karyotype along with the characteristics identified above. This combination of defining properties will identify the cell lines of the invention regardless of the method used for their isolation. According to another aspect of the invention, the GLSC lines differentiate into cells derived from mesoderm, endoderm, and ectoderm germ layers when the cells are injected into an immunocompromised mouse. The methods used to inject into an immunocompromised mouse are well known to those in the art.

Methods of identifying the characteristics of the cells of the invention are well known to the skilled addressee. Methods such as (but not limited to) indirect immunofluorescence or immunocytochemical staining may be carried out on colonies of GLSCs which are fixed by conventional fixation protocols then stained using antibodies against stem cell specific antibodies and visualized using secondary antibodies conjugated to fluorescent dyes or enzymes which can produce insoluble colored products. Alternatively, RNA may be isolated from the stem cells and RT-PCR, Northern blot analysis or gene array may be carried out to determine expression of stem cell specific genes such as Oct-4.

In a particularly advantageous embodiment of the present invention, the cells of the invention can be propagated for an indefinite period of time in continuous culture in an undifferentiated state. The term “undifferentiated” refers to cells that have not become specialized cell types. The cells may be grown in an undifferentiated state for as long as desired and can then be cultured under certain conditions to allow progression to a differentiated state. The term “differentiation” is meant by the process whereby an unspecialized cell acquires the features of a specialized cell such as but not limited to fat cells, cardiac muscle cells, epithelial cells, liver cells, brain cells, blood cells, neurons, glial cells, pancreatic cells, and the like.

General methods relating to stem cell differentiation techniques that may be useful for differentiating the GLSCs of this invention can be found in general texts such as: Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in vitro (M. V. Wiles, Meth. Enzymol. 225: 900, 1993); Properties and uses of Embryonic Stem Cells Prospects for Application to Human Biology and Gene Therapy (P. D. Rathjen et al., Reprod. Fertil. Dev. 10: 31, 1998); and in Stem cell biology (L. M. Reid, Curr. Opinion Cell Biol. 2: 121, 1990), each of which is incorporated by reference herein in its entirety.

As stated previously, differentiation-inducing agents, maturation agents, or maturation factors may be useful to allow progression to certain cell types. Examples of differentiation inducing agents, that may be used include but are not limited to agents, such as N-butyrate, which are useful for differentiating embryonic stem cells to liver cells are described in U.S. Pat. No. 6,506,574, to Rambhatla et al. Optionally, maturation agents, or maturation factors, such as, for example, growth factors, peptide hormones, cytokines, ligand receptor complexes, corticosteroids, retinoic acid, and even organic solvents like DMSO have been found to effect differentiation of embryonic stem cells (U.S. Pat. No. 6,506,574). Other suitable differentiating or maturation agents which may be used include but are not limited to a glucocorticoid with cAMP-elevating agents, methyl-isobutylxanthine, indomethacin, and the like.

The pluripotent embryonic stem cells of the invention expressing DAZL provide an excellent model system to understand the differentiation, development and functioning of gonads. For instance, the cells may be differentiated into oocytes or spermatocytes using techniques well known by those in the art. Once oocytes are obtained, they may be enucleated. Somatic cell nuclei are obtained from an infertile female patient to be treated and somatic cell transfer is then performed. Blastocysts are then obtained from which stem cells which are genetically identical to the infertile female are isolated. Such stem cells are then treated as described herein to generate a second generation of germ cells. The germ cells are subjected to culture conditions which promote the formation of oocytes which can then be used in in vitro fertilization methods.

Gametes derived from the cells of the invention may be made relatively inexpensively and may be scientifically and socially invaluable for biomedical research. Customized gametes may offer new reproductive choices to individuals who desire to have children. Gametes derived from the differentiation of the cells may be created and cultured in large quantities using bioreactors. Thus the cells of the invention can be a valuable ethical and practical cell source for fetal tissue engineering.

In another aspect of the present invention, the invention also discloses cell culture medium and methods for growing and maintaining cultures of AFC, which includes the pluripotent embryonic stem cells of the invention. The Stefanidis medium also provides for the growth and maintenance of stem cells expressing DAZL and can be used to screen for additional growth factors and useful combinations of growth factors. The ability to grow the cells in a substantially undifferentiated state using the cell culture media, growth factors, and methods provided herein provides important benefits including the ability to produce cell lines.

According to an embodiment of the present invention, the pluripotent embryonic stem cells may be grown in the presence of feeder cells. In aspects of the invention, the feeder cells can be first grown to confluence and then mitotically inactivated (e.g., by irradiation) to prevent further growth of the feeder cells. Such an approach has the advantage of simplifying the management of the cell culture as the growth of only one set of cells, the EG cells, need only be monitored.

Once established, the cells can be cultured under the above-described conditioned medium using a variety of techniques. According to an embodiment of the invention, a container holds feeder cells in a non-conditioned medium. A matrix of lysed feeder cells is prepared using standard methods well known to those of skill in the art. The pluripotent embryonic stem cells expressing DAZL to be cultured are then added atop the matrix along with the conditioned medium. Alternatively, the pluripotent embryonic stem cells expressing DAZL can be grown on living feeder cells using methods known in the art. The growth of the pluripotent embryonic stem cells expressing DAZL is then monitored to determine the degree to which the cultured cells have become differentiated. A marker for alkaline phosphatase is used to ascertain which cells have differentiated, all of which are commonly known and practiced by those of skill in the art (Kaplan, O. L. et al Stem Cells. 2006 February; 24(2):266-73; Itskovitz-Eldor 3, et al. Mol. Med. 2000 February; 6(2):88-95). When a sufficient number of cells have differentiated, or when the culture has grown to confluence, at least a portion of the undifferentiated cells can be passaged. The determination to passage the cells and the techniques for accomplishing such passaging can be performed using standard techniques well known in the art.

While not being limited to any theory, it is believed that the pluripotent embryonic stem cells expressing DAZL are mainly found in the non-adherent portion after about 96 to about 128 hours of plating in Stefanidis medium. Interestingly, embryonic stem cells have been obtained using the adherent cell portion when grown in the presence of fibroblast feeder lines (at 128 hours). Further, these embryonic stem cells also attach to the fibroblast feeder lines and may themselves be further differentiated into fibroblasts.

According to another aspect of the invention, the methods disclosed permit the culture and the formation of fibroblasts from AFC. It has been known that fibroblastic cells cannot be cultivated from every amniocentesis sample (Hengatschläger. J Reproduktionsmed Endocrinol 2005; 4:233-8). The applicants have disclosed compositions and methods to culture and grow fibroblasts from every amniocentesis sample. The applicants successfully split fibroblasts for many generations. The newly formed fibroblasts may be cryopreserved and thawed with about a 60% survival rate. These differentiated fibroblasts have a normal karyotype and may be used as a feeder line to grow the inner cell mass from mouse blastocysts and finally human inner cell mass from a blastocyst.

According to another embodiment of the present invention, GLSC may be injected into SCID mice such as by subcutaneous injection into the legs. The injection of GLSC of the present invention will result in the formation of teratocarcinomas.

According to another embodiment of the invention, the GLSC may also be cryopreserved in a cell bank for potential future use. The methods of cryopreserving embryonic stem cells are well known by those skilled in the art as exemplified by WO 2005/017117 and may be used to cryopreserve the GLSC of the present invention.

Essentially all of the uses known or envisioned in the prior art for stem cells, can be accomplished with the amniotic fluid derived GLSC of the present invention. These uses include diagnostic, prophylactic and therapeutic techniques.

### Treatment

The isolated pluripotent embryonic stem cells expressing DAZL cells from the amniotic fluid cells or their derivatives may in various regimes to treat diseases in humans or animals. As used herein the term “treat” or “treatment” refer to both therapeutic treatment and prophylactic or preventative measures, wherein the object is to prevent, slow down (lessen), or reverse an undesired physiological change or disorder. The term “treat” also refers to the characterization of the type or severity of disease which may have ramifications for future prognosis, or need for specific treatments. For purposes of this invention, beneficial or desired clinical results include, but are not limited to, alleviation of symptoms, diminishment of extent of disease, stabilized (i.e. not worsening) state of disease, delay or slowing of disease progression, amelioration or palliation of the disease state, and remission (whether partial or total), whether detectable or undetectable.

“Treatment” can also mean prolonging survival as compared to expected survival if not receiving treatment. Those in need of treatment include those already with the condition or disorder as well as those prone to have the condition or disorder or those in which the condition or disorder is to be prevented.

To treat a human or animal in need of treatment, the cells can be either regenerated into segments of a desired tissue, then transplanted into the patient, or can be regenerated into a whole tissue that will be used to replace the failing tissue, or can be injected into a tissue of interest as whole cells, where they will regenerate at the injected location.

It may be possible to replace any type of failing tissue with the cells of the present invention. Pluripotent embryonic stem cells expressing DAZL may be differentiated into tissues such as liver, endocrine tissues, lung, blood cells, neuronal or astroglial cells, spermatocytes, oocytes or others, which may then be used for transplantation to cure or treat diseases.

Examples of diseases that may be treated with the cells of the invention and tissues include but are not limited to infertility, cirrhosis of the liver, pancreatitis, diabetes, Parkinson's disease, spinal cord injury, stroke, burns, heart disease, certain types of cancer, osteoarthritis, rheumatoid arthritis, leukemia, lymphoma, genetic blood disorders, and brain disorders such as Alzheimer's disease. Additional examples of diseases that can be treated with amniotic fluid-derived GLSCs include but are not limited to Acute Lymphoblastic Leukemia, Acute Myelogenous Leukemia, Acute Biphenotypic Leukemia, and Acute Undifferentiated Leukemia; Chronic Myelogenous Leukemia, Chronic Lymphocytic Leukemia, Juvenile Chronic Myelogenous Leukemia, Juvenile Myelomonocytic Leukemia, Refractory Anemia, Refractory Anemia with Ringed Sideroblasts, Refractory Anemia with Excess Blasts, Refractory Anemia with Excess Blasts in Transformation, Chronic Myelomonocytic Leukemia, Aplastic Anemia, Fanconi Anemia, Paroxysmal Nocturnal Hemoglobinuria, Pure Red Cell Aplasia, Acute Myelofibrosis, Agnogenic Myeloid Metaplasia, myelofibrosis, Polycythemia Vera, Essential Thrombocythemia, Non-Hodgkin's Lymphoma, Hodgkin's Disease, Chediak-Higashi Syndrome, Chronic Granulomatous Disease, Neutrophil Actin Deficiency, Reticular Dysgenesis, Mucopolysaccharidoses, Hurler's Syndrome, Scheie Syndrome, Hunter's Syndrome, Sanfilippo Syndrome, Morquio Syndrome, Maroteaux-Lamy Syndrome, Sly Syndrome, Beta-Glucuronidase Deficiency, Adrenoleukodystrophy, Mucolipidosis II, Krabbe Disease, Gaucher's Disease, Niemann-Pick Disease, Wolman Disease, Metachromatic Leukodystrophy, Familial Erythrophagocytic Lymphohistiocytosis, Histiocytosis-X, Hemophagocytosis, Inherited Erythrocyte Abnormalities, Beta Thalassemia Major, Sickle Cell Disease, Inherited Immune System Disorders, Ataxia-Telangiectasia, Kostmann Syndrome, Leukocyte Adhesion Deficiency, DiGeorge Syndrome, Bare Lymphocyte Syndrome, Omenn's Syndrome, Severe Combined Immunodeficiency, Common Variable Immunodeficiency, Wiskott-Aldrich Syndrome, X-Linked Lymphoproliferative Disorder, Other Inherited Disorders, Lesch-Nyhan Syndrome, Cartilage-Hair Hypoplasia, Glanzmann Thrombasthenia, Osteopetrosis, Inherited Platelet Abnormalities, Amegakaryocytosis, Congenital Thrombocytopenia, Plasma Cell Disorders, Multiple Myeloma, Plasma Cell Leukemia, Waldenstrom's Macroglobulinemia, Breast Cancer, Ewing Sarcoma, Neuroblastoma, Renal Cell Carcinoma, brain disorders such as Alzheimer's disease, and the like (see, for example, hypertext transfer protocol (http) on the world wide web at: marrow. org/index. html, which is incorporated by reference herein in its entirety).

Many different types of tissues may be replaced, in full or in part, using the differentiated cells derived from the GLSC as described herein. Examples of tissues which may be (at least partially) replaced include, but are not limited to, lung tissue, heart tissue, ocular tissue, nerve tissue, brain tissue, muscle tissue, skin, pancreatic beta cells, and the like.

The isolated cells of the invention may also be genetically modified by transfection with any suitable gene of interest. General techniques useful to genetically modify the GLSC (or their derivatives) can be found, for example, in standard textbooks and reviews in cell biology, tissue culture, and embryology. Methods in molecular genetics and genetic engineering are described, for example, in Molecular Cloning: A Laboratory Manual, 2nd Ed. (Sambrook et al., 1989); Oligonucleotide Synthesis (M. J. Gait, ed., 1984); Animal Cell Culture (R. I. Freshney, ed., 1987); the series Methods in Enzymology (Academic Press, Inc.) Gene Transfer Vectors for Mammalian Cells (I. M. Miller & M. P. Calos, eds., 1987); Current Protocols in Molecular Biology and Short Protocols in Molecular Biology, 3rd Edition (F. M. Ausubel et al., eds., 1987 & 1995); and Recombinant DNA Methodology II (R. Wu ed., Academic Press 1995); each of which is incorporated by reference herein in its entirety.

The methods used to perform the genetic modifications to the cells can be any of those known in the molecular biological arts for making genetic alternations. Such methods include, but are not limited to, the use of positive-negative selector vectors as described in U.S. Pat. Nos. 5,464,764; 5,487,992; 5,627,059; and 5,631,153 to Capecchi, et al.; and U.S. patent application Ser. No. 08/781,559. In addition, yeast artificial chromosomes (YACs) can be employed to perform genetic modifications as described in U.S. patent application Ser. Nos. 08/597,532; 08/397,547; 08/187,161; 08/276,565; 08/375,482; 08/485,505; and 08/372,482.

Furthermore, isogenic DNA constructs can be used with the GLSC cultured using the methods and materials provided by the present invention as described in U.S. patent application Ser. No. 08/563,138. Still other methods include those described in U.S. Pat. No. 5,591,625 to Gerson, et al. for the preparation stem cells capable of augmented expression of certain gene products, signal transduction molecules, cell surface proteins and the like for therapeutic applications.

In another aspect, the present invention provides useful pharmaceutical products produced by the cells or cell lines of the present invention, including cells and cell lines derived from GLSC comprising one or more genetic modifications and/or their gene products. In aspects of the invention, inhibitors of reverse transcriptase such as nevirapine may be used to introduce a genetic modification in the cells. One skilled in the art would understand that there may be other means introduce genetic modifications, such as but not limited to, the insertion of the TERT gene (telomerase reverse transcriptase). Cells that have been transfected with vector expressing the TERT sequence have become immortal and can be propagated for an unlimited period of time (PCT publication WO2005/017117).

In one aspect, the invention provides a method for screening to identify compounds that affect the function of the cells of the invention. In one embodiment, the method includes incubating at least one compound and at least one pluripotent embryonic stem cell expressing DAZL under conditions sufficient to allow the compound and cell to interact; and determining the effect of the compound on cell function before and after incubating in the presence of the compound. Cell function that may be modulated (e.g. inhibited or stimulated) by the compound and includes, but is not limited to, differentiation, gene expression, production of growth factors, response to growth factors and modulation of cell membrane permeability.

Additionally, the fetal stem cells of the present invention may be used as autologous/heterologous transgene carriers in gene therapy to correct inborn errors of metabolism affecting the cardiovascular, respiratory, gastrointestinal, reproductive, and nervous systems, or to treat cancer and other pathological conditions.

The pluripotent embryonic stem cells of the present invention can be used in autologous/heterologous tissue regeneration/replacement therapy, including but not limited to treatment of corneal epithelial defects, cartilage repair, facial dermabrasion, burn and wound dressing for traumatic injuries of skin, mucosal membranes, tympanic membranes, intestinal linings, and neurological structures. For example, augmentation of myocardial performance can be achieved by the transplantation of exogenous fetal stem cells into damaged myocardium, a procedure known as cellular cardiomyoplasty (CCM) which can be used for enhancing myocardial performance and treating end-stage cardiac disease. Fetal stem cells according to the present invention can also be used as a tool for the repair of a number of CNS disorders as described in a review by Cao et al. (Stem cell repair of central nervous system injury, J. Neuroscience Res. 68:501-510, 2002). The cells of the present invention can also be used in reconstructive treatment of damaged tissue by surgical implantation of cell sheets, disaggregated cells, and cells embedded in carriers for regeneration of tissues for which differentiated cells have been produced. The cells may also be used in tissue engineered constructs. Such constructs comprise a biocompatible polymer formed into a scaffold suitable for cell growth. The scaffold can be shaped into a heat valve, vessel (tubular), planar construct or any other suitable shape. Such constructs are well known in the art (see, e.g., WO02/035992, U.S. Pat. Nos. 6,479,064, 6,461,628). The amniotic fluid, chorionic villus, placenta tissue and embryonic stem cells, before or after differentiation, may be cryopreserved in a cryoprotective solution comprising a medium or buffer and a cryoprotective agent. Examples of media are Dulbecco's Modified Eagle Medium (DMEM), Medium 199 (M199), F-12 Medium, and RPMI Medium. An example of a buffer is phosphate buffered saline (PBS). Examples of cryoprotective agents are dimethylsulfoxide (DMSO) and glycerol. Examples of cryoprotective solutions are: DMEM/glycerol (1:1), DMEM/7.5% DMSO, M199/7.5% DMSO, and PBS/3.5 M DMSO. Optionally, the samples may be treated with antibiotics such as penicillin or streptomycin prior to cryopreservation. Cryopreservation may be accomplished using a rapid, flash-freeze method or by more conventional controlled rate-freeze methods. Rapid freezing of amniotic tissue may be accomplished by placing sample(s) in a freezing tube containing a cryoprotective solution and then rapidly immersing the freezing tube in liquid nitrogen. General slow freezing may be accomplished by placing sample(s) in a freezing tube containing a cryoprotective solution and then placing the freezing tube in a −70.degree. C. freezer. Alternatively, the sample(s) may be subjected to controlled rate freezing using a standard cryogenic rate controlled system. Products of the stem cells of the present invention may be used in reconstructive treatment, either in vivo or ex vivo. Examples of agents that can be produced using fetal stem cells of the present invention include growth factors, cytokines, and other biological response modifiers.

All references cited herein are hereby incorporated by reference in their entirety. Nothing herein is to be construed as an admission that the invention is not entitled to antedate such disclosure by virtue of prior invention. Throughout this description, the examples shown should be considered as exemplars, rather than as limitations on the present invention. Since modification of the specific embodiments will be apparent to those of skill in the art, it is intended that this invention be limited only by the spirit and scope of the appended claims.

### EXAMPLES

In the examples the term “GLSC” is used to refer to the novel pluripotent embryonic stem cell of the invention that expresses DAZL.

### Example 1

### Amniotic Fluid Cell Isolation and Expansion of GLSCs in Cell Culture

Human AFCs sample were collected and plated in a 75 cm² flask and incubated at 37° C. with 5% humidified CO₂ (FIGS. 1A-I). The culture medium of the present invention is Stefanidis medium which comprises about 80% basal medium [80% KnockOut™ Dulbeco's modified Eagle's medium (DMEM) (Gibco BRL, Rockville, Md.), 1 mM L-Glutamine, 1% nonessential amino acids stock (Gibco BRL, Rockville, Md.), supplemented with 20% KnockOut SR™, a serum-free replacement originally optimized for human ES cells (Gibco BRL, Rockville, Md.)], penicillin and streptomycin, and 20% amniotic fluid supplemented with 4 ng/ml basic fibroblast growth factor (bFGF).

The novel culture medium of the invention comprises amniotic fluid and a culture medium as described herein. In aspects the medium comprises amniotic fluid and a growth factor such as but not limited to beta-FGF.

After about 96 to about 128 hours or sufficient period of time to permit a portion of amniotic fluid cells to adhere to the substrate, a non-adhering portion of amniotic fluid cells which are believed to mainly contain embryonic like stem cells such as GLSC in the supernatant medium were collected. The non-adherent cells then were at 800-1000 rpm and plated in a) 5 ml of Stefanidis medium in 25 cm² flask and incubated at 37° C. with 5% humidified CO₂. The cells were cultured with replacement of Stefanidis medium every 2-3 days until cells morphology consistent with EG cells were observed, typically, 10-30 days. On the 20th day of culture, a subset of cells growing on the 96-well culture dish were fixed and stained for the presence of alkaline phosphatase by using a commercially available diagnostic kit (Sigma Chemicals, product number 86-R). The cells were washed 2 times with phosphate buffered saline (PBS) then fixed for 30 seconds in a mixture of 25 ml citrate solution (18 mM sodium citrate, 9 mM sodium chloride, pH 3.6), 65 ml acetone and 8 ml of 37% formaldehyde. Fixed cells were then incubated in the dark for 15 min. in alkaline-dye mixture. The cells were then rinsed with deionized water for 2 min. and allowed to dry. Alkaline phosphatase positive primordial germ cell (PGC) and EG cells stained red, while cells that lack alkaline phosphatase activity, such as human fibroblasts, remained clear.

Cells were photographed throughout the initial 20 days of culture using phase contrast microscopy and selected cells were processed for alkaline phosphatase staining as described herein. Cells were also photographed using electron microscopy.

It will be appreciated by those of skill in the art that should the non-adherent portion of embryonic like stem cells and germline-like stem cells not be removed after about 96-128 hours, these embryonic like stem cells and germline-like stem cells may attach to the fibroblast layer formed from the mesenchymal adherent population of cells present in the amniotic fluid as clearly shown in FIG. 1F.

### Example 2

### Morphological Characterization GLSCs

Cultured amniotic fluid-derived cells were karyotyped using methods well known to those in the art. These cells could be passaged for at least 5-10 times and were found to be near-immortal and were named germline-like stem cells (GLSC).

All of 50 amniotic fluid sample harvests of 5 ml gave rise to at least one adherent GLSC colony and continuous culture. The GLSCs were cloneable into single cell clones and were non-senescing. The majority of sample harvests gave rise to 3-4 individual clones. Among the individual clones, different colonies/cultures had diverse colony morphologies. Some colonies were adherent while other colonies were floating. About half of the amniotic fluid samples cultured under condition A (Stefanidis medium) gave rise to GLSC clones/cultures that behaved like immortal cell lines, as shown in FIGS. 2A and 2B, while the other half were fibroblasts and differentiated cell types.

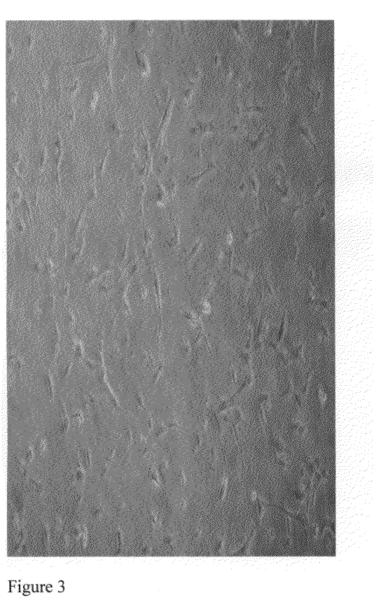

In embodiments of the invention, some GLSCs may be differentiated into fibroblasts and have a typical fibroblastic morphology (FIGS. 3A-C).

The GLSC cultures grew vigorously, with a doubling time of 28-34 hours.

When confluent, the cells piled up in multilayered fashion and numerous round, semi-detached cells grew on top of a swirling, non-contact-inhibited layer of cells. These GLSC cultures expressed the telomerase gene/protein. The techniques used to determine telomerase activity are common and well known to those skilled in the art (N. W. Kim, et al, Science 266 (1994), pp. 2011-2015; S. L. Weinrich, et al Nat Genet 17 (1997), pp. 498-502.

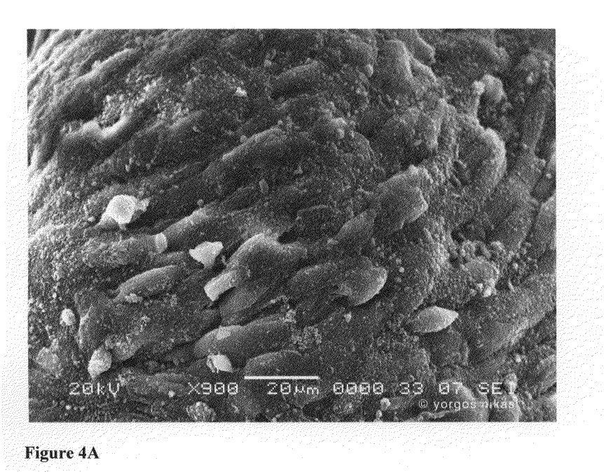

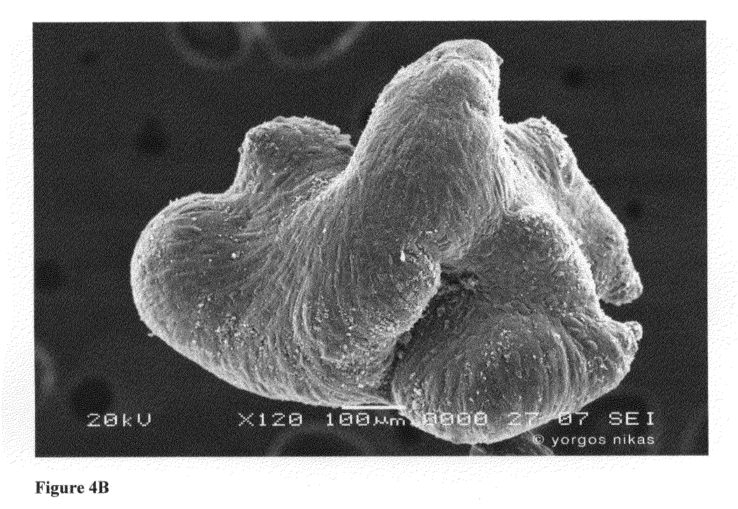

Furthermore, the GLSCs were photographed using electron microscopy as shown in FIGS. 4A-B. Shown in FIG. 4A is a GLSC under electron microscopy and in FIG. 4B is an embryoid body formed from a GLSC line.

The GLSCs vigorously grew and the GLSC lines expressed very high levels of a set of cell surface determinants known to be present on undifferentiated embryonic stem cells as explained below.

### Example 3

### FACS Analysis of GLSCs

Fresh amniotic fluid cells from 3 donors were prepared for FACS analysis by the following protocol: Amniotic Fluid samples were stained with 3 surface markers/tube. Each time two tubes were analyzed, one isotype control or an unstained sample and one with the antibodies. The analysis includes the percentages of the cells that express each marker.

Amniotic fluid samples were obtained from amniocentesis all performed after the 18 week of pregnancy for routine prenatal diagnosis. Fresh amniotic fluid samples were analyzed within 6 hours of collection. Cells were washed twice with washing buffer (phosphate buffered saline (PBS), bovine serum albumin (BSA; 0.1%) and sodium azide (NaN₃; 0.1%)) and stained with antibody to c-kit conjugated to phycoerythrin (PE) fluorochrome, 7-Amino-Actinomycin (7AAD) and to DAZL indirectly conjugated to fluoresecin isothiocyanate (FITC) (Table 1). Labeled cells were then washed and resuspended in parafolmadehyde (PFA; 1%) and kept in the dark at 4° C. until acquisition. Fluorochromes FITC and PE, and 7AAD were detected by flow cytometric analysis as fluorescence 1, 2 and 3, respectively. Mouse monoclonal antibody against c-kit-PE and its isotype control were purchased from Abcam (Cambridge, UK); goat polyclonal antibody against DAZL, its secondary donkey anti-goat IgG-FITC antibody and isotype control were obtained from Santa Cruz Biotechnology Inc.; and 7AAD was purchased from Becton Dickinson Biosciences.

Acquisition of samples was performed with FACSort cytometer (Becton Dickinson). The instrument was set for three-colour analysis using CaliBRITE beads (Becton Dickinson) with FSC PMT gain on 0.1 (Log) to visualize all cells, on the FSC vs. SSC dot plot (FIG. 5). Between 20,000 and 30,000 events were collected for each sample and stored at list mode data using CellQuest software (Becton Dickinson). Samples were analysed using CellQuest software.

Initially, two main cell subpopulations, R1 and R2, were distinguished according to their forward and side scatter characteristics (FIG. 5A). Although both populations autofluorescenced, the second, R2 population that represented the larger cells, showed an extremely high degree of autofluorescence that interfered with the results (FIGS. 5C and 5D). Trypan blue viability test was performed in amniotic fluid samples to estimate the percentage of dead cells. Almost half of the cells were found to be dead (49.64%). These results could not be confirmed by 7AAD staining because of autofluorescence interference. However, it is believed that cells in the R2 gate mostly represent the dead cell population.

Thus, the assessment of DAZL and c-kit expression was performed from the R1 gated cells. All samples including the isotype controls were stained with the dead cell marker 7AAD and only 7AAD⁻ cells were then selected for further analysis (FIG. 5B). The expression of the surface markers was then assessed as the percentage of positive cells of the 7AAD⁻ cell population by subtracting their expression of their isotype controls (Table 2). As can be seen in Table 2, of the cells in this 7AAD− population 34.18% expressed DAZL and 21.73% expressed c-Kit.

### Example 4

### Cell Surface Markers on GLSCs

GLSC expressed very high levels of a set of cell surface determinants known to be present on non-differentiated human Embryo Stem Cells (hES) and expressed a set of surface determinants known to be associated with non-differentiated human Mesenchymal Stem Cells (MSC). GLSC did not express markers characteristic of hematopoietic cells, e.g. CD45 and CD34. The flow cytometry was performed as described above in Example 3.

Mass cultures of the GLSC were characterized by very high expression of DAZL, SSEA-4, c-Kit, and of the keratin sulphate-related antigens Tra-1-60 and Tra-1-81 as shown in FIG. 6.

The GLSCs also expressed the transcription factor OCT-4. The human embryonic stem cell markers typically found on GLSC are shown in Table 1. From each amniocentesis sample, at least 2-10 colonies that express Oct-4, SSEA-4. TRA-1-81 could be achieved. The GLSCs also expressed oxytocin receptor. Colony formation was prevented when the oxytocin receptor was blocked using an oxytocin antagonist, atociban.

### Example 5

### RNA Extraction and RT-PCR

Total RNA was extracted from approximately 3×10⁶ germline like stem cells and 1×10⁶ fresh amniotic fluid cells by employing a commercially available kit (RNAeasy micro kit; Qiagen, Valencia, Calif., USA) according to manufacturer's instructions. The use of RNase-free DNase I and carrier RNA, offered highly purified RNA.

Total RNA from germline like stem cells and fresh amniotic fluid cells were used for cDNA synthesis by reverse transcription (RT). For the RT reaction a commercially available kit was employed (Retroscript kit, Ambion, Austin, Tex. USA). Reverse transcription was followed by two rounds of nested PCR for Oct-4 mRNA and by one round of PCR for DAZL mRNA. Primer sequences used in PCRs for DAZL and Oct-4 mRNA amplification were designed with the Primer 3 program (Rosen and Skaletsy, 1997). All primers were ordered from MWG Biotech (Table 3). The first round PCR mastermix contained 3 μl cDNA of Oct-4 in a total 50 μl volume. Five μl of 10×PCR buffer, 1.5 mmol MgCl₂/l, 0.2 μmol of 3′ and 5′ outer primer, 0.2 mmol of each dNTP/l and 1.5 u Taq polymerase were used (Invitrogen Life Technologies). All reactions were overlaid with light white oil. Polymerase chain reaction was performed for 30 cycles. Cycling conditions were 94° C. denaturation, 55° C. annealing and 72° C. extension, with each step lasting 1 minute. Reaction was terminated at 72° C. for 10 minute. First round PCR products were stored at −20° C. For the second PCR round, 3 μl of the first round PCR product were added to 47 μl of freshly prepared mastermix containing PCR buffer, MgCl₂, dNTPs, Taq polymerase and inner primers in the same quantities as the first round. The cycling conditions were also the same as in the first round PCR.

For DAZL the PCR reaction mixture contained 5 μl cDNA in a total volume of 50 μl. The concentrations of PCR buffer, MgCl₂, dNTPs, Taq polymerase and DAZL specific primers were the same as in the PCR reaction mixture of Oct-4. Polymerase chain reaction was performed for 45 cycles and the cycling conditions were the same as described above. Products were stored at −20° C.

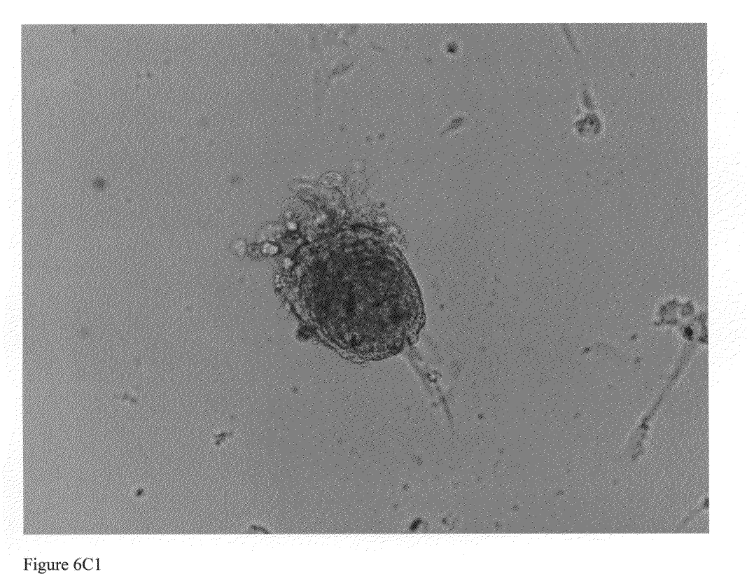

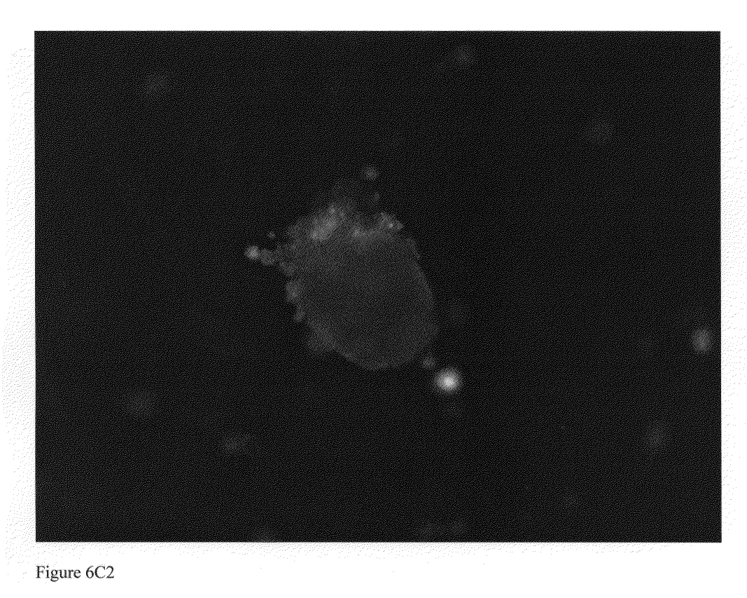

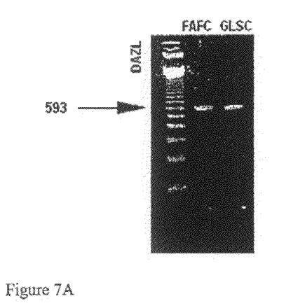

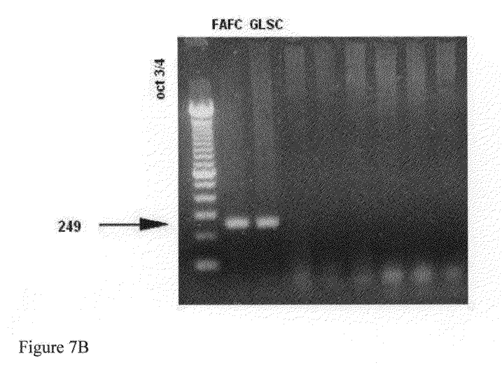

The amplified products were analyzed by electrophoresis on 2% agarose gel containing ethidium bromide. Seven μl of each PCR product run in parallel with a 100 bp DNA ladder (Invitrogen Life Technologies). As shown in FIG. 7A, both the fetal amniotic fluid cells (FAFC) and the GLSCs express DAZL. As shown in FIG. 7B both the fetal amniotic fluid cells (FAFC) and the GLSCs express Oct3/4.

### Example 6

### Cryopreservation and Banking of Fresh Amniocentesis-Derived Cells and of Cultured GLSC

Both fresh amniocentesis-derived cells and cultured GLSC were cryopreserved for banking purposes. Techniques for cryopreservation are well known and practiced by those of skill in the art as disclosed in PCT publication WO2005017117. Briefly, samples of amniotic fluid ranging from 2 to 5 ml were harvested. The cells were centrifuged to remove excess amniotic fluid. The cells were then frozen in medium containing 10% dimethyl sulfoxide and 25% fresh, filtered (0.10 micron) amniotic fluid (DMSO/AF freezing medium). Alternatively, the cells were grown to produce GLSC cultures, which were then frozen. The fresh amniotic fluid derived cells and cultured GLSCs were frozen in DMSO/amniotic fluid freezing medium in a controlled-rate liquid nitrogen freezer at 1° C./min to about 10° C./min. Frozen samples were stored under liquid nitrogen in freezing ampoules.

### Example 7

### Differentiation of GLSCs

As mentioned previously, the GLSCs may be differentiated into many cell types. For example, GLSCs cells can be differentiated into cells of ectoderm, mesoderm and endoderm. In addition to the differentiation paths exemplified below, GLSCs cells are capable of other, pluripotent differentiation paths GLSC were cultured, and were differentiated into various cell types, such as neural cells, adipogenic cells, and chondrogenic cells.

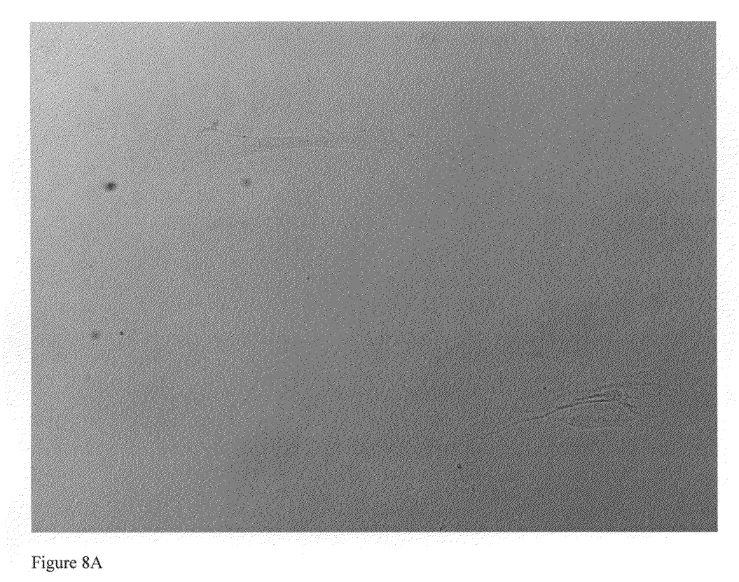

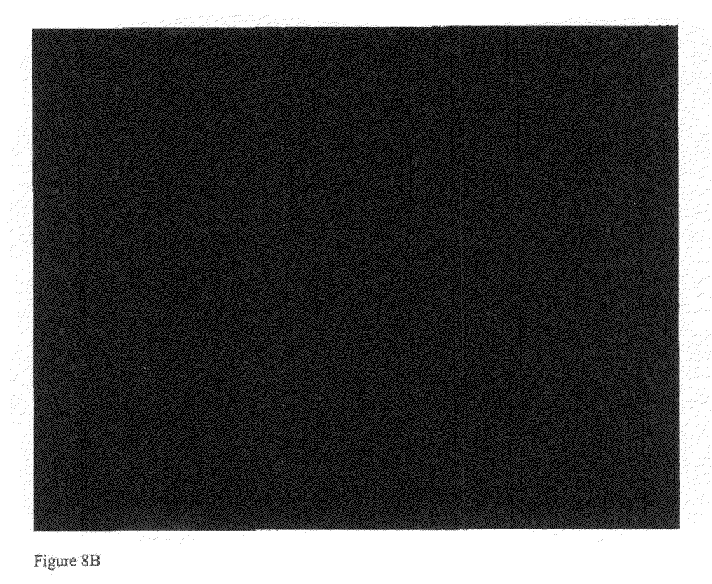

As shown in FIGS. 8A and 8B, GLSCs may be differentiated into neural glial cells and express the neuro-marker s-100.

### Example 8

### Differentiation of GLSC into Spermatogenesis-Like-Structures

Both human and mouse embryonic stem cells are capable of forming primordial germ cell in vitro (Kehler, J. Seminars in Reproductive Medicine 23:222-233, 2005). These germ cells are capable of undergoing meiosis and forming both male and female gametes by gametogenesis in vitro. For example, GLSCs may be differentiated to spermatogonia in a testicular environment by a) transplantation in xenogenic testes and b) in vitro culture using retinoic acid (RA) at about a final concentration of 10⁻⁵ M. GLSCs of the present invention are to be differentiated into male gametes according to the following method as outlined by Navernia K. et al. Dev. Cell; 11(1):125-32, 2006, where mouse embryonic stem cell line R1 (XY) was cultured in an undifferentiated state on a feeder layer of mitomycin C-inactivated mouse embryonic fibroblasts with Dulbecco's modified Eagle's medium (DMEM, GIBCO-BRL) supplemented with 15% FCS, 2 mM L-glutamine (GIBCO-BRL), 50 μM β-mercaptoethanol (β-ME; Promega), 1× non essential amino acids (NEM; GIBCO-BRL), and 103 U/ml LIF as described previously. Linearized plasmid DNA (30 μg) was electroporated into ES cells. Colonies resistant to G418 (400 μg/ml) were selected. Resistant colonies were tested by PCR, and colonies that contain the Stra8-EGFP construct were selected and cultured in an undifferentiated state. Cultures were proliferated in the above described medium for an additional 2 months (four passages) and were then frozen. Thereafter, cells were cultured on a feeder layer of mitomycin C-inactivated mouse embryonic fibroblasts with basic ES cell medium. To induce differentiation, medium was changed to medium containing retinoic acid (RA) at a final concentration of 10⁻⁵ M, and the cells were cultured for 10 days. Positive cells (60%) were sorted by FACS. Briefly, cells were dissociated with 0.25% trypsin/EDTA, neutralized with DMEM with 10% FCS, washed twice with PBS, and then resuspended in PBS containing 0.5% BSA. Approximately 2×10⁶ cells/ml in PBS/BSA were used for sorting. The flow cytometry was performed on a FAC-Star Plus (Becton Dickinson) equipped with dual 488 nm argon and 633 nm helium neon lasers. Sorted cells were cultured in RA-free medium. After 8-10 weeks (4 passages), medium was changed with medium supplemented with RA (10-6 M) and after 12 h, GFP positive cells (90%) were sorted by FACS. Thereafter, the cells were cultured in basic medium supplemented with LIF on fibroblast feeder layers and transfected with the Prm1-DsRed construct. Positive cells colonies were selected after PCR analysis. Two cell lines were established and designated as SSC7 and SSC12. For differentiation, the cells were cultured on gelatine-coated dishes, without LIF. The cells were characterized by determining the expression of different markers for PGCs, premeiotic, meiotic, and postmeiotic male germ cells by RT-PCR analysis. To investigate SSC capacity and the further development of SSC7 and SSC12 cell lines in vivo, cells were transplanted into one of the testes of germ cell-depleted recipient mice. The other testis served as an internal control. Histological analysis of testes after 4 months showed the appearance of spermatogenesis-like-structures and sperm in the lumen of two of ten transplanted mice (for further details please refer to the relevant article.)

### Example 9

### Comparison of Gene Expression Profiles Between Amniotic Fluid Cells Cultured in Stefanidis Medium and Germ Cells from 18-20 Week Embryos

Gene expression profiles between amniotic fluid cell samples (obtained for routine prenatal diagnostic amniocentesis after the 18th week of pregnancy) were cultured with Stefanidis' medium according the present invention (Control cells) and cells derived from human gonadal ridges and dorsal mesenteries (primordial germ cells) from 18th-20th week old embryos (from an aborted pregnancy due to Down syndrome) (Experimental cells) were compared using DNA microarray analysis.

Cells derived from primordial germ cells (PGCs) are termed human embryonic germ cells (EG) cells, can undergo self-renewal in vitro and maintain an undifferentiated phenotype. As described above, DAZL, Oct-4, Nanog, SSEA-4, SSEA-1 represent characteristic markers of human EG cells. DAZL belongs to DAZ gene family which is expressed in prenatal and postnatal germ cells of males and females. Oct-4 POU transcription factor is expressed in totipotent embryonic stem and germ cells and rapidly disappears when cells differentiate. The stage-specific embryonic antigen 4 (SSEA4) is expressed in undifferentiated human ES cells and is downregulated during differentiation, while SSEA1 is expressed only in later stages of human ES cells differentiation.

Expression of DAZL, Oct-4, Nanog, SSEA-4, SSEA-1 in Control and Experimental cells was assessed by semiquantitative RT-PCR and immunofluorescence (IF) analysis. It was determined that that both amniotic fluid stem cells (cultured according to the composition and methods of the present invention) and human embryonic germ cells positively expressed similar levels of DAZL, Oct-4, Nanog, SSEA-4. Interestingly embryonic germ cells were found negative for SSEA-1 which underlines their undifferentiated status and strengthens the evidence for their germ cell identity. Considering their origin from 18-20 week embryos, it should be expected to positively express SSEA-1, but Down syndrome has been reported to associate with delayed gonadal maturation, therefore explaining the absence of SSEA-1.

To further investigate the similarities in gene expression between germ cells and amniotic fluid stem cells, two vials containing the human cell samples kept under dry ice were provided. RNA was isolated using standard RNA extraction protocols (NucleoSpin™ RNA II, Macherey-Nagel). The gene expression was assessed using the PIQOR microarray, as briefly described:

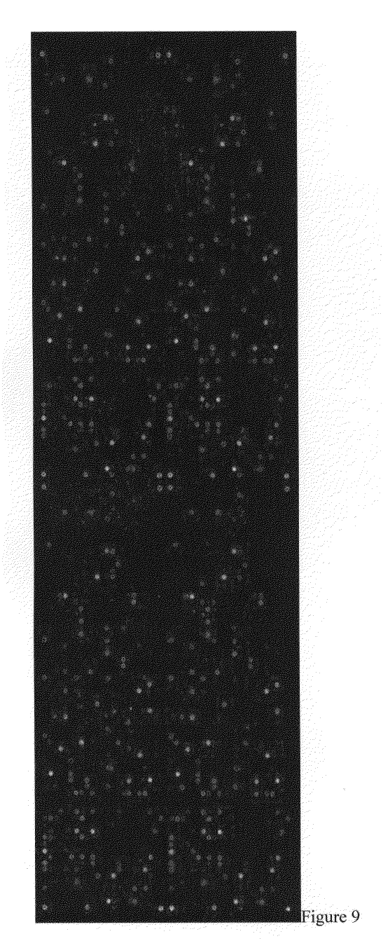

Sample labelling was performed according to the PIQOR™ User manual. Subsequently, the fluorescently labelled samples were hybridized overnight to topic-defined PIQOR™ Stem Cell Microarrays Human Antisense using the a-Hyb™ Hybridization Station. In general, Control samples (Amniotic Fluid stem cells) are labeled with Cy3 and Experimental samples (Germ cells from Down Syndrome) are labeled with Cy5. Fluorescence signals of the hybridized PIQOR™ Microarrays were detected using the laser scanner ScanArray™ Lite (PerkinElmer Life Sciences). Shown in FIG. 9 is a false colour image of the microarray experiment is shown: Red colour indicates that the Cy5 signal intensity is higher than the Cy3 signal intensity. Therefore, the corresponding gene is overexpressed in the Experimental sample. Green spots, however, indicate that the fluorescence intensity in the control sample is stronger than in the experimental sample. Yellow spots indicate that the signal intensities are equal for both samples. Spots located in areas in which hybridization artefacts such as air bubbles occur, are flagged and excluded from further analysis. Even if one or two spots are flagged, sufficient replicates for valid data analysis remain on the slide since each gene is spotted on four different positions on the microarray.

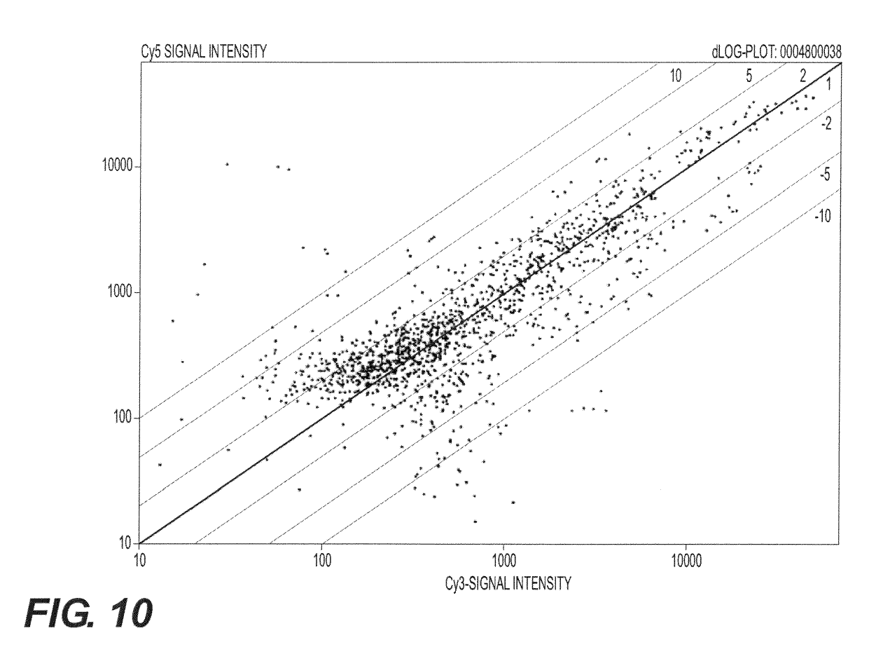

Mean signal and mean local background intensities were obtained for each spot of the microarray images using the ImaGene software (Biodiscovery). The PIQOR™ Analyzer allows automated data processing of the raw data text files derived from the ImaGene software. This includes background subtraction to obtain the net signal intensity, data normalization, and calculation of the Cy5/Cy3 ratios. As an additional quality filtering step, only spots/genes are taken into account for the calculation of the Cy5/Cy3 ratio that have at least in one channel a signal intensity that is at least 2-fold higher than the mean background. The result of this data analysis is visualized in a doublelog scatter plot (FIG. 10):

As seen in the scatter plot above the vast majority of the genes examined share similar expression patterns in GLSC and EG cells.

PIQOR™ Analyzer calculates all normalized mean Cy5/Cy3 ratios of the four replicates per gene (Table 4). In addition to the ratio, the respective coefficient of variation (cv, in %) is listed in the gene ratio list. This coefficient of variation refers to the average of the Cy5/Cy3 ratios for the gene replicates. However, a negative value (−%) indicates that only one out of four spots could be evaluated and, therefore, no cv could be determined.

Genes that are >1.7-fold up- or downregulated represent putative candidate genes and are highlighted by green and red color in the gene ratio list. Green colour indicates a <0.58-fold down-regulation of gene expression in Experimental Cells (Germ cells from Down Syndrome), corresponding to a fold change <−1.7 of a certain gene in comparison to the Control sample (Amniotic fluid stem cells). Red colour indicates a more than 1.7-fold up-regulation of the respective gene in comparison to the control (Amniotic fluid stem cells). The cells of spots/genes that did not pass the quality filtering because they are either flagged or have very low signal intensities are blanked in Table 4, in order to discriminate questionable results from relevant results in the gene ratio list.

Of all the 937 spots/genes that passed the quality filtering 80 (i.e. 8.5%) were found up-regulated and 57 (i.e. 6.1%) were found down-regulated in germ cell line compared to amniotic fluid stem cells. Genes that were found differentially expressed between the two examined cell lines, mostly associate with formation of cytoskeleton and adhesion to their surrounding matrix (such as VCAM1, ALCAM, ITGB1, ITGA1_{—}2, COL1A1, COL18A1_{—}2, COL2A1, TIMP3, LAMA1, FN1, MMP16_{—}1, KRT18, KRT8, TPM1, FN1_REPEAT-1TO6, FN1_REPEAT-A, FN1_REPEAT-B, MMP21-22-23), communication with their microenvironment (such as EDN1, VEGC, HTR2B, EDNRB, HBEGF, FGF5, VEGFA, VGR1, IGFBP2, IGFBP5), regulation of cell cycle and proliferation (such as CCNB2, CCNE1, MAPK3, CXCR4, CDK4, CDC25C, MAPK13, C20ORF1, MAD2L1, BUB1B, BUB3, MAD2L2, REC1) and immune response (IL6, CD9, CXCL12, PGH2). These differences respectively, could be attributed to the differential origin of the donor subjects (different human donors), to specific adaptation mechanisms of the progenitor cells in their original microenvironment, differences in cell cycle regulation and proliferation potential since amniotic fluid cells are known to proliferate slower, and possible contamination of the original sample (especially the germ cells from obtained from embryonic testis) with immune system elements during the isolation process.

POU5F1 encoding OCT3/4, TDGF, GABRB3, FGF4, and TERT represent genes particularly known to be expressed in stem cells and germ cells become down-regulated upon differentiation. Among them POU5F1 (Nichols, J. et al. Formation of pluripotent stem cells in the mammalian embryo depends on the POU transcription factor OCT4. Cell 95, 379-391, 1998), Nanog (Mitsui, K. et al. The homeoprotein Nanog is required for maintenance of pluripotency in mouse epiblast and ES cells. Cell 113, 631-642, 2003, Chambers, I. et al. Functional expression cloning of Nanog, a pluripotency sustaining factor in embryonic stem cells. Cell 113, 643-655, 2003), TDGF Teratocarcinoma-derived growth factor-1 (Sato N, Sanjuan I M, Heke M, Uchida M, Naef F, Brivanlou A H. Molecular signature of human embryonic stem cells and its comparison with the mouse. Dev Biol. 2003 Aug. 15; 260(2):404-13, Baldassarre, G. et al. Transfection with a CRIPTO anti-sense plasmid suppresses endogenous CRIPTO expression and inhibits transformation in a human embryonal carcinoma cell line. Int. J. Cancer 66, 538-543, 1996, Sperger, J. M. et al. Gene expression patterns in human embryonic stem cells and human pluripotent germ cell tumors. Proc. Natl. Acad. Sci. USA 100, 13350-13355, 2003, Gerecht-Nir S, Dazard J E, Golan-Mashiach M, Osenberg S, Botvinnik A, Amariglio N, Domany E, Rechavi G, Givol D, Itskovitz-Eldor J. Vascular gene expression and phenotypic correlation during differentiation of human embryonic stem cells. Dev Dyn. 2005 February; 232(2):487-97.) are considered to be “core stemness genes”, because they are found overexpressed in almost all stem cell lines.

GABRB3 (GABRB3, GABA A receptor, b3) (Sperger, J. M. et al. Gene expression patterns in human embryonic stem cells and human pluripotent germ cell tumors. Proc. Natl. Acad. Sci. USA 100, 13350-13355, 2003), FGF4 (International Stem Cell Initiative, Characterization of human embryonic stem cell lines by the International Stem Cell Initiative. Nat Biotechnol. 2007 July; 25(7):803-16) and TERT (Li S S, Liu Y H, Tseng C N, Chung T L, Lee T Y, Singh S ‘Characterization and gene expression profiling of five new human embryonic stem cell lines derived in Taiwan.’ Stem Cells Dev. 2006 August; 15(4):532-55) are also represent stemness-related genes. FGF4 is a downstream target of the FGF (Fibroblast Growth factor) cascade and downregulated upon stem cells differentiation. TERT is related to telomerase function and telomeres maintenance during stem cells renewal.

Importantly, the DNA microarray analysis revealed that many other genes not previously examined with RT-PCR or IF, were found to be equally expressed between the two types of cells, namely, the GLSCs and the human embryonic germ cells. These are POU5F1, TDGF, GABRB3, FGF4 and TERT. POU5F1 and TDGF. These genes are commonly known to associate with pluripotency and they become strongly downregulated upon differentiation of stem cells, providing reliable stem cell markers.

Conclusively, the results suggest that amniotic fluid cells cultured according to the composition and methods of the present invention, specifically the GLSCs and human embryonic germ cells share common expression patterns, particularly for genes associated with undifferentiated pluripotent status of embryonic stem and embryonic germ cells.

The above-described embodiments are intended to be examples of the present invention and alterations and modifications may be effected thereto, by those of skill in the art, without departing from the scope of the invention which is defined solely by the claims appended hereto.

TABLE 1

| Purified | | | | |
|---|---|---|---|---|
| Antibody | Secondary | Isotype | Applications | Company |
| c-kit-PE | | PE-conjugated | Flow cytometry | abcam |
| Mouse monoclonal | | mouse IgG1 | | |
| (ab11290) | | isotype | | |
| | | control | | |
| | | (ab18429) | | |
| Oct-3/4-PE | | Rat IgG2B | Flow cytometry | R&D |
| Monoclonal | | (IC013P) | | |
| (IC1759) | | | | |
| SSE1-PE | | IgM-PE | Flow cytometry | Santa Cruz |
| (sc-21702) | | (sc-2870) | | |
| SSEA-4 (813-70) | Goat-anti-mouse-FITC | Normal | Flow cytometry | Santa Cruz |
| (sc-21704) | (sc2081) | Mouse IgG3- | | |
| | | FITC | | |
| | | (sc-2858) | | |
| DAZL (Y-15) | donkey anti-goat IgG- | Goat IgG- | Flow cytometry | Santa Cruz |
| Goat | FITC | FITC | | |
| polyclonal | (sc-2024) | (sc-3988) | | |
| (sc-27332) | | | | |
| Tra-1-81 | Goat anti-mouse IgM- | Normal mouse | Flow cytometry | Santa Cruz |
| Mouse | FITC | IgM-FITC | | |
| monoclonal | (sc-2082) | (sc-2859) | | |
| (sc-21706) | | | | |

| | | |
|---|---|---|
| FcR Blocking reagent | Blocks FcRs | MACS |
| (130 059901) | | |

TABLE 2

| | % pos. cells | STDEV |
|---|---|---|
| c-kit | 21.73 | 1.99 |
| DAZL | 34.18 | 8.17 |

TABLE 3

| | | | Annealing | |
|---|---|---|---|---|
| | | | Temperature | Product |
| mRNA | Primers 5′-3′ | Sequence | (° C.) | size |
| DAZL | forward | CCA CCA CAG TTT CAG AAT GTC | 55 | 593 |
| | | (SEQ ID No: 1) | | |
| | reverse | CAA AGT TTG AGT GTG ATT TAC CA | 55 | |
| | | (SEQ ID No: 2) | | |
| Oct-4 | forward outer | GAG GAA GCT GAC AAC AAT GAA | 55 | 249 |
| | | (SEQ ID No: 3) | | |
| | reverse outer | GGT TTT CTT TCC CTA GCT CCT | 55 | |
| | | (SEQ ID No: 4) | | |
| | forward inner | CAG GAG ATA TGC AAA GCA GAA | 55 | |
| | | (SEQ ID No: 5) | | |
| | reverse inner | AGC CTC AAA ATC CTC TCG TT | 55 | |
| | | (SEQ ID No: 6) | | |

TABLE 4

| | | | | Array # |
|---|---|---|---|---|
| Gene# | Name | UniProt/trEMBL | RefSeq | 4800038 |
| 43 | TNFR1: (TNFRSF1A OR TNFR1 OR TNFAR | P19438 | NM_001065 | 0.76/10% |
| | OR TNFR-1) TUMOR NECROSIS FACTOR | | | |
| | RECEPTOR SUPERFAMILY MEMBER 1A | | | |
| | PRECURSOR (TUMOR NECROSIS FACTOR | | | |
| | RECEPTOR 1) (TUMOR NECROSIS FACTOR | | | |
| | BINDING PROTEIN 1) (TBPI) (P60) (TNF-R1) | | | |
| | (TNF-RI) (P55) (CD120A). | | | |
| 47 | ACTA2: (ACTA2 OR ACTSA OR ACTVS) | P62736 | NM_001613 | 0.62/12% |
| | AORTIC SMOOTH MUSCLE (ALPHA-ACTIN | | | |
| | 2). | | | |
| 49 | TUBA_HUMAN: ((TUBA1B) AND (TUBA1A) | P68363 | NM_006009 | 1.52/12% |
| | AND (TUBA1C)) TUBULIN ALPHA- | Q9BQE3 | NM_006082 | |
| | UBIQUITOUS CHAIN (ALPHA-TUBULIN | Q71U36 | NM_032704 | |
| | UBIQUITOUS) (TUBULIN K-ALPHA-1) | | NR_003063 | |
| | (TUBA6) (TUBULIN ALPHA-6 CHAIN) | | | |
| | (ALPHA-TUBULIN 6) (TUBA3) (TUBULIN | | | |
| | ALPHA-3 CHAIN) (ALPHA-TUBULIN 3) | | | |
| | (TUBULIN B-ALPHA-1). | | | |
| 55 | TUBB_HUMAN: (TUBB OR TUBB5) | P07437 | NM_178014 | |
| | TUBULIN BETA CHAIN (TUBULIN BETA-5 | | | |
| | CHAIN). | | | |
| 67 | BRCA1: (BRCA1 OR RNF53) BREAST | P38398 | NM_007294 | |
| | CANCER TYPE 1 SUSCEPTIBILITY | | NM_007295 | |
| | PROTEIN (RING FINGER PROTEIN 53). | | NM_007296 | |
| | | | NM_007297 | |
| | | | NM_007298 | |
| | | | NM_007299 | |
| | | | NM_0 | |
| 87 | CDKN1A: (CDKN1A OR CDKN1 OR CIP1 OR | Q9BUT4 | NM_000389 | 0.69/9% |
| | WAF1 OR MDA6 OR SDI1 OR PIC1 OR | P38936 Q14010 | NM_078467 | |
| | CAP20) CYCLIN-DEPENDENT KINASE | | | |
| | INHIBITOR 1 (MELANOMA | | | |
| | DIFFERENTIATION ASSOCIATED PROTEIN | | | |
| | 6) (MDA-6) (P21) (CDK-INTERACTING | | | |
| | PROTEIN 1). | | | |
| 89 | CDKN1B: (CDKN1B OR KIP1) CYCLIN- | Q9BUS6 | NM_004064 | |
| | DEPENDENT KINASE INHIBITOR 1B | P46527 Q16307 | | |
| | (CYCLIN-DEPENDENT KINASE INHIBITOR | | | |
| | P27) (P27KIP1). | | | |
| 91 | P53: (TP53 OR P53) CELLULAR TUMOR | Q15086 Q15088 | NM_000546 | 1.04/20% |
| | ANTIGEN P53 (TUMOR SUPPRESSOR P53) | Q16535 Q16807 | | |
| | (PHOSPHOPROTEIN P53). | Q16808 Q16809 | | |
| | | Q16810 Q16811 | | |
| | | Q86UG1 Q | | |
| 106 | TNFSF11: (TNFSF11 OR RANKL OR | Q9P2Q3 | NM_003701 | |
| | TRANCE OR OPGL) TUMOR NECROSIS | O14788 O14723 | NM_033012 | |
| | FACTOR LIGAND SUPERFAMILY MEMBER | Q96Q17 | | |
| | 11 (RECEPTOR ACTIVATOR OF NUCLEAR | | | |
| | FACTOR KAPPA B LIGAND) (RANKL) (TNF- | | | |
| | RELATED ACTIVATION-INDUCED | | | |
| | CYTOKINE) (TRANCE) | | | |
| | (OSTEOPROTEGERIN LIGAND) (OPGL) | | | |
| | (OSTEOCLAST D | | | |
| 118 | CCNB2: (CCNB2) CYCLIN B2 G2/MITOTIC | O95067 | NM_004701 | |
| | SPECIFIC CYCLIN B2. | | | |
| 120 | CCNC_1: (CCNC) CYCLIN C. | P24863 Q9H543 | NM_001013399 | 1.13/22% |
| | | | NM_005190 | |
| 128 | CCNE1: (CCNE1 OR CCNE) CYCLIN E G1/S | P24864 Q14091 | NM_001238 | |
| | SPECIFIC CYCLIN E1. | Q92501 | NM_057182 | |
| | | Q8NFG1 | | |
| 134 | CCNG2: (CCNG2) CYCLIN G2. | Q16589 | NM_004354 | |
| 138 | CCNE2: (CCNE2) G1/S-SPECIFIC CYCLIN E2. | O96020 O95439 | NM_004702 | |
| | | | NM_057735 | |
| | | | NM_057749 | |
| 139 | MAPK3: (MAPK3 OR PRKM3 OR ERK1) | P27361 | NM_002746 | |
| | MITOGEN-ACTIVATED PROTEIN KINASE 3 | | | |
| | (EC 2.7.1.—) (EXTRACELLULAR SIGNAL- | | | |
| | REGULATED KINASE 1) (ERK-1) (INSULIN- | | | |
| | STIMULATED MAP2 KINASE) (MAP | | | |
| | KINASE 1) (MAPK 1) (P44-ERK1) (ERT2) | | | |
| | (P44-MAPK) (MICROTUBULE-ASSOCIATED | | | |
| | PROTEIN-2 KINAS | | | |
| 143 | MAPK6: (MAPK6 OR PRKM6 OR ERK3) | Q16659 | NM_002748 | |
| | MITOGEN-ACTIVATED PROTEIN KINASE 6 | Q8IYN8 | | |
| | (EC 2.7.1.—) (EXTRACELLULAR SIGNAL- | Q68DH4 | | |
| | REGULATED KINASE 3) (ERK3) (P55- | | | |
| | MAPK). | | | |
| 149 | MAPK12: (MAPK12 OR SAPK3) MITOGEN- | P53778 Q14260 | NM_002969 | 1.03/8% |
| | ACTIVATED PROTEIN KINASE 12 | Q99588 Q99672 | | |
| | (EXTRACELLULAR SIGNAL-REGULATED | | | |
| | KINASE 6) (EC 2.7.1.—) (ERK6) (STRESS- | | | |
| | ACTIVATED PROTEIN KINASE-3) | | | |
| | (MITOGEN-ACTIVATED PROTEIN KINASE | | | |
| | P38 GAMMA) (MAP KINASE P38 GAMMA). | | | |
| 163 | MAPK14: (MAPK14 OR CSBP1 OR CSBP2 OR | Q16539 Q14084 | NM_001315 | 0.94/43% |
| | CSBP OR MXI2) MITOGEN-ACTIVATED | Q13083 O60776 | NM_139012 | |
| | PROTEIN KINASE 14 (EC 2.7.1.37) | Q8TDX0 | NM_139013 | |
| | (MITOGEN-ACTIVATED PROTEIN KINASE | | NM_139014 | |
| | P38ALPHA) (MAP KINASE P38ALPHA) | | | |
| | (CYTOKINE SUPPRESSIVE ANTI- | | | |
| | INFLAMMATORY DRUG BINDING | | | |
| | PROTEIN) (CSAID BINDING PROTEIN) (C | | | |
| 165 | MAPK11: (MAPK11 OR PRKM11 OR SAPK2) | O15472 Q15759 | NM_002751 | 0.79/52% |
| | MITOGEN-ACTIVATED PROTEIN KINASE | O00284 | NM_138993 | |
| | 11 (EC 2.7.1.37) (MITOGEN-ACTIVATED | Q2XNF2 | | |
| | PROTEIN KINASE P38 BETA) (MAP KINASE | | | |
| | P38 BETA) (P38B) (P38-2) (STRESS- | | | |
| | ACTIVATED PROTEIN KINASE-2). | | | |
| 211 | CASP8_1: (MCH5 OR CASP8) CASPASE 8 | Q9UQ81 | NM_001228 | |
| | PRECURSOR (EC 3.4.22.—) (ICE-LIKE | O14676 Q8TDI1 | NM_033355 | |
| | APOPTOTIC PROTEASE 5) (MORT1- | Q8TDI2 | NM_033356 | |
| | ASSOCIATED CED-3 HOMOLOG) (MACH) | Q8TDI3 | | |
| | (FADD HOMOLOGOUS ICE/CED-3-LIKE | Q8TDI4 | | |
| | PROTEASE) (FLICE) (APOPTOTIC | Q8TDI5 | | |
| | CYSTEINE PROTEASE) (APOPTOTIC | Q96T22 | | |
| | PROTEASE MCH-5) (CAP4). | Q9C0K4 Q | | |
| 234 | NGFR: (NGFR OR TNFRSF16) LOW- | P08138 | NM_002507 | 1.05/20% |
| | AFFINITY NERVE GROWTH FACTOR | | | |
| | RECEPTOR PRECURSOR (NGF RECEPTOR) | | | |
| | (GP80-LNGFR) (P75 ICD) (LOW AFFINITY | | | |
| | NEUROTROPHIN RECEPTOR P75NTR) | | | |
| | (CD271 ANTIGEN). | | | |
| 241 | TNFSF4: (TNFSF4 OR TXGP1) OX40 LIGAND | Q9HCN9 | NM_003326 | |
| | (OX40L) (GLYCOPROTEIN GP34) (TAX- | P23510 | | |
| | TRANSCRIPTIONALLY ACTIVATED | | | |
| | GLYCOPROTEIN 1) (CD252 ANTIGEN). | | | |
| 251 | TNFRSF1B: (TNFRSF1B OR TNFR2 OR | Q6YI29 Q16042 | NM_001066 | |
| | TNFBR OR TNFR-2) TUMOR NECROSIS | Q9UIH1 P20333 | | |
| | FACTOR RECEPTOR SUPERFAMILY | | | |
| | MEMBER 1B PRECURSOR (TUMOR | | | |
| | NECROSIS FACTOR RECEPTOR 2) (TUMOR | | | |
| | NECROSIS FACTOR BINDING PROTEIN 2) | | | |
| | (TBPII) (P80) (TNF-R2) (P75) (CD120B) | | | |
| | (ETANERCEPT). | | | |
| 301 | CYPA: (PPIA OR CYPA) CYCLOPHILIN 1 | P62937 Q6IBU5 | NM_021130 | |
| | PEPTIDYL-PROLYL CIS-TRANS | Q3KQW3 | | |
| | ISOMERASE A (EC 5.2.1.8) (PPIASE) | | | |
| | (ROTAMASE) (CYCLOPHILIN A) | | | |
| | (CYCLOSPORIN A-BINDING PROTEIN). | | | |
| 303 | ICAM2: (ICAM2 OR ICAM-2) | Q14600 P13598 | NM_000873 | 1.17/21% |
| | INTERCELLULAR ADHESION MOLECULE-2 | | | |
| | PRECURSOR (ICAM-2) (CD102) | | | |
| | (LYMPHOCYTE FUNCTION-ASSOCIATED | | | |
| | AG-1 COUNTER-RECEPTOR). | | | |
| 305 | ITGAE: (ITGAE) INTEGRIN ALPHA-E | Q9NZU9 | NM_002208 | 1.21/4% |
| | PRECURSOR (MUCOSAL LYMPHOCYTE-1 | P38570 | | |
| | ANTIGEN) (HML-1 ANTIGEN) (CD103 | | | |
| | ANTIGEN) (INTEGRIN ALPHA-IEL) | | | |
| | (INTEGRIN ALPHA M290). | | | |
| 307 | ITGB4: (ITGB4) INTEGRIN BETA-4 | O15339 O15340 | NM_000213 | |
| | PRECURSOR (GP150) (CD104). | O15341 O14691 | NM_001005619 | |
| | | Q9UIQ4 | NM_001005731 | |
| | | O14690 P16144 | | |
| 309 | ENG: (ENG OR END) ENDOGLIN | Q14926 P17813 | NM_000118 | |
| | PRECURSOR (CD105 ANTIGEN) (CELL | Q14248 | | |
| | SURFACE MJ7/18 ANTIGEN). | | | |
| 311 | VCAM1: (VCAM1 OR L1CAM OR VCAM-1) | Q6NUP8 | NM_001078 | 0.24/66% |
| | VASCULAR CELL ADHESION PROTEIN 1 | P19320 | NM_080682 | |
| | PRECURSOR (V-CAM 1) (CD106 ANTIGEN) | | | |
| | (INCAM-100). | | | |
| 322 | KIT: (KIT OR SL) MAST/STEM CELL | P10721 | NM_000222 | 1.20/13% |
| | GROWTH FACTOR RECEPTOR PRECURSOR | Q9UM99 | | |
| | (EC 2.7.1.112) (SCFR) (PROTO-ONCOGENE | | | |
| | TYROSINE-PROTEIN KINASE KIT) (C-KIT) | | | |
| | (CD117 ANTIGEN) (C-KIT RECEPTOR | | | |
| | TYROSINE KINASE). | | | |
| 324 | IFNGR1: (IFNGR1 OR IFNGR) INTERFERON- | P15260 | NM_000416 | 0.50/25% |
| | GAMMA RECEPTOR ALPHA CHAIN | | | |
| | PRECURSOR (CDW119) (CD119). | | | |
| 326 | IL1R1: (IL1R1 OR IL1RA OR IL1R) | P14778 | NM_000877 | |
| | INTERLEUKIN-1 RECEPTOR, TYPE I | | | |
| | PRECURSOR (IL-1R-1) (IL-1R-ALPHA) (P80) | | | |
| | (ANTIGEN CD121A). | | | |
| 328 | IL1R2: (IL1R2 OR IL1RB) INTERLEUKIN-1 | Q9UE68 P27930 | NM_004633 | |
| | RECEPTOR, TYPE II PRECURSOR (IL-1R-2) | | NM_173343 | |
| | (IL-1R-BETA) (ANTIGEN CDW121B). | | | |
| 332 | IL3RA: ((IL3RAX OR IL3RA OR IL3R OR | P26951 | NM_002183 | |
| | IL3RX) AND (IL3RAY OR IL3RA OR IL3R OR | | | |
| | IL3RY)) INTERLEUKIN-3 RECEPTOR | | | |
| | ALPHA CHAIN PRECURSOR (IL-3R-ALPHA) | | | |
| | (CD123 ANTIGEN). | | | |
| 333 | IL4R: (IL4R OR IL4RA OR 582J2.1) | Q9H181 | NM_000418 | 1.16/42% |
| | INTERLEUKIN-4 RECEPTOR ALPHA CHAIN | Q9H182 | | |
| | PRECURSOR (IL-4R-ALPHA) (CD124 | Q9H183 | | |
| | ANTIGEN) [CONTAINS: SOLUBLE | Q9H184 | | |
| | INTERLEUKIN-4 RECEPTOR ALPHA CHAIN | Q9H185 | | |
| | (SIL4RALPHA/PROT) (IL-4-BINDING | Q9H186 | | |
| | PROTEIN) (IL4-BP)]. | Q9H187 | | |
| | | Q9H188 | | |
| | | Q96P01 P | | |
| 337 | IL6R: (L6RA OR IL6R) INTERLEUKIN-6 | Q16202 P08887 | NM_000565 | |
| | RECEPTOR ALPHA CHAIN PRECURSOR (IL- | Q53EQ7 | NM_181359 | |
| | 6R-ALPHA) (CD126 ANTIGEN) (IL-6R 1). | Q5FWG2 | | |
| | | Q5VZ23 | | |
| 339 | IL7R: (IL7R) INTERLEUKIN-7 RECEPTOR | Q9UPC1 | NM_002185 | 0.81/— % |
| | ALPHA CHAIN PRECURSOR (IL-7R-ALPHA) | P16871 Q6SV45 | | |
| | (CDW127) (CD127 ANTIGEN). | | | |
| 343 | IL6ST: (IL6ST) INTERLEUKIN-6 RECEPTOR | Q9UQ41 | NM_002184 | 0.73/2% |
| | BETA CHAIN PRECURSOR (IL-6R-BETA) | P40189 | NM_175767 | |
| | (INTERLEUKIN 6 SIGNAL TRANSDUCER) | | | |
| | (MEMBRANE GLYCOPROTEIN 130) (GP130) | | | |
| | (ONCOSTATIN M RECEPTOR) (CDW130) | | | |
| | (CD130 ANTIGEN). | | | |
| 345 | CSF2RB: (CSF2RB OR IL5RB OR IL3RB OR | P32927 | NM_000395 | 0.96/10% |
| | RIL-3ROR CSF2RB1 OR AIC2B OR IL3RB1) | | | |
| | CYTOKINE RECEPTOR COMMON BETA | | | |
| | CHAIN PRECURSOR (CDW131 ANTIGEN) | | | |
| | (CD131) (GM-CSF/IL-3/IL-5 RECEPTOR | | | |
| | COMMON BETA-CHAIN) (RIL-3R<BETA>) | | | |
| | (INTERLEUKIN-3 RECEPTOR BETA- | | | |
| | SUBUNIT) (CSF2RB2 OR | | | |
| 349 | FLT3: (FLT3 OR STK1 OR FLT-3 OR FLK-2) | P36888 Q13414 | NM_004119 | |
| | FL CYTOKINE RECEPTOR PRECURSOR (EC | | | |
| | 2.7.1.112) (TYROSINE-PROTEIN KINASE | | | |
| | RECEPTOR FLT3) (STEM CELL TYROSINE | | | |
| | KINASE 1) (STK-1) (CD135 ANTIGEN) | | | |
| | (TYROSINE-PROTEIN KINASE RECEPTOR | | | |
| | FLK-2) (FETAL LIVER KINASE 2). | | | |
| 355 | PDGFRA: (PDGFRA) ALPHA PLATELET- | Q96KZ7 P16234 | NM_006206 | |
| | DERIVED GROWTH FACTOR RECEPTOR | | | |
| | PRECURSOR (EC 2.7.1.112) (PDGF-R- | | | |
| | ALPHA) (CD140A ANTIGEN). | | | |
| 357 | PDGFRB: (PDGFRB OR PDGFR) BETA | Q8N5L4 P09619 | NM_002609 | 1.19/24% |
| | PLATELET-DERIVED GROWTH FACTOR | | | |
| | RECEPTOR PRECURSOR (EC 2.7.1.112) | | | |
| | (PDGF-R-BETA) (CD140B ANTIGEN). | | | |
| 359 | THBD: (THBD OR THRM) | P07204 Q9UC32 | NM_000361 | |
| | THROMBOMODULIN PRECURSOR | | | |
| | (FETOMODULIN) (TM) (CD141 ANTIGEN) | | | |
| | (BDCA-3) (BDCA3). | | | |
| 361 | F3: (F3 OR CF3 OR CF-3) TISSUE FACTOR | P13726 | NM_001993 | 0.30/10% |
| | PRECURSOR (TF) (COAGULATION FACTOR | Q6FHG2 | | |
| | III) (THROMBOPLASTIN) (CD142 | | | |
| | ANTIGEN). | | | |
| 365 | CDH5: (CDH5) VASCULAR ENDOTHELIAL- | P33151 | NM_001795 | 1.07/10% |
| | CADHERIN PRECURSOR (VE-CADHERIN) | | | |
| | (CADHERIN-5) (7B4 ANTIGEN) (CD144 | | | |
| | ANTIGEN) (CDH5). | | | |
| 367 | MCAM: (MCAM OR MUC18) CELL | P43121 O95812 | NM_006500 | 1.15/19% |
| | SURFACE GLYCOPROTEIN MUC18 | Q59E86 | | |
| | PRECURSOR (MELANOMA-ASSOCIATED | Q6PHR3 | | |
| | ANTIGEN MUC18) (MELANOMA- | Q6ZTR2 | | |
| | ASSOCIATED ANTIGEN A32) (S-ENDO 1 | | | |
| | ENDOTHELIAL-ASSOCIATED ANTIGEN) | | | |
| | (CD146 ANTIGEN) (MELANOMA | | | |
| | ADHESION MOLECULE) (S- | | | |
| | GICERIN/MUC18) (L-GICERIN/MUC18 | | | |
| 385 | SELPLG: (SELPLG) P-SELECTIN | Q14242 Q12775 | NM_003006 | 1.63/20% |
| | GLYCOPROTEIN LIGAND 1 PRECURSOR | | | |
| | (PSGL-1) (SELECTIN P LIGAND) (CD162 | | | |
| | ANTIGEN). | | | |
| 387 | ALCAM: (ALCAM OR MEMD) CD166 | Q13740 O60892 | NM_001627 | 0.50/12% |
| | ANTIGEN PRECURSOR (ACTIVATED | Q1HGM8 | | |
| | LEUKOCYTE-CELL ADHESION | Q1HGM9 | | |
| | MOLECULE) (ALCAM) (HB2) (KG-CAM) | | | |
| | (DM-GRASP PROTEIN). | | | |
| 389 | ITGAV: (ITGAV OR VNRA) VITRONECTIN | P06756 | NM_002210 | |
| | RECEPTOR ALPHA SUBUNIT PRECURSOR | | | |
| | (INTEGRIN ALPHA-V) (CD51). | | | |
| 397 | NCAM1_1: (NCAM1 OR NCAM) NEURAL | P13592 P13593 | NM_000615 | |
| | CELL ADHESION MOLECULE, 140 KDA | Q16180 Q15829 | NM_001076682 | |
| | ISOFORM PRECURSOR (N-CAM 140) | P13591 | NM_181351 | |
| | (NCAM-140) (CD56 ANTIGEN) (NEURAL | | | |
| | CELL ADHESION MOLECULE, | | | |
| | PHOSPHATIDYLINOSITOL-LINKED | | | |
| | ISOFORM PRECURSOR) (N-CAM 120) | | | |
| | (NCAM-120) (CD56 ANTIGEN) (NEURAL | | | |
| | CELL ADHES | | | |
| 399 | CD58_HUMAN: (CD58 OR LFA3) | Q96KI9 P19256 | NM_001779 | |
| | LYMPHOCYTE FUNCTION-ASSOCIATED | | | |
| | ANTIGEN 3 PRECURSOR (AG3) (ANTIGEN | | | |
| | CD58) (SURFACE GLYCOPROTEIN LFA-3). | | | |
| 401 | ITGB3: (ITGB3 OR GP3A) INTEGRIN BETA-3 | Q14648 O15495 | NM_000212 | |
| | PRECURSOR (PLATELET MEMBRANE | P05106 Q13413 | | |
| | GLYCOPROTEIN IIIA) (GPIIIA) (CD61 | Q16499 Q12806 | | |
| | ANTIGEN). | | | |
| 403 | SELE: (SELE OR ELAM1 OR ELAM-1) E- | P16581 P16111 | NM_000450 | |
| | SELECTIN PRECURSOR (ENDOTHELIAL | | | |
| | LEUKOCYTE ADHESION MOLECULE 1) | | | |
| | (ELAM-1) (LEUKOCYTE-ENDOTHELIAL | | | |
| | CELL ADHESION MOLECULE 2) (LECAM2) | | | |
| | (CD62E). | | | |
| 405 | SELL: (SELL OR LYAM1 OR LNHR OR LY- | P14151 P15023 | NM_000655 | |
| | 22) L-SELECTIN PRECURSOR (LYMPH | | | |
| | NODE HOMING RECEPTOR) (LEUKOCYTE | | | |
| | ADHESION MOLECULE-1) (LAM-1) | | | |
| | (LEUKOCYTE SURFACE ANTIGEN LEU-8) | | | |
| | (TQ1) (GP90-MEL) (LEUKOCYTE- | | | |
| | ENDOTHELIAL CELL ADHESION | | | |
| | MOLECULE 1) (LECAM1) (CD62L) (LY-22) | | | |
| 416 | CD68: (CD68) MACROSIALIN PRECURSOR | Q96BI7 P34810 | NM_001040059 | 0.84/— % |
| | (CD68 ANTIGEN) (GP110). | | NM_001251 | |
| 422 | TFRC_MIDDLE: (TFRC) TRANSFERRIN | Q9UK21 | NM_003234 | 1.00/— % |
| | RECEPTOR PROTEIN (TFR1) (TR) (TFR) | Q9UCU5 | | |
| | (TRFR) (CD71 ANTIGEN) (T9) (P90). | Q9UDF9 | | |
| | | Q9UCN0 | | |
| | | P02786 Q59G55 | | |
| 426 | NT5: (NT5E OR NT5 OR NTE) 5′- | O75520 P21589 | NM_002526 | 0.56/13% |
| | NUCLEOTIDASE PRECURSOR (EC 3.1.3.5) | | | |
| | (ECTO-NUCLEOTIDASE) (5′-NT) (CD73 | | | |
| | ANTIGEN). | | | |
| 438 | KAI1: (KAI1 OR CD82 OR SAR2) CD82 | P27701 | NM_001024844 | 0.28/10% |
| | ANTIGEN (INDUCIBLE MEMBRANE | | NM_002231 | |
| | PROTEIN R2) (C33 ANTIGEN) (IA4) | | | |
| | (METASTASIS SUPPRESSOR KANGAI 1) | | | |
| | (SUPPRESSOR OF TUMORIGENICITY-6). | | | |
| 446 | PLAUR: (PLAUR OR UPAR OR MO3) | Q03405 Q12876 | NM_002659 | 1.50/7% |
| | UROKINASE PLASMINOGEN ACTIVATOR | Q15845 Q16887 | | |
| | SURFACE RECEPTOR, GPI-ANCHORED | Q9NYC8 | | |
| | FORM PRECURSOR (U-PAR) (UPAR) | Q9UD69 | | |
| | (MONOCYTE ACTIVATION ANTIGEN MO3) | Q9UEA6 | | |
| | (CD87 ANTIGEN). | Q9UM92 | | |
| | | Q9UMV0 Q | | |
| 451 | THY1: (THY1) THY-1 MEMBRANE | P04216 Q16008 | NM_006288 | 1.14/11% |
| | GLYCOPROTEIN PRECURSOR (THY-1 | Q9NSP1 | | |
| | ANTIGEN) (CDW90) (CD90 ANTIGEN). | | | |
| 464 | MME: (MME OR EPN) NEPRILYSIN (EC | P08473 | NM_000902 | 0.73/— % |
| | 3.4.24.11) (NEUTRAL ENDOPEPTIDASE) | | NM_007287 | |
| | (NEP) (ENKEPHALINASE) (COMMON | | NM_007288 | |
| | ACUTE LYMPHOCYTIC LEUKEMIA | | NM_007289 | |
| | ANTIGEN) (CALLA) (NEUTRAL | | | |
| | ENDOPEPTIDASE 24.11) (CD10). | | | |
| 466 | ITGAL: (ITGAL OR CD11A OR LFA-1) | O43746 P20701 | NM_002209 | |
| | INTEGRIN ALPHA-L PRECURSOR | Q9UBC8 | | |
| | (LEUKOCYTE ADHESION GLYCOPROTEIN | Q45H73 | | |
| | LFA-1 ALPHA CHAIN) (LEUKOCYTE | | | |
| | FUNCTION ASSOCIATED MOLECULE 1, | | | |
| | ALPHA CHAIN) (CD11A) (INTEGRIN | | | |
| | ALPHA-L). | | | |
| 469 | ANPEP: (ANPEP OR PEPN OR APN OR CD13 | P15144 Q16728 | NM_001150 | |
| | OR LAP1 OR LAP-1) AMINOPEPTIDASE N | Q8IUK3 | | |
| | (EC 3.4.11.2) (MICROSOMAL | Q8IVH3 | | |
| | AMINOPEPTIDASE) (GP150) (MYELOID | Q9UCE0 | | |
| | PLASMA MEMBRANE GLYCOPROTEIN | | | |
| | CD13) (P161 MEMBRANE PROTEIN) | | | |
| | (MAPN) (RAPN) (ALANYL | | | |
| | AMINOPEPTIDASE) (AMINOPEPTIDASE M) | | | |
| | (APM) ( | | | |
| 475 | ITGB2: (ITGB2 OR CD18) INTEGRIN BETA- | P05107 Q16418 | NM_000211 | |
| | 2 PRECURSOR (CELL SURFACE ADHESION | | | |
| | GLYCOPROTEINS LFA-1/CR3/P150,95 | | | |
| | BETA-SUBUNIT) (CD18) (COMPLEMENT | | | |
| | RECEPTOR C3 BETA-SUBUNIT). | | | |
| 489 | CD24: (CD24 OR CD24A) SIGNAL | Q16257 P25063 | NM_013230 | 0.12/10% |
| | TRANSDUCER CD24 PRECURSOR (M1/69- | | | |
| | J11D HEAT STABLE ANTIGEN) (HSA) | | | |
| | (NECTADRIN) (LY-52) (X62 HEAT STABLE | | | |
| | ANTIGEN) (R13-AG). | | | |
| 499 | ITGB1: (ITGB1 OR FNRB) INTEGRIN BETA- | P78466 P78467 | NM_002211 | 0.49/12% |
| | 1 PRECURSOR (FIBRONECTIN RECEPTOR | Q13089 Q14647 | NM_033666 | |
| | BETA SUBUNIT) (CD29 ANTIGEN) | Q13090 Q13212 | NM_033667 | |
| | (INTEGRIN VLA-4 BETA SUBUNIT). | Q13091 Q14622 | NM_033668 | |
| | | P05556 | NM_033669 | |
| | | | NM_133376 | |
| 501 | PECAM1: (PECAM1 OR PECAM-1 OR | Q6LDA9 | NM_000442 | |
| | PECAM) PLATELET ENDOTHELIAL CELL | Q8TBH1 | | |
| | ADHESION MOLECULE PRECURSOR | Q96RF5 | | |
| | (PECAM-1) (CD31 ANTIGEN) (ENDOCAM) | Q96RF6 | | |
| | (GPIIA′). | Q9NP65 | | |
| | | Q9NPB7 | | |
| | | Q9NPG9 | | |
| | | Q9NQS9 | | |
| | | Q9NQT0 Q | | |
| 505 | CD33: (CD33) MYELOID CELL SURFACE | Q8TD24 P20138 | NM_001772 | |
| | ANTIGEN CD33 PRECURSOR (GP67) | | | |
| | (SIGLEC-3). | | | |
| 506 | CD34: (CD34) HEMATOPOIETIC | P28906 Q15970 | NM_001773 | 1.14/50% |
| | PROGENITOR CELL ANTIGEN CD34 | Q15971 | | |
| | PRECURSOR. | | | |
| 512 | CD37: (CD37) LEUKOCYTE ANTIGEN CD37. | P11049 | NM_001774 | 0.45/15% |
| 514 | CD38: (CD38) ADP-RIBOSYL CYCLASE 1 | Q96HY4 | NM_001775 | |
| | (EC 3.2.2.5) (CYCLIC ADP-RIBOSE | O00121 O00122 | | |
| | HYDROLASE 1) (CADPR HYDROLASE 1) | P28907 | | |
| | (LYMPHOCYTE DIFFERENTIATION | | | |
| | ANTIGEN CD38) (T10) (ACUTE | | | |
| | LYMPHOBLASTIC LEUKEMIA CELLS | | | |
| | ANTIGEN CD38) (NIM-R5 ANTIGEN) (I-19) | | | |
| | (CD38 HOMOLOG) (CD38H). | | | |
| 526 | ITGA2B: (ITGA2B OR ITGAB OR GP2B) | Q14443 O95366 | NM_000419 | 1.32/— % |
| | PLATELET MEMBRANE GLYCOPROTEIN | P08514 | | |
| | IIB PRECURSOR (GPIIB) (GPALPHA IIB) | | | |
| | (INTEGRIN ALPHA-IIB) (CD41). | | | |
| 538 | CD44_EX10-12_HUMAN: (CD44 OR LHR) | Q96J24 Q92493 | NM_000610 | |
| | CD44 ANTIGEN PRECURSOR | Q13961 Q13967 | NM_001001389 | |
| | (PHAGOCYTIC GLYCOPROTEIN I) (PGP-1) | Q13968 Q13980 | | |
| | (HUTCH-I) (EXTRACELLULAR MATRIX | Q15861 Q16064 | | |
| | RECEPTOR-III) (ECMR-III) (GP90 | Q16065 Q | | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTE | | | |
| 543 | CD47: (CD47 OR IAP) LEUKOCYTE | Q96A60 Q08722 | NM_001025079 | 1.01/9% |
| | SURFACE ANTIGEN CD47 PRECURSOR | Q53Y71 | NM_001777 | |
| | (ANTIGENIC SURFACE DETERMINANT | | NM_198793 | |
| | PROTEIN OA3) (INTEGRIN ASSOCIATED | | | |
| | PROTEIN) (IAP) (MER6) (ITGP) (INTEGRIN- | | | |
| | ASSOCIATED PROTEIN PRECURSOR). | | | |
| 547 | ITGA1_1: (ITGA1) INTEGRIN ALPHA-1 | P56199 | NM_181501 | 0.76/22% |
| | (LAMININ AND COLLAGEN RECEPTOR) | | | |
| | (VLA-1) (CD49A). | | | |
| 549 | ITGA1_2: (ITGA1) INTEGRIN ALPHA-1 | P56199 | NM_181501 | 0.09/13% |
| | (LAMININ AND COLLAGEN RECEPTOR) | | | |
| | (VLA-1) (CD49A). | | | |
| 550 | ITGA2: (ITGA2) INTEGRIN ALPHA-2 | Q14595 P17301 | NM_002203 | 1.26/8% |
| | PRECURSOR (PLATELET MEMBRANE | | | |
| | GLYCOPROTEIN IA) (GPIA) (COLLAGEN | | | |
| | RECEPTOR) (VLA-2 ALPHA CHAIN) | | | |
| | (CD49B). | | | |
| 552 | ITGA3: (ITGA3) INTEGRIN ALPHA-3 | P26006 | NM_002204 | 0.75/12% |
| | PRECURSOR (GALACTOPROTEIN B3) | | NM_005501 | |
| | (GAPB3) (VLA-3 ALPHA CHAIN) (CD49C). | | | |
| 554 | ITGA4: (ITGA4 OR VLA-4) INTEGRIN | P13612 | NM_000885 | |
| | ALPHA-4 PRECURSOR (INTEGRIN ALPHA- | | | |
| | IV) (VLA-4) (CD49D) (LYMPHOCYTE- | | | |
| | PEYER′S PATCH ADHESION MOLECULES | | | |
| | ALPHA SUBUNIT) (LPAM ALPHA | | | |
| | SUBUNIT). | | | |
| 556 | ITGA5: (ITGA5 OR FNRA) INTEGRIN | Q96HA5 | NM_002205 | 0.70/11% |
| | ALPHA-5 PRECURSOR (FIBRONECTIN | P08648 | | |
| | RECEPTOR ALPHA SUBUNIT) (INTEGRIN | | | |
| | ALPHA-F) (VLA-5) (CD49E). | | | |
| 558 | ITGA6: (ITGA6) INTEGRIN ALPHA-6 | P23229 Q14646 | NM_000210 | |
| | PRECURSOR (VLA-6) (CD49F) (INTA6) | Q16508 Q08443 | | |
| | (INTEGRIN ALPHA 6 SUBCHAIN). | Q9UN03 | | |
| 563 | ICAM1: (ICAM1 OR ICAM-1) | Q96B50 P05362 | NM_000201 | |
| | INTERCELLULAR ADHESION MOLECULE 1 | | | |
| | PRECURSOR (ICAM-1) (MAJOR GROUP | | | |
| | RHINOVIRUS RECEPTOR) (CD54) (MALA- | | | |
| | 2). | | | |
| 567 | CD7: (CD7) T-CELL ANTIGEN CD7 | P09564 | NM_006137 | |
| | PRECURSOR (GP40) (T-CELL LEUKEMIA | | | |
| | ANTIGEN) (TP41) (LEU-9). | | | |
| 573 | CD9: (CD9 OR MIC3) CD9 ANTIGEN (P24) | Q96ES4 P21926 | NM_001769 | |
| | (LEUKOCYTE ANTIGEN MIC3) (MOTILITY- | | | |
| | RELATED PROTEIN) (MRP-1). | | | |
| 591 | HTR1A: (HTR1A) 5- | Q6LAE7 | NM_000524 | |
| | HYDROXYTRYPTAMINE 1A RECEPTOR (5- | P08908 | | |
| | HT-1A) (SEROTONIN RECEPTOR) (5-HT1A) | | | |
| | (G-21). | | | |
| 593 | HTR1B: (HTR1B OR HTR1DB) 5- | P28222 | NM_000863 | 1.65/— % |
| | HYDROXYTRYPTAMINE 1B RECEPTOR (5- | Q4VAY7 | | |
| | HT-1B) (SEROTONIN RECEPTOR) (5-HT-1D- | | | |
| | BETA) (S12). | | | |
| 595 | HTR1D: (HTR1D OR HTR1DA) 5- | P28221 | NM_000864 | |
| | HYDROXYTRYPTAMINE 1D RECEPTOR (5- | | | |
| | HT-1D) (SEROTONIN RECEPTOR) (5-HT-1D- | | | |
| | ALPHA) (GPCR14) 5- | | | |
| | HYDROXYTRYPTAMINE 1D RECEPTOR (5- | | | |
| | HT-1D) (SEROTONIN RECEPTOR) (GPCR14) | | | |
| | (HTR1DB) (5-HYDROXYTRYPTAMINE 1D | | | |
| | BETA RECEPTOR) (SEROTONIN | | | |
| | RECEPTOR). | | | |
| 598 | HTR1F: (HTR1F OR HTR1EL) 5- | P30939 | NM_000866 | |
| | HYDROXYTRYPTAMINE 1F RECEPTOR (5- | | | |
| | HT-1F) (SEROTONIN RECEPTOR). | | | |
| 602 | HTR2B: (HTR2B) 5- | P41595 Q62221 | NM_000867 | |
| | HYDROXYTRYPTAMINE 2B RECEPTOR (5- | Q53TI1 Q6P523 | | |
| | HT-2B) (SEROTONIN RECEPTOR). | | | |
| 606 | HTR4: (HTR4) 5-HYDROXYTRYPTAMINE 4 | Q9UBM6 | NM_000870 | |
| | RECEPTOR (5-HT-4) (SEROTONIN | Q9UQR6 | NM_199453 | |
| | RECEPTOR) (5-HT4) (FRAGMENT). | Q9UE22 | | |
| | | Q9UE23 | | |
| | | Q9UBT4 | | |
| | | Q9NY73 | | |
| | | Q9H199 | | |
| | | Q96KH9 | | |
| | | Q96KI0 Q | | |
| 614 | HTR6: (HTR6) 5-HYDROXYTRYPTAMINE 6 | P50406 Q13640 | NM_000871 | 0.92/5% |
| | RECEPTOR (5-HT-6) (SEROTONIN | | | |
| | RECEPTOR). | | | |
| 616 | HTR7: (HTR7) 5-HYDROXYTRYPTAMINE 7 | P34969 P78516 | NM_000872 | |
| | RECEPTOR (5-HT-7) (5-HT-X) (SEROTONIN | P78336 P78372 | NM_019859 | |
| | RECEPTOR) (5HT7). | | NM_019860 | |
| 632 | CHRM1: (CHRM1) MUSCARINIC | P11229 | NM_000738 | |
| | ACETYLCHOLINE RECEPTOR M1. | | | |
| 634 | CHRM2: (CHRM2) MUSCARINIC | P08172 Q9P1X9 | NM_000739 | 1.27/4% |
| | ACETYLCHOLINE RECEPTOR M2. | | | |
| 703 | DRD2: (DRD2) D(2) DOPAMINE RECEPTOR | P14416 | NM_000795 | |
| | | Q9NZR3 | NM_016574 | |
| | | Q9UPA9 | | |
| 705 | DRD3: (DRD3) D(3) DOPAMINE RECEPTOR. | P35462 | NM_000796 | |
| | | Q4VBM8 | NM_033658 | |
| | | | NM_033659 | |
| | | | NM_033660 | |
| | | | NM_033663 | |
| 711 | DRD5: (DRD5 OR DRD1B) D(1B) DOPAMINE | Q8NEQ8 | NM_000798 | |
| | RECEPTOR (D(5) DOPAMINE RECEPTOR) | P21918 | | |
| | (D1BETA DOPAMINE RECEPTOR). | | | |
| 713 | EDNRA: (EDNRA OR ETRA) ENDOTHELIN-1 | O43441 Q16432 | NM_001957 | |
| | RECEPTOR PRECURSOR (ET-A). | Q16433 | | |
| | | Q8TBH2 | | |
| | | P25101 | | |
| 715 | EDNRB: (EDNRB OR ETRB) ENDOTHELIN B | Q9UQK3 | NM_000115 | |
| | RECEPTOR PRECURSOR (ET-B) | P24530 O15343 | NM_003991 | |
| | (ENDOTHELIN RECEPTOR NON- | | | |
| | SELECTIVE TYPE). | | | |
| 812 | GJA1: (GJA1) GAP JUNCTION ALPHA-1 | Q9Y5I8 P17302 | NM_000165 | 1.03/17% |
| | PROTEIN (CONNEXIN 43) (CX43) (GAP | | | |
| | JUNCTION 43 KDA HEART PROTEIN) | | | |
| 816 | GJA4: (GJA4) GAP JUNCTION ALPHA-4 | Q9P106 P35212 | NM_002060 | 1.58/45% |
| | PROTEIN (CONNEXIN 37) (CX37). | Q9UNA9 | | |
| | | Q9UNB0 | | |
| | | Q9UNB1 | | |
| | | Q9Y5N7 | | |
| | | Q9UBL1 | | |
| 818 | GJA5: (GJA5) GAP JUNCTION ALPHA-5 | P36382 Q5T3B6 | NM_181703 | |
| | PROTEIN (CONNEXIN 40) (CX40). | Q5U0N6 | | |
| 820 | GJA7: (GJA7 OR CXN-45) GAP JUNCTION | P36383 | NM_005497 | 0.99/20% |
| | ALPHA-7 PROTEIN (CONNEXIN 45) (CX45) | | | |
| 829 | GJB5: (GJB5 OR CXN-31.1) GAP JUNCTION | Q9UPA3 | NM_005268 | |
| | BETA-5 PROTEIN (CONNEXIN 31.1) | O95377 | | |
| | (CX31.1). | | | |
| 845 | EAAT4: (SLC1A6 OR EAAT4) EXCITATORY | P48664 | NM_005071 | |
| | AMINO ACID TRANSPORTER 4 (SODIUM- | | | |
| | DEPENDENT GLUTAMATE/ASPARTATE | | | |
| | TRANSPORTER). | | | |
| 1088 | PTHR2: (PTHR2) PARATHYROID | Q8N429 P49190 | NM_005048 | |
| | HORMONE RECEPTOR PRECURSOR (PTH2 | | | |
| | RECEPTOR). | | | |
| 1089 | PTHR1: (PTHR1 OR PTHR) PARATHYROID | Q03431 | NM_000316 | 1.53/23% |
| | HORMONE/PARATHYROID HORMONE- | | | |
| | RELATED PEPTIDE RECEPTOR | | | |
| | PRECURSOR (PTH/PTHR RECEPTOR). | | | |
| 1191 | COL18A1_1: (COL18A1) COLLAGEN ALPHA | Q9Y6Q8 | NM_030582 | 1.11/7% |
| | 1(XVIII) CHAIN [CONTAINS: | Q9Y6Q7 | NM_130445 | |
| | ENDOSTATIN]. | Q9UK38 | | |
| | | P39060 | | |
| 1201 | FZD3: (FZD3) FRIZZLED 3 PRECURSOR | Q9NPG1 | NM_017412 | |
| | (FRIZZLED-3) (FZ-3) (HFZ3) (MFZ3) (RFZ3). | | | |
| 1210 | FZD4: (FZD4) WNT RECEPTOR FRIZZLED-4, | Q9ULV1 | NM_012193 | 1.34/32% |
| | FRIZZLED 4 PRECURSOR (FRIZZLED-4) (FZ- | Q6S9E4 | | |
| | 4) (HFZ4) (FZE4) (MFZ4) (RFZ4) (CD344 | | | |
| | ANTIGEN). | | | |
| 1248 | TP53BP1: (TP53BP1) TUMOR SUPPRESSOR | Q12888 | NM_005657 | 0.85/12% |
| | P53-BINDING PROTEIN 1 (P53-BINDING | Q5FWZ3 | | |
| | PROTEIN 1) (53BP1). | Q2M1Z7 | | |
| | | Q4LE46 | | |
| | | Q7Z3U4 | | |
| 1259 | SFRP2: (SFRP2 OR FKSG12 OR FRP2 OR | O14778 | NM_003013 | |
| | SARP1) SECRETED FRIZZLED-RELATED | Q9HAP5 | | |
| | PROTEIN 2 PRECURSOR (SFRP-2) | Q96HF1 | | |
| | (SECRETED APOPTOSIS-RELATED | | | |
| | PROTEIN 1) (SARP-1) (FRIZZLED-RELATED | | | |
| | PROTEIN 2) (FRP-2) (PANCREAS TUMOR- | | | |
| | RELATED PROTEIN FKSG12) | | | |
| | (UNQ361/PRO697). | | | |
| 1282 | BMP2: (BMP2 OR BMP2A OR BMP-2) BONE | P12643 | NM_001200 | 1.67/17% |
| | MORPHOGENETIC PROTEIN 2 PRECURSOR | | | |
| | (BMP-2) (BMP-2A). | | | |
| 1284 | BMP3: (BMP3 OR BMP-3) BONE | P12645 | NM_001201 | |
| | MORPHOGENETIC PROTEIN 3 PRECURSOR | | | |
| | (BMP-3) (OSTEOGENIN) (BMP-3A). | | | |
| 1287 | BMP6: (BMP6 OR BMP-6 OR VGR1) BONE | P22004 | NM_001718 | |
| | MORPHOGENETIC PROTEIN 6 PRECURSOR | | | |
| | (BMP 6). | | | |
| 1291 | BMP8A-BMP8B_HUMAN: (BMP8) BONE | P34820 | NM_001720 | 1.12/13% |
| | MORPHOGENETIC PROTEIN 8 PRECURSOR | Q9NUF0 | NM_181809 | |
| | (BMP-8) (BMP-8A) (BMP-8B) (OSTEOGENIC | Q53ZM7 | | |
| | PROTEIN 2) (OP 2). | | | |
| 1294 | CTGF: (CTGF OR HCS24) CONNECTIVE | P29279 | NM_001901 | 0.87/15% |
| | TISSUE GROWTH FACTOR PRECURSOR | Q96QX2 | | |
| | (HYPERTROPHIC CHONDROCYTE- | Q6LCY0 | | |
| | SPECIFIC PROTEIN 24). | Q96A79 | | |
| 1302 | GDF1: (GDF1 OR GDF-1) EMBRYONIC | P27539 O43344 | NM_001492 | |
| | GROWTH/DIFFERENTIATION FACTOR 1 | | | |
| | PRECURSOR (GDF 1). | | | |
| 1304 | GDF3: (GDF3) GROWTH DIFFERENTIATION | Q8NEJ4 | NM_020634 | |
| | FACTOR 3. (GDF3 OR GDF-3 OR VGR-2) | Q9NR23 | | |
| | GROWTH/DIFFERENTIATION FACTOR 3 | | | |
| | PRECURSOR (GDF-3) (VG-1-RELATED | | | |
| | PROTEIN 2). | | | |
| 1312 | GDF8: (GDF8 OR MSTN) | Q6B0H2 | NM_005259 | 1.34/29% |
| | GROWTH/DIFFERENTIATION FACTOR 8 | O14793 | | |
| | PRECURSOR (GDF-8) (MYOSTATIN). | | | |
| 1314 | GDF9: (GDF9) GROWTH/DIFFERENTIATION | O60383 | NM_005260 | |
| | FACTOR 9 PRECURSOR (GDF-9). | | | |
| 1316 | GDNF: (GDNF) GLIAL CELL LINE-DERIVED | Q9UP97 | NM_000514 | |
| | NEUROTROPHIC FACTOR PRECURSOR. | Q9UD33 | NM_199231 | |
| | | Q96L44 P39905 | NM_199234 | |
| 1326 | INHBB: (INHBB) INHIBIN BETA B CHAIN | Q8N1D3 | NM_002193 | |
| | PRECURSOR (ACTIVIN BETA-B CHAIN). | P09529 | | |
| 1348 | PDGFA: (PDGFA OR RPA1 OR PDGF1) PDGA | P04085 | NM_002607 | |
| | PLATELET-DERIVED GROWTH FACTOR, A | | NM_033023 | |
| | CHAIN PRECURSOR (PDGF A-CHAIN) | | | |
| | (PDGF-1) (PLATELET-DERIVED GROWTH | | | |
| | FACTOR ALPHA POLYPEPTIDE) (PDGF A- | | | |
| | CHAIN). | | | |
| 1350 | PDGFB: (PDGFB OR PDGF2 OR SIS) | Q9UF23 P01127 | NM_002608 | |
| | PLATELET-DERIVED GROWTH FACTOR B | P78431 | NM_033016 | |
| | CHAIN PRECURSOR (PDGF B-CHAIN) | | | |
| | (PLATELET-DERIVED GROWTH FACTOR | | | |
| | BETA POLYPEPTIDE) (PDGF-2) (C-SIS) | | | |
| | (BECAPLERMIN). | | | |
| 1430 | VEGFB: (VEGFB OR VRF) VASCULAR | Q16528 P49765 | NM_003377 | 1.16/35% |
| | ENDOTHELIAL GROWTH FACTOR B | | | |
| | PRECURSOR (VEGF-B) (VEGF RELATED | | | |
| | FACTOR). | | | |
| 1436 | VEGF: (VEGF OR VEGFA) VASCULAR | Q16889 O60720 | NM_001025366 | |
| | ENDOTHELIAL GROWTH FACTOR | O75875 | NM_001025367 | |
| | PRECURSOR (VEGF) (VASCULAR | Q9UL23 | NM_001025368 | |
| | PERMEABILITY FACTOR) (VPF)(VEGF A) | Q9UH58 | NM_001025369 | |
| | | Q9H1W9 | NM_001025370 | |
| | | Q9H1W8 | | |
| | | Q96L82 | | |
| | | Q96NW5 P | | |
| 1438 | VWF: (F8VWF OR VWF) VON | P04275 Q99806 | NM_000552 | |
| | WILLEBRAND FACTOR PRECURSOR. | | | |
| 1440 | CER1: (CER1 OR CER-L) CERBERUS 1 | O95813 Q6ISJ1 | NM_005454 | 1.49/11% |
| | (CERBERUS-LIKE). (CER1 OR CER-1 OR | Q6ISJ6 Q6ISQ2 | | |
| | CERR1) CERBERUS 1 (CERBERUS | Q6ISS1 | | |
| | HOMOLOG) CERBERUS-RELATED | | | |
| | PROTEIN (CERBERUS-RELATED 1. | | | |
| 1442 | WISP3: (WISP3 OR CCN6 OR DJ142L7.3 OR | O95389 | NM_003880 | |
| | LIBC) WNT1 INDUCIBLE SIGNALING | Q6UXH6 | NM_130396 | |
| | PATHWAY PROTEIN 3 PRECURSOR (WISP- | | NM_198239 | |
| | 3) (CONNECTIVE TISSUE GROWTH | | | |
| | FACTOR (NOV, GIG) LIKE PROTEIN | | | |
| | (WISP3) (CONNECTIVE TISSUE GROWTH | | | |
| | FACTOR RELATED PROTEIN WISP-3) | | | |
| | (LOST IN INFLAMMATORY BREAS | | | |
| 1447 | SLIT1: (SLIT1 OR KIAA0813 OR MEGF4) | Q8WWZ2 | NM_003061 | |
| | SLIT HOMOLOG 1 PROTEIN PRECURSOR | Q9UIL7 O75093 | | |
| | (SLIT-1) (MULTIPLE EPIDERMAL GROWTH | | | |
| | FACTOR-LIKE DOMAINS 4). | | | |
| 1465 | VEGFD: (FIGF OR VEGF-D) VASCULAR | O43915 | NM_004469 | |
| | ENDOTHELIAL GROWTH FACTOR D (C- | | | |
| | FOS INDUCED GROWTH FACTOR). | | | |
| 1485 | SYT11: (SYT11 OR KIAA0080) | Q9BT88 | NM_152280 | |
| | SYNAPTOTAGMIN-11 (SYNAPTOTAGMIN | Q68CT5 | | |
| | XI) (SYTXI). | Q8IXU3 | | |
| | | Q96SU2 | | |
| | | Q14998 | | |
| 1497 | PRKCB_1: (PRKCB1 OR PRKCB OR PKCB) | O43744 Q15138 | NM_002738 | |
| | PROTEIN KINASE C, BETA TYPE (EC | Q93060 Q9UJ33 | NM_212535 | |
| | 2.7.1.37) (PKC-BETA) (PKC-B). | Q9UJ30 | | |
| | | Q9UEH8 | | |
| | | Q9UE49 | | |
| | | Q9UE50 P05127 P | | |
| 1499 | PRKCB_2: (PRKCB1 OR PRKCB OR PKCB) | O43744 Q15138 | NM_002738 | |
| | PROTEIN KINASE C, BETA TYPE (EC | Q93060 Q9UJ33 | NM_212535 | |
| | 2.7.1.37) (PKC-BETA) (PKC-B). | Q9UJ30 | | |
| | | Q9UEH8 | | |
| | | Q9UE49 | | |
| | | Q9UE50 P05127 P | | |
| 1503 | PRKCE: (PRKCE OR PKCE) PROTEIN | Q9UE81 | NM_005400 | |
| | KINASE C, EPSILON TYPE (EC 2.7.1.—) | Q02156 Q53SL4 | | |
| | (NPKC-EPSILON). | Q53SM5 | | |
| 1507 | PRKCH: (PRKCH OR PKCL) PROTEIN | Q16246 P24723 | NM_006255 | 1.36/26% |
| | KINASE C, ETA TYPE (EC 2.7.1.—) (NPKC- | | | |
| | ETA) (PKC-L). | | | |
| 1552 | SYT1: (SYT1 OR SYT) SYNAPTOTAGMIN I | P21579 | NM_005639 | 1.45/43% |
| | (P65). | | | |
| 1623 | TIAM: (TIAM) T-LYMPHOMA INVASION | Q13009 | NM_003253 | |
| | AND METASTASIS INDUCING PROTEIN 1 | | | |
| | (TIAM1 PROTEIN). | | | |
| 1691 | ACTB: (ACTB) BETA1, CYTOPLASMIC | P60709 | NM_001101 | 0.90/5% |
| | (BETA-ACTIN) ACTIN, CYTOPLASMIC 1. | Q75MN2 | | |
| | | Q96B34 | | |
| 1710 | MAPT: (MAPT OR MTBT1 OR TAU) | P10636 P18518 | NM_005910 | |
| | MICROTUBULE-ASSOCIATED PROTEIN | Q14799 Q15551 | NM_016834 | |
| | TAU (NEUROFIBRILLARY TANGLE | Q9UQ96 | NM_016835 | |
| | PROTEIN) (PAIRED HELICAL FILAMENT- | Q15549 Q15550 | NM_016841 | |
| | TAU) (PHF-TAU). | Q9UDJ3 | | |
| | | Q9UMH0 | | |
| 1743 | APOE: (APOE) APOLIPOPROTEIN E | Q9P2S4 P02649 | NM_000041 | 0.23/106% |
| | PRECURSOR (APO-E). | | | |
| 1761 | SNCA: (SNCA OR NACP) ALPHA- | Q6IAU6 P37840 | NM_000345 | |
| | SYNUCLEIN (NON-A BETA COMPONENT | Q13701 Q4JHI3 | NM_007308 | |
| | OF AD AMYLOID) (NACP). | | | |
| 1765 | SNCG: (SNCG OR BCSG1) GAMMA- | O76070 O15104 | NM_003087 | 1.30/16% |
| | SYNUCLEIN (PERSYN) (BREAST CANCER- | Q96P61 | | |
| | SPECIFIC GENE 1 PROTEIN). | | | |
| 1811 | CYP2C9_HUMAN: (CYP2C9) CYTOCHROME | P11713 Q16756 | NM_000771 | 1.53/11% |
| | P450 2C9 (EC 1.14.14.1) (CYPIIC9) (P450 PB- | Q16872 P11712 | | |
| | 1) (P450 MP-4) (S-MEPHENYTOIN 4- | | | |
| | HYDROXYLASE) (P-450MP). | | | |
| 1915 | CSK: (CSK) TYROSINE-PROTEIN KINASE | P41240 Q6FGZ6 | NM_004383 | 1.51/17% |
| | CSK (EC 2.7.1.112) (C-SRC KINASE) | | | |
| | (PROTEIN-TYROSINE KINASE CYL). | | | |
| 1930 | PKCD: (PRKCD OR PKCD) PROTEIN | Q05655 Q15144 | NM_006254 | 1.36/15% |
| | KINASE C, DELTA TYPE (EC 2.7.1.—) (NPKC- | | NM_212539 | |
| | DELTA). | | | |
| 1953 | PIK3CG: (PIK3CG) | P48736 Q8IV23 | NM_002649 | |
| | PHOSPHATIDYLINOSITOL 3-KINASE | Q9BZC8 | | |
| | CATALYTIC SUBUNIT, GAMMA ISOFORM | | | |
| | (EC 2.7.1.137) (PI3-KINASE P110 SUBUNIT | | | |
| | GAMMA) (PTDINS-3-KINASE P110) (PI3K). | | | |
| 2009 | CXCR4: (CXCR4 OR LESTR OR CMKAR4 OR | Q9UKN2 | NM_003467 | |
| | SDF1R) C-X-C CHEMOKINE RECEPTOR | O60835 P61073 | | |
| | TYPE 4 (CXC-R4) (CXCR-4) (SDF-1 | P30991 P56438 | | |
| | RECEPTOR) (STROMAL CELL-DERIVED | | | |
| | FACTOR 1 RECEPTOR) (FUSIN) | | | |
| | (LEUKOCYTE-DERIVED SEVEN | | | |
| | TRANSMEMBRANE DOMAIN RECEPTOR) | | | |
| | (LCR1) (FB22) (NPYRL) (HM89) (CD184 | | | |
| | ANTI | | | |
| 2031 | GAD1_1: (GAD1 OR GAD) GLUTAMATE | Q99259 | NM_000817 | |
| | DECARBOXYLASE, 67 KDA ISOFORM (EC | Q9BU91 | | |
| | 4.1.1.15) (GAD-67) (67 KDA GLUTAMIC | Q9UHH4 | | |
| | ACID DECARBOXYLASE). | | | |
| 2035 | CALB1: (CALB1 OR CAB27) CALBINDIN | P05937 | NM_004929 | |
| | (VITAMIN D-DEPENDENT CALCIUM- | | | |
| | BINDING PROTEIN, AVIAN-TYPE) | | | |
| | (CALBINDIN D28) (D-28K). | | | |
| 2039 | EAAT1: (SLC1A3 OR EAAT1) EXCITATORY | P43003 | NM_004172 | |
| | AMINO ACID TRANSPORTER 1 (SODIUM- | | | |
| | DEPENDENT GLUTAMATE/ASPARTATE | | | |
| | TRANSPORTER 1) (GLIAL GLUTAMATE | | | |
| | TRANSPORTER) (GLAST1) | | | |
| 2043 | EAAT2_1: (SLC1A2 OR EAAT2 OR GLT1) | P43004 Q14417 | NM_004171 | |
| | EXCITATORY AMINO ACID | | | |
| | TRANSPORTER 2 (SODIUM-DEPENDENT | | | |
| | GLUTAMATE/ASPARTATE TRANSPORTER | | | |
| | 2). | | | |
| 2047 | GRIK1: (GRIK1 OR GLUR5) GLUTAMATE | Q86SU9 P39086 | NM_000830 | |
| | RECEPTOR, IONOTROPIC KAINATE 1 | Q13001 | NM_175611 | |
| | PRECURSOR (GLUTAMATE RECEPTOR 5) | | | |
| | (GLUR-5) (EXCITATORY AMINO ACID | | | |
| | RECEPTOR 3) (EAA3). | | | |
| 2049 | GRIK2: (GRIK2 OR GLUR6) GLUTAMATE | Q13002 | NM_021956 | |
| | RECEPTOR, IONOTROPIC KAINATE 2 | Q8WWS1 | NM_175768 | |
| | PRECURSOR (GLUTAMATE RECEPTOR 6) | Q96KS6 | | |
| | (GLUR-6) (EXCITATORY AMINO ACID | Q96KS7 | | |
| | RECEPTOR 4) (EAA4). | Q96KS8 | | |
| 2053 | GRIA1: (GRIA1 OR GLUR1 OR GLUH1) | P42261 | NM_000827 | 1.17/5% |
| | GLUTAMATE RECEPTOR 1 PRECURSOR | | | |
| | (GLUR-1) (GLUR-A) (GLUR-K1) | | | |
| | (GLUTAMATE RECEPTOR IONOTROPIC, | | | |
| | AMPA 1). | | | |
| 2055 | GRIA2: (GRIA2 OR GLUR2) GLUTAMATE | P42262 Q96FP6 | NM_000826 | |
| | RECEPTOR 2 PRECURSOR (GLUR-2) (GLUR- | | | |
| | B) (GLUR-K2) (GLUTAMATE RECEPTOR | | | |
| | IONOTROPIC, AMPA 2). | | | |
| 2104 | GDF10: (GDF10 OR BMP3B) BONE | Q9UCX6 | NM_004962 | |
| | MORPHOGENETIC PROTEIN 3B | P55107 | | |
| | PRECURSOR (BMP-3B) | | | |
| | (GROWTH/DIFFERENTIATION FACTOR | | | |
| | GDF-10) (BONE INDUCING PROTEIN) (BIP). | | | |
| 2106 | GDF5: (GDF5 OR CDMP1) | Q96SB1 P43026 | NM_000557 | |
| | GROWTH/DIFFERENTIATION FACTOR 5 | | | |
| | PRECURSOR (GDF-5) (CARTILAGE- | | | |
| | DERIVED MORPHOGENETIC PROTEIN 1) | | | |
| | (CDMP-1). | | | |
| 2108 | INHBA: (INHBA) INHIBIN BETA A CHAIN | P08476 Q14599 | NM_002192 | 0.38/8% |
| | PRECURSOR (ACTIVIN BETA-A CHAIN) | | | |
| | (ERYTHROID DIFFERENTIATION | | | |
| | PROTEIN) (EDF). | | | |
| 2179 | TGFB2: (TGFB2) TRANSFORMING | P61812 | NM_003238 | |
| | GROWTH FACTOR BETA 2 PRECURSOR | Q4VAV9 | | |
| | (TGF-BETA 2) (GLIOBLASTOMA-DERIVED | | | |
| | T-CELL SUPPRESSOR FACTOR) (G-TSF) | | | |
| | (BSC-1 CELL GROWTH INHIBITOR) | | | |
| | (POLYERGIN) (CETERMIN). | | | |
| 2183 | VEGC: (VEGFC) VASCULAR ENDOTHELIAL | P49767 | NM_005429 | 0.58/12% |
| | GROWTH FACTOR C PRECURSOR (VEGF- | | | |
| | C) (VASCULAR ENDOTHELIAL GROWTH | | | |
| | FACTOR RELATED PROTEIN) (VRP) (FLT4 | | | |
| | LIGAND) (FLT4-L). | | | |
| 2189 | PLCB1: (PLCB1) 1- | Q9NQ65 | NM_015192 | 1.40/4% |
| | PHOSPHATIDYLINOSITOL-4,5- | Q9NQH9 | NM_182734 | |
| | BISPHOSPHATE PHOSPHODIESTERASE | Q9NTH4 | | |
| | BETA 1 (EC 3.1.4.11) (PLC-BETA-1) | O60325 | | |
| | (PHOSPHOLIPASE C-BETA-1) (PLC-I) (PLC- | Q9H4H2 | | |
| | 154). KIAA0581 PROTEIN DKFZP434A0814 | Q9BQW2 | | |
| | | Q9UJP6 | | |
| | | Q9UM26 | | |
| | | Q8IV93 Q | | |
| 2193 | PLCG1: (PLCG1 OR PLC1) 1- | P19174 Q2V575 | NM_002660 | 1.22/— % |
| | PHOSPHATIDYLINOSITOL-4,5- | | NM_182811 | |
| | BISPHOSPHATE PHOSPHODIESTERASE | | | |
| | GAMMA 1 (EC 3.1.4.11) (PLC-GAMMA-1) | | | |
| | (PHOSPHOLIPASE C-GAMMA-1) (PLC-II) | | | |
| | (PLC-148). | | | |
| 2195 | PLCG2: (PLCG2) 1- | P16885 Q969T5 | NM_002661 | |
| | PHOSPHATIDYLINOSITOL-4,5- | | | |
| | BISPHOSPHATE PHOSPHODIESTERASE | | | |
| | GAMMA 2 (EC 3.1.4.11) (PLC-GAMMA-2) | | | |
| | (PHOSPHOLIPASE C-GAMMA-2) (PLC-IV). | | | |
| 2196 | PLCD1: (PLCD1) 1- | P51178 | NM_006225 | |
| | PHOSPHATIDYLINOSITOL-4,5- | | | |
| | BISPHOSPHATE PHOSPHODIESTERASE | | | |
| | DELTA 1 (EC 3.1.4.11) (PLC-DELTA-1) | | | |
| | (PHOSPHOLIPASE C-DELTA-1) (PLC-III). | | | |
| 2202 | CYP3A7_HUMAN: (CYP3A7) | P24462 | NM_000765 | |
| | CYTOCHROME P450 3A7 (EC 1.14.14.1) | | | |
| | (CYPIIIA7) (P450-HFLA). | | | |
| 2211 | GAPDH: (GAPDH OR GAPD) | P04406 P00354 | NM_002046 | |
| | GLYCERALDEHYDE-3-PHOSPHATE | | | |
| | DEHYDROGENASE (EC 1.2.1.12) (GAPDH). | | | |
| 2223 | PLP1: (PLP1 OR PLP) MYELIN | P60201 P06905 | NM_000533 | 1.61/18% |
| | PROTEOLIPID PROTEIN (PLP) | P04400 Q502Y1 | NM_199478 | |
| | (LIPOPHILIN) [CONTAINS: MYELIN | | | |
| | PROTEIN DM-20]. | | | |
| 2260 | COL1A1: (COL1A1) COLLAGEN ALPHA 1(I) | P78441 Q13896 | NM_000088 | 0.31/11% |
| | CHAIN PRECURSOR. | Q13902 Q13903 | | |
| | | Q14992 Q15201 | | |
| | | Q16050 | | |
| | | Q7KZ30 | | |
| | | Q7KZ34 Q | | |
| 2262 | COL10A1: (COL10A1) COLLAGEN ALPHA | Q03692 | NM_000493 | |
| | 1(X) CHAIN PRECURSOR. | | | |
| 2264 | COL11A1: (COL11A1) COLLAGEN ALPHA | P12107 Q14034 | NM_001854 | |
| | 1(XI) CHAIN PRECURSOR. | Q9UIT4 | NM_080629 | |
| | | Q9UIT5 | NM_080630 | |
| | | Q9UIT6 | | |
| 2266 | COL12A1: (COL12A1) COLLAGEN ALPHA | Q99716 Q99715 | NM_004370 | |
| | 1(XII) CHAIN PRECURSOR. | | NM_080645 | |
| 2271 | COL14A1: (COL14A1 OR UND) COLLAGEN | O00261 Q05707 | NM_021110 | |
| | ALPHA 1(XIV) CHAIN PRECURSOR | O00260 O00262 | | |
| | (UNDULIN). | Q05708 Q5XJ18 | | |
| | | Q96C67 | | |
| 2275 | COL15A1: (COL15A1) COLLAGEN ALPHA | P39059 Q5T6J4 | NM_001855 | |
| | 1(XV) CHAIN PRECURSOR. | Q9Y4W4 | | |
| 2277 | COL16A1: (COL16A1) COLLAGEN ALPHA | Q07092 | NM_001856 | 0.32/12% |
| | 1(XVI) CHAIN PRECURSOR. | | | |
| 2281 | COL18A1_2: (COL18A1) COLLAGEN ALPHA | P39060 | NM_030582 | |
| | 1(XVIII) CHAIN [CONTAINS: | Q9Y6Q8 | NM_130445 | |
| | ENDOSTATIN]. | Q9Y6Q7 | | |
| | | Q9UK38 | | |
| 2285 | COL2A1: (COL2A1) COLLAGEN ALPHA 1(II) | Q12985 Q14009 | NM_001844 | |
| | CHAIN PRECURSOR [CONTAINS: | Q14044 Q14046 | NM_033150 | |
| | CHONDROCALCIN] (T1) COLLAGEN | Q14056 Q14058 | | |
| | ALPHA 1 TYPE II (T1 MRNA). | Q16672 | | |
| | | Q6LBY1 | | |
| | | Q6LBY2 Q | | |
| 2289 | COL4A1: (COL4A1) COLLAGEN ALPHA | P02462 | NM_001845 | 0.42/5% |
| | 1(IV) CHAIN PRECURSOR (ARRESTEN). | Q9NYC5 | | |
| 2293 | COL6A1: (COL6A1) COLLAGEN (VI) | P12109 Q14041 | NM_001848 | 1.10/9% |
| | ALPHA-1 CHAIN (FRAGMENT) COLLAGEN | Q14040 Q16258 | | |
| | ALPHA 1(VI) CHAIN PRECURSOR. | O00117 O00118 | | |
| 2295 | COL7A1: (COL7A1) COLLAGEN ALPHA | Q02388 Q14054 | NM_000094 | 0.17/9% |
| | 1(VII) CHAIN PRECURSOR (LONG-CHAIN | Q16507 | | |
| | COLLAGEN) (LC COLLAGEN). | | | |
| 2297 | COL8A1: (COL8A1) COLLAGEN ALPHA | P27658 Q96D07 | NM_001850 | 0.82/14% |
| | 1(VIII) CHAIN PRECURSOR (ENDOTHELIAL | | NM_020351 | |
| | COLLAGEN). | | | |
| 2299 | COL9A1_1: (COL9A1) COLLAGEN ALPHA | Q9Y6P2 | NM_001851 | |
| | 1(IX) CHAIN PRECURSOR. | Q9Y6P3 | NM_078485 | |
| | | Q9H151 | | |
| | | Q9H152 Q99225 | | |
| | | Q13699 Q13700 | | |
| | | P20849 Q5TF52 Q | | |
| 2301 | COL1A2: (COL1A2) COLLAGEN ALPHA 2(I) | Q9UEB6 | NM_000089 | 0.40/10% |
| | CHAIN PRECURSOR. | Q9UPH0 | | |
| | | P08123 P02464 | | |
| | | Q13897 Q13997 | | |
| | | Q13998 Q14038 | | |
| | | Q14057 Q | | |
| 2303 | COL11A2: (COL11A2) COLLAGEN ALPHA | Q99866 Q9UIP9 | NM_080679 | 1.27/61% |
| | 2(XI) CHAIN PRECURSOR. | P13942 Q13273 | NM_080680 | |
| | | Q13271 Q13272 | NM_080681 | |
| | | Q07751 | | |
| 2307 | COL5A2: (COL5A2) COLLAGEN ALPHA 2(V) | P05997 | NM_000393 | 0.58/14% |
| | CHAIN PRECURSOR. | | | |
| 2309 | COL6A2_1: (COL6A2) COLLAGEN ALPHA | P12110 Q14049 | NM_001849 | 1.43/2% |
| | 2(VI) CHAIN PRECURSOR | Q9UML3 | | |
| | (DKFZP586E1322). | Q13909 Q13910 | | |
| | | Q13911 Q16259 | | |
| | | Q16597 | | |
| 2313 | COL9A2: (COL9A2) COLLAGEN TYPE IX | Q14055 | NM_001852 | |
| | ALPHA 2 CHAIN (ALPHA-2 IX COLLAGEN). | | | |
| 2315 | COL4A3: (COL4A3) COLLAGEN ALPHA | Q01955 | NM_000091 | 1.11/23% |
| | 3(IV) CHAIN PRECURSOR | Q9BQT2 | | |
| 2319 | COL9A3: (COL9A3) ALPHA-3 TYPE IX | Q9UPE2 | NM_001853 | 1.24/27% |
| | COLLAGEN. | Q9H4G9 | | |
| | | Q13681 Q14050 | | |
| 2321 | COL4A4: (COL4A4) COLLAGEN ALPHA | P53420 | NM_000092 | 1.59/61% |
| | 4(IV) CHAIN PRECURSOR. | | | |
| 2324 | ITGA7: (ITGA7) INTEGRIN ALPHA-7 | Q9UET0 | NM_002206 | 1.16/— % |
| | (INTEGRIN ALPHA 7 CHAIN) (INTEGRIN | Q13683 O43197 | | |
| | ALPHA-7) (INTEGRINA7). | Q9UEV2 | | |
| | | Q9NY89 | | |
| 2326 | ITGA8: (ITGA8) INTEGRIN ALPHA-8 | P53708 | NM_003638 | 1.39/18% |
| | (INTEGRINA8). | Q5VX94 | | |
| 2328 | ITGA9: (ITGA9) INTEGRTN ALPHA-9 | Q13797 Q14638 | NM_002207 | 1.18/13% |
| | PRECURSOR (INTEGRIN ALPHA-RLC) | | | |
| | (INTEGRINA9). | | | |
| 2330 | ITGB5: (ITGB5) INTEGRIN BETA-5 | P18084 | NM_002213 | 0.96/9% |
| | PRECURSOR (INTEGRINB5). | | | |
| 2332 | ITGB6: (ITGB6) INTEGRIN BETA-6 | P18564 Q16500 | NM_000888 | |
| | PRECURSOR (INTEGRINB6). | Q0VA95 | | |
| | | Q53RG5 | | |
| | | Q53RR6 | | |
| 2334 | ITGB7: (ITGB7) INTEGRIN BETA-7 | P26010 | NM_000889 | |
| | PRECURSOR (INTEGRINB7). | | | |
| 2336 | ITGB8: (ITGB8) INTEGRIN BETA-8 | P26012 | NM_002214 | |
| | PRECURSOR. | | | |
| 2338 | PAI1: (SERPINE1 OR PAI1 OR PLANH1) | P05121 | NM_000602 | 0.13/7% |
| | PLASMINOGEN ACTIVATOR INHIBITOR-1 | | | |
| | PRECURSOR (PAI-1) (ENDOTHELIAL | | | |
| | PLASMINOGEN ACTIVATOR INHIBITOR) | | | |
| | (PAI). | | | |
| 2340 | SERPINB2: (SERPINB2 OR PAI2 OR | P05120 Q96E96 | NM_002575 | |
| | PLANH2) PLASMINOGEN ACTIVATOR | | | |
| | INHIBITOR-2 PRECURSOR (PAI-2) | | | |
| | (PLACENTAL PLASMINOGEN ACTIVATOR | | | |
| | INHIBITOR) (MONOCYTE ARG-SERPIN) | | | |
| | (UROKINASE INHIBITOR). | | | |
| 2342 | ADAM17: (ADAM17 OR TACE OR CSVP) | O60226 P78536 | NM_003183 | |
| | ADAM 17 PRECURSOR (EC 3.4.24.—) (A | | NM_021832 | |
| | DISINTEGRIN AND METALLOPROTEINASE | | | |
| | DOMAIN 17) (TNF-ALPHA CONVERTING | | | |
| | ENZYME) (TNF-ALPHA CONVERTASE) | | | |
| | (SNAKE VENOM-LIKE PROTEASE) (CD156B | | | |
| | ANTIGEN). | | | |
| 2344 | TIMP1: (TIMP1 OR TIMP OR CLGI) | P01033 Q14252 | NM_003254 | 0.76/13% |
| | METALLOPROTEINASE INHIBITOR 1 | Q9UCU1 | | |
| | PRECURSOR (TIMP-1) (ERYTHROID | | | |
| | POTENTIATING ACTIVITY) (EPA) (TISSUE | | | |
| | INHIBITOR OF METALLOPROTEINASES) | | | |
| | (FIBROBLAST COLLAGENASE INHIBITOR) | | | |
| | (COLLAGENASE INHIBITOR). | | | |
| 2346 | TIMP2: (TIMP2) METALLOPROTEINASE | Q9UDF7 | NM_003255 | 0.89/1% |
| | INHIBITOR 2 PRECURSOR (TIMP-2) | P16035 Q93006 | | |
| | (TISSUE INHIBITOR OF | Q16121 | | |
| | METALLOPROTEINASES-2) (CSC-21K). | | | |
| 2348 | TIMP3: (TIMP3) METALLOPROTEINASE | Q9UGS2 | NM_000362 | 0.31/6% |
| | INHIBITOR 3 PRECURSOR (TIMP-3) | Q9UC74 P35625 | | |
| | (TISSUE INHIBITOR OF | Q5THV4 | | |
| | METALLOPROTEINASES-3) (MIG-5 | | | |
| | PROTEIN). | | | |
| 2352 | PLAT: (PLAT) TISSUE-TYPE | Q7Z7N2 | NM_000930 | 0.28/11% |
| | PLASMINOGEN ACTIVATOR PRECURSOR | Q86YK8 | NM_000931 | |
| | (EC 3.4.21.68) (TPA) (T-PA) (T- | P00750 Q15103 | NM_033011 | |
| | PLASMINOGEN ACTIVATOR) (ALTEPLASE) | Q9BU99 | | |
| | (RETEPLASE) [CONTAINS: TISSUE-TYPE | | | |
| | PLASMINOGEN ACTIVATOR CHAIN A; | | | |
| | TISSUE-TYPE PLASMINOGEN ACTIVATOR | | | |
| | CHAIN B]. | | | |
| 2354 | UPA: (PLAU) UROKINASE-TYPE | Q15844 Q16618 | NM_002658 | 1.31/4% |
| | PLASMINOGEN ACTIVATOR PRECURSOR | P00749 | | |
| | (EC 3.4.21.73) (UPA) (U-PLASMINOGEN | Q969W6 | | |
| | ACTIVATOR). | | | |
| 2356 | BMP7: (BMP7 OR BMP-7 OR OP1) BONE | Q9NTQ7 | NM_001719 | |
| | MORPHOGENETIC PROTEIN 7 PRECURSOR | Q9H512 P18075 | | |
| | (BMP-7) (OSTEOGENIC PROTEIN 1) (OP-1). | | | |
| 2360 | LAMA1: (LAMA1 OR LAMA) LAMININ | P25391 | NM_005559 | |
| | ALPHA-1 CHAIN PRECURSOR (LAMININ A | | | |
| | CHAIN). | | | |
| 2362 | LAMA2: (LAMA2 OR LAMM) LAMININ | Q93022 Q14736 | NM_000426 | |
| | ALPHA-2 CHAIN PRECURSOR (LAMININ M | P24043 | | |
| | CHAIN) (MEROSIN HEAVY CHAIN). | | | |
| 2364 | LAMA3: (LAMA3) LAMININ ALPHA-3 | Q96TG0 | NM_000227 | 0.60/— % |
| | CHAIN PRECURSOR (EPILIGRIN 170 KDA | Q16787 Q13679 | NM_198129 | |
| | SUBUNIT) (E170). | Q13680 | | |
| 2366 | LAMA4: (LAMA4) LAMININ ALPHA-4 | Q9UE18 | NM_002290 | |
| | CHAIN PRECURSOR. | Q9UJN9 | | |
| | | Q16363 Q15335 | | |
| | | Q14735 Q14731 | | |
| | | Q4LE44 | | |
| | | Q5SZG8 | | |
| 2368 | LAMA5: (KIAA0533 OR LAMA5) KIAA0533 | Q9H1P1 | NM_005560 | 1.18/36% |
| | PROTEIN (LAMININ ALPHA 5 CHAIN) | O15230 | | |
| | (FRAGMENT). | Q8WZA7 | | |
| 2370 | LAMB1: (LAMB1) LAMININ BETA-1 CHAIN | P07942 | NM_002291 | 0.75/3% |
| | PRECURSOR (LAMININ B1 CHAIN). | | | |
| 2375 | LAMB3: (LAMB3) LAMININ BETA-3 CHAIN | O14947 | NM_000228 | 0.36/15% |
| | PRECURSOR (LAMININ B1K CHAIN) | Q9UJK4 | | |
| | (KALININ B1 CHAIN). | Q9UJL1 Q13751 | | |
| | | Q14733 | | |
| 2377 | LAMG1: (LAMC1 OR LAMB2) LAMININ | P11047 | NM_002293 | 1.01/11% |
| | GAMMA-1 CHAIN PRECURSOR (LAMININ | | | |
| | B2 CHAIN). | | | |
| 2385 | EPIPHYCAN: (DSPG3) SMALL | Q99645 Q8NEJ5 | NM_004950 | |
| | CHONDROITIN/DERMATAN SULFATE | | | |
| | PROTEOGLYCAN PRECURSOR (PG-LB) | | | |
| | (PGLB) (EPIPHYCAN) (DERMATAN | | | |
| | SULFATE PROTEOGLYCAN 3) (DSPG3). | | | |
| 2400 | COL4A6: (COL4A6) COLLAGEN TYPE IV A6 | Q9UMG6 | NM_001847 | |
| | CHAIN. | Q14031 Q12823 | NM_033641 | |
| | | Q14053 | | |
| | | Q9NQM5 | | |
| | | Q9NTX3 | | |
| | | Q9UJ76 | | |
| | | Q9Y4L4 | | |
| | | Q5JYH6 | | |
| 2403 | COL4A5: (COL4A5) COLLAGEN ALPHA | Q6LD84 | NM_000495 | |
| | 5(IV) CHAIN PRECURSOR. | Q16006 P29400 | NM_033380 | |
| | | Q16126 | NM_033381 | |
| 2423 | AGC1: (AGC1 OR CSPG1 OR AGC) | Q13650 P16112 | NM_001135 | |
| | AGGRECAN CORE PROTEIN PRECURSOR | Q9UCP4 | NM_013227 | |
| | (CARTILAGE-SPECIFIC PROTEOGLYCAN | Q9UCP5 | | |
| | CORE PROTEIN) (CSPCP) (CHONDROITIN | Q9UDE0 | | |
| | SULFATE PROTEOGLYCAN CORE | | | |
| | PROTEIN 1) (AGGRECAN1). | | | |
| 2425 | AGRIN: (AGRN) AGRIN PRECURSOR. | Q7KYS8 | NM_198576 | 0.45/13% |
| | | Q8N4J5 Q96IC1 | | |
| | | Q9BTD4 | | |
| | | O00468 | | |
| | | Q5SVA2 | | |
| | | Q60FE1 | | |
| 2429 | BAMACAN: (BAM OR SMCD OR HCAP OR | O60464 | NM_005445 | 1.51/5% |
| | CSPG6 OR SMC3 OR SMC3L1 OR BMH) | Q9UQE7 | | |
| | STRUCTURAL MAINTENANCE OF | | | |
| | CHROMOSOME 3 (CHONDROITIN | | | |
| | SULFATE PROTEOGLYCAN 6) | | | |
| | (CHROMOSOME SEGREGATION PROTEIN | | | |
| | SMCD) (BAMACAN) BASEMENT | | | |
| | MEMBRANE-ASSOCIATED CHONDROITIN | | | |
| | PROTEOGLYCAN) (HCAP). | | | |
| 2433 | BMP1_1: (BMP1 OR PCP-3) BONE | Q13292 Q99421 | NM_006129 | 0.49/14% |
| | MORPHOGENETIC PROTEIN 1 PRECURSOR | Q99422 Q99423 | | |
| | (EC 3.4.24.—) (BMP-1) PROCOLLAGEN C- | Q14874 Q13872 | | |
| | PROTEINASE 3. | Q9UL38 P46721 | | |
| | | Q9UGP7 P | | |
| 2435 | BMP5: (BMP5) BONE MORPHOGENETIC | Q9NTM5 | NM_021073 | |
| | PROTEIN 5 PRECURSOR (BMP-5). | Q9H547 P22003 | | |
| 2439 | BCAN: (BCAN) BREVICAN CORE PROTEIN | Q8TBB9 | NM_021948 | |
| | PRECURSOR (CHONDROITIN SULFATE | Q9HBK1 | NM_198427 | |
| | PROTEOGLYCAN BEHAB/BREVICAN). | Q9HBK4 | | |
| | | Q9NT67 | | |
| | | Q96GW7 | | |
| 2451 | IBROMODULIN: (FMOD OR FM) | Q06828 Q15331 | NM_002023 | 1.30/8% |
| | FIBROMODULIN PRECURSOR (FM) | | | |
| | (COLLAGEN-BINDING 59 KDA PROTEIN). | | | |
| 2453 | FN1: (FN1 OR FN) FIBRONECTIN | O95609 O95610 | NM_002026 | 0.25/2% |
| | PRECURSOR (FN) (COLD-INSOLUBLE | Q14312 Q14325 | NM_212474 | |
| | GLOBULIN) (CIG). | Q86T27 Q8IVI8 | NM_212475 | |
| | | Q96KP7 | NM_212476 | |
| | | Q96KP8 | NM_212478 | |
| | | Q96KP9 Q | NM_212482 | |
| 2459 | IBSP: (IBSP OR BNSP) BONE | P21815 | NM_004967 | |
| | SIALOPROTEIN II PRECURSOR (BSP II) | | | |
| | (CELL-BINDING SIALOPROTEIN) | | | |
| | (INTEGRIN-BINDING SIALOPROTEIN). | | | |
| 2473 | LUMICAN: (LDC) LUMICAN PRECURSOR | Q96QM7 | NM_002345 | 1.66/17% |
| | (LUM) (KERATAN SULFATE | P51884 | | |
| | PROTEOGLYCAN). | | | |
| 2487 | MMP10: (MMP10 OR STMY2) | P09238 | NM_002425 | |
| | STROMELYSIN-2 PRECURSOR (EC | | | |
| | 3.4.24.22) (MATRIX | | | |
| | METALLOPROTEINASE-10) (MMP-10) | | | |
| | (TRANSIN-2) (SL-2). | | | |
| 2489 | MMP11: (MMP11 OR STMY3) | P24347 Q5FX24 | NM_005940 | 0.78/— % |
| | STROMELYSIN-3 PRECURSOR (EC 3.4.24.—) | Q6PEZ6 | | |
| | (MATRIX METALLOPROTEINASE-11) | Q9UC26 | | |
| | (MMP-11) (ST3) (SL-3). | | | |
| 2491 | MMP12: (MMP12 OR HME) MACROPHAGE | P39900 | NM_002426 | 1.37/17% |
| | METALLOELASTASE PRECURSOR (EC | | | |
| | 3.4.24.65) (HME) (MATRIX | | | |
| | METALLOPROTEINASE-12) (MMP-12). | | | |
| 2493 | MMP13: (MMP13) COLLAGENASE 3 | P45452 | NM_002427 | |
| | PRECURSOR (EC 3.4.24.—) (MATRIX | | | |
| | METALLOPROTEINASE-13) (MMP-13). | | | |
| 2495 | MMP14: (MMP14 OR MMP-X1) MATRIX | Q92678 P50281 | NM_004995 | 1.12/6% |
| | METALLOPROTEINASE-14 PRECURSOR | | | |
| | (EC 3.4.24.—) (MMP-14) (MEMBRANE-TYPE | | | |
| | MATRIX METALLOPROTEINASE 1) (MT- | | | |
| | MMP 1) (MTMMP1). | | | |
| 2497 | MMP15: (MMP15) MATRIX | Q14111 P51511 | NM_002428 | 0.82/13% |
| | METALLOPROTEINASE-15 PRECURSOR | | | |
| | (EC 3.4.24.—) (MMP-15) (MEMBRANE-TYPE | | | |
| | MATRIX METALLOPROTEINASE 2) (MT- | | | |
| | MMP 2) (MTMMP2). | | | |
| 2499 | MMP16_1: (MMP16 OR MMPX2) MATRIX | Q14824 P51512 | NM_005941 | |
| | METALLOPROTEINASE-16 PRECURSOR | Q52H48 | | |
| | (EC 3.4.24.—) (MMP-16) (MEMBRANE-TYPE | | | |
| | MATRIX METALLOPROTEINASE 3) (MT- | | | |
| | MMP 3) (MTMMP3) (MMP-X2). | | | |
| 2501 | MMP2: (MMP2 OR CLG4A) 72 KDA TYPE IV | P08253 | NM_004530 | 0.94/5% |
| | COLLAGENASE PRECURSOR (EC 3.4.24.24) | | | |
| | (72 KDA GELATINASE) (MATRIX | | | |
| | METALLOPROTEINASE-2) (MMP-2) | | | |
| | (GELATINASE A) (TBE-1) | | | |
| 2503 | MMP3: (MMP3 OR STMY1) STROMELYSIN- | P08254 Q3B7S0 | NM_002422 | |
| | 1 PRECURSOR (EC 3.4.24.17) (MATRIX | Q6GRF8 | | |
| | METALLOPROTEINASE-3) (MMP-3) | | | |
| | (TRANSIN-1) (SL-1). | | | |
| 2505 | MMP7: (MMP7 OR MPSL1 OR PUMP1) | P09237 | NM_002423 | |
| | MATRILYSIN PRECURSOR (EC 3.4.24.23) | Q9BTK9 | | |
| | (PUMP-1 PROTEASE) (UTERINE | | | |
| | METALLOPROTEINASE) (MATRIX | | | |
| | METALLOPROTEINASE-7) (MMP-7) | | | |
| | (MATRIN). | | | |
| 2509 | MMP9: (MMP9 OR CLG4B) 92 KDA TYPE IV | Q9H4Z1 | NM_004994 | |
| | COLLAGENASE PRECURSOR (EC 3.4.24.35) | Q8N725 P14780 | | |
| | (92 KDA GELATINASE) (MATRIX | Q3LR70 | | |
| | METALLOPROTEINASE-9) (MMP-9) | | | |
| | (GELATINASE B) (GELB). | | | |
| 2511 | L1CAM: (L1CAM OR CAML1 OR MIC5) | P32004 Q8TA87 | NM_000425 | |
| | NEURAL CELL ADHESION MOLECULE L1 | | NM_024003 | |
| | PRECURSOR (N-CAM L1) (CD171 | | | |
| | ANTIGEN). | | | |
| 2513 | NEUROCAN: (CSPG3 OR NEUR) | O14594 | NM_004386 | 1.17/16% |
| | NEUROCAN (PGCN_HUMAN). | Q9UPK6 | | |
| 2515 | NIDOGEN: (NID) NIDOGEN PRECURSOR | P14543 Q14942 | NM_002508 | |
| | (ENTACTIN). | Q59FL2 | | |
| | | Q5TAF2 | | |
| | | Q5TAF3 | | |
| | | Q86XD7 | | |
| 2517 | SPP1: (SPP1 OR OPN) OSTEOPONTIN | Q8NBK2 | NM_000582 | |
| | PRECURSOR (BONE SIALOPROTEIN 1) | Q96IZ1 P10451 | NM_001040058 | |
| | (URINARY STONE PROTEIN) (SECRETED | Q15681 Q15682 | NM_001040060 | |
| | PHOSPHOPROTEIN 1) (SPP-1) | Q15683 | | |
| | (NEPHROPONTIN) (UROPONTIN). | | | |
| 2519 | OSF: (OSTF1 OR SH3D3 OR SH3P2) | Q92882 | NM_012383 | 1.28/34% |
| | OSTEOCLAST STIMULATING FACTOR 1 | Q5W126 Q96IJ4 | | |
| | (SH3 DOMAIN PROTEIN 3). | | | |
| 2521 | BGLAP: ((BGLAP1 AND BGLAP2) AND | P02818 | NM_199173 | 0.98/44% |
| | (BGLAP-RS1)) OSTEOCALCIN PRECURSOR | | | |
| | (GAMMA-CARBOXYGLUTAMIC ACID- | | | |
| | CONTAINING PROTEIN) (BONE GLA- | | | |
| | PROTEIN) (BGP) (OSTEOCALCIN- | | | |
| | RELATED PROTEIN PRECURSOR (OC-X) | | | |
| | (NEPHROCALCIN). | | | |
| 2527 | DCN: (DCN) BONE PROTEOGLYCAN II | Q9P0Z0 | NM_001920 | |
| | PRECURSOR (PG-S2) (DECORIN) (PG40) | Q9P0Z1 P07585 | NM_133503 | |
| | (PGS2) | Q9Y5N9 | NM_133504 | |
| | | Q9Y5N8 | NM_133505 | |
| 2531 | SDC4: (SDC4) SYNDECAN-4 PRECURSOR | P31431 Q16833 | NM_002999 | |
| | (AMPHIGLYCAN) (SYND4) (RYUDOCAN | O00773 | | |
| | CORE PROTEIN). | | | |
| 2541 | TNC: (TNC OR HXB) TENASCIN | P24821 Q15567 | NM_002160 | 0.61/17% |
| | PRECURSOR (TN) (HEXABRACHION) | Q14583 | | |
| | (CYTOTACTIN) (NEURONECTIN) (GMEM) | | | |
| | (JI) (MIOTENDINOUS ANTIGEN) (GLIOMA- | | | |
| | ASSOCIATED-EXTRACELLULAR MATRIX | | | |
| | ANTIGEN) (GP 150-225) (TENASCIN-C) (TN- | | | |
| | C). | | | |
| 2545 | TENASCINX: (TNXB OR TNX OR XB OR | Q9NPK9 | NM_019105 | |
| | HXBL) TENASCIN-X PRECURSOR (TN-X) | P22105 P78530 | NM_032470 | |
| | (HEXABRACHION-LIKE) (TNXB ISOFORM | P78531 Q08424 | | |
| | 1) (TNXA). | Q9UMG7 | | |
| 2549 | THBS2: (THBS2 OR TSP2) | P35442 | NM_003247 | 0.90/1% |
| | THROMBOSPONDIN 2 PRECURSOR | | | |
| | (THROMBOSPONDIN2). | | | |
| 2555 | THBS1: (THBS1 OR TSP1 OR TSP) | P07996 Q15667 | NM_003246 | |
| | THROMBOSPONDIN 1 PRECURSOR | | | |
| | (THROMBOSPONDIN1). | | | |
| 2557 | COMP: (COMP) CARTILAGE OLIGOMERIC | Q8N4T2 | NM_000095 | |
| | MATRIX PROTEIN PRECURSOR (COMP) | Q16389 P49747 | | |
| | (THROMBOSPONDIN5). | Q16388 O14592 | | |
| 2560 | MMP19: (MMP19 OR MMP18 OR RASI) | O15278 O95606 | NM_001032360 | 0.80/12% |
| | MATRIX METALLOPROTEINASE-19 | Q99580 Q99542 | NM_002429 | |
| | PRECURSOR (EC 3.4.24.—) (MMP-19) | | | |
| | (MATRIX METALLOPROTEINASE RASI) | | | |
| | (MMP-18). | | | |
| 2936 | IL6: (IL6 OR IFNB2 OR IL-6) INTERLEUKIN- | P05231 | NM_000600 | 0.04/19% |
| | 6 PRECURSOR (IL-6) (B-CELL | Q9UCU2 | | |
| | STIMULATORY FACTOR 2) (BSF-2) | Q9UCU3 | | |
| | (INTERFERON BETA-2) (HYBRIDOMA | Q9UCU4 | | |
| | GROWTH FACTOR). | | | |
| 2965 | BMP4: (BMP4 OR BMP2B OR DVR4 OR | Q9UM80 | NM_001202 | |
| | BMP-4 OR DVR-4) BONE MORPHOGENETIC | P12644 | NM_130850 | |
| | PROTEIN 4 PRECURSOR (BMP-4) (BMP-2B). | | NM_130851 | |
| 3018 | HPRT: (HPRT1 OR HPRT) HYPOXANTHINE- | P00492 | NM_000194 | |
| | GUANINE | | | |
| | PHOSPHORIBOSYLTRANSFERASE (EC | | | |
| | 2.4.2.8) (HGPRT) (HGPRTASE). | | | |
| 3058 | GREM2: (GREM2 OR CKTSF1B2 OR DAND3 | Q9H772 | NM_022469 | |
| | OR PRDC) GREMLIN-2 PRECURSOR | Q86UD9 | | |
| | (CYSTEINE KNOT SUPERFAMILY 1, BMP | | | |
| | ANTAGONIST 2) (PROTEIN RELATED TO | | | |
| | DAN AND CERBERUS) (FLJ21195). | | | |
| 3385 | CSPG2_1: (CSPG2) VERSICAN CORE | Q9UNW5 | NM_004385 | 0.24/7% |
| | PROTEIN PRECURSOR (LARGE | P13611 P20754 | | |
| | FIBROBLAST PROTEOGLYCAN) | Q13010 Q13189 | | |
| | (CHONDROITIN SULFATE | Q15123 | | |
| | PROTEOGLYCAN CORE PROTEIN 2) | Q9UCL9 | | |
| | (GLIAL HYALURONATE-BINDING | | | |
| | PROTEIN) (GHAP). | | | |
| 3454 | ITGAM: (ITGAM OR CR3A OR CD11B) | P11215 | NM_000632 | |
| | INTEGRIN ALPHA-M PRECURSOR (CELL | | | |
| | SURFACE GLYCOPROTEIN MAC-1 ALPHA | | | |
| | SUBUNIT) (CR-3 ALPHA CHAIN) (CD11B) | | | |
| | (LEUKOCYTE ADHESION RECEPTOR MO1) | | | |
| | (INTEGRIN ALPHA-M) (NEUTROPHIL | | | |
| | ADHERENCE RECEPTOR). | | | |
| 3535 | JUN: (JUN) TRANSCRIPTION FACTOR AP-1 | P05412 Q96G93 | NM_002228 | 0.76/38% |
| | (ACTIVATOR PROTEIN 1) (AP1) (PROTO- | | | |
| | ONCOGENE C-JUN) (V-JUN AVIAN | | | |
| | SARCOMA VIRUS 17 ONCOGENE | | | |
| | HOMOLOG) (P39). | | | |
| 3540 | ATF2: (ATF2 OR CREB2 OR CREBP1) | Q13000 P15336 | NM_001880 | |
| | CYCLIC-AMP-DEPENDENT | | | |
| | TRANSCRIPTION FACTOR ATF-2 | | | |
| | (ACTIVATING TRANSCRIPTION FACTOR 2) | | | |
| | (CAMP RESPONSE ELEMENT BINDING | | | |
| | PROTEIN CRE-BP1) (HB16). | | | |
| 3562 | ATF4: (ATF4) CYCLIC-AMP-DEPENDENT | Q9UH31 | NM_001675 | 1.11/3% |
| | TRANSCRIPTION FACTOR ATF-4 (DNA- | P18848 | NM_182810 | |
| | BINDING PROTEIN TAXREB67) (CYCLIC | | | |
| | AMP RESPONSE ELEMENT-BINDING | | | |
| | PROTEIN 2) (CREB2). | | | |
| 3591 | HSPA4_1: (HSPA4 OR HSP110 OR IRP94) | P34932 O95756 | NM_002154 | 1.13/10% |
| | HEAT SHOCK 70 KDA PROTEIN 4 (HEAT | | | |
| | SHOCK 70-RELATED PROTEIN APG-2) | | | |
| | (HSP70RY) (ISCHEMIA RESPONSIVE 94 KDA | | | |
| | PROTEIN). | | | |
| 3594 | HSPA9: (HSPA9B OR HSPA9 OR GRP75) | P31932 P38646 | NM_004134 | 0.83/6% |
| | MITOCHONDRIAL STRESS-70 PROTEIN | P30036 | | |
| | PRECURSOR (75 KDA GLUCOSE | Q9BWB7 | | |
| | REGULATED PROTEIN) (GRP 75) (PEPTIDE- | | | |
| | BINDING PROTEIN 74) (PBP74) | | | |
| | (MORTALIN) (MOT). | | | |
| 3600 | HYOU1: (HYOU1 OR ORP150) 150 KDA | Q9Y4L1 | NM_006389 | 0.63/10% |
| | OXYGEN REGULATED HSP70 FAMILY | | | |
| | PROTEIN (ORP150) (CAB140 OR GRP170) | | | |
| | (HYPOXIA UP-REGULATED 1). | | | |
| 3608 | CEBPB: (CEBPB OR TCF5) | Q9H4Z5 | NM_005194 | 0.96/12% |
| | CCAAT/ENHANCER BINDING PROTEIN | Q96IH2 P17676 | | |
| | BETA (C/EBP BETA) (NUCLEAR FACTOR | | | |
| | NF-IL6) (TRANSCRIPTION FACTOR 5). | | | |
| 3614 | CEBPG: (CEBPG) CCAAT/ENHANCER | P53567 Q5U052 | NM_001806 | 0.72/5% |
| | BINDING PROTEIN GAMMA (C/EBP | | | |
| | GAMMA). | | | |
| 3622 | CREBL1: (CREBL1 OR G13) CYCLIC AMP- | Q99635 Q99637 | NM_004381 | 0.99/19% |
| | DEPENDENT TRANSCRIPTION FACTOR | Q9H3V9 | | |
| | ATF-6 BETA (ACTIVATING | Q9H3W1 | | |
| | TRANSCRIPTION FACTOR 6 BETA) (ATF6- | Q99941 Q13269 | | |
| | BETA) (CAMP-RESPONSIVE ELEMENT- | Q14343 | | |
| | BINDING PROTEIN-LIKE 1) (CAMP | Q9NPL0 | | |
| | RESPONSE ELEMENT-BINDING PROTEIN- | Q9NWF0 Q | | |
| | RELATED PROTEIN) (CREB-RP) (G13 | | | |
| | PROTE | | | |
| 3644 | JUNB: (JUNB) TRANSCRIPTION FACTOR | P17275 | NM_002229 | 1.11/10% |
| | JUN-B (G0S3). | Q96GH3 | | |
| 3676 | FOXG1A-FOXG1B: (FOXG1B OR FKHL1) | P55315 P55316 | NM_005249 | |
| | FORKHEAD PROTEIN G1B (FORKHEAD- | | | |
| | RELATED PROTEIN FKHL1) | | | |
| | (TRANSCRIPTION FACTOR BF-1) (BRAIN | | | |
| | FACTOR 1) (BF1) (HFK1) (FOXG1A OR | | | |
| | FKHL2) FORKHEAD BOX PROTEIN G1A | | | |
| | (FORKHEAD-RELATED PROTEIN FKHL2) | | | |
| | (TRANSCRIPTION FACTOR BF-2). | | | |
| 3680 | FAST1: (FOXH1 OR FAST1) FORKHEAD | O75593 | NM_003923 | |
| | BOX PROTEIN H1 (FORKHEAD ACTIVIN | | | |
| | SIGNAL TRANSDUCER 1) (FAST-1). (FOXH1 | | | |
| | OR FAST2) FORKHEAD ACTIVIN SIGNAL | | | |
| | TRANSDUCER 2. TGF-BETA/ACTIVIN | | | |
| | SIGNAL TRANSDUCER FAST-1P. | | | |
| 3684 | FKHL16: (FOXM1 OR FKHL16 OR HFH11 OR | O43260 Q08050 | NM_021953 | |
| | WIN OR MPP2) FORKHEAD PROTEIN M1 | O43258 O43259 | NM_202002 | |
| | (FORKHEAD-RELATED PROTEIN FKHL16) | Q9BRL2 | NM_202003 | |
| | (HEPATOCYTE NUCLEAR FACTOR 3 | Q4ZGG7 | | |
| | FORKHEAD HOMOLOG 11) (HNF-3/FORK- | | | |
| | HEAD HOMOLOG-3) (HFH-11) (WINGED | | | |
| | HELIX FACTOR FROM INS-1 CELLS) (M- | | | |
| | PHASE PHOSPHOPROTEIN 2 | | | |
| 3686 | FKHR: (FOXO1A OR FKHR) FORKHEAD | Q12778 O43523 | NM_002015 | |
| | PROTEIN O1A (FORKHEAD IN | Q6NSK6 | | |
| | RHABDOMYOSARCOMA). | Q5VYC7 | | |
| 3707 | HNF3A: (FOXA1 OR HNF3A OR TCF3A) | P55317 | NM_004496 | |
| | HEPATOCYTE NUCLEAR FACTOR 3- | Q9H2A0 | | |
| | ALPHA (HNF-3A). | | | |
| 3709 | HNF3B: (FOXA2 OR HNF3B OR TCF3B) | Q9Y261 | NM_021784 | |
| | HEPATOCYTE NUCLEAR FACTOR 3-BETA | Q96DF7 | NM_153675 | |
| | (HNF-3B). | Q8WUW4 | | |
| 3711 | HNF3G: (FOXA3 OR TCF-3G OR HNF3G OR | P55318 | NM_004497 | |
| | TCF3G) HEPATOCYTE NUCLEAR FACTOR | Q9UMW9 | | |
| | 3-GAMMA (HNF-3G) (FORK HEAD- | | | |
| | RELATED PROTEIN FKH H3). | | | |
| 3719 | FOXC2: (FOXC2 OR FKHL14 OR MFH1) | Q99958 | NM_005251 | |
| | FORKHEAD BOX PROTEIN C2 (FORKHEAD- | | | |
| | RELATED PROTEIN FKHL14) | | | |
| | (MESENCHYME FORK HEAD PROTEIN 1) | | | |
| | (MFH-1 PROTEIN) (TRANSCRIPTION | | | |
| | FACTOR FKH-14) | | | |
| 3896 | CNP: (CNP) 2′,3′-CYCLIC NUCLEOTIDE 3′- | P09543 | NM_033133 | 1.05/31% |
| | PHOSPHODIESTERASE (EC 3.1.4.37) (CNP) | | | |
| | (CNPASE) (CNPI) (CNPII). | | | |
| 3919 | MAP2: (MAP2 OR MTAP2) MICROTUBULE- | P11137 Q99976 | NM_001039538 | |
| | ASSOCIATED PROTEIN 2 (MAP 2) (MAP-2). | Q99975 | NM_002374 | |
| | | | NM_031845 | |
| | | | NM_031847 | |
| 3929 | RPSA: (RPSA OR LAMR1 OR LAMBR OR | P08865 P11085 | NM_002295 | 1.31/5% |
| | P40-8) 40S RIBOSOMAL PROTEIN SA (P40) | P12030 Q16471 | | |
| | (34/67 KDA LAMININ RECEPTOR) (COLON | Q6IPD1 | | |
| | CARCINOMA LAMININ-BINDING PROTEIN) | | | |
| | (NEM/1CHD4) (MULTIDRUG RESISTANCE- | | | |
| | ASSOCIATED PROTEIN MGR1-AG) | | | |
| | (MUSLAMR). | | | |
| 3945 | OSP: (OTM OR OSP OR CLDN11) CLAUDIN- | Q5U0P3 | NM_005602 | 0.25/22% |
| | 11 (OLIGODENDROCYTE SPECIFIC | O75508 | | |
| | PROTEIN) (OLIGODENDROCYTE | | | |
| | TRANSMEMBRANE PROTEIN). | | | |
| 3953 | S100B: (S100B) S-100 PROTEIN, BETA | P04271 | NM_006272 | |
| | CHAIN. | | | |
| 3963 | SNAP25A: (SNAP25 OR SNAP) | P60880 | NM_003081 | |
| | SYNAPTOSOMAL-ASSOCIATED PROTEIN | | NM_130811 | |
| | 25 (SNAP-25) (SUPER PROTEIN) (SUP). | | | |
| 4042 | IKKA: (IKK ALPHA OR CHUK) INHIBITOR | Q13132 Q92467 | NM_001278 | 1.05/19% |
| | OF NUCLEAR FACTOR KAPPA-B KINASE | O14666 O15111 | | |
| | ALPHA SUBUNIT (EC 2.7.1.—) (I KAPPA-B | | | |
| | KINASE ALPHA) (IKBKA) (IKK-ALPHA) | | | |
| | (IKK-A) (IKAPPAB KINASE) (I-KAPPA-B | | | |
| | KINASE 1) (IKK1) (CONSERVED HELIX- | | | |
| | LOOP-HELIX UBIQUITOUS KINASE) | | | |
| | (NUCLEAR FACTO | | | |
| 4045 | IKKB: (IKKB OR IKBKB) INHIBITOR OF | O14920 O75327 | NM_001556 | 0.77/16% |
| | NUCLEAR FACTOR KAPPA B KINASE | | | |
| | BETA SUBUNIT (EC 2.7.1.—) (I-KAPPA-B- | | | |
| | KINASE BETA) (IKBKB) (IKK-BETA) (IKK- | | | |
| | B) (I-KAPPA-B KINASE 2) (IKK2) (NUCLEAR | | | |
| | FACTOR NF-KAPPA-B INHIBITOR KINASE | | | |
| | BETA) (NFKBIKB). | | | |
| 4064 | NFATCB_1: (NFATC1 OR NFATC OR | Q15793 O95644 | NM_006162 | 1.01/— % |
| | NFAT2) NUCLEAR FACTOR OF | Q12865 | NM_172387 | |
| | ACTIVATED T-CELLS, CYTOPLASMIC 1 | | NM_172389 | |
| | (NFAT TRANSCRIPTION COMPLEX | | NM_172390 | |
| | CYTOSOLIC COMPONENT) (NF-ATC1) (NF- | | | |
| | ATC). | | | |
| 4068 | NFKB1: (NFKB1) NUCLEAR FACTOR NF- | P19838 | NM_003998 | 0.53/8% |
| | KAPPA-B P105 SUBUNIT (DNA-BINDING | Q9NZC0 | | |
| | FACTOR KBF1) (EBP-1) [CONTAINS: | Q68D84 | | |
| | NUCLEAR FACTOR NF-KAPPA-B P50 | Q86V43 | | |
| | SUBUNIT]. | Q8N4X7 | | |
| 4070 | NFKB2: (NFKB2) NUCLEAR FACTOR NF- | Q9H472 Q00653 | NM_002502 | 0.35/6% |
| | KAPPA-B P100 SUBUNIT (H2TF1) | Q9BU75 | | |
| | (ONCOGENE LYT-10) (LYT10) [CONTAINS: | Q9H471 Q04860 | | |
| | NUCLEAR FACTOR NF-KAPPA-B P52 | | | |
| | SUBUNIT]. | | | |
| 4072 | NFKB3: (RELA OR NFKB3) | Q04206 | NM_021975 | 0.90/10% |
| | TRANSCRIPTION FACTOR P65 (NUCLEAR | Q6SLK1 | | |
| | FACTOR NF-KAPPA-B P65 SUBUNIT). | | | |
| 4181 | ASCL1: (ASCL1 OR ASH1 OR MASH1 OR | P50553 | NM_004316 | |
| | MASH-1) ACHAETE-SCUTE HOMOLOG 1 | Q9BQ30 | | |
| | (MASH-1) (HASH1). | | | |
| 4185 | ATH1: (ATOH1 OR ATH1) ATONAL | Q92858 | NM_005172 | |
| | PROTEIN HOMOLOG 1 (HELIX-LOOP- | | | |
| | HELIX PROTEIN HATH-1). | | | |
| 4197 | HIF1A: (HIF1A) HYPOXIA-INDUCIBLE | Q16665 Q96PT9 | NM_001530 | 0.71/8% |
| | FACTOR 1 ALPHA (HIF-1 ALPHA) (ARNT | Q9UPB1 | NM_181054 | |
| | INTERACTING PROTEIN) (MEMBER OF | | | |
| | PAS PROTEIN 1) (MOP1) (HIF1 ALPHA). | | | |
| 4199 | ID1: (ID1 OR ID) DNA-BINDING PROTEIN | P41134 O00651 | NM_002165 | |
| | INHIBITOR ID-1 (ID) | O00652 Q16371 | NM_181353 | |
| | | Q16377 | | |
| | | Q5TE66 | | |
| | | Q5TE67 | | |
| | | Q969Z7 | | |
| | | Q9H0Z5 Q | | |
| 4201 | ID2: (ID2) DNA-BINDING PROTEIN | Q02363 | NM_002166 | |
| | INHIBITOR ID-2. | | | |
| 4203 | ID3: (ID3 OR 1R21 OR HEIR-1) DNA- | Q02535 O75641 | NM_002167 | 1.33/18% |
| | BINDING PROTEIN INHIBITOR ID-3 (ID- | | | |
| | LIKE PROTEIN INHIBITOR HLH 1R21) | | | |
| | (HELIX-LOOP-HELIX PROTEIN HEIR-1). | | | |
| 4233 | NEUROD1: (NEUROD1 OR NEUROD) | Q13562 Q13340 | NM_002500 | |
| | NEUROGENIC DIFFERENTIATION FACTOR | Q99455 O00343 | | |
| | 1. | Q96TH0 | | |
| | | Q5U095 | | |
| | | Q9UEC8 | | |
| 4237 | NEUROG1: (NEUROG1 OR NGN1 OR NGN | Q96HE1 | NM_006161 | |
| | OR NEUROD3 OR ATH4C) NEUROGENIN 1 | Q92886 | | |
| | (NEUROGENIC DIFFERENTIATION | | | |
| | FACTOR 3) (NEUROD3) (NEUROGENIC | | | |
| | BASIC-HELIX-LOOP-HELIX PROTEIN). | | | |
| 4241 | NMYC: (MYCN OR NMYC) N-MYC PROTO- | Q6LDT9 | NM_005378 | |
| | ONCOGENE PROTEIN. | P04198 | | |
| 4251 | TAL1: (TAL1 OR SCL OR TCL5) T-CELL | P17542 | NM_003189 | |
| | ACUTE LYMPHOCYTIC LEUKEMIA-1 | | | |
| | PROTEIN (TAL-1 PROTEIN) (STEM CELL | | | |
| | PROTEIN) (T-CELL | | | |
| | LEUKEMIA/LYMPHOMA-5 PROTEIN). | | | |
| 4255 | TCF3: (TCF3 OR E2A OR ITF1 OR TCFE2A | Q9UPI9 Q14635 | NM_003200 | 0.87/17% |
| | OR ALF2 OR ME2) TRANSCRIPTION | Q14636 Q14208 | | |
| | FACTOR E2-ALPHA (IMMUNOGLOBULIN | P15923 P15883 | | |
| | ENHANCER BINDING FACTOR E12/E47) | | | |
| | (TRANSCRIPTION FACTOR-3) (TCF-3) | | | |
| | (IMMUNOGLOBULIN TRANSCRIPTION | | | |
| | FACTOR-1) (TRANSCRIPTION FACTOR ITF- | | | |
| | 1) (TRANSCRIPTIONAL REGU | | | |
| 4257 | TCF4: (TCF4 OR ITF2 OR SEF2) | P15884 Q15439 | NM_003199 | |
| | TRANSCRIPTION FACTOR 4 | Q15440 | | |
| | (IMMUNOGLOBULIN TRANSCRIPTION | | | |
| | FACTOR 2) (RITF-2) (ITF-2) (SL3-3 | | | |
| | ENHANCER FACTOR 2) (SEF-2) (CLASS A | | | |
| | HELIX-LOOP-HELIX TRANSCRIPTION | | | |
| | FACTOR ME2). | | | |
| 4275 | EBCTF: (EBF) EARLY B-CELL | Q8IW11 | NM_024007 | |
| | TRANSCRIPTION FACTOR (FRAGMENT). | Q9UH73 | | |
| | (COE1 OR OLF1) TRANSCRIPTION FACTOR | | | |
| | COE1 (OE-1) (O/E-1) (OLFACTORY | | | |
| | NEURONAL TRANSCRIPTION FACTOR) | | | |
| | (OLF-1). | | | |
| 4279 | HAND1: (HAND1 OR EHAND) HEART- AND | O96004 | NM_004821 | |
| | NEURAL CREST DERIVATIVES- | | | |
| | EXPRESSED PROTEIN 1 | | | |
| | (EXTRAEMBRYONIC TISSUES, HEART, | | | |
| | AUTONOMIC NERVOUS SYTEM AND | | | |
| | NEURAL CREST DERIVATIVES- | | | |
| | EXPRESSED PROTEIN 1) (EHAND) (BASIC | | | |
| | HELIX-LOOP-HELIX PROTEIN HAND1) | | | |
| | (THING1). | | | |
| 4289 | HESR1: (HESR-1 OR CHF2 OR HEY1) HAIRY | Q9NYP4 | NM_012258 | |
| | AND ENHANCER OF SPLIT RELATED-1 | Q9Y5J3 | | |
| | (HEY1 PROTEIN). | Q5TZS3 | | |
| 4321 | NGN3: (NEUROG3 OR NGN3 OR ATOH5 OR | Q9Y4Z2 | NM_020999 | |
| | ATH4B) NEUROGENIN 3 (ATONAL | Q9BY24 | | |
| | PROTEIN HOMOLOG 5) (HELIX-LOOP- | | | |
| | HELIX PROTEIN MATH-4B) (MATH4B) | | | |
| | (RELAX). | | | |
| 4331 | RACK17: (OLIG2 OR BHLHB1 OR PRKCBP2 | Q86X04 Q13516 | NM_005806 | 1.18/10% |
| | OR RACK17) OLIGODENDROCYTE | Q9NZ14 | | |
| | TRANSCRIPTION FACTOR 2 (BASIC HELIX- | | | |
| | LOOP-HELIX PROTEIN CLASS B 1) | | | |
| | (PROTEIN KINASE C-BINDING PROTEIN | | | |
| | RACK17) (PROTEIN KINASE C BINDING | | | |
| | PROTEIN 2). | | | |
| 4334 | SCX: (SCX) SCLERAXIS | — | — | |
| 4398 | BRACHYURY: (T) BRACHYURY PROTEIN | O15178 | NM_003181 | |
| | (T PROTEIN). | | | |
| 4418 | MEF-2C: (MEF2C) MYOCYTE-SPECIFIC | Q06413 | NM_002397 | |
| | ENHANCER FACTOR 2C. | | | |
| 4436 | TBX1: (TBX1) T-BOX TRANSCRIPTION | O43435 O43436 | NM_005992 | |
| | FACTOR TBX1 (T-BOX PROTEIN 1) | Q96RJ2 | NM_080646 | |
| | (TESTIS-SPECIFIC T-BOX PROTEIN). | | NM_080647 | |
| 4446 | TBX3: (TBX3) T-BOX TRANSCRIPTION | O15119 | NM_005996 | 1.31/11% |
| | FACTOR TBX3 (T-BOX PROTEIN 3). | Q9UKF8 | NM_016569 | |
| | | Q8TB20 | | |
| 4448 | TBX5_1: (TBX5) T-BOX TRANSCRIPTION | Q99593 Q9Y4I2 | NM_000192 | |
| | FACTOR TBX5 (T-BOX PROTEIN 5). | O15301 | NM_080717 | |
| | | | NM_181486 | |
| 4528 | CXCL12: (CXCL12 OR SDF1) STROMAL | P48061 | NM_000609 | |
| | CELL-DERIVED FACTOR 1 PRECURSOR | | | |
| | (SDF-1) (CXCL12) (PRE-B CELL GROWTH | | | |
| | STIMULATING FACTOR) (PBSF) (12-O- | | | |
| | TETRADECANOYLPHORBOL 13-ACETATE | | | |
| | REPRESSED PROTEIN 1) (TPAR1) (THYMIC | | | |
| | LYMPHOMA CELL STIMULATING | | | |
| | FACTOR) (TLSF). | | | |
| 4683 | FGFR1_1_HUMAN: (FGFR1 OR FLG OR | Q02063 Q02065 | NM_000604 | |
| | FGFBR OR FLT2) BASIC FIBROBLAST | Q14306 Q14307 | NM_015850 | |
| | GROWTH FACTOR RECEPTOR 1 | Q8N685 P11362 | NM_023105 | |
| | PRECURSOR (BFGF-R) EC 2.7.1.112) (FMS- | P17049 | NM_023106 | |
| | LIKE TYROSINE KINASE-2) (C-FGR) | | NM_023107 | |
| | (BFGFR) (CD331 ANTIGEN). | | NM_023108 | |
| | | | NM_0 | |
| 4690 | EGF: (EGF) PRO-EPIDERMAL GROWTH | P01133 | NM_001963 | |
| | FACTOR PRECURSOR (EGF) [CONTAINS: | | | |
| | EPIDERMAL GROWTH FACTOR | | | |
| | (UROGASTRONE)]. | | | |
| 4693 | EGFR_1: (EGFR OR ERBB1) EPIDERMAL | Q9UMD7 | NM_005228 | 1.61/24% |
| | GROWTH FACTOR RECEPTOR PRECURSOR | Q9UMD8 | | |
| | (EC 2.7.1.112) (RECEPTOR PROTEIN- | Q9UMG5 | | |
| | TYROSINE KINASE ERBB-1). | Q92795 O00732 | | |
| | | O00688 | | |
| | | Q9BZS2 | | |
| | | Q9H2C9 | | |
| | | Q9GZX1 P | | |
| 4695 | FN1_EIIIA: (FN1 OR FN) FIBRONECTIN | O95609 O95610 | NM_002026 | |
| | PRECURSOR (FIBRONECTIN EIIIA | Q14312 Q14325 | NM_212475 | |
| | DOMAIN). | Q86T27 Q8IVI8 | NM_212478 | |
| | | Q96KP7 | NM_212482 | |
| | | Q96KP8 | | |
| | | Q96KP9 Q | | |
| 4696 | ENOS: (NOS3) NITRIC-OXIDE SYNTHASE, | P29474 Q14251 | NM_000603 | 0.91/17% |
| | ENDOTHELIAL (EC 1.14.13.39) (EC-NOS) | Q14434 Q13662 | | |
| | (NOS, TYPE III) (NOSIII) (ENDOTHELIAL | | | |
| | NOS) (ENOS) (CONSTITUTIVE NOS) | | | |
| | (CNOS). | | | |
| 4699 | EDN1: (EDN1) ENDOTHELIN-1 PRECURSOR | P05305 | NM_001955 | |
| | (ET-1). | Q96DA1 | | |
| 4701 | EDN2: (EDN2) ENDOTHELIN-2 PRECURSOR | P20800 | NM_001956 | |
| | (ET-2) (VASOACTIVE INTESTINAL | | | |
| | CONTRACTOR) (VIC). | | | |
| 4705 | GFAP_1_HUMAN: (GFAP) GLIAL | P14136 Q53H98 | NM_002055 | |
| | FIBRILLARY ACIDIC PROTEIN, | Q5D055 | | |
| | ASTROCYTE (GFAP). | Q6ZQS3 | | |
| | | Q7Z5J6 Q7Z5J7 | | |
| | | Q96KS4 | | |
| | | Q96P18 | | |
| | | Q9UFD0 | | |
| 4711 | HGF: (HGF OR HPTA) HEPATOCYTE | Q9UDU6 | NM_000601 | |
| | GROWTH FACTOR PRECURSOR (SCATTER | Q9BYL9 | | |
| | FACTOR) (SF) (HEPATOPOEITIN A). | Q02935 Q13494 | | |
| | | Q14519 | | |
| | | Q8TCE2 | | |
| | | Q9BYM0 | | |
| | | P14210 | | |
| 4715 | SERPINH1-SERPINH2: (SERPINH1 OR CBP1 | Q8IY96 P29043 | NM_001235 | 0.64/10% |
| | OR HSP47) HEAT SHOCK PROTEIN 47 | Q9NP88 P50454 | | |
| | COLLAGEN BINDING PROTEIN 1 (CBP1) | Q5XPB4 | | |
| | (COLLIGIN 1) (SERPINH2 OR CBP2) | Q6NSJ6 | | |
| | (COLLAGEN-BINDING PROTEIN 2 | | | |
| | PRECURSOR) (COLLIGIN 2) (RHEUMATOID | | | |
| | ARTHRITIS RELATED ANTIGEN RA-A47). | | | |
| 4727 | IGF1R: (IGF1R) INSULIN-LIKE GROWTH | P08069 | NM_000875 | 1.26/22% |
| | FACTOR I RECEPTOR PRECURSOR (EC | | | |
| | 2.7.1.112) (CD221 ANTIGEN). | | | |
| 4736 | LIF: (LIF OR HILDA) LEUKEMIA | P15018 Q52LZ2 | NM_002309 | 0.35/13% |
| | INHIBITORY FACTOR PRECURSOR (LIF) | | | |
| | (DIFFERENTIATION-STIMULATING | | | |
| | FACTOR) (D FACTOR) (MELANOMA- | | | |
| | DERIVED LPL INHIBITOR) (MLPLI) | | | |
| | (CHOLINERGIC NEURONAL | | | |
| | DIFFERENTIATION FACTOR). | | | |
| 4739 | LIFR: (LIFR) LEUKEMIA INHIBITORY | P42702 | NM_002310 | |
| | FACTOR RECEPTOR PRECURSOR (LIF-R) | Q6LCD9 | | |
| | (CD118 ANTIGEN) (LIFRA). | | | |
| 4747 | IGF2R: (IGF2R OR MPRI) CATION- | P11717 Q96PT5 | NM_000876 | 0.51/8% |
| | INDEPENDENT MANNOSE-6-PHOSPHATE | Q7Z7G9 | | |
| | RECEPTOR PRECURSOR (CI MAN-6-P | | | |
| | RECEPTOR) (CI-MPR) (M6PR) (INSULIN- | | | |
| | LIKE GROWTH FACTOR 2 RECEPTOR) | | | |
| | (INSULIN-LIKE GROWTH FACTOR II | | | |
| | RECEPTOR) (IGF-II RECEPTOR) (M6P/IGF2 | | | |
| | RECEPTOR) (M6P/IGF2R) (300 | | | |
| 4764 | RARB2_1: (RARB OR NR1B2 OR HAP) | Q00989 Q15298 | NM_000965 | |
| | RETINOIC ACID RECEPTOR BETA-2 (RAR- | Q9UN48 | | |
| | BETA-2) (RAR-EPSILON). | P12891 P10826 | | |
| 4765 | SMAD2: (MADH2 OR SMAD2 OR MADR2) | Q15796 | NM_005901 | 1.16/5% |
| | MOTHERS AGAINST DECAPENTAPLEGIC | | | |
| | HOMOLOG 2 (SMAD 2) (MOTHERS | | | |
| | AGAINST DPP HOMOLOG 2) (MAD- | | | |
| | RELATED PROTEIN 2) (HMAD-2) (JV18-1) | | | |
| | (HSMAD2). | | | |
| 4767 | SMAD3: (MADH3 OR SMAD3 OR MAD33) | P84022 | NM_005902 | 0.48/16% |
| | MOTHERS AGAINST DECAPENTAPLEGIC | | | |
| | HOMOLOG 3 (SMAD 3) (MOTHERS | | | |
| | AGAINST DPP HOMOLOG 3) (MAD3) | | | |
| | (HMAD-3) (MMAD3) (JV15-2) | | | |
| 4770 | SMAD4: (MADH4 OR SMAD4 OR DPC4) | Q13485 | NM_005359 | 0.89/16% |
| | MOTHERS AGAINST DECAPENTAPLEGIC | | | |
| | HOMOLOG 4 (SMAD 4) (MOTHERS | | | |
| | AGAINST DPP HOMOLOG 4) (DELETION | | | |
| | TARGET IN PANCREATIC CARCINOMA 4) | | | |
| | (HSMAD4). | | | |
| 4772 | SMAD7: (MADH7 OR SMAD7 OR MADH8) | O14740 | NM_005904 | |
| | MOTHERS AGAINST DECAPENTAPLEGIC | Q6DK23 | | |
| | HOMOLOG 7 (SMAD 7) (MOTHERS | O15105 | | |
| | AGAINST DPP HOMOLOG 7) (SMAD7) | | | |
| | (HSMAD7). | | | |
| 4775 | TGFBR1: (TGFBR1) TGF-BETA RECEPTOR | P36897 | NM_004612 | |
| | TYPE I PRECURSOR (EC 2.7.1.37) (TGFR-1) | | | |
| | (TGF-BETA TYPE I RECEPTOR) | | | |
| | (SERINE/THREONINE-PROTEIN KINASE | | | |
| | RECEPTOR R4) (SKR4) (ACTIVIN | | | |
| | RECEPTOR-LIKE KINASE 5) (ALK-5). | | | |
| 4777 | TGFBR2: (TGFBR2) TGF-BETA RECEPTOR | Q15580 | NM_001024847 | |
| | TYPE II PRECURSOR (EC 2.7.1.37) (TGFR-2) | Q6DKT6 | NM_003242 | |
| | (TGF-BETA TYPE II RECEPTOR). | P37173 Q99474 | | |
| 4835 | GCNF: (NR6A1 OR GCNF) ORPHAN | Q8NHQ0 | NM_001489 | |
| | NUCLEAR RECEPTOR NR6A1 (GERM CELL | O00551 Q15406 | NM_033334 | |
| | NUCLEAR FACTOR) (GCNF) RETINOID | Q99802 Q92898 | NM_033335 | |
| | RECEPTOR-RELATED TESTIS SPECIFIC | O00603 | | |
| | RECEPTOR) (RTR). | | | |
| 4839 | HNF4A: (HNF4A OR NR2A1 OR TCF14 OR | Q9NQH0 | NM_178850 | 0.81/4% |
| | HNF4) HEPATOCYTE NUCLEAR FACTOR 4- | P41235 Q92653 | | |
| | ALPHA (HNF-4-ALPHA) (TRANSCRIPTION | Q92654 Q92655 | | |
| | FACTOR HNF-4) (TRANSCRIPTION FACTOR | Q14540 Q99864 | | |
| | 14). | O00723 O00659 | | |
| 4841 | HNF4G: (HNF4G OR NR2A2) HEPATOCYTE | Q9UIS6 | NM_004133 | |
| | NUCLEAR FACTOR 4-GAMMA (HNF4- | Q9UH81 | | |
| | GAMMA) | Q14541 | | |
| 4855 | LXR-ALPHA: (NR1H3 OR LXRA) | Q13133 Q96H87 | NM_005693 | |
| | OXYSTEROLS RECEPTOR LXR-ALPHA | | | |
| | (LIVER X RECEPTOR ALPHA) (NUCLEAR | | | |
| | ORPHAN RECEPTOR LXR-ALPHA). | | | |
| 4893 | PPARG_1: (PPARG OR NR1C3) | Q15832 O00684 | NM_005037 | |
| | PEROXISOME PROLIFERATOR | Q15180 O00710 | NM_015869 | |
| | ACTIVATED RECEPTOR GAMMA (PPAR- | Q86U60 P37231 | NM_138711 | |
| | GAMMA) (PPARG2). | O14515 Q15178 | NM_138712 | |
| | | Q15179 Q | | |
| 4895 | RARG1: (RARG OR NR1B3) RETINOIC ACID | P13631 | NM_000966 | 1.14/9% |
| | RECEPTOR GAMMA-1 (RAR-GAMMA-1). | | | |
| 4909 | RXRA: (RXRA OR NR2B1) RETINOIC ACID | P19793 Q2V504 | NM_002957 | |
| | RECEPTOR RXR-ALPHA. | | | |
| 4911 | RXRB: (RXRB OR NR2B2) RETINOIC ACID | P28702 P28703 | NM_021976 | |
| | RECEPTOR RXR-BETA. | | | |
| 4913 | RXRG: (RXRG OR NR2B3) RETINOIC ACID | P48443 | NM_006917 | |
| | RECEPTOR RXR-GAMMA. | | | |
| 4923 | TFCOUP1: (TFCOUP1 OR NR2F1 OR | P10589 | NM_005654 | 1.47/— % |
| | ERBAL3 OR EAR3) COUP TRANSCRIPTION | | | |
| | FACTOR 1 (COUP-TF1) (COUP-TF I) (V- | | | |
| | ERBA RELATED PROTEIN EAR-3). | | | |
| 4978 | CNTF-ZFP91_1: (CNTF) CILIARY | Q86V47 Q96JP5 | NM_000614 | |
| | NEUROTROPHIC FACTOR (ZFP91) (ZINC | Q96QA3 | NM_170768 | |
| | FINGER PROTEIN ZFP91) (PZF) (ZINK | Q96JP4 P26441 | | |
| | FINGER PROTEIN PZF). | | | |
| 4982 | CTF1: (CTF1) CARDIOTROPHIN-1 (CT-1). | Q16619 | NM_001330 | |
| 4986 | HBEGF: (HBEGF OR DTR OR HEGFL) | Q99075 | NM_001945 | 0.40/20% |
| | HEPARIN-BINDING EGF-LIKE GROWTH | | | |
| | FACTOR PRECURSOR (HB-EGF) (HBEGF) | | | |
| | (DIPHTERIA TOXIN RECEPTOR) (DT-R). | | | |
| 4990 | NRG1: (NRG1 OR HGL OR NDF OR HRGA | Q7RTV9 | NM_013956 | 1.44/— % |
| | OR GGF OR SMDF) PRO-NEUREGULIN-1 | Q7RTW0 | NM_013957 | |
| | PRECURSOR (PRO-NRG1) [CONTAINS: | Q7RTW1 | NM_013964 | |
| | NEUREGULIN-1 (NEU DIFFERENTIATION | Q7RTW2 | | |
| | FACTOR) (HEREGULIN) (HRG) (BREAST | Q8NFN1 | | |
| | CANCER CELL DIFFERENTIATION FACTOR | Q8NFN2 | | |
| | P45) (ACETYLCHOLINE RECEPTOR | Q8NFN3 | | |
| | INDUCING ACTIVITY) (ARIA | Q02297 Q02298 Q | | |
| 4995 | NRG3: (NRG3) PRO-NEUREGULIN-3 | P56975 | NM_001010848 | 1.06/— % |
| | PRECURSOR (PRO-NRG3) [CONTAINS: | Q0PEH2 | | |
| | NEUREGULIN-3 (NRG-3)]. | Q5VYH3 | | |
| 4999 | NRG4: (NRG4) PRO-NEUREGULIN-4, SHORT | Q8WWG1 | NM_138573 | |
| | ISOFORM (PRO-NRG4) [CONTAINS: | | | |
| | NEUREGULIN-4 (NRG-4)]. | | | |
| 5000 | NRG2_1: (NRG2 OR NTAK) PRO- | O14511 | NM_004883 | |
| | NEUREGULIN-2, MEMBRANE-BOUND | | NM_013981 | |
| | ISOFORM PRECURSOR (PRO-NRG2) | | NM_013982 | |
| | [CONTAINS: NEUREGULIN-2 (NRG-2) | | NM_013983 | |
| | (NEURAL- AND THYMUS-DERIVED | | | |
| | ACTIVATOR FOR ERBB KINASES) (NTAK) | | | |
| | (DIVERGENT OF NEUREGULIN-1) (DON-1)]. | | | |
| 5004 | SMAD1: (MADH1 OR SMAD1 OR MADR1 | Q15797 Q16636 | NM_005900 | 0.80/4% |
| | OR BSP1) MOTHERS AGAINST | | | |
| | DECAPENTAPLEGIC HOMOLOG 1 (SMAD | | | |
| | 1) (MOTHERS AGAINST DPP HOMOLOG 1) | | | |
| | (MAD-RELATED PROTEIN 1) | | | |
| | (TRANSFORMING GROWTH FACTOR-BETA | | | |
| | SIGNALING PROTEIN-1) (BSP-1) (HSMAD1) | | | |
| | (JV4-1). | | | |
| 5006 | SMAD5: (MADH5 OR SMAD5) MOTHERS | Q99717 Q15798 | NM_005903 | |
| | AGAINST DECAPENTAPLEGIC HOMOLOG | Q9UQA1 | | |
| | 5 (SMAD 5) (MOTHERS AGAINST DPP | O14688 | | |
| | HOMOLOG 5) (SMAD5) (HSMAD5) (JV5-1). | | | |
| 5010 | SMAD9: (MADH9 OR SMAD9 OR MADH6 | O15198 O14989 | NM_005905 | |
| | OR SMAD8) MOTHERS AGAINST | | | |
| | DECAPENTAPLEGIC HOMOLOG 9 (SMAD | | | |
| | 9) (MOTHERS AGAINST DPP HOMOLOG 9) | | | |
| | (SMAD9) (MADH6) (SMAD8). | | | |
| 5014 | AKT1: (AKT1 OR RAC OR PKB) RAC-ALPHA | P31749 | NM_001014431 | 1.40/5% |
| | SERINE/THREONINE KINASE (EC 2.7.1.—) | Q9BWB6 | NM_001014432 | |
| | (RAC-PK-ALPHA) (PROTEIN KINASE B) | | NM_005163 | |
| | (PKB) (C-AKT). | | | |
| 5016 | ATM_1: (ATM) SERINE-PROTEIN KINASE | Q16551 Q12758 | NM_000051 | 0.45/6% |
| | ATM (EC 2.7.1.37) (ATAXIA | Q9NP02 | NM_138292 | |
| | TELANGIECTASIA MUTATED) (A-T, | Q9UCX7 | | |
| | MUTATED) (ATDC). | O15429 Q93007 | | |
| | | Q13315 | | |
| 5018 | CTNNB1: (CTNNB1 OR CTNNB) BETA- | P35222 | NM_001904 | |
| | CATENIN. | Q8NEW9 | | |
| | | Q8NI94 | | |
| | | Q9H391 | | |
| 5032 | MDM2: (MDM2) UBIQUITIN-PROTEIN | Q00987 Q13226 | NM_002392 | |
| | LIGASE E3 MDM2 (EC 6.3.2.—) (P53-BINDING | Q13297 Q13298 | | |
| | PROTEIN MDM2) (ONCOPROTEIN MDM2) | Q13299 Q13300 | | |
| | (DOUBLE MINUTE 2 PROTEIN) (HDM2). | Q13301 | | |
| | | Q9UGI3 | | |
| | | Q9UMT8 Q | | |
| 5036 | MYB: (MYB) MYB PROTO-ONCOGENE | Q14023 P10242 | NM_005375 | |
| | PROTEIN (C-MYB). | Q14024 | | |
| | | Q9UE83 P78525 | | |
| | | P78526 P78392 | | |
| | | P78391 | | |
| 5040 | PTCH: (PTCH) PATCHED PROTEIN | Q13635 Q13463 | NM_000264 | |
| | HOMOLOG 1 (PTC1) (PTC). | Q5VZC0 | | |
| 5042 | PTEN1-PTENP1_HUMAN: ((PTEN OR | O14781 P60484 | NM_000314 | 1.07/— % |
| | MMAC1 OR TEP1) AND (PTEN2 OR PTENP1 | O43460 Q6ICT7 | | |
| | OR PTH2)) PHOSPHATIDYLINOSITOL-3,4,5- | | | |
| | TRISPHOSPHATE 3-PHOSPHATASE AND | | | |
| | DUAL-SPECIFICITY PROTEIN | | | |
| | PHOSPHATASE PTEN (EC 3.1.3.67) (EC | | | |
| | 3.1.3.16) (EC 3.1.3.48) (PHOSPHATASE AND | | | |
| | TENSIN HOMOLOG) (MU | | | |
| 5044 | RB: (RB1 OR RB-1) RETINOBLASTOMA- | Q8IZL4 P06400 | NM_000321 | |
| | ASSOCIATED PROTEIN (PP110) (P105-RB) | P78499 | | |
| | (RB). | Q5VW46 | | |
| 5056 | IGF1_1: (IGF1 OR IBP1) INSULIN-LIKE | Q14620 P01343 | NM_000618 | |
| | GROWTH FACTOR IA PRECURSOR (IGF-IA) | P05019 | | |
| | (SOMATOMEDIN C) (INSULIN-LIKE | | | |
| | GROWTH FACTOR IB PRECURSOR) (IGF- | | | |
| | IB). | | | |
| 5131 | CDK1: (CDC2) CELL DIVISION CONTROL | O60764 P06493 | NM_001786 | |
| | PROTEIN 2 HOMOLOG (EC 2.7.1.—) (P34 | | NM_033379 | |
| | PROTEIN KINASE) (CYCLIN-DEPENDENT | | | |
| | KINASE 1) (CDK1). | | | |
| 5137 | CDK4: (CDK4) CELL DIVISION PROTEIN | P11802 O00576 | NM_000075 | |
| | KINASE 4 (EC 2.7.1.—) (CYCLIN-DEPENDENT | | | |
| | KINASE4) (PSK-J3). | | | |
| 5149 | CDKN2A_1: (CDKN2A OR CDKN2) CYCLIN- | P42771 Q15191 | NM_000077 | |
| | DEPENDENT KINASE 4 INHIBITOR A | O95440 | NM_058195 | |
| | (CDK4I) (P16-INK4) (P16-INK4A) | Q5VVJ5 | NM_058197 | |
| | (MULTIPLE TUMOR SUPPRESSOR 1) | Q96B52 | | |
| | (MTS1) (P14ARF OR ARF) (CELL CYCLE | Q9NP05 | | |
| | REGULATOR). | | | |
| 5153 | CDKN2B: (CDKN2B OR MTS2) CYCLIN- | P42772 Q6FI09 | NM_004936 | |
| | DEPENDENT KINASE 4 INHIBITOR B (P14- | | NM_078487 | |
| | INK4B) (P15-INK4B) (MULTIPLE TUMOR | | | |
| | SUPPRESSOR 2) (MTS2). | | | |
| 5159 | CDKN2D: (CDKN2D) CYCLIN-DEPENDENT | P55273 Q13102 | NM_001800 | |
| | KINASE 4 INHIBITOR D (P19-INK4D). | Q6FGE9 | NM_079421 | |
| 5171 | ERBB2: (ERBB2 OR HER2 OR NGL OR NEU) | Q6LDV1 | NM_001005862 | 1.24/21% |
| | RECEPTOR PROTEIN-TYROSINE KINASE | Q9UMK4 | NM_004448 | |
| | ERBB-2 PRECURSOR (EC 2.7.1.112) | P04626 Q14256 | | |
| | (P185ERBB2) (NEU PROTO-ONCOGENE) (C- | | | |
| | ERBB-2) (TYROSINE KINASE-TYPE CELL | | | |
| | SURFACE RECEPTOR HER2) (MLN 19) | | | |
| | (CD340 ANTIGEN). | | | |
| 5177 | FGF1: (FGF1 OR FGFA) HEPARIN-BINDING | P05230 P07502 | NM_000800 | 1.02/— % |
| | GROWTH FACTOR 1 PRECURSOR (HBGF-1) | | NM_033136 | |
| | (ACIDIC FIBROBLAST GROWTH FACTOR) | | NM_033137 | |
| | (AFGF) (BETA-ENDOTHELIAL CELL | | | |
| | GROWTH FACTOR) (ECGF-BETA). | | | |
| 5179 | FGF10: (FGF10) FIBROBLAST GROWTH | Q96P59 O15520 | NM_004465 | |
| | FACTOR-10 PRECURSOR (FGF-10) | Q6FHT6 | | |
| | (KERATINOCYTE GROWTH FACTOR 2). | | | |
| 5181 | FGF11: (FGF11 OR FHF3) FIBROBLAST | Q92914 | NM_004112 | |
| | GROWTH FACTOR-11 (FGF-11) | Q2YDX8 | | |
| | (FIBROBLAST GROWTH FACTOR | | | |
| | HOMOLOGOUS FACTOR 3) (FHF-3) | | | |
| 5185 | FGF14: (FGF14 OR FHF4) FIBROBLAST | Q92915 | NM_004115 | |
| | GROWTH FACTOR-14 (FGF-14) | Q96QX6 | NM_175929 | |
| | (FIBROBLAST GROWTH FACTOR | | | |
| | HOMOLOGOUS FACTOR 4) (FHF-4). | | | |
| 5187 | FGF16: (FGF16) FIBROBLAST GROWTH | O43320 | NM_003868 | |
| | FACTOR-16 (FGF-16). | | | |
| 5193 | FGF19_HUMAN: (FGF19) FIBROBLAST | O95750 | NM_005117 | 1.37/13% |
| | GROWTH FACTOR-19 PRECURSOR (FGF- | | | |
| | 19). | | | |
| 5195 | FGF3: (FGF3) FIBROBLAST GROWTH | P11487 | NM_005247 | |
| | FACTOR 3 INT-2 PROTO-ONCOGENE | | | |
| | PROTEIN [PRECURSOR] | | | |
| 5199 | FGF5: (FGF5) FIBROBLAST GROWTH | O75846 P12034 | NM_004464 | |
| | FACTOR-5 PRECURSOR (FGF-5) (HBGF-5). | Q3Y8M3 | NM_033143 | |
| 5201 | FGF6: (FGF6 OR HST2) FIBROBLAST | P10767 | NM_020996 | |
| | GROWTH FACTOR-6 PRECURSOR (FGF-6) | | | |
| | (HBGF-6) (HST-2). | | | |
| 5203 | FGF7: (FGF7 OR KGF) KERATINOCYTE | P21781 | NM_002009 | |
| | GROWTH FACTOR PRECURSOR (KGF) | | | |
| | (FIBROBLAST GROWTH FACTOR-7) (FGF-7) | | | |
| | (HBGF-7). | | | |
| 5207 | FGF9: (FGF9) GLIA-ACTIVATING FACTOR | P31371 Q3SY32 | NM_002010 | 0.83/4% |
| | PRECURSOR (GAF) (FIBROBLAST | | | |
| | GROWTH FACTOR-9) (FGF-9) (HBGF-9). | | | |
| 5209 | FGFR2: (FGFR2 OR ECT1 OR BEK OR | Q01742 P21802 | NM_000141 | |
| | KSAM) FIBROBLAST GROWTH FACTOR | P18443 Q12922 | NM_022970 | |
| | RECEPTOR 2 PRECURSOR (EC 2.7.10.1) | Q14300 Q14301 | | |
| | (FGFR-2) KERATINOCYTE GROWTH | Q14302 Q14303 | | |
| | FACTOR RECEPTOR 2) (CD332 ANTIGEN) | Q14305 Q | | |
| | (HEPARIN-BINDING FIBROBLAST | | | |
| | GROWTH FACTOR RECEPTOR 2). | | | |
| 5211 | IGF2_1: (IGF2) INSULIN-LIKE GROWTH | P78449 Q14299 | NM_000612 | |
| | FACTOR II PRECURSOR (IGF-II) | Q9UC69 P01344 | NM_001007139 | |
| | (SOMATOMEDIN A). | Q1WM26 | NR_003512 | |
| | | Q9UC68 | | |
| 5213 | LEPR: (OBR OR LEPR OR DB OR FA) | P48357 Q92920 | NM_002303 | |
| | LEPTIN RECEPTOR PRECURSOR (LEP-R) | Q92921 Q13592 | | |
| | (OB RECEPTOR) (OB-R) (B219RECEPTOR) | Q13593 Q13594 | | |
| | (HUB219) (B219) (CD295 ANTIGEN). | Q92919 | | |
| 5219 | NGFB: (NGFB) BETA-NERVE GROWTH | P01138 Q9P2Q8 | NM_002506 | |
| | FACTOR PRECURSOR (BETA-NGF). | Q96P60 | | |
| | | Q9UKL8 | | |
| | | Q6FHA0 | | |
| 5221 | NTRK1: (NTRK1 OR TRK) HIGH AFFINITY | Q9UIU7 | NM_002529 | |
| | NERVE GROWTH FACTOR RECEPTOR | Q15656 Q92734 | | |
| | PRECURSOR (EC 2.7.1.112) | P04629 P08119 | | |
| | (NEUROTROPHIC TYROSINE KINASE | Q15655 Q5D056 | | |
| | RECEPTOR TYPE 1) (TRK1 | Q5VZS2 | | |
| | TRANSFORMING TYROSINE KINASE | | | |
| | PROTEIN) (P140-TRKA) (TRK-A). | | | |
| 5223 | NTRK2: (NTRK2 OR TRKB) BDNF/NT-3 | Q8WXJ6 | NM_006180 | |
| | GROWTH FACTORS RECEPTOR | Q16620 Q16675 | | |
| | PRECURSOR (EC 2.7.1.112) (TRKB | | | |
| | TYROSINE KINASE) (GP145-TRKB) (TRK- | | | |
| | B). | | | |
| 5225 | NTRK3: (NTRK3 OR TRKC) NT-3 GROWTH | Q16288 Q16289 | NM_002530 | |
| | FACTOR RECEPTOR PRECURSOR (EC | Q12827 | | |
| | 2.7.1.112) (TRKC TYROSINE KINASE) | | | |
| | (GP145-TRKC) (TRK-C). | | | |
| 5229 | FLK1: (KDR OR FLK1) VASCULAR | O60723 Q14178 | NM_002253 | |
| | ENDOTHELIAL GROWTH FACTOR | P35968 | | |
| | RECEPTOR 2 PRECURSOR (EC 2.7.10.1) | | | |
| | (VEGFR-2) (KINASE INSERT DOMAIN | | | |
| | RECEPTOR) (PROTEIN-TYROSINE KINASE | | | |
| | RECEPTOR FLK-1) (CD309 ANTIGEN) | | | |
| | (VGR2). | | | |
| 5231 | FLT4: (FLT4) VASCULAR ENDOTHELIAL | P35916 | NM_002020 | |
| | GROWTH FACTOR RECEPTOR 3 | | | |
| | PRECURSOR (EC 2.7.1.112) (VEGFR-3) | | | |
| | (TYROSINE-PROTEIN KINASE RECEPTOR | | | |
| | FLT4) (VGR3). | | | |
| 5233 | CDH1: (CDH1 OR UVO OR CDHE) | Q15855 Q16194 | NM_004360 | |
| | EPITHELIAL-CADHERIN PRECURSOR (E- | Q13799 P12830 | | |
| | CADHERIN) (UVOMORULIN) (CAM 120/80) | Q14216 Q4PJ14 | | |
| | (CD324 ANTIGEN) [CONTAINS: E- | | | |
| | CAD/CTF1; E-CAD/CTF2; E-CAD/CTF3]. | | | |
| 5239 | MMP21-22-23: (MMP-23 OR MMP21/22 OR | Q9UBR9 | NM_006983 | 0.31/6% |
| | MIFR-1 OR MIFR OR DJ283E3.2) MMP-23 | O75900 | NR_002946 | |
| | (MIFR/FEMALYSIN) (DJ283E3.2.1) (MATRIX | Q9UJK8 | | |
| | METALLOPROTEINASE MMP21/22A | O75086 O75894 | | |
| | (MIFR1)) (MATRIX METALLOPROTEINASE | O75895 | | |
| | 23B) (MIFR-2 OR DJ283E3.2) MIFR-2 | Q5QPQ8 | | |
| | (DJ283E3.2.2) (MIFR2 MATRIX | Q76P96 | | |
| | METALLOPROTEINASE I | Q7LDM6 Q | | |
| 5241 | MMP6: (MPHOSPH6 OR MPP6) M-PHASE | Q99547 | NM_005792 | |
| | PHOSPHOPROTEIN 6. | | | |
| 5360 | APB: (APOB) APOLIPOPROTEIN B-100 | P78479 P78480 | NM_000384 | |
| | PRECURSOR (APO B-100) [CONTAINS: | P78481 Q13779 | | |
| | APOLIPOPROTEIN B-48 (APO B-48)]. | Q13785 Q13786 | | |
| | | Q13788 | | |
| | | Q9UMN0 | | |
| | | P04114 O | | |
| 5366 | APC3: (APOC3) APOLIPOPROTEIN C-III | P02656 Q08E83 | NM_000040 | |
| | PRECURSOR (APO-CIII). | Q6Q786 | | |
| 5434 | EDN3: (EDN3) ENDOTHELIN-3 PRECURSOR | P14138 Q03229 | NM_000114 | |
| | (ET-3). | | NM_207032 | |
| | | | NM_207033 | |
| | | | NM_207034 | |
| 5439 | FABP4: (FABP4 OR AP2 OR FABA) FATTY | P15090 | NM_001442 | |
| | ACID-BINDING PROTEIN, ADIPOCYTE | | | |
| | (AFABP) (ADIPOCYTE LIPID-BINDING | | | |
| | PROTEIN) (ALBP) (A-FABP) (P2 | | | |
| | ADIPOCYTE PROTEIN) (MYELIN P2 | | | |
| | PROTEIN HOMOLOG) (3T3-L1 LIPID | | | |
| | BINDING PROTEIN) (422 PROTEIN) (P15). | | | |
| 5440 | FABP7: (FABP7 OR FABB OR FABPB OR | O15540 O14951 | NM_001446 | |
| | BLBP OR MRG) FATTY ACID-BINDING | Q6IAU7 | | |
| | PROTEIN, BRAIN (B-FABP) (BRAIN LIPID- | | | |
| | BINDINGPROTEIN) (BLBP) (MAMMARY | | | |
| | DERIVED GROWTH INHIBITOR RELATED). | | | |
| 5442 | FABE: (FABP5 OR MAL1 OR KLBP OR | Q01469 | NM_001444 | 1.10/7% |
| | FABPE) FATTY ACID-BINDING PROTEIN, | | | |
| | EPIDERMAL (E-FABP) (PSORIASIS- | | | |
| | ASSOCIATED FATTY ACID-BINDING | | | |
| | PROTEIN HOMOLOG) (PA-FABP). | | | |
| 5446 | FABI: (FABP2) FATTY ACID-BINDING | P12104 | NM_000134 | |
| | PROTEIN, INTESTINAL (I-FABP) (FABPI). | | | |
| 5456 | FGF2_1: (FGF2 OR FGFB) HEPARIN- | P09038 | NM_002006 | |
| | BINDING GROWTH FACTOR 2 PRECURSOR | | | |
| | (HBGF-2) (BASIC FIBROBLAST GROWTH | | | |
| | FACTOR) (BFGF) (PROSTATROPIN). | | | |
| 5498 | VLDLR: (VLDLR OR LDVR) VERY LOW- | P98155 | NM_003383 | |
| | DENSITY LIPOPROTEIN RECEPTOR | Q5VVF6 | | |
| | PRECURSOR (VLDL RECEPTOR). | | | |
| 5544 | LEP_2: (LEP OR OB) LEPTIN PRECURSOR | P41159 O15158 | NM_000230 | 1.43/— % |
| | (OBESITY FACTOR) (OBESE PROTEIN). | Q56A88 | | |
| 5574 | PGH2: (PTGS2 OR COX2) PROSTAGLANDIN | P35354 Q16876 | NM_000963 | 0.31/8% |
| | G/H SYNTHASE 2 PRECURSOR (EC | | | |
| | 1.14.99.1) (CYCLOOXYGENASE-2) (COX-2) | | | |
| | (PROSTAGLANDIN-ENDOPEROXIDE | | | |
| | SYNTHASE 2) (PROSTAGLANDIN | | | |
| | H2SYNTHASE 2) (PGH SYNTHASE 2) | | | |
| | (PGHS-2) (PHS II) (PTGS2). | | | |
| 6118 | COX7A2: (COX7A2 OR COX7AL) | P14406 Q5TF59 | NM_001865 | 1.17/7% |
| | CYTOCHROME C OXIDASE POLYPEPTIDE | Q6FGI2 | | |
| | VIIA-LIVER/HEART, MITOCHONDRIAL | | | |
| | PRECURSOR (EC 1.9.3.1) (CYTOCHROME C | | | |
| | OXIDASE SUBUNIT VIIA-L). | | | |
| 6124 | CPS1: (CPS1) CARBAMOYL-PHOSPHATE | Q7Z5I5 P31327 | NM_001875 | |
| | SYNTHASE [AMMONIA], | O43774 | | |
| | MITOCHONDRIAL PRECURSOR (EC | | | |
| | 6.3.4.16) (CARBAMOYL-PHOSPHATE | | | |
| | SYNTHETASE I) (CPSASE I). | | | |
| 6146 | GCK: (GCK) HEXOKINASE D, PANCREATIC | Q05810 P35557 | NM_000162 | 1.52/24% |
| | ISOZYME (EC 2.7.1.1) (HEXOKINASE TYPE | | NM_033507 | |
| | IV) (HK4) (GLUCOKINASE). | | NM_033508 | |
| 6194 | MTHFD2: (MTGFD2 OR NMDMC) | P13995 Q53G90 | NM_006636 | |
| | BIFUNCTIONAL | Q53GV5 | | |
| | METHYLENETETRAHYDROFOLATE | Q53S36 | | |
| | DEHYDROGENASE/CYCLOHYDROLASE, | | | |
| | MITOCHONDRIAL PRECURSOR | | | |
| | [INCLUDES: NAD-DEPENDENT | | | |
| | METHYLENETETRAHYDROFOLATE | | | |
| | DEHYDROGENASE (EC 1.5.1.15); | | | |
| | METHENYLTETRAHYDROFOLATE | | | |
| | CYCLOHYDROLASE (EC 3.5.4.9)]. | | | |
| 6204 | PCK2: (PCK2 OR PEPCK2) | Q9BV62 | NM_004563 | 0.80/6% |
| | PHOSPHOENOLPYRUVATE | Q16822 O43253 | | |
| | CARBOXYKINASE, MITOCHONDRIAL | | | |
| | PRECURSOR [GTP] (EC 4.1.1.32) | | | |
| | (PHOSPHOENOLPYRUVATE | | | |
| | CARBOXYLASE) (PEPCK-M). | | | |
| 6515 | AMBP: (AMBP OR ITIL OR HCP) AMBP | P02760 P02759 | NM_001633 | |
| | PROTEIN PRECURSOR [CONTAINS: | P00977 P78491 | | |
| | ALPHA-1-MICROGLOBULIN (PROTEIN HC) | | | |
| | (COMPLEX-FORMING GLYCOPROTEIN | | | |
| | HETEROGENEOUS IN CHARGE); INTER- | | | |
| | ALPHA-TRYPSIN INHIBITOR LIGHT CHAIN | | | |
| | (ITI-LC) (BIKUNIN) (HI-30)]. | | | |
| 6887 | F2: (F2) PROTHROMBIN PRECURSOR (EC | P00734 | NM_000506 | |
| | 3.4.21.5) (COAGULATION FACTOR II). | | | |
| 6890 | F5: (F5) COAGULATION FACTOR V | P12259 Q14285 | NM_000130 | |
| | PRECURSOR (ACTIVATED PROTEIN C | Q6UPU6 | | |
| | COFACTOR). | | | |
| 6893 | F8C: (CF8 OR F8C) COAGULATION FACTOR | P00451 | NM_000132 | |
| | VIII PRECURSOR (PROCOAGULANT | | | |
| | COMPONENT) (ANTIHEMOPHILIC | | | |
| | FACTOR) (AHF) | | | |
| 7010 | TTR: (TTR OR PALB) TRANSTHYRETIN | P02766 | NM_000371 | |
| | PRECURSOR (PREALBUMIN) (TBPA) (TTR) | Q9UBZ6 | | |
| | (ATTR). | Q9UCM9 | | |
| | | Q6IB96 | | |
| 7043 | CYP3A4_HUMAN: (CYP3A4) | Q16757 | NM_017460 | |
| | CYTOCHROME P450 3A4 (EC 1.14.14.1) | Q9UK50 | | |
| | (CYPIIIA4) (NIFEDIPINE OXIDASE) (NF-25) | P08684 | | |
| | (P450-PCN1). | | | |
| 7082 | CCT8: (CCT8 OR CCTQ) T-COMPLEX | P50990 | NM_006585 | |
| | PROTEIN 1, THETA SUBUNIT (TCP-1- | | | |
| | THETA) (CCT-THETA) (KIAA0002). | | | |
| 7088 | CDC25B: (CDC25B OR CDC25HU2) M- | P30305 | NM_004358 | 0.75/36% |
| | PHASE INDUCER PHOSPHATASE 2 (EC | Q9BRA6 | NM_021872 | |
| | 3.1.3.48). | Q13971 O43551 | NM_021873 | |
| | | Q6RSS1 | | |
| | | Q5JX77 | | |
| 7091 | CDC25C: (CDC25C) M-PHASE INDUCER | P30307 Q9H2E8 | NM_022809 | |
| | PHOSPHATASE 3 (EC 3.1.3.48). | Q9H2E9 | | |
| | | Q9H2F1 | | |
| | | Q96PL3 | | |
| | | Q9H168 | | |
| 7346 | PSMA2: (PSMA2 OR PSC3) PROTEASOME | P25787 | NM_002787 | 1.11/11% |
| | SUBUNIT ALPHA TYPE 2 (EC 3.4.25.1) | Q9BU45 | | |
| | (PROTEASOME COMPONENT C3) | | | |
| | (MACROPAIN SUBUNIT C3) | | | |
| | (MULTICATALYTIC ENDOPEPTIDASE | | | |
| | COMPLEX SUBUNIT C3). | | | |
| 7352 | PSMA3: (PSMA3 OR PSC8) PROTEASOME | P25788 Q86U83 | NM_002788 | 1.01/2% |
| | SUBUNIT ALPHA TYPE 3 (EC 3.4.99.46) | Q9BS70 | NM_152132 | |
| | (PROTEASOME COMPONENT C8) | Q8N1D8 | | |
| | (MACROPAIN SUBUNIT C8) | | | |
| | (MULTICATALYTIC ENDOPEPTIDASE | | | |
| | COMPLEX SUBUNIT C8) (INGENSIN). | | | |
| 7382 | ABCC8: (ABCC8 OR SUR1 OR SUR) (ABC- | Q09428 O75948 | NM_000352 | |
| | TRANSPORTER) SULFONYLUREA | Q16583 | | |
| | RECEPTOR 1. | | | |
| 7523 | ABCG2: (ABCG2 OR ABCP OR BCRP OR | Q9UNQ0 | NM_004827 | |
| | BCRP1) ATP-BINDING CASSETTE, SUB- | Q9NUS0 | | |
| | FAMILY G, MEMBER 2 (PLACENTA- | Q9BY73 | | |
| | SPECIFIC ATP-BINDING CASSETTE | Q95374 | | |
| | TRANSPORTER) (BREAST CANCER | Q53ZQ1 | | |
| | RESISTANCE PROTEIN) (MITOXANTRONE | Q569L4 | | |
| | RESISTANCE-ASSOCIATED PROTEIN) | Q5YLG4 | | |
| | (CD338 ANTIGEN) (CDW338). | Q86V64 | | |
| | | Q8IX16 Q | | |
| 7634 | EMX-2: (EMX2 OR EMX-2) HOMEOBOX | Q04743 | NM_004098 | |
| | PROTEIN EMX2 (FRAGMENT) | Q9BQF4 | | |
| | | Q96NN8 | | |
| 7804 | KRT14: (KRT14) KERATIN, TYPE I | P02533 | NM_000526 | 1.23/— % |
| | CYTOSKELETAL 14 (CYTOKERATIN 14) | Q9BUE3 | | |
| | (K14) (CK 14). | Q14715 | | |
| | | Q53XY3 | | |
| | | Q9UBN2 | | |
| | | Q9UBN3 | | |
| | | Q9UCY4 | | |
| 7807 | KRT17: (KRT17) KERATIN, TYPE I | Q04695 | NM_000422 | |
| | CYTOSKELETAL 17 (CYTOKERATIN 17) | | | |
| | (K17) (CK 17) (39.1) (VERSION 1). | | | |
| 7837 | CDH2: (CDH2 OR CDHN OR NCAD) | P19022 Q14923 | NM_001792 | 0.54/21% |
| | CADHERIN-2 PRECURSOR (NEURAL- | | | |
| | CADHERIN) (N-CADHERIN) (CD325 | | | |
| | ANTIGEN) (CDW325). | | | |
| 7873 | ODC1: (ODC1) ORNITHINE | P11926 Q6LDS9 | NM_002539 | 1.42/3% |
| | DECARBOXYLASE (EC 4.1.1.17) (ODC). | | | |
| 7924 | RAC1: (RAC1) RAS-RELATED C3 | P63000 | NM_018890 | 1.00/15% |
| | BOTULINUM TOXIN SUBSTRATE 1 (P21- | Q3Y4D3 | | |
| | RAC1) (RAS-LIKE PROTEIN TC25). | | | |
| 7939 | RHOA: (ARHA OR ARH12 OR RHOA OR | P61586 | NM_001664 | 1.15/7% |
| | RHO12) TRANSFORMING PROTEIN RHOA | | | |
| | (H12). | | | |
| 7951 | RPL7A: (RPL7A OR SURF3 OR SURF-3) 60S | P11518 P62424 | NM_000972 | 1.28/9% |
| | RIBOSOMAL PROTEIN L7A (SURFEIT | | | |
| | LOCUS PROTEIN 3) (PLA-X POLYPEPTIDE). | | | |
| 7987 | ABCC9: (ABCC9 OR SUR2) ATP-BINDING | O60707 O60706 | NM_005691 | |
| | CASSETTE TRANSPORTER SUB-FAMILY C | | NM_020297 | |
| | MEMBER 9 (SULFONYLUREA RECEPTOR | | NM_020298 | |
| | 2). | | | |
| 7996 | TK1: (TK1) THYMIDINE KINASE, | Q9UMG9 | NM_003258 | |
| | CYTOSOLIC (EC 2.7.1.21). | P04183 Q969V0 | | |
| 8035 | AFP: (AFP) ALPHA-FETOPROTEIN | P02771 | NM_001134 | |
| | PRECURSOR (ALPHA-FETOGLOBULIN) | | | |
| | (ALPHA-1-FETOPROTEIN). | | | |
| 8071 | CTNNA1: (CTNNA1) ALPHA-1 CATENIN | P35221 Q12795 | NM_001903 | 0.87/10% |
| | (CADHERIN-ASSOCIATED PROTEIN) | | | |
| | (ALPHA E-CATENIN) (NY-REN-13 | | | |
| | ANTIGEN). | | | |
| 8080 | ENO2: (ENO2) GAMMA ENOLASE (EC | P09104 Q96J33 | NM_001975 | 0.85/24% |
| | 4.2.1.11) (2-PHOSPHO-D-GLYCERATE | | | |
| | HYDRO-LYASE) (NEURAL ENOLASE) | | | |
| | (NEURON-SPECIFIC ENOLASE) (NSE) | | | |
| | (ENOLASE 2). | | | |
| 8188 | RBL2: (RBL2 OR RB2) RETINOBLASTOMA- | Q08999 Q15073 | NM_005611 | |
| | LIKE PROTEIN 2 (130 KDA | Q92812 Q16084 | | |
| | RETINOBLASTOMA-ASSOCIATED | | | |
| | PROTEIN) (PRB2) (P130) (RBR-2). | | | |
| 8612 | TRF1: (TRF1 OR TRF) TELOMERIC REPEAT | P54274 Q93029 | NM_003218 | |
| | BINDING FACTOR 1. | Q15553 | NM_017489 | |
| 8680 | ZBTB24: (ZBTB24 OR KIAA0441 OR ZNF450) | O43167 | NM_014797 | |
| | ZINC FINGER AND BTB DOMAIN | Q5TED5 | | |
| | CONTAINING PROTEIN 24 (ZINC FINGER | Q8N455 | | |
| | PROTEIN 450) (BIF1) (BMP-INDUCED | | | |
| | FACTOR 1). | | | |
| 9037 | JAG1: (JAG1 OR JAGL1) JAGGED 1 | P78504 O14902 | NM_000214 | |
| | PRECURSOR (JAGGED1) (HJ1) (NOTCH | O15122 Q15816 | | |
| | LIGAND JAGGED 1) (CD339 ANTIGEN). | | | |
| 9046 | NOTCH1: (NOTCH1 OR TAN1) | P46531 | NM_017617 | |
| | NEUROGENIC LOCUS NOTCH PROTEIN | | | |
| | HOMOLOG 1 PRECURSOR | | | |
| 9060 | ACTG2: (ACTG2 OR ACTA3 OR ACTSG) | P63267 Q6FI22 | NM_001615 | |
| | ACTIN, GAMMA-ENTERIC SMOOTH | | | |
| | MUSCLE (ALPHA-ACTIN 3). | | | |
| 9099 | FGFR3: (FGFR3 OR JTK4) FIBROBLAST | P22607 Q16608 | NM_000142 | |
| | GROWTH FACTOR RECEPTOR 3 | Q14308 Q16294 | NM_022965 | |
| | PRECURSOR (EC 2.7.10.1) (FGFR-3) (CD333 | | | |
| | ANTIGEN). | | | |
| 9102 | FLN1: (FLNA OR FLN1 OR FLN) FILAMIN A | P21333 | NM_001456 | 0.88/3% |
| | (ALPHA-FILAMIN) (FILAMIN 1) | | | |
| | (ENDOTHELIAL ACTIN-BINDING PROTEIN) | | | |
| | (ABP-280) (NONMUSCLE FILAMIN). | | | |
| 9132 | MYH11: (MYH11 OR KIAA0866) MYOSIN-11 | P35749 P78422 | NM_002474 | |
| | (MYOSIN HEAVY CHAIN, SMOOTH | O94944 O00396 | NM_022844 | |
| | MUSCLE ISOFORM) (SMMHC). | | | |
| 9144 | SERPINF1: (SERPINF1 OR PEDF OR SDF3) | P36955 Q96R01 | NM_002615 | 1.11/13% |
| | PIGMENT EPITHELIUM-DERIVED FACTOR | Q13236 | | |
| | PRECURSOR (PEDF) (EPC-1) (STROMAL | Q9BWA4 | | |
| | CELL-DERIVED FACTOR 3) (SDF-3) | Q96CT1 | | |
| | (CASPIN). | | | |
| 9165 | SLC2A1: (SLC2A1 OR GLUT1) GLUCOSE | P11166 O75535 | NM_006516 | 0.31/14% |
| | TRANSPORTER TYPE 1, | | | |
| | ERYTHROCYTE/BRAIN. | | | |
| 9225 | ECE1: (ECE1) ENDOTHELIN-CONVERTING | Q14217 | NM_001397 | 0.87/11% |
| | ENZYME 1 (EC 3.4.24.71) (ECE-1). | Q9UJQ6 | | |
| | | Q9UPF4 | | |
| | | Q9UPM4 | | |
| | | Q9Y501 P42892 | | |
| | | Q58GE7 | | |
| | | Q5THM5 | | |
| | | Q5THM7 Q | | |
| 9249 | GGT5: (GGT5 OR GGTLA1) GAMMA- | P36269 | NM_004121 | 1.04/10% |
| | GLUTAMYLTRANSPEPTIDASE 5 | Q9UFM5 | | |
| | PRECURSOR (EC 2.3.2.2) (GAMMA- | Q96FC1 | | |
| | GLUTAMYLTRANSFERASE 5) (GGT-REL). | | | |
| 9261 | LDHB: (LDHB) L-LACTATE | P07195 | NM_002300 | 1.64/12% |
| | DEHYDROGENASE H CHAIN (EC 1.1.1.27) | | | |
| | L-LACTATE DEHYDROGENASE B CHAIN | | | |
| | (EC 1.1.1.27) (LDH-B) (LDH HEART | | | |
| | SUBUNIT) (LDH-H). | | | |
| 9302 | BMP10: (BMP10) BONE MORPHOGENETIC | O95393 | NM_014482 | |
| | PROTEIN 10. | | | |
| 9308 | GDF2: (GDF2 OR BMP9) | Q9Y571 | NM_016204 | 1.59/24% |
| | GROWTH/DIFFERENTIATION FACTOR 2 | Q9UK05 | | |
| | PRECURSOR (GDF-2) (BONE | | | |
| | MORPHOGENETIC PROTEIN 9) (BMP-9). | | | |
| 9311 | CBFA1: (RUNX2 OR CBFA1 OR AML3 OR | Q13950 O14615 | NM_001015051 | 1.67/18% |
| | PEBP2A OR OSF2) RUNT-RELATED | O95181 O14614 | NM_001024630 | |
| | TRANSCRIPTION FACTOR 2 (CORE- | | NM_004348 | |
| | BINDING FACTOR, ALPHA 1 SUBUNIT) | | | |
| | (CBF-ALPHA 1) (ACUTE MYELOID | | | |
| | LEUKEMIA 3 PROTEIN) (ONCOGENE AML- | | | |
| | 3) (POLYOMAVIRUS ENHANCER BINDING | | | |
| | PROTEIN 2 ALPHA A SUBUNIT). | | | |
| 9314 | CRABP2: (CRABP2) RETINOIC ACID- | P29373 Q6ICN6 | NM_001878 | |
| | BINDING PROTEIN II, CELLULAR (CRABP- | | | |
| | II). | | | |
| 9326 | ONECUT1: (ONECUT1 OR HNF6A OR HNF6) | Q9UMR6 | NM_004498 | |
| | HEPATOCYTE NUCLEAR FACTOR 6 (HNF- | Q99744 | | |
| | 6) (ONE CUT DOMAIN FAMILY MEMBER | Q9UBC0 | | |
| | 1). | | | |
| 9338 | HOXA2: (HOXA2) HOMEOBOX PROTEIN | O43364 | NM_006735 | |
| | HOX-A2. | | | |
| 9362 | PRRX1: (PRRX1 OR PMX1 OR PRX1) | P54821 O60807 | NM_006902 | 0.75/11% |
| | PAIRED MESODERM HOMEOBOX | | NM_022716 | |
| | PROTEIN 1 (PRX-1) (PAIRED RELATED | | | |
| | HOMEOBOX PROTEIN 1) (HOMEOBOX | | | |
| | PROTEIN PHOX1). | | | |
| 9377 | RAD17: (RAD17 OR R24L) CELL CYCLE | O75714 O75943 | NM_002873 | 1.42/— % |
| | CHECKPOINT PROTEIN RAD17 (HRAD17) | Q7Z3S4 | NM_133338 | |
| | (RF-C/ACTIVATOR 1 HOMOLOG) (HRAD17) | Q9UNK7 | NM_133339 | |
| | (DKFZP434A1135). | Q9UNR7 | NM_133341 | |
| | | Q9UNR8 | NM_133342 | |
| | | Q9UPF5 | NM_133343 | |
| | | | NM_1 | |
| 9386 | RBL1: (RBL1) RETINOBLASTOMA-LIKE | P28749 Q9H1L5 | NM_002895 | |
| | PROTEIN 1 (107 KDA RETINOBLASTOMA- | Q9H1M1 | | |
| | ASSOCIATED PROTEIN) (PRB1) (P107). | Q4VXA0 | | |
| | | Q8N5K6 | | |
| 9407 | SOX9: (SOX9) TRANSCRIPTION FACTOR | P48436 | NM_000346 | |
| | SOX-9. | | | |
| 9422 | WNT10B: (WNT10B OR WNT10 OR WNT12 | O00744 O00747 | NM_003394 | |
| | OR WNT-10B) WNT-10B PROTEIN | Q8WZ97 | | |
| | PRECURSOR (WNT-12). | | | |
| 9425 | WNT11: (WNT11 OR WNT-11) WNT-11 | O96014 | NM_004626 | 1.18/— % |
| | PROTEIN PRECURSOR. | Q8WZ98 | | |
| 9437 | WNT2: (WNT2 OR IRP OR WNT-2) WNT-2 | P09544 | NM_003391 | |
| | PROTEIN PRECURSOR (IRP PROTEIN) (INT- | Q9UDP9 | | |
| | 1 RELATED PROTEIN). | Q75N05 | | |
| 9443 | WNT3: (WNT3 OR WNT-3 OR INT4) WNT-3 | P56703 Q9H1J9 | NM_030753 | |
| | PROTO-ONCOGENE PROTEIN PRECURSOR. | | | |
| 9449 | WNT4: (WNT4 OR WNT-4) WNT-4 PROTEIN | P56705 Q9H1J8 | NM_030761 | |
| | PRECURSOR (UNQ426/PRO864). | Q9UJM2 | | |
| | | Q96T81 | | |
| | | Q9BXF5 | | |
| | | Q5TZQ0 | | |
| 9452 | WNT5A: (WNT5A OR WNT-5A) WNT-5A | P41221 | NM_003392 | |
| | PROTEIN PRECURSOR. | | | |
| 9456 | WNT5B: (WNT5B OR WNT-5B) WNT-5B | Q9BV04 | NM_030775 | 1.05/20% |
| | PROTEIN PRECURSOR. | Q96S49 Q9H1J7 | NM_032642 | |
| 9459 | WNT6: (WNT6 OR WNT-6) WNT-6 PROTEIN | Q9Y6F9 | NM_006522 | |
| | PRECURSOR. | Q9H238 | | |
| | | Q9H1J6 | | |
| 9461 | WNT7A: (WNT7A OR WNT-7A) WNT-7A | Q9Y560 O00755 | NM_004625 | |
| | PROTEIN PRECURSOR. | | | |
| 9572 | ELOVL6: (ELOVL6 OR BALDSPOT OR FAE | Q9H5J4 | NM_024090 | |
| | OR RELO2) LONG-CHAIN FATTY-ACYL | | | |
| | ELONGASE (ELOVL6 PROTEIN) (FATTY | | | |
| | ACID ELONGASE 2) (MYELINATION | | | |
| | ASSOCIATED SUR4-LIKE PROTEIN) | | | |
| | (FLJ23378). | | | |
| 9638 | F2R: (F2R OR PAR1 OR TR OR CF2R) | P25116 Q96RF7 | NM_001992 | 1.27/5% |
| | PROTEINASE ACTIVATED RECEPTOR 1 | Q9BUN4 | | |
| | PRECURSOR (PAR-1) (THROMBIN | | | |
| | RECEPTOR) (COAGULATION FACTOR II | | | |
| | RECEPTOR). | | | |
| 9663 | HERMES: (HERMES OR RBPMS) RNA- | Q92516 Q92517 | NM_006867 | 0.97/14% |
| | BINDING PROTEIN WITH MULTIPLE | Q92518 Q96J26 | | |
| | SPLICING (RBP-MS). | Q93062 | | |
| 9707 | NRP1: (NRP1 OR NRP OR VEGF165R) | Q96IH5 O60461 | NM_003873 | 1.23/44% |
| | NEUROPILIN-1 PRECURSOR (VASCULAR | O14786 | | |
| | ENDOTHELIAL CELL GROWTH FACTOR | | | |
| | 165 RECEPTOR) (CD304 ANTIGEN). | | | |
| 9713 | PAFAH1B1: (PAFAH1B1 OR PAFAHA OR | P43034 | NM_000430 | 0.91/9% |
| | LIS1 OR MDCR) PLATELET-ACTIVATING | Q8WZ88 | | |
| | FACTOR ACETYLHYDROLASE IB ALPHA | Q8WZ89 | | |
| | SUBUNIT (EC 3.1.1.47) (PAF | | | |
| | ACETYLHYDROLASE 45 KDA SUBUNIT) | | | |
| | (PAF-AH 45 KDA SUBUNIT) (PAF-AH | | | |
| | ALPHA) (PAFAH ALPHA) | | | |
| | (LISSENCEPHALY-1 PROTEIN) (LIS-1). | | | |
| 9992 | MGST1: (MGST1 OR MGST OR GST12) | P10620 | NM_020300 | |
| | GLUTATHIONE S-TRANSFERASE, | | NM_145764 | |
| | MICROSOMAL (EC 2.5.1.18). | | NM_145791 | |
| | | | NM_145792 | |
| 10164 | SIAT8A: (SIAT8A OR SIAT8) ALPHA-N- | Q93064 Q92185 | NM_003034 | |
| | ACETYL-NEURAMINNIDE ALPHA-2,8- | | | |
| | SIALYLTRANSFERASE (EC 2.4.99.8) | | | |
| | (GANGLIOSIDE GD3/GT3 SYNTHASE) | | | |
| | (SIALYLTRANSFERASE 8) (ST8SIAI). | | | |
| 10265 | ANXA2: (ANXA2 OR ANX2) ANNEXIN II | P07355 | NM_001002857 | 0.65/10% |
| | (LIPOCORTIN II) (CALPACTIN I HEAVY | Q96DD5 | NM_001002858 | |
| | CHAIN) (CHROMOBINDIN 8) (P36) | | NM_004039 | |
| | (PROTEIN I) (PLACENTAL | | | |
| | ANTICOAGULANT PROTEIN IV) (PAP-IV). | | | |
| 10455 | PEG1-MEST: (PEG1/MEST) PEG1/MEST | O15007 O14973 | NM_002402 | 1.27/6% |
| | PROTEIN (MESODERM SPECIFIC | Q92571 | NM_177524 | |
| | TRANSCRIPT (MOUSE) HOMOLOG) | | NM_177525 | |
| | (HYPOTHETICAL 38.8 KDA PROTEIN) | | | |
| | (UNKNOWN) (PROTEIN FOR MGC: 20321). | | | |
| 10467 | PLCB4: (PLCB4) PHOSPHOLIPASE C BETA | Q9UJQ2 | NM_000933 | |
| | 4. | Q9BQW5 | NM_182797 | |
| | | Q9BQW6 | | |
| | | Q9BQW8 | | |
| | | Q15147 Q5JYS8 | | |
| | | Q5JYT0 | | |
| | | Q5JYT4 | | |
| 10470 | PLCE: (PLCE OR PLCE1 OR PLC-EPSILON) | Q9HC53 | NM_016341 | |
| | PHOSPHOINOSITIDE-SPECIFIC | Q9HBX6 | | |
| | PHOSPHOLIPASE C PLC-EPSILON | Q9UHV3 | | |
| | (KIAA1516) (PANCREAS-ENRICHED | Q9H9X8 | | |
| | PHOSPHOLIPASE C) (FLJ12481). | Q9P212 | | |
| | | Q1X6H8 | | |
| | | Q5VWL5 | | |
| 10934 | CHEK1: (CHEK1 OR CHK1) | O14757 | NM_001274 | |
| | SERINE/THREONINE-PROTEIN KINASE | | | |
| | CHK1 (EC 2.7.1.—) CHECKPOINT KINASE 1. | | | |
| 10937 | CHEK2: (CHEK2 OR CHK2) | O96017 | NM_007194 | |
| | SERINE/THREONINE-PROTEIN KINASE | Q9UGF0 | NM_145862 | |
| | CHK2 (EC 2.7.1.—) (CDS1). | Q9UGF1 | | |
| | | Q6QA03 | | |
| | | Q6QA04 | | |
| | | Q6QA05 | | |
| | | Q6QA06 | | |
| | | Q6QA07 | | |
| | | Q6QA08 Q | | |
| 10970 | FZD1_2: (FZD1) FRIZZLED 1 PRECURSOR | Q9UP38 | NM_003505 | |
| | (FRIZZLED-1) (FZ-1) (HFZ1) (FZE1) (RFZ1) | O94815 Q549T8 | | |
| | (MFZ1). | | | |
| 10985 | HMGB2: (HMGB2 OR HMG2) HIGH | P26583 Q5U072 | NM_002129 | |
| | MOBILITY GROUP PROTEIN HMG2 (HMG- | | | |
| | 2). | | | |
| 10991 | HUS1 + -LIKE: (HUS1 + -LIKE OR HUS1) | O60921 | NM_004507 | |
| | HUS1 + -LIKE PROTEIN (HUS1 (S. POMBE) | | | |
| | CHECKPOINT HOMOLOG) (HUS1 | | | |
| | CHECKPOINT HOMOLOG). | | | |
| 11044 | RAD9: (RAD9) RADIO- | Q99638 Q6FI29 | NM_004584 | |
| | RESISTANCE/CHEMO-RESISTANCE/CELL | Q96C41 | | |
| | CYCLE CHECKPOINT CONTROL PROTEIN. | | | |
| 11071 | TEP1: (TEP1 OR TP1 OR TLP1) | Q99973 | NM_007110 | |
| | TELOMERASE PROTEIN-1. TELOMERASE- | | | |
| | ASSOCIATED PROTEIN TP-1. (TLP1) | | | |
| | TELOMERASE PROTEIN COMPONENT 1. | | | |
| 11074 | TERT: (TERT OR TRT OR EST2 OR TCS1) | O14783 O14746 | NM_003219 | 1.55/— % |
| | TELOMERASE REVERSE TRANSCRIPTASE | Q2XS35 | NM_198253 | |
| | (EC 2.7.7.—) (TELOMERASE CATALYTIC | Q8N6C3 | NM_198254 | |
| | SUBUNIT) (HEST2). | Q8NG46 | NM_198255 | |
| 11115 | ITGA3_5PRIME: (ITGA3) INTEGRIN ALPHA- | P26006 | NM_002204 | |
| | 3 PRECURSOR (GALACTOPROTEIN B3) | | NM_005501 | |
| | (GAPB3) (VLA-3 ALPHA CHAIN) (CD49C). | | | |
| 11151 | FGFR4: (FGFR4 OR JTK2 OR TKF) | Q14309 O43785 | NM_002011 | |
| | FIBROBLAST GROWTH FACTOR | P22455 | NM_022963 | |
| | RECEPTOR 4 PRECURSOR (EC 2.7.10.1) | | NM_213647 | |
| | (FGFR-4) (CD334 ANTIGEN). | | | |
| 11175 | KRT15: (KRT15 OR KRTB) KERATIN, TYPE | Q9BUG4 | NM_002275 | 1.10/— % |
| | I CYTOSKELETAL 15 (CYTOKERATIN 15) | P19012 | | |
| | (K15) (CK 15). | Q53XV8 | | |
| 11181 | KRT18: (KRT18 OR CYK18) KERATIN, TYPE | P05783 | NM_000224 | |
| | I CYTOSKELETAL 18 (CYTOKERATIN-18) | Q9BW26 | NM_199187 | |
| | (K18) (CK-18) (KERATIN-18). | | | |
| 11204 | KRT8: (KRT8 OR CYK8) KERATIN, TYPE II | Q14716 Q14717 | NM_002273 | 0.39/2% |
| | CYTOSKELETAL 8 (CYTOKERATIN 8) (K8) | Q14099 P05787 | | |
| | (CK 8) (KRT2-8). | Q96J60 Q53GJ0 | | |
| | | Q6DHW5 | | |
| | | Q6GMY0 | | |
| 11295 | CSPG4: (CSPG4 OR MCSP OR AN2 OR | Q6UVK1 | NM_001897 | 0.64/17% |
| | KIAA4232 OR NG2) CHONDROITIN | Q92675 | | |
| | SULFATE PROTEOGLYCAN 4 PRECURSOR | | | |
| | (CHONDROITIN SULFATE | | | |
| | PROTEOGLYCAN NG2) (MELANOMA | | | |
| | CHONDROITIN SULFATE | | | |
| | PROTEOGLYCAN) (MELANOMA- | | | |
| | ASSOCIATED CHONDROITIN SULFATE | | | |
| | PROTEOGLYCAN) (AN2 PROTEOGLYCAN). | | | |
| 11319 | SILV: (SILV OR SI OR PMEL17 OR D12S53E) | Q16565 Q14817 | NM_006928 | 1.20/— % |
| | MELANOCYTE PROTEIN PMEL 17 | Q12763 Q14448 | | |
| | PRECURSOR (MELANOCYTE LINEAGE- | P40967 | | |
| | SPECIFIC ANTIGEN GP100) (MELANOMA- | | | |
| | ASSOCIATED ME20 ANTIGEN) | | | |
| | (ME20M/ME20S) (ME20-M/ME20-S) (95 KDA | | | |
| | MELANOCYTE-SPECIFIC SECRETED | | | |
| | GLYCOPROTEIN). | | | |
| 11328 | TDGF1_2_HUMAN: ((TDGF1 OR CRIPTO) | P51864 P13385 | NM_003212 | 1.58/23% |
| | AND (TDGF3 OR TDGF2)) | | NR_002718 | |
| | TERATOCARCINOMA-DERIVED GROWTH | | | |
| | FACTOR 1 (EPIDERMAL GROWTH | | | |
| | FACTOR-LIKE CRIPTO PROTEIN CR1) | | | |
| | (CRIPTO-1 GROWTH FACTOR) (CRGF) | | | |
| | (TERATOCARCINOMA-DERIVED GROWTH | | | |
| | FACTOR 2) (EPIDERMAL GROWTH | | | |
| | FACTOR-LIKE CRIPT | | | |
| 11334 | TPM1: (TPM1 OR TPMA OR TMSA) | P09494 P09493 | NM_000366 | 0.29/9% |
| | TROPOMYOSIN ALPHA CHAIN. | P10469 Q96IK2 | | |
| | | Q9UCY9 | | |
| | | Q86W64 | | |
| 11362 | LRP8: (LRP8 OR APOER2) LOW-DENSITY | O14968 Q86V27 | NM_004631 | 1.58/— % |
| | LIPOPROTEIN RECEPTOR-RELATED | Q99876 | NM_017522 | |
| | PROTEIN 8 PRECURSOR | Q9BR78 | NM_033300 | |
| | (APOLIPOPROTEIN E RECEPTOR 2). | Q14114 | | |
| 11389 | SCARB1: (SCARB1 OR CD36L1 OR CLA1) | Q8WTV0 | NM_005505 | |
| | SCAVENGER RECEPTOR CLASS B | Q6KFX4 | | |
| | MEMBER 1 (SRB1) (SR-BI) (CD36 ANTIGEN- | Q14016 | | |
| | LIKE 1) (CD36 AND LIMPII ANALOGOUS 1) | | | |
| | (CLA-1) (COLLAGEN TYPE I RECEPTOR, | | | |
| | THROMBOSPONDIN RECEPTOR-LIKE 1). | | | |
| 11404 | CRABP1: (CRABP1 OR RBP5) RETINOIC | Q6IAY7 P29762 | NM_004378 | |
| | ACID-BINDING PROTEIN I, CELLULAR | Q8WTV5 | | |
| | (CRABP-I). | | | |
| 11635 | ACVR1: (ACVR1 OR ACVRLK2) ACTIVIN | Q04771 | NM_001105 | |
| | RECEPTOR TYPE I PRECURSOR (EC 2.7.1.—) | | | |
| | (ACTR-I) (SERINE/THREONINE-PROTEIN | | | |
| | KINASE RECEPTOR R1) (SKR1) (ACTIVIN | | | |
| | RECEPTOR-LIKE KINASE 2) (ALK-2) (TGF-B | | | |
| | SUPERFAMILY RECEPTOR TYPE I) (TSR-I). | | | |
| 11641 | ACVR2: (ACVR2) ACTIVIN RECEPTOR | Q92474 P27037 | NM_001616 | |
| | TYPE II PRECURSOR (EC 2.7.1.—) (ACTR-II) | Q53TH4 | | |
| | (ACTRIIA). | Q6NWV2 | | |
| 11644 | ACVR2B: (ACVR2B) ACTIVIN RECEPTOR | Q13705 | NM_001106 | |
| | TYPE IIB PRECURSOR (EC 2.7.1.—) (ACTR- | Q4VAV0 | | |
| | IIB). | | | |
| 11647 | ACVRL1: (ACVRL1 OR ACVRLK1 OR ALK1) | P37023 | NM_000020 | |
| | SERINE/THREONINE-PROTEIN KINASE | | | |
| | RECEPTOR R3 PRECURSOR (EC 2.7.1.37) | | | |
| | (SKR3) (ACTIVIN RECEPTOR-LIKE KINASE | | | |
| | 1) (ALK-1) (TGF-B SUPERFAMILY | | | |
| | RECEPTOR TYPE I) (TSR-I). | | | |
| 11683 | BMP15: (BMP15 OR GDF9B) BONE | Q9UMS1 | NM_005448 | |
| | MORPHOGENETIC PROTEIN 15 | O95972 Q5JST1 | | |
| | PRECURSOR (BMP-15) | | | |
| | (GROWTH/DIFFERENTIATION FACTOR 9B) | | | |
| | (GDF-9B). | | | |
| 11686 | BMPR1A: (BMPR1A OR ACVRLK3) BONE | P36894 | NM_004329 | |
| | MORPHOGENETIC PROTEIN RECEPTOR | Q8NEN8 | | |
| | TYPE IA PRECURSOR (EC 2.7.1.—) | | | |
| | (SERINE/THREONINE-PROTEIN KINASE | | | |
| | RECEPTOR R5) (SKR5) (ACTIVIN | | | |
| | RECEPTOR-LIKE KINASE 3) (ALK-3) | | | |
| 11689 | BMPR1B: (BMPR1B OR ACVRLK6) BONE | P78366 O00238 | NM_001203 | |
| | MORPHOGENETIC PROTEIN RECEPTOR | | | |
| | TYPE IB PRECURSOR (EC 2.7.11.30) | | | |
| | (CDW293 ANTIGEN) (SERINE/THREONINE- | | | |
| | PROTEIN KINASE RECEPTOR R6) (SKR6) | | | |
| | (ACTIVIN RECEPTOR-LIKE KINASE 6) | | | |
| | (ALK-6). | | | |
| 11692 | BMPR2: (BMPR2) BONE MORPHOGENETIC | Q16569 Q13873 | NM_001204 | |
| | PROTEIN RECEPTOR TYPE II PRECURSOR | | NM_033346 | |
| | (EC 2.7.1.—) (BMP TYPE II RECEPTOR) | | | |
| | (BMPR-II). | | | |
| 11698 | CD164: (CD164 OR MMGC-24) PUTATIVE | Q04900 Q5JSU6 | NM_006016 | 0.61/11% |
| | MUCIN CORE PROTEIN 24 PRECURSOR | | | |
| | (MULTI-GLYCOSYLATED CORE PROTEIN | | | |
| | 24) (MGC-24) (MUC-24) (CD164 ANTIGEN) | | | |
| | (CELL-SURFACE SIALOMUCIN MGC-24) | | | |
| | (ENDOLYN PRECURSOR). | | | |
| 11701 | CD44_EX16-20_HUMAN: (CD44 OR LHR) | Q16066 Q16522 | NM_000610 | 1.07/5% |
| | CD44 ANTIGEN PRECURSOR | P16070 P22511 | NM_001001389 | |
| | (PHAGOCYTIC GLYCOPROTEIN I) (PGP-1) | Q04858 Q13419 | NM_001001390 | |
| | (HUTCH-I) (EXTRACELLULAR MATRIX | Q13957 Q13958 | NM_001001391 | |
| | RECEPTOR-III) (ECMR-III) (GP90 | Q13959 Q | NM_001001392 | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTE | | | |
| 11704 | CD44_EX13-15_HUMAN: (CD44 OR LHR) | P16070 P22511 | NM_000610 | |
| | CD44 ANTIGEN PRECURSOR | Q04858 Q13419 | NM_001001389 | |
| | (PHAGOCYTIC GLYCOPROTEIN I) (PGP-1) | Q13957 Q13958 | NM_001001390 | |
| | (HUTCH-I) (EXTRACELLULAR MATRIX | Q13959 Q13960 | | |
| | RECEPTOR-III) (ECMR-III) (GP90 | Q13961 Q | | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTE | | | |
| 11707 | CD44_EX3-5_HUMAN: (CD44 OR LHR) CD44 | P16070 P22511 | NM_000610 | 1.08/6% |
| | ANTIGEN PRECURSOR (PHAGOCYTIC | Q04858 Q13419 | NM_001001389 | |
| | GLYCOPROTEIN I) (PGP-1) (HUTCH-I) | Q13957 Q13958 | NM_001001390 | |
| | (EXTRACELLULAR MATRIX RECEPTOR- | Q13959 Q13960 | NM_001001391 | |
| | III) (ECMR-III) (GP90 LYMPHOCYTE | Q13961 Q | | |
| | HOMING/ADHESION RECEPTOR) (HERMES | | | |
| | ANTIGEN) (HYALURONATE RECEPTOR) | | | |
| | (HEPARAN SULFATE PROTEOG | | | |
| 11710 | CD44_EX7-9_HUMAN: (CD44 OR LHR) CD44 | P22511 Q04858 | NM_000610 | 0.98/22% |
| | ANTIGEN PRECURSOR (PHAGOCYTIC | Q13419 Q13957 | NM_001001389 | |
| | GLYCOPROTEIN I) (PGP-1) (HUTCH-I) | Q13958 Q13959 | | |
| | (EXTRACELLULAR MATRIX RECEPTOR- | Q13960 Q13961 | | |
| | III) (ECMR-III) (GP90 LYMPHOCYTE | Q13967 Q | | |
| | HOMING/ADHESION RECEPTOR) (HERMES | | | |
| | ANTIGEN) (HYALURONATE RECEPTOR) | | | |
| | (HEPARAN SULFATE PROTEOG | | | |
| 11725 | CDH3: (CDH3 OR CDHP) CADHERIN-3 | P22223 | NM_001793 | |
| | PRECURSOR (PLACENTAL-CADHERIN) (P- | | | |
| | CADHERIN). | | | |
| 11761 | EGR2: (EGR2 OR KROX20) EARLY | Q9UNA6 | NM_000399 | |
| | GROWTH RESPONSE PROTEIN 2 (EGR-2) | Q8IV26 P11161 | | |
| | (KROX-20 PROTEIN) (AT591). | | | |
| 11767 | EPHB2: (EPHB2 OR EPTH3 OR ERK OR DRT | O43477 P29323 | NM_004442 | |
| | OR HEK5) EPHRIN TYPE-B RECEPTOR 2 | Q5T0U6 | NM_017449 | |
| | PRECURSOR (EC 2.7.1.112) (TYROSINE- | Q5T0U7 | | |
| | PROTEIN KINASE RECEPTOR EPH-3) (DRT) | Q5T0U8 | | |
| | (RECEPTOR PROTEIN-TYROSINE KINASE | | | |
| | HEK5) (ERK). | | | |
| 11797 | GATA2: (GATA2) ENDOTHELIAL | Q9BUJ6 P23769 | NM_032638 | 1.18/10% |
| | TRANSCRIPTION FACTOR GATA-2. | | | |
| 11804 | GATA4: (GATA4) TRANSCRIPTION | P43694 | NM_002052 | |
| | FACTOR GATA-4 (GATA BINDING | | | |
| | FACTOR-4). | | | |
| 11827 | HHEX: (HHEX OR PRHX OR PRH OR HEX) | Q03014 | NM_002729 | 1.37/6% |
| | HOMEOBOX PROTEIN PRH (HOMEOBOX | | | |
| | PROTEIN HEX). | | | |
| 11878 | IRF2: (IRF2) INTERFERON REGULATORY | Q96B99 P14316 | NM_002199 | 1.42/23% |
| | FACTOR 2 (IRF-2). | | | |
| 11920 | LMO2: (LMO2 OR RBTN2 OR RHOM2 OR | Q9HD58 | NM_005574 | |
| | TTG2) RHOMBOTIN-2 (CYSTEINE RICH | P25791 | | |
| | PROTEIN TTG-2) (T-CELL | | | |
| | TRANSLOCATION PROTEIN 2) (LIM-ONLY | | | |
| | PROTEIN 2). | | | |
| 11953 | MAP3K5: (MAP3K5 OR MAPKKK5 OR | Q99461 Q99683 | NM_005923 | |
| | MEKK5 OR ASK1) MITOGEN-ACTIVATED | Q5THN3 | | |
| | PROTEIN KINASE KINASE KINASE 5 (EC | | | |
| | 2.7.1.—) (MAPK/ERK KINASE KINASE 5) | | | |
| | (MEK KINASE 5) (MEKK 5) (APOPTOSIS | | | |
| | SIGNAL-REGULATING KINASE 1) (ASK-1). | | | |
| 11986 | NOG: (NOG) NOGGIN PRECURSOR. | Q13253 | NM_005450 | |
| 11998 | PAX5: (PAX5) PAIRED BOX PROTEIN PAX-5 | O75933 Q02548 | NM_016734 | |
| | (B-CELL SPECIFIC TRANSCRIPTION | | | |
| | FACTOR) (BSAP). | | | |
| 12034 | PRKCZ: (PRKCZ OR PKC2) PROTEIN | Q15207 Q969S4 | NM_002744 | |
| | KINASE C, ZETA TYPE (EC 2.7.1.—) (NPKC- | Q05513 | | |
| | ZETA). | Q5SYT5 | | |
| 12082 | SELP: (SELP OR GMRP) P-SELECTIN | Q8IVD1 P16109 | NM_003005 | 1.26/— % |
| | PRECURSOR (GRANULE MEMBRANE | | | |
| | PROTEIN 140) (GMP-140) (PADGEM) | | | |
| | (CD62P) (LEUKOCYTE-ENDOTHELIAL | | | |
| | CELL ADHESION MOLECULE 3) (LECAM3). | | | |
| 12085 | SEMA4D: (SEMA4D OR CD100) | Q7Z5S4 Q92854 | NM_006378 | 1.11/51% |
| | SEMAPHORIN 4D PRECURSOR | | | |
| | (LEUKOCYTE ACTIVATION ANTIGEN | | | |
| | CD100) (BB18) (A8) (GR3). (SEMA4D OR | | | |
| | SEMAJ OR SEMACL2) SEMAPHORIN 4D | | | |
| | PRECURSOR (SEMAPHORIN J) (SEMA J) | | | |
| | (SEMAPHORIN C-LIKE 2) (M-SEMA G). | | | |
| 12088 | SEMA7A: (SEMA7A OR SEMAL OR CD108) | O75326 | NM_003612 | |
| | SEMAPHORIN 7A PRECURSOR | | | |
| | (SEMAPHORIN L) (SEMA L) (SEMAPHORIN | | | |
| | K1) (SEMA K1) (JOHN-MILTON-HARGEN | | | |
| | HUMAN BLOOD GROUP AG) (JMH BLOOD | | | |
| | GROUP ANTIGEN) (CD108 ANTIGEN) | | | |
| | (CDW108). | | | |
| 12091 | SHH: (SHH) SONIC HEDGEHOG PROTEIN | Q15465 | NM_000193 | |
| | PRECURSOR (SHH) (HHG-1) | Q75MC9 | | |
| 12169 | ZNFN1A1: (ZNFN1A1 OR IKAROS OR IK1 | O00598 | NM_006060 | 1.24/— % |
| | OR LYF1) DNA-BINDING PROTEIN IKAROS | Q8WVA3 | | |
| | (LYMPHOID TRANSCRIPTION FACTOR | Q13422 | | |
| | LYF-1). | | | |
| 12281 | GCG: (GCG) GLUCAGON PRECURSOR. | P01275 | NM_002054 | 1.55/5% |
| 12350 | INSRR: (INSRR OR IRR) INSULIN | O60724 P14616 | NM_014215 | 0.93/12% |
| | RECEPTOR-RELATED PROTEIN | | | |
| | PRECURSOR (EC 2.7.1.112) (IRR) (IR- | | | |
| | RELATED RECEPTOR). | | | |
| 12359 | PDX1: (PDX1 OR IPF1) INSULIN PROMOTER | O60594 P52945 | NM_000209 | |
| | FACTOR-1 (IPF-1) (PANCREAS/DUODENUM | | | |
| | HOMEOBOX-1) (PDX-1) | | | |
| | (ISLET/DUODENUM HOMEOBOX-1) (IDX-1) | | | |
| | (SOMATOSTATIN TRANSACTIVATING | | | |
| | FACTOR-1) (STF-1) (INSULIN UPSTREAM | | | |
| | FACTOR-1) (IUF-1) (GLUCOSE-SENSITIVE | | | |
| | FACTOR) (GSF). | | | |
| 12386 | SLC16A1: (SLC16A1 OR MCT1) | Q9NSJ9 P53985 | NM_003051 | 0.95/5% |
| | MONOCARBOXYLATE TRANSPORTER 1 | | | |
| | (MCT 1). | | | |
| 12428 | PCK1: (PCK1 OR PEPCK1) | Q9UJD2 | NM_002591 | 1.39/10% |
| | PHOSPHOENOLPYRUVATE | Q8TCA3 | | |
| | CARBOXYKINASE, CYTOSOLIC (EC | P35558 | | |
| | 4.1.1.32) (PHOSPHOENOLPYRUVATE | | | |
| | CARBOXYLASE) (PEPCK-C). | | | |
| 12452 | CD45_EX10-11: (PTPRC OR CD45) | Q16614 | NM_002838 | |
| | LEUKOCYTE COMMON ANTIGEN | Q9H0Y6 | NM_080921 | |
| | PRECURSOR (EC 3.1.3.48) (L-CA) (CD45 | P08575 | NM_080922 | |
| | ANTIGEN) (T200). | | | |
| 12627 | CRIP1: (CRIP1 OR CRIP) CYSTEINE-RICH | Q13628 Q96J34 | NM_001311 | 0.11/4% |
| | PROTEIN 1 (CYSTEINE-RICH INTESTINAL | P50238 | | |
| | PROTEIN) (CRIP) (CYSTEINE-RICH HEART | | | |
| | PROTEIN) (HCRHP). | | | |
| 12690 | S100A11: (S100A11 OR S100C) | P31949 | NM_005620 | 0.84/10% |
| | CALGIZZARIN (ENDOTHELIAL | Q5VTK0 | | |
| | MONOCYTE-ACTIVATING POLYPEPTIDE) | | | |
| | (EMAP). | | | |
| 12704 | CD44_EX11-13_HUMAN: (CD44 OR LHR) | P16070 P22511 | NM_000610 | |
| | CD44 ANTIGEN PRECURSOR | Q04858 Q13419 | NM_001001389 | |
| | (PHAGOCYTIC GLYCOPROTEIN I) (PGP-1) | Q13957 Q13958 | NM_001001390 | |
| | (HUTCH-I) (EXTRACELLULAR MATRIX | Q13959 Q13960 | | |
| | RECEPTOR-III) (ECMR-III) (GP90 | Q13961 Q | | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTE | | | |
| 12707 | CD44_EX12-14_HUMAN: (CD44 OR LHR) | Q13961 Q13967 | NM_000610 | 1.69/52% |
| | CD44 ANTIGEN PRECURSOR | Q13968 Q13980 | NM_001001389 | |
| | (PHAGOCYTIC GLYCOPROTEIN I) (PGP-1) | Q15861 Q16064 | NM_001001390 | |
| | (HUTCH-I) (EXTRACELLULAR MATRIX | Q16065 Q16066 | | |
| | RECEPTOR-III) (ECMR-III) (GP90 | Q16208 Q | | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTE | | | |
| 12710 | CD44_EX8-10_HUMAN: (CD44 OR LHR) | P22511 Q04858 | NM_000610 | |
| | CD44 ANTIGEN PRECURSOR | Q13419 Q13957 | NM_001001389 | |
| | (PHAGOCYTIC GLYCOPROTEIN I) (PGP-1) | Q13958 Q13959 | | |
| | (HUTCH-I) (EXTRACELLULAR MATRIX | Q13960 Q13961 | | |
| | RECEPTOR-III) (ECMR-III) (GP90 | Q13967 Q | | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTEO | | | |
| 12713 | CD44_EX9-11_HUMAN: (CD44 OR LHR) | P16070 P22511 | NM_000610 | |
| | CD44 ANTIGEN PRECURSOR | Q04858 Q13419 | NM_001001389 | |
| | (PHAGOCYTIC GLYCOPROTEIN I) (PGP-1) | Q13957 Q13958 | | |
| | (HUTCH-I) (EXTRACELLULAR MATRIX | Q13959 Q13960 | | |
| | RECEPTOR-III) (ECMR-III) (GP90 | Q13961 Q | | |
| | LYMPHOCYTE HOMING/ADHESION | | | |
| | RECEPTOR) (HERMES ANTIGEN) | | | |
| | (HYALURONATE RECEPTOR) (HEPARAN | | | |
| | SULFATE PROTEO | | | |
| 12809 | EGFR_2: (EGFR OR ERBB1) EPIDERMAL | Q9UMD7 | — | |
| | GROWTH FACTOR RECEPTOR PRECURSOR | Q9UMD8 | | |
| | (EC 2.7.1.112) (RECEPTOR PROTEIN- | Q9UMG5 | | |
| | TYROSINE KINASE ERBB-1). | Q92795 O00732 | | |
| | | O00688 | | |
| | | Q9BZS2 | | |
| | | Q9H2C9 | | |
| | | Q9GZX1 P | | |
| 12917 | PRKCM: (PRKCM) PROTEIN KINASE C, MU | Q15139 | NM_002742 | |
| | TYPE (EC 2.7.1.—) (NPKC-MU). | | | |
| 12920 | PRKCQ: (PRKCQ OR PRKCT) PROTEIN | Q9H508 | NM_006257 | 1.00/75% |
| | KINASE C, THETA TYPE (EC 2.7.1.—) (NPKC- | Q9H549 Q04759 | | |
| | THETA). | Q3MJF1 | | |
| | | Q64FY5 | | |
| 12956 | BEX2-BEX1: ((BEX2) AND (BEX1 OR REX3)) | Q9HBH7 | NM_018476 | |
| | PROTEIN BEX1 (BRAIN-EXPRESSED X- | Q9NZ33 | NM_032621 | |
| | LINKED PROTEIN 1) (PROTEIN BEX2) | Q9BXY8 | | |
| | (BRAIN-EXPRESSED X-LINKED PROTEIN 2) | Q5JVV9 | | |
| | (HBEX2) (REDUCED EXPRESSION PROTEIN | | | |
| | 3) (REX-3). | | | |
| 12968 | CDH12: (CDH12) BRAIN-CADHERIN | P55289 | NM_004061 | |
| | PRECURSOR (BR-CADHERIN) (CADHERIN- | | | |
| | 12) (N-CADHERIN 2) (CADHERIN, NEURAL | | | |
| | TYPE, 2). | | | |
| 13004 | ELAVL2: (ELAVL2 OR HUB) ELAV-LIKE | Q13235 | NM_004432 | |
| | PROTEIN 2 (HU-ANTIGEN B) (HUB) (ELAV- | Q9H1Q8 | | |
| | LIKE NEURONAL PROTEIN 1) (NERVOUS | Q12926 Q59G15 | | |
| | SYSTEM-SPECIFIC RNA BINDING PROTEIN | Q8NEM4 | | |
| | HEL-N1). | | | |
| 13049 | HNKA: (HNKA) NEURONAL POTASSIUM | Q9UHJ4 | NM_014379 | 1.33/88% |
| | CHANNEL ALPHA SUBUNIT (POTASSIUM | | | |
| | CHANNEL KV8.1) (KCNV1 OR | | | |
| | 2700023A03RIK). | | | |
| 13079 | KCNJ4: (KCNJ4) INWARD RECTIFIER | P48050 | NM_004981 | 1.32/41% |
| | POTASSIUM CHANNEL 4 (POTASSIUM | | NM_152868 | |
| | CHANNEL, INWARDLY RECTIFYING, | | | |
| | SUBFAMILY J, MEMBER 4) (HIPPOCAMPAL | | | |
| | INWARD RECTIFIER) (HIR) (HRK1) (HIRK2) | | | |
| | (KIR2.3). | | | |
| 13106 | NTF3: (NTF3) NEUROTROPHIN-3 | P20783 | NM_002527 | |
| | PRECURSOR (NT-3) (NEUROTROPHIC | | | |
| | FACTOR) (HDNF) (NERVE GROWTH | | | |
| | FACTOR 2) (NGF-2). | | | |
| 13118 | PMX2B: (PMX2B) PAIRED MESODERM | Q99453 Q6PJD9 | NM_003924 | 1.59/44% |
| | HOMEOBOX PROTEIN 2B (PAIRED-LIKE | | | |
| | HOMEOBOX 2B) (PHOX2B | | | |
| | HOMEODOMAIN PROTEIN) | | | |
| | (NEUROBLASTOMA PHOX) (NBPHOX). | | | |
| 13124 | POU6F1: (POU6F1 OR MPOU OR BRN5 OR | Q14863 Q15944 | NM_002702 | |
| | TCFB1) POU DOMAIN, CLASS 6, | | | |
| | TRANSCRIPTION FACTOR 1 (MPOU | | | |
| | HOMEOBOX PROTEIN) (BRAIN-SPECIFIC | | | |
| | HOMEOBOX/POU DOMAIN PROTEIN 5) | | | |
| | (BRN-5 PROTEIN). | | | |
| 13163 | SYP: (SYP) SYNAPTOPHYSIN (MAJOR | P08247 Q6P2F7 | NM_003179 | |
| | SYNAPTIC VESICLE PROTEIN P38). | | | |
| 13219 | AHNAK: (AHNAK OR PM227) | Q09666 | NM_001620 | 0.99/12% |
| | NEUROBLAST DIFFERENTIATION | | | |
| | ASSOCIATED PROTEIN AHNAK | | | |
| | (DESMOYOKIN) (FLJ33834). | | | |
| 13234 | BDNF: (BDNF) BRAIN-DERIVED | P23560 | NM_001709 | 0.06/35% |
| | NEUROTROPHIC FACTOR PRECURSOR | Q9BYY7 | NM_170731 | |
| | (BDNF). | Q9UC24 | NM_170732 | |
| | | | NM_170733 | |
| | | | NM_170734 | |
| | | | NM_170735 | |
| 13246 | CACNA1A: (CACNA1A OR CACNL1A4 OR | P78511 O00555 | NM_000068 | |
| | CACH4 OR CACN3) VOLTAGE-DEPENDENT | Q92690 Q16290 | NM_023035 | |
| | P/Q-TYPE CALCIUM CHANNEL ALPHA-1A | Q99790 Q99791 | | |
| | SUBUNIT (CALCIUM CHANNEL, L TYPE, | Q99792 Q99793 | | |
| | ALPHA-1 POLYPEPTIDE ISOFORM 4) | P78510 | | |
| | (BRAIN CALCIUM CHANNEL I) (BI). | | | |
| 13249 | CACNA1B: (CACNA1B OR CACNL1A5 OR | Q00975 | NM_000718 | |
| | CACH5) VOLTAGE-DEPENDENT N-TYPE | | | |
| | CALCIUM CHANNEL ALPHA-1B SUBUNIT | | | |
| | (CALCIUM CHANNEL, L TYPE, ALPHA-1 | | | |
| | POLYPEPTIDE ISOFORM 5) (BRAIN | | | |
| | CALCIUM CHANNEL III) (BIII). | | | |
| 13252 | CACNA1E: (CACNA1E OR CACNL1A6 OR | Q15878 Q14581 | NM_000721 | |
| | CACH6) VOLTAGE-DEPENDENT R-TYPE | Q14580 | | |
| | CALCIUM CHANNEL ALPHA-1E SUBUNIT | | | |
| | (CALCIUM CHANNEL, L TYPE, ALPHA-1 | | | |
| | POLYPEPTIDE, ISOFORM 6) (BRAIN | | | |
| | CALCIUM CHANNEL II) (BII). | | | |
| 13258 | CDC42_1: (CDC42) G25K GTP-BINDING | P60953 Q7L8R5 | NM_001791 | 1.28/3% |
| | PROTEIN, PLACENTAL ISOFORM (GP) | | | |
| | (CDC42 HOMOLOG). | | | |
| 13303 | GABBR1: (GABBR1) GAMMA- | Q9UBS5 | NM_001470 | 0.93/5% |
| | AMINOBUTYRIC ACID TYPE B RECEPTOR, | O95375 | NM_021903 | |
| | SUBUNIT 1 PRECURSOR (GABA-B | Q9UQQ0 | NM_021904 | |
| | RECEPTOR 1) (GABA-B-R1) (GB1). | O96022 O95975 | NM_021905 | |
| | | O95468 | | |
| | | Q86W60 | | |
| 13309 | GABRA1: (GABRA1) GAMMA- | P14867 Q8N629 | NM_000806 | |
| | AMINOBUTYRIC-ACID RECEPTOR ALPHA- | | | |
| | 1 SUBUNIT PRECURSOR (GABA(A) | | | |
| | RECEPTOR). | | | |
| 13312 | GABRA2: (GABRA2) GAMMA- | P47869 | NM_000807 | |
| | AMINOBUTYRIC-ACID RECEPTOR ALPHA- | | | |
| | 2 SUBUNIT PRECURSOR (GABA(A) | | | |
| | RECEPTOR). | | | |
| 13327 | GABRB3: (GABRB3) GAMMA- | P28472 Q14352 | NM_000814 | |
| | AMINOBUTYRIC-ACID RECEPTOR BETA-3 | Q96FM5 | NM_021912 | |
| | SUBUNIT PRECURSOR (GABA(A) | | | |
| | RECEPTOR). | | | |
| 13354 | INA: (INA) ALPHA-INTERNEXIN (ALPHA- | Q16352 | NM_032727 | |
| | INX) (NEUROFILAMENT-66) (NF-66). | Q9BRC5 | | |
| 13402 | CACNA1D: (CACNA1D OR CACNL1A2 OR | Q13916 Q13931 | NM_000720 | |
| | CCHL1A2 OR CACH3 OR CACN4) | Q01668 | | |
| | VOLTAGE-DEPENDENT L-TYPE CALCIUM | | | |
| | CHANNEL ALPHA-1D SUBUNIT (CALCIUM | | | |
| | CHANNEL, L TYPE, ALPHA-1 | | | |
| | POLYPEPTIDE, ISOFORM 2). | | | |
| 13411 | CACNB1: (CACNB1 OR CACNLB1) | Q02641 Q02639 | NM_000723 | |
| | DIHYDROPYRIDINE-SENSITIVE L-TYPE, | Q02640 Q9C085 | NM_199247 | |
| | CHANNEL BETA-1-B2 SUBUNIT (BETA-1 | O15331 | NM_199248 | |
| | ISOFORM A). | | | |
| 13486 | NEFH: (NEFH OR NFH) NEUROFILAMENT | P12036 Q9UJS7 | NM_021076 | 1.65/— % |
| | TRIPLET H PROTEIN (200 KDA | Q9UQ14 | | |
| | NEUROFILAMENT PROTEIN) | | | |
| | (NEUROFILAMENT HEAVY POLYPEPTIDE) | | | |
| | (NF-H). | | | |
| 13489 | NEFL: (NEFL OR NFL OR NF68) | Q16154 P07196 | NM_006158 | |
| | NEUROFILAMENT TRIPLET L PROTEIN (68 | Q8IU72 | | |
| | KDA NEUROFILAMENT PROTEIN) | | | |
| | (NEUROFILAMENT LIGHT POLYPEPTIDE) | | | |
| | (NF-L). | | | |
| 13492 | NEF3: (NEF3 OR NEFM OR NFM) | P07197 | NM_005382 | |
| | NEUROFILAMENT TRIPLET M PROTEIN | | | |
| | (160 KDA NEUROFILAMENT PROTEIN) | | | |
| | (NEUROFILAMENT MEDIUM | | | |
| | POLYPEPTIDE) (NF-M) (NEUROFILAMENT | | | |
| | 3). | | | |
| 13516 | NRP2_1: (NRP2 OR VEGF165R2) | O14820 O14821 | NM_003872 | 0.72/1% |
| | NEUROPILIN-2 PRECURSOR (VASCULAR | O60462 | NM_018534 | |
| | ENDOTHELIAL CELL GROWTH FACTOR | | NM_201266 | |
| | 165 RECEPTOR 2). | | NM_201267 | |
| | | | NM_201279 | |
| 13543 | POU3F2: (POU3F2 OR BRN2 OR OTF7 OR | Q14960 P20265 | NM_005604 | |
| | OCT7) NERVOUS-SYSTEM SPECIFIC | Q9UJL0 | | |
| | OCTAMER-BINDING TRANSCRIPTION | Q86V54 | | |
| | FACTOR N-OCT 3 (BRAIN-SPECIFIC | | | |
| | HOMEOBOX/POU DOMAIN PROTEIN 2) | | | |
| | (BRN-2 PROTEIN). | | | |
| 13582 | SMDF: (NRG1 OR HGL OR NDF OR HRGA | Q15491 | NM_013959 | 0.79/— % |
| | OR GGF OR SMDF) NEUREGULIN-1, | | | |
| | SENSORY AND MOTOR NEURON-DERIVED | | | |
| | FACTOR ISOFORM. | | | |
| 13591 | STX1A: (STX1A OR STX1) SYNTAXIN 1A | O15448 | NM_004603 | 1.21/12% |
| | (NEURON-SPECIFIC ANTIGEN HPC-1). | Q9BPZ6 | | |
| | | Q12936 O15447 | | |
| | | Q16623 | | |
| 13597 | SYN2: (SYN2) SYNAPSIN II. | Q92777 | NM_003178 | |
| | | | NM_133625 | |
| 14615 | CLCN3: (CLCN3) CHLORIDE CHANNEL | P51790 O14918 | NM_001829 | |
| | PROTEIN 3 (CLC-3) (CLC-3) | Q86Z21 | | |
| 14618 | CLCN7: (CLCN7) CHLORIDE CHANNEL | Q9NYX5 | NM_001287 | |
| | PROTEIN 7 (CLC-7). | P51798 | | |
| 14716 | COL9A1_2: (COL9A1) COLLAGEN ALPHA | Q9Y6P2 | NM_001851 | 1.31/22% |
| | 1(IX) CHAIN PRECURSOR. | Q9Y6P3 | | |
| | | Q9H151 | | |
| | | Q9H152 Q99225 | | |
| | | Q13699 Q13700 | | |
| | | P20849 Q5TF52 Q | | |
| 14734 | PRKCB_3: (PRKCB1 OR PRKCB OR PKCB) | O43744 P05771 | NM_212535 | |
| | PROTEIN KINASE C, BETA TYPE (EC | P05127 Q15138 | | |
| | 2.7.1.37) (PKC-BETA) (PKC-B). | Q93060 Q9UJ33 | | |
| | | Q9UJ30 | | |
| | | Q9UEH8 | | |
| | | Q9UE49 Q | | |
| 14740 | PPARG_2: (PPARG OR NR1C3) | O00710 O14515 | NM_015869 | |
| | PEROXISOME PROLIFERATOR | Q15178 Q15179 | | |
| | ACTIVATED RECEPTOR GAMMA (PPAR- | Q15832 O00684 | | |
| | GAMMA) (PPARG2). | Q15180 P37231 | | |
| | | Q86U60 Q | | |
| 14746 | PRKCA: (PRKCA OR PKCA) PROTEIN | P17252 Q15137 | NM_002737 | |
| | KINASE C, ALPHA TYPE (EC 2.7.1.—) (PKC- | Q96RE4 | | |
| | ALPHA). | | | |
| 14749 | VEGFA: (VEGF OR VEGFA) VASCULAR | Q16889 O60720 | NM_001025366 | 0.33/25% |
| | ENDOTHELIAL GROWTH FACTOR | O75875 | NM_001025367 | |
| | PRECURSOR (VEGF-A) (VASCULAR | Q9UL23 | NM_001025368 | |
| | PERMEABILITY FACTOR) (VPF). | Q9UH58 | NM_001025369 | |
| | | Q9H1W9 | NM_001025370 | |
| | | Q9H1W8 | | |
| | | P15692 Q96L82 Q | | |
| 14752 | VGR1: (FLT1 OR FLT OR FRT) VASCULAR | P16057 O60722 | NM_002019 | 0.01/108% |
| | ENDOTHELIAL GROWTH FACTOR | P17948 Q12954 | | |
| | RECEPTOR 1 PRECURSOR (EC 2.7.1.112) | | | |
| | (VEGFR-1) (TYROSINE-PROTEIN KINASE | | | |
| | RECEPTOR FLT) (FLT-1) (TYROSINE- | | | |
| | PROTEIN KINASE FRT). | | | |
| 14773 | AKT2: (AKT2) RAC-BETA | P31751 Q68GC0 | NM_001626 | |
| | SERINE/THREONINE PROTEIN KINASE (EC | | | |
| | 2.7.1.—) (RAC-PK-BETA) (PROTEIN KINASE | | | |
| | AKT-2) (PROTEIN KINASE B, BETA) (PKB | | | |
| | BETA). | | | |
| 14776 | AKT3: (AKT3) RAC-GAMMA | Q9UFP5 | NM_005465 | |
| | SERINE/THREONINE PROTEIN KINASE (EC | Q9Y243 | | |
| | 2.7.1.—) (RAC-PK-GAMMA) (PROTEIN | Q96QV3 | | |
| | KINASE AKT-3) (PROTEIN KINASE B, | | | |
| | GAMMA) (PKB GAMMA). | | | |
| 14809 | BUB1: (BUB1 OR BUB1L) MITOTIC | O60626 O43643 | NM_004336 | |
| | CHECKPOINT SERINE/THREONINE- | O43430 O43683 | | |
| | PROTEIN KINASE BUB1 (EC 2.7.1.—) | | | |
| | (HBUB1) (BUB1A). | | | |
| 15028 | MAPK13: (MAPK13 OR PRKM13 OR SAPK4) | O14739 O15124 | NM_002754 | |
| | MITOGEN-ACTIVATED PROTEIN KINASE | Q9UNU0 | | |
| | 13 (EC 2.7.1.—) (STRESS-ACTIVATED | O15264 | | |
| | PROTEIN KINASE-4) (MITOGEN- | | | |
| | ACTIVATED PROTEIN KINASE P38 DELTA) | | | |
| | (MAP KINASE P38 DELTA). | | | |
| 15092 | EPHA1: (EPHA1 OR EPHT1 OR EPHT OR | Q15405 P21709 | NM_005232 | |
| | EPH) EPHRIN TYPE-A RECEPTOR 1 | | | |
| | PRECURSOR (EC 2.7.1.112) (TYROSINE- | | | |
| | PROTEIN KINASE RECEPTOR EPH). | | | |
| 15101 | EPHA4: (EPHA4 OR SEK OR HEK8) EPHRIN | P54764 | NM_004438 | |
| | TYPE-A RECEPTOR 4 PRECURSOR (EC | | | |
| | 2.7.1.112) (TYROSINE-PROTEIN KINASE | | | |
| | RECEPTOR SEK) (RECEPTOR PROTEIN- | | | |
| | TYROSINE KINASE HEK8). | | | |
| 15116 | EPHB4: (EPHB4 OR HTK) EPHRIN TYPE-B | Q9BXP0 | NM_004444 | 1.31/26% |
| | RECEPTOR 4 PRECURSOR (EC 2.7.1.112) | Q9BTA5 | | |
| | (TYROSINE-PROTEIN KINASE RECEPTOR | P54760 | | |
| | HTK). | | | |
| 15194 | PAK1: (PAK1) SERINE/THREONINE- | Q13567 Q13153 | NM_002576 | |
| | PROTEIN KINASE PAK 1 (EC 2.7.1.—) (P21- | O75561 | | |
| | ACTIVATED KINASE 1) (PAK-1) (P65-PAK) | Q32M53 | | |
| | (ALPHA-PAK). | Q32M54 | | |
| | | Q86W79 | | |
| 15296 | TEK: (TEK OR TIE2) ANGIOPOIETIN 1 | Q02763 | NM_000459 | |
| | RECEPTOR PRECURSOR (EC 2.7.1.112) | | | |
| | (TYROSINE-PROTEIN KINASE RECEPTOR | | | |
| | TIE-2) (TYROSINE-PROTEIN KINASE | | | |
| | RECEPTOR TEK) (P140 TEK) (TUNICA | | | |
| | INTERNA ENDOTHELIAL CELL KINASE) | | | |
| | (CD202B ANTIGEN). | | | |
| 15308 | TIE1: (TIE1 OR TIE) TYROSINE-PROTEIN | P35590 | NM_005424 | |
| | KINASE RECEPTOR TIE-1 PRECURSOR (EC | | | |
| | 2.7.1.112). | | | |
| 15492 | MAP3K3: (MAP3K3 OR MAPKKK3 OR | Q99759 | NM_002401 | |
| | MEKK3) MITOGEN-ACTIVATED PROTEIN | | NM_203351 | |
| | KINASE KINASE KINASE 3 (EC 2.7.1.—) | | | |
| | (MAPK/ERK KINASE KINASE 3) (MEK | | | |
| | KINASE 3) (MEKK 3). | | | |
| 15495 | MAP3K4: (MAP3K4 OR MAPKKK4 OR | Q92612 | NM_005922 | 0.93/77% |
| | MEKK4 OR MTK1 OR KIAA0213) MITOGEN- | Q9Y6R4 | NM_006724 | |
| | ACTIVATED PROTEIN KINASE KINASE | | | |
| | KINASE 4 (EC 2.7.1.—) (MAPK/ERK KINASE | | | |
| | KINASE 4) (MEK KINASE 4) (MEKK 4) (MAP | | | |
| | THREE KINASE 1). | | | |
| 15609 | PRKCN: (PRKCN OR EPK2) PROTEIN | O94806 | NM_005813 | |
| | KINASE C, NU TYPE (EC 2.7.1.—) (NPKC-NU) | | | |
| | (PROTEIN KINASE EPK2). | | | |
| 15723 | CSX: (NKX2E OR CSX OR NKX2-5) | P52952 | NM_004387 | |
| | HOMEOBOX PROTEIN NKX-2.5 (CARDIAC- | | | |
| | SPECIFIC HOMEOBOX) (HOMEOBOX | | | |
| | PROTEIN CSX). | | | |
| 15741 | TGIF2: (TGIF2) HOMEOBOX PROTEIN | Q9GZN2 | NM_021809 | |
| | TGIF2 (TGFB-INDUCED FACTOR 2) (5′-TG-3′ | | | |
| | INTERACTING FACTOR 2) (TGF(BETA)- | | | |
| | INDUCED TRANSCRIPTION FACTOR 2) | | | |
| | (DJ977B1.4). | | | |
| 15750 | DLX2: (DLX2) HOMEOBOX PROTEIN DLX- | Q07687 | NM_004405 | |
| | 2. | | | |
| 15762 | DLX5: (DLX5) HOMEOBOX PROTEIN DLX-5 | Q9UPL1 P56178 | NM_005221 | |
| | (DLX-5 BETA). | | | |
| 15783 | EN1: (EN1) HOMEOBOX PROTEIN | Q05925 | NM_001426 | |
| | ENGRAILED-1 (HU-EN-1). | | | |
| 15852 | HOXB3: (HOXB3 OR HOX2G) HOMEOBOX | P17484 O95615 | NM_002146 | |
| | PROTEIN HOX-B3 (HOX-2G) (HOX-2.7). | P14651 | | |
| 15906 | PITX2: (PITX2 OR RIEG1 OR RIEG OR RGS | Q9BY17 | NM_000325 | 0.62/23% |
| | OR ARP1) PITUITARY HOMEOBOX 2 (RIEG | O60578 O60579 | NM_153426 | |
| | BICOID-RELATED HOMEOBOX | O60580 Q99697 | NM_153427 | |
| | TRANSCRIPTION FACTOR) (SOLURSHIN) | | | |
| | (ALL1 RESPONSIVE PROTEIN ARP1). | | | |
| 15915 | POU2F2: (POU2F2 OR OTF2 OR OCT2) | P09086 Q9BRS4 | NM_002698 | |
| | OCTAMER-BINDING TRANSCRIPTION | Q16648 | | |
| | FACTOR 2 (OTF-2) (LYMPHOID- | | | |
| | RESTRICTED IMMUNOGLOBULIN | | | |
| | OCTAMER BINDING PROTEIN NF-A2) | | | |
| | (OCT-2 FACTOR). | | | |
| 15918 | POU5F_1: (POU5F1 OR OTF3 OR OCT3 OR | Q15167 Q15168 | NM_002701 | 1.48/42% |
| | OCT4) OCTAMER-BINDING | Q16422 Q01860 | | |
| | TRANSCRIPTION FACTOR 3A (OCT-3A) | Q9BZV9 | | |
| | (OCT-4) (POU5FLC20) (POU 5 DOMAIN | Q9BZV7 | | |
| | PROTEIN) (POU5FLC8) (OTF3C) | Q06416 | | |
| | (OCTAMER-BINDING TRANSCRIPTION | Q9BZW0 | | |
| | FACTOR 3C) (OCT-3C) (POU5FLC1) | Q9BZV8 P | | |
| | (POU5FLC12). | | | |
| 15945 | TCF2_1: (TCF2 OR HNF1B) HEPATOCYTE | P35680 | NM_000458 | |
| | NUCLEAR FACTOR 1-BETA (HNF-1B) | | | |
| | (VARIANT HEPATIC NUCLEAR FACTOR 1) | | | |
| | (VHNF1) (HOMEOPROTEIN LFB3) | | | |
| | (TRANSCRIPTION FACTOR 2) (TCF-2). | | | |
| 15999 | HOXB2: (HOXB2 OR HOX2H) HOMEOBOX | P17485 P10913 | NM_002145 | |
| | PROTEIN HOX-B2 (HOX-2H) (HOX-2.8) (K8). | P14652 Q14548 | | |
| 16035 | HOXD3: (HOXD3 OR HOX4A) HOMEOBOX | Q9BSC5 | NM_006898 | 1.02/— % |
| | PROTEIN HOX-D3 (HOX-4A). | Q99955 P31249 | | |
| 16074 | ISL1: (ISL1) INSULIN GENE ENHANCER | P47894 P20663 | NM_002202 | 1.30/6% |
| | PROTEIN ISL-1 (ISLET-1). | P61371 | | |
| 16089 | LHX2: (LHX2 OR LH2) LIM/HOMEOBOX | O95860 P50458 | NM_004789 | 1.10/23% |
| | PROTEIN LHX2 (HOMEOBOX PROTEIN LH- | Q8N1Z3 | | |
| | 2). | | | |
| 16116 | NKX2-2: (NKX2-2 OR NKX2B OR NKX2.2) | O95096 | NM_002509 | |
| | HOMEOBOX PROTEIN NKX-2.2 | | | |
| | (HOMEOBOX PROTEIN NK-2 HOMOLOG B). | | | |
| 16138 | OTX2: (OTX2) HOMEOBOX PROTEIN OTX2. | P32243 | NM_021728 | |
| | | Q9HAW3 | NM_172337 | |
| | | Q9P2R1 | | |
| | | Q6GTV3 | | |
| 16140 | PAX6: (PAX6 OR AN2) PAIRED BOX | Q99413 P26367 | NM_001604 | 1.22/— % |
| | PROTEIN PAX-6 (OCULORHOMBIN) | Q6N006 | | |
| | (ANIRIDIA, TYPE II PROTEIN). | | | |
| 16143 | PAX7: (PAX7 OR HUP1) PAIRED BOX | P23759 | NM_002584 | |
| | PROTEIN PAX-7 (HUP1). | | NM_013945 | |
| 16534 | AIF1: (AIF1 OR IBA1) ALLOGRAFT | P55008 | NM_001623 | 1.09/— % |
| | INFLAMMATORY FACTOR-1 (AIF-1) | Q9UKS9 | NM_004847 | |
| | (IONIZED CALCIUM-BINDING ADAPTER | | NM_032955 | |
| | MOLECULE 1). | | | |
| 16555 | CALU: (CALU) CALUMENIN PRECURSOR. | O60456 O43852 | NM_001219 | 0.46/8% |
| | | Q96RL3 | | |
| | | Q9NR43 | | |
| | | Q6FHB9 | | |
| 16663 | MYL2: (MYL2) MYOSIN REGULATORY | Q16123 P10916 | NM_000432 | |
| | LIGHT CHAIN 2, VENTRICULAR/CARDIAC | | | |
| | MUSCLE ISOFORM (MLC-2). | | | |
| 16666 | MYL7: (MYL7 OR MYLC2A) MYOSIN | Q01449 | NM_021223 | 1.61/46% |
| | MYOSIN REGULATORY LIGHT CHAIN 2, | | | |
| | ATRIAL ISOFORM (MYOSIN LIGHT CHAIN | | | |
| | 2A) (MLC-2A) (MLC2A) (MYOSIN | | | |
| | REGULATORY LIGHT CHAIN 7) (MYL2- | | | |
| | Q01449). | | | |
| 16675 | MYL4: (MYL4 OR MLC1) MYOSIN LIGHT | P11783 P12829 | NM_002476 | |
| | CHAIN 1, EMBRYONIC MUSCLE/ATRIAL | | | |
| | ISOFORM (PRO1957). MYOSIN LIGHT | | | |
| | CHAIN ALKALI, GT-1 ISOFORM | | | |
| | (FRAGMENT). | | | |
| 16826 | ANKRD17_1: (4933425K22RIK OR GTAR) | Q9H6J9 | NM_032217 | |
| | GENE TRAP ANKYRIN REPEAT | Q9H288 O75179 | | |
| | CONTAINING PROTEIN (KIAA0697) | | | |
| | (ANKYRIN REPEAT DOMAIN 17) | | | |
| | (ANKRD17) (SEROLOGICALLY DEFINED | | | |
| | BREAST CANCER ANTIGEN NY-BR-16) | | | |
| | (FLJ22206) (DKFZP547D039). | | | |
| 16888 | RTN4: (RTN4 OR NOGO OR ASY OR | Q9NQC3 | NM_007008 | 0.57/4% |
| | KIAA0886) RETICULON 4 (NEURITE | Q9H212 | NM_020532 | |
| | OUTGROWTH INHIBITOR) (NOGO | Q9H3I3 | NM_153828 | |
| | PROTEIN) (FOOCEN) (NEUROENDOCRINE- | Q9BXG5 | NM_207520 | |
| | SPECIFIC PROTEIN) (NSP) | Q9Y2Y7 | NM_207521 | |
| | (NEUROENDOCRINE SPECIFIC PROTEIN C | Q9UQ42 | | |
| | HOMOLOG) (RTN-X) (RETICULON 5) | Q9Y293 | | |
| | (MY043 PROTEIN). | Q9Y5U6 | | |
| | | O94962 | | |
| 16891 | S100A10: (S100A10 OR CAL1L OR ANX2LG | P60903 | NM_002966 | 0.89/6% |
| | OR CLP11) CALPACTIN I LIGHT CHAIN | | | |
| | (P10 PROTEIN) (P11) (CELLULAR LIGAND | | | |
| | OF ANNEXIN II). | | | |
| 16897 | SEMA3C: (SEMA3C OR SEMAE) | Q99985 | NM_006379 | 1.07/12% |
| | SEMAPHORIN 3C PRECURSOR | | | |
| | (SEMAPHORIN E) (SEMA E). | | | |
| 16903 | SET: (SET) SET PROTEIN (HLA-DR | Q01105 Q15541 | NM_003011 | 0.68/— % |
| | ASSOCIATED PROTEIN II) (PHAPII) | Q5VXV1 | | |
| | (PHOSPHATASE 2A INHIBITOR I2PP2A). | | | |
| 16924 | SOX15: (SOX15 OR SOX12 OR SOX20 OR | Q9Y6W7 | NM_006942 | |
| | SOX26 OR SOX27) SOX-15 PROTEIN (SOX- | O60248 P35717 | | |
| | 20 PROTEIN) (SOX-12 PROTEIN). | | | |
| 17113 | GJB3: (GJB3 OR CX31) GAP JUNCTION | O75712 | NM_024009 | |
| | BETA-3 PROTEIN (CONNEXIN 31) (CX31). | | | |
| 17161 | IGHA1-IGHA2_HUMAN: (IGHA1) IG ALPHA- | P01876 P01877 | — | |
| | 1 CHAIN C REGION (IGHA2) IG ALPHA-2 | | | |
| | CHAIN C REGION. | | | |
| 17641 | TNNC1: (TNNC1 OR TNNC) TROPONIN C, | P63316 | NM_003280 | 1.31/10% |
| | SLOW SKELETAL AND CARDIAC | | | |
| | MUSCLES (TN-C). | | | |
| 17674 | SERPINA1_2_HUMAN: (SERPINA1 OR PI OR | Q9P1P0 Q13672 | NM_000295 | 1.09/— % |
| | AAT) ALPHA-1-ANTITRYPSIN PRECURSOR | P01009 Q96BF9 | NM_001002235 | |
| | (ALPHA-1 PROTEASE INHIBITOR) (ALPHA- | Q96ES1 | NM_001002236 | |
| | 1-ANTIPROTEINASE). | Q5U0M1 | | |
| 17843 | DLK1: (DLK1 OR DLK OR PREF1 OR SCP-1) | P15803 P80370 | NM_003836 | |
| | DELTA-LIKE PROTEIN PRECURSOR (DLK) | Q96DW5 | | |
| | (PREADIPOCYTE FACTOR 1) (PREF-1) | | | |
| | (ADIPOCYTE DIFFERENTIATION | | | |
| | INHIBITOR PROTEIN). (ZOG) ZOG. | | | |
| 17848 | DNMT1: (DNMT1 OR DNMT OR AIM) DNA | Q9UHG5 | NM_001379 | |
| | (CYTOSINE-5)-METHYLTRANSFERASE | Q9ULA2 | | |
| | HSAI (EC 2.1.1.37) (DNA | Q9UMZ6 | | |
| | METHYLTRANSFERASE HSAI) (DNA | P26358 | | |
| | MTASE HSAI) (MCMT) (M.HSAI). | | | |
| 17851 | DNMT2: (DNMT2) MODIFICATION | O43669 O14717 | NM_004412 | 1.63/28% |
| | METHYLASE (EC 2.1.1.73) (CYTOSINE- | | NM_176081 | |
| | SPECIFIC METHYLTRANSFERASE). | | NM_176083 | |
| | | | NM_176084 | |
| | | | NM_176085 | |
| | | | NM_176086 | |
| 17854 | DNMT3A_1: (DNMT3A) MODIFICATION | Q9Y6K1 | NM_022552 | |
| | METHYLASE (EC 2.1.1.73) (CYTOSINE- | Q8WXU9 | NM_153759 | |
| | SPECIFIC METHYLTRANSFERASE). | | NM_175629 | |
| 17857 | DNMT3B: (DJ1085F17.1 OR DNMT3B) | Q9UBD4 | NM_006892 | |
| | MODIFICATION METHYLASE ISOFORM 1 | Q9UKA6 | NM_175848 | |
| | (EC 2.1.1.73) (CYTOSINE-SPECIFIC | Q9UJQ5 | NM_175849 | |
| | METHYLTRANSFERASE). | Q9Y5S0 | NM_175850 | |
| | | Q9Y5R9 | | |
| | | Q9UNE5 | | |
| | | Q9UBC3 | | |
| 17860 | DNMT3L: (DNMT3L) CYTOSINE-5- | Q9BUJ4 | NM_013369 | |
| | METHYLTRANSFERASE 3-LIKE PROTEIN. | Q9UJW3 | NM_175867 | |
| 17864 | EFNB1: (EFNB1 OR EPLG2 OR LERK2 OR | P98172 | NM_004429 | |
| | STRA1 OR EPL2) EPHRIN-B1 PRECURSOR | | | |
| | (EPH-RELATED RECEPTOR TYROSINE | | | |
| | KINASE LIGAND 2) (LERK-2) (ELK | | | |
| | LIGAND) (ELK-L) (STRA1 PROTEIN) (CEK5 | | | |
| | RECEPTOR LIGAND) (CEK5-L) (EFL2). | | | |
| 17920 | HDC: (HDC) HISTIDINE DECARBOXYLASE | P19113 | NM_002112 | |
| | (EC 4.1.1.22) (HDC). | | | |
| 17935 | IFNGR2: (IFNGR2 OR IFNGT1) | P38484 | NM_005534 | 0.67/5% |
| | INTERFERON-GAMMA RECEPTOR BETA | Q9BTL5 | | |
| | CHAIN PRECURSOR (INTERFERON- | | | |
| | GAMMA RECEPTOR ACCESSORY FACTOR- | | | |
| | 1) (AF-1) (INTERFERON-GAMMA | | | |
| | TRANSDUCER-1). | | | |
| 17954 | KCNQ1: (KCNQ1 OR KCNA9 OR KVLQT1) | Q92960 O00347 | NM_000218 | 1.52/22% |
| | VOLTAGE-GATED POTASSIUM CHANNEL | O60607 | NM_181797 | |
| | PROTEIN KQT-LIKE 1 (KVLQT1) (KV1.9). | Q9UMN8 | NM_181798 | |
| | | Q9UMN9 | | |
| | | O94787 P51787 | | |
| | | Q7Z6G9 | | |
| 17969 | MAP1B: (MAP1B OR MTAP5) | P46821 | NM_005909 | 0.90/— % |
| | MICROTUBULE-ASSOCIATED PROTEIN 1B | | NM_032010 | |
| | (MAP1.2) (MAP1(X)). | | | |
| 17987 | NES: (NES) INTERMEDIATE FILAMENT | O00552 P48681 | NM_006617 | |
| | PROTEIN NESTIN. | | | |
| 18017 | PTCH2: (PTCH2) PATCHED PROTEIN | O95341 O95856 | NM_003738 | |
| | HOMOLOG 2 (PTC2). | Q9Y6C5 | | |
| | | Q6UX14 | | |
| | | Q5QP87 | | |
| 18020 | RAMP2: (RAMP2) RECEPTOR ACTIVITY- | O60895 | NM_005854 | |
| | MODIFYING PROTEIN 2 PRECURSOR | Q8N1F2 | | |
| | (CRLR ACTIVITY-MODIFYING-PROTEIN 2) | | | |
| | (CALCITONIN-RECEPTOR-LIKE | | | |
| | RECEPTOR-ACTIVITY-MODIFYING- | | | |
| | PROTEIN 2). | | | |
| 18160 | HDAC2: (HDAC2) HISTONE DEACETYLASE | Q92769 Q5SRI8 | NM_001527 | 1.14/8% |
| | 2 (HD2). | Q8NEH4 | | |
| | | Q5SZ86 | | |
| 18164 | HMGIY: (HMGIY OR HMGA1 OR HMGI) | P10910 P17096 | NM_002131 | 1.56/17% |
| | HIGH MOBILITY GROUP PROTEIN HMG-Y | Q9UKB0 | NM_145899 | |
| | (HIGH MOBILITY GROUP AT-HOOK 1). | | NM_145901 | |
| | | | NM_145902 | |
| | | | NM_145903 | |
| | | | NM_145904 | |
| | | | NM_1 | |
| 18167 | HRAS: (HRAS1 OR HRAS) GTPASE HRAS | Q14080 P01112 | NM_005343 | 1.31/41% |
| | PRECURSOR (TRANSFORMING PROTEIN | Q6FHV9 | NM_176795 | |
| | P21) (H-RAS-1) (C-H-RAS). | | | |
| 18358 | ESM1: (ESM1) ENDOTHELIAL CELL- | Q15330 Q96ES3 | NM_007036 | |
| | SPECIFIC MOLECULE 1 PRECURSOR (ESM- | Q9NQ30 | | |
| | 1 SECRETORY PROTEIN) (ESM-1). | | | |
| 18379 | UBE2T: (UBE2T OR HSPC150) UBIQUITIN- | Q9NPD8 | NM_014176 | |
| | CONJUGATING ENZYME E2 T (EC 6.3.2.19) | | | |
| | (UBIQUITIN-PROTEIN LIGASE T) | | | |
| | (UBIQUITIN CARRIER PROTEIN T) | | | |
| | (FLJ20497) (2700084L22RIK). | | | |
| 18385 | IGFBP2: (IGFBP2 OR BP2) INSULIN-LIKE | Q14619 P18065 | NM_000597 | |
| | GROWTH FACTOR BINDING PROTEIN 2 | Q9UCL3 | | |
| | PRECURSOR (IGFBP-2) (IBP-2) (IGF- | | | |
| | BINDING PROTEIN 2). | | | |
| 18388 | IGFBP5: (IGFBP5 OR IBP5) INSULIN-LIKE | P24593 | NM_000599 | |
| | GROWTH FACTOR BINDING PROTEIN 5 | Q5U0A3 | | |
| | PRECURSOR (IGFBP-5) (IBP-5) (IGF- | | | |
| | BINDING PROTEIN 5). | | | |
| 19048 | ALB: (ALB OR ALB1 OR ALB-1) SERUM | P02768 Q13140 | NM_000477 | |
| | ALBUMIN PRECURSOR. | Q9UJZ0 | | |
| | | Q9UHS3 | | |
| | | Q9P1I7 Q9P157 | | |
| | | O95574 | | |
| | | Q6UXK4 | | |
| | | Q68DN5 Q | | |
| 19408 | RNF138: (RNF138) RING FINGER PROTEIN | Q9UKI6 | NM_016271 | |
| | 138 (STRIN) (TRIF) (RSD-4) (FLJ13517) (HSD- | Q9H8K2 | NM_198128 | |
| | 4) (DKFZP434I1714) (2410015A17RIK). | Q8WVD3 | | |
| | | Q9UF87 | | |
| 19669 | EPRS: (EPRS OR QPRS OR GLNS OR PARS) | P07814 | NM_004446 | |
| | BIFUNCTIONAL AMINOACYL-TRNA | | | |
| | SYNTHETASE [INCLUDES: GLUTAMYL- | | | |
| | TRNA SYNTHETASE (EC 6.1.1.17) | | | |
| | (GLUTAMATE--TRNA LIGASE); PROLYL- | | | |
| | TRNA SYNTHETASE (EC 6.1.1.15) | | | |
| | (PROLINE--TRNA LIGASE)]. | | | |
| 19690 | RPLP0: (RPLP0) 60S ACIDIC RIBOSOMAL | P05388 | NM_001002 | 1.08/3% |
| | PROTEIN P0 (L10E). | Q9BVK4 | NM_053275 | |
| 19759 | F7: (F7) COAGULATION FACTOR VII | P08709 Q14339 | NM_000131 | |
| | PRECURSOR (EC 3.4.21.21) (SERUM | Q5JVF2 | NM_019616 | |
| | PROTHROMBIN CONVERSION | | | |
| | ACCELERATOR) (EPTACOG ALFA). | | | |
| 19919 | SMURF1: (SMURF1 OR KIAA1625) SMAD | Q9UJT8 | NM_020429 | |
| | UBIQUITINATION REGULATORY FACTOR | Q9HCE7 | NM_181349 | |
| | 1 (EC 6.3.2.—) (UBIQUITIN--PROTEIN LIGASE | O75853 | | |
| | SMURF1) (SMAD-SPECIFIC E3 UBIQUITIN | | | |
| | LIGASE 1) (HSMURF1) (4930431E10RIK). | | | |
| 19922 | SMURF2: (SMURF2) SMAD | Q9HAU4 | NM_022739 | |
| | UBIQUITINATION REGULATORY FACTOR | Q9H260 | | |
| | 2 (EC 6.3.2.—) (UBIQUITIN--PROTEIN LIGASE | | | |
| | SMURF2) (SMAD-SPECIFIC E3 UBIQUITIN | | | |
| | LIGASE 2) (HSMURF2) (2810411E22RIK). | | | |
| 20039 | GATA6: (GATA6) TRANSCRIPTION | Q92908 P78327 | NM_005257 | |
| | FACTOR GATA-6 (GATA BINDING | | | |
| | FACTOR-6)(DNA BINDINGPROTEIN GATA- | | | |
| | GT2). | | | |
| 20324 | ADAM15: (ADAM15 OR MDC15) ADAM 15 | Q13444 Q13493 | NM_003815 | 0.90/8% |
| | PRECURSOR (EC 3.4.24.—) (A DISINTEGRIN | Q96C78 | NM_207191 | |
| | AND METALLOPROTEINASE DOMAIN 15) | | NM_207194 | |
| | (METALLOPROTEINASE-LIKE, | | NM_207195 | |
| | DISINTEGRIN-LIKE, AND CYSTEINE-RICH | | NM_207196 | |
| | PROTEIN 15) (MDC-15) | | NM_207197 | |
| | (METALLOPROTEASE RGD DISINTEGRIN | | | |
| | PROTEIN) (METARGIDIN) (AD56) (CRII | | | |
| 20511 | PLXNA3: (PLXNA3 OR PLXN4 OR SEX) | P51805 | NM_017514 | |
| | PLEXIN A3 PRECURSOR (PLEXIN 4) | Q5HY36 | | |
| | (TRANSMEMBRANE PROTEIN SEX) | | | |
| | (PLXN3) (PLEXIN 3). | | | |
| 20526 | SEM2: (SEM2) SEMAPHORIN SEM2. | Q9NS98 | NM_020163 | |
| | | Q9H7Q3 | | |
| | | Q7L9D9 | | |
| 20529 | SEMA3A: (SEMA3A) SEMAPHORIN 3A | Q14563 | NM_006080 | |
| | PRECURSOR (SEMAPHORIN III) (SEMA III). | | | |
| | (SEMA3A OR SEMAD OR SEMD) | | | |
| | SEMAPHORIN 3A PRECURSOR | | | |
| | (SEMAPHORIN III) (SEMA III) | | | |
| | (SEMAPHORIN D) (SEMA D). | | | |
| 20532 | SEMA3B: (SEMA3B OR SEMA5) | Q13214 Q93018 | NM_004636 | |
| | SEMAPHORIN 3B PRECURSOR | Q8TDV7 | | |
| | (SEMAPHORIN V) (SEMA V). (SEMA3B OR | Q8TB71 | | |
| | SEMAA OR SEMA) SEMAPHORIN 3B | Q96GX0 | | |
| | PRECURSOR (SEMAPHORIN A) (SEMA A). | | | |
| 20538 | SEMA3E: (SEMA3E OR KIAA0331) | O15041 | NM_012431 | |
| | SEMAPHORIN 3E PRECURSOR. (SEMA3E | Q75M94 | | |
| | OR SEMAH OR SEMH) SEMAPHORIN 3E | Q75M97 | | |
| | PRECURSOR (SEMAPHORIN H) (SEMA H). | | | |
| 20541 | SEMA3F: (SEMA3F) SEMAPHORIN 3F | Q13275 Q15704 | NM_004186 | |
| | PRECURSOR (SEMAPHORIN IV) (SEMA IV) | Q13372 Q13274 | | |
| | (SEMA III/F). | | | |
| 20547 | SEMA4F: (SEMA4F OR SEMAW OR SEMAM) | O95754 | NM_004263 | |
| | SEMAPHORIN 4F PRECURSOR | Q9NS35 | | |
| | (SEMAPHORIN W) (SEMA W). | | | |
| 20559 | SEMA6A1: (SEMA6A1) SEMAPHORIN | Q9H2E6 | NM_020796 | |
| | SEMA6A1. (SEMA6A OR SEMAQ) | Q9P2H9 | | |
| | SEMAPHORIN 6A PRECURSOR | | | |
| | (SEMAPHORIN VIA) (SEMA VIA) | | | |
| | (SEMAPHORIN Q) (SEMA Q). | | | |
| 20586 | SEMA4C: (SEMA4C OR KIAA1739) | Q9C0C4 | NM_017789 | |
| | SEMAPHORIN 4C PRECURSOR (SEMAI) | Q7Z5X0 | | |
| | (SEMACL1) (SEMAPHORIN C-LIKE 1) | | | |
| | (UNQ5855/PRO34487). | | | |
| 20616 | ASPIC1: (ASPIC1 OR CEP-68) ASPIC | Q9NQ79 | NM_018058 | |
| | PRECURSOR (CHONDROCYTE EXPRESSED | Q9NQ80 | | |
| | PROTEIN 68 KDA) ((2810454P21RIK OR | Q9NQ78 | | |
| | CRTAC1) (CRTAC1-B PROTEIN) | Q8TE52 | | |
| | (CARTILAGE ACIDIC PROTEIN 1) | Q8N4H6 | | |
| | (FLJ10320). | Q9NW46 | | |
| 20646 | PIK3C2B: (PIK3C2B) | O00750 O95666 | NM_002646 | |
| | PHOSPHATIDYLINOSITOL 3-KINASE C2 | Q5SW99 | | |
| | DOMAIN-CONTAINING BETA | | | |
| | POLYPEPTIDE (EC 2.7.1.137) | | | |
| | (PHOSPHOINOSITIDE 3-KINASE-C2-BETA) | | | |
| | (PTDINS-3-KINASE C2 BETA) (PI3K- | | | |
| | C2BETA) (C2-PI3K). | | | |
| 21270 | SCARB2: (SCARB2 OR CD36L2 OR LIMPII) | Q14108 | NM_005506 | 0.64/— % |
| | LYSOSOME MEMBRANE PROTEIN II (LIMP | | | |
| | II) (85 KDA LYSOSOMAL MEMBRANE | | | |
| | SIALOGLYCOPROTEIN) (LGP85) (CD36 | | | |
| | ANTIGEN-LIKE 2). | | | |
| 21478 | VIM: (VIM) VIMENTIN. | P08670 Q15867 | NM_003380 | 0.80/10% |
| | | Q6LER9 | | |
| | | Q8N850 | | |
| | | Q96ML2 | | |
| | | Q9NTM3 | | |
| | | Q15869 Q15868 | | |
| 21481 | VTN: (VTN) VITRONECTIN PRECURSOR | P04004 P01141 | NM_000638 | |
| | (SERUM SPREADING FACTOR) (S- | Q9BSH7 | | |
| | PROTEIN). | | | |
| 21778 | CDC42_2: (CDC42B OR CDC42) G25K GTP- | P60953 Q7L8R5 | NM_044472 | |
| | BINDING PROTEIN, BRAIN ISOFORM (GP) | | | |
| | (CDC42 HOMOLOG). | | | |
| 21835 | KRAS_1: (KRAS OR KRAS2 OR RASK2) | P01118 P01116 | NM_004985 | 0.51/27% |
| | GTPASE KRAS (K-RAS 2) (KI-RAS) (C-K- | Q96D10 | NM_033360 | |
| | RAS). | | | |
| 22015 | TC10-PIGF: (RHOQ OR ARHQ OR TC10) | P17081 | NM_002643 | 0.99/2% |
| | RHO-RELATED GTP-BINDING PROTEIN | Q8WW20 | NM_012249 | |
| | RHOQ (RAS-RELATED GTP-BINDING | Q07326 | NM_173074 | |
| | PROTEIN TC10) (RHO-LIKE GTP-BINDING | Q6NS39 | | |
| | PROTEIN TC10) (PIGF) | Q6P146 Q7Z480 | | |
| | (PHOSPHATIDYLINOSITOL-GLYCAN | Q52LS8 Q53SJ1 | | |
| | BIOSYNTHESIS, CLASS F PROTEIN) (PIG-F). | | | |
| 22039 | CSN1_3PRIME: (CSN1 OR GPS1 OR COPS1) | Q13098 | NM_004127 | 1.16/10% |
| | COP9 SIGNALOSOME COMPLEX SUBUNIT | Q8NA10 | NM_212492 | |
| | 1 (SIGNALOSOME SUBUNIT 1) (SGN1) | Q9BWL1 | | |
| | (JAB1-CONTAINING SIGNALOSOME | | | |
| | SUBUNIT 1) (G PROTEIN PATHWAY | | | |
| | SUPPRESSOR 1) (GPS1 PROTEIN) (MFH | | | |
| | PROTEIN) (GPS1_3PRIME). | | | |
| 22114 | HBZ: (HBZ OR HBZ2) HEMOGLOBIN ZETA | P02008 | NM_005332 | |
| | CHAIN. | | | |
| 22441 | GAL: (GAL OR GAL1 OR GALN OR GLNN) | P22466 Q14413 | NM_015973 | |
| | GALANIN PRECURSOR. | | | |
| 22453 | KCNQ3: (KCNQ3) VOLTAGE-GATED | O43525 | NM_004519 | |
| | POTASSIUM CHANNEL PROTEIN KQT- | | | |
| | LIKE 3. | | | |
| 22462 | RAMP1: (RAMP1) RECEPTOR ACTIVITY | O60894 | NM_005855 | |
| | MODIFYING PROTEIN 1. | | | |
| 22465 | RAMP3: (RAMP3) RECEPTOR ACTIVITY | O60896 | NM_005856 | |
| | MODIFYING PROTEIN 3. | | | |
| 22584 | GNL3: (GNL3 OR E2IG3 OR NS) GUANINE | Q9UJY0 | NM_014366 | |
| | NUCLEOTIDE BINDING PROTEIN-LIKE 3 | Q9BVP2 | NM_206825 | |
| | (NUCLEOLAR GTP-BINDING PROTEIN 3) | Q96SV6 | NM_206826 | |
| | (NUCLEOSTEMIN) (E2-INDUCED GENE 3- | Q96SV7 | | |
| | PROTEIN) (NOVEL NUCLEOLAR PROTEIN | | | |
| | 47) (NNP47) (FLJ14610) (FLJ14608) (C77032). | | | |
| 22644 | GBP2: (GBP2) INTERFERON-INDUCED | P32456 Q86TB0 | NM_004120 | |
| | GUANYLATE-BINDING PROTEIN 2 | | | |
| | (GUANINE NUCLEOTIDE-BINDING | | | |
| | PROTEIN 2) (MGBP-2). | | | |
| 22663 | HNRPA1: (HNRPA1) HETEROGENEOUS | P09651 Q6PJZ7 | NM_002136 | |
| | NUCLEAR RIBONUCLEOPROTEIN A1 | | NM_031157 | |
| | (HELIX-DESTABILIZING PROTEIN) | | | |
| | (SINGLE-STRAND BINDING PROTEIN) | | | |
| | (HNRNP CORE PROTEIN A1). | | | |
| 22693 | MYH7: (MYH7 OR MYHCB) MYOSIN | P12883 Q14904 | NM_000257 | |
| | HEAVY CHAIN, CARDIAC MUSCLE BETA | Q16579 | | |
| | ISOFORM (MYHC-BETA). | Q9H1D5 | | |
| | | Q14836 Q14837 | | |
| | | Q92679 | | |
| | | Q9UMM8 | | |
| 22699 | NASP_1: (NASP) NUCLEAR | P49321 Q96A69 | NM_002482 | |
| | AUTOANTIGENIC SPERM PROTEIN (NASP). | Q9BTW2 | NM_172164 | |
| | | Q53GW5 | | |
| 22801 | RPL6: (RPL6) 60S RIBOSOMAL PROTEIN L6 | Q02878 | NM_000970 | 1.23/17% |
| | (TAX-RESPONSIVE ENHANCER ELEMENT | Q2M3Q3 | | |
| | BINDING PROTEIN 107) (TAXREB107) | Q8WW97 | | |
| | (NEOPLASM-RELATED PROTEIN C140). | | | |
| 22935 | DDX21: (DDX21) NUCLEOLAR RNA | Q9NR30 | NM_004728 | |
| | HELICASE II (NUCLEOLAR RNA HELICASE | Q13436 | | |
| | GU) (RH II/GU) (DEAD BOX PROTEIN 21). | Q5VX41 | | |
| | | Q68D35 | | |
| 23209 | KCNJ11: (KCNJ11) ATP-SENSITIVE | Q14654 | NM_000525 | |
| | INWARD RECTIFIER POTASSIUM | Q58EX3 | | |
| | CHANNEL 11 (POTASSIUM CHANNEL, | | | |
| | INWARDLY RECTIFYING, SUBFAMILY J, | | | |
| | MEMBER 11) (INWARD RECTIFIER K+ | | | |
| | CHANNEL KIR6.2) (IKATP). | | | |
| 23212 | KCNJ3: (KCNJ3 OR GIRK1) G PROTEIN- | P48549 Q8TBI0 | NM_002239 | |
| | ACTIVATED INWARD RECTIFIER | | | |
| | POTASSIUM CHANNEL 1 (GIRK1) | | | |
| | (POTASSIUM CHANNEL, INWARDLY | | | |
| | RECTIFYING, SUBFAMILY J, MEMBER 3) | | | |
| | (INWARD RECTIFIER K+ CHANNEL | | | |
| | KIR3.1). | | | |
| 23215 | KCNJ6: (KCNJ6 OR KCNJ7 OR GIRK2 OR | P48051 | NM_002240 | |
| | KATP2) G PROTEIN-ACTIVATED INWARD | Q53WW6 | | |
| | RECTIFIER POTASSIUM CHANNEL 2 | | | |
| | (GIRK2) (POTASSIUM CHANNEL, | | | |
| | INWARDLY RECTIFYING, SUBFAMILY J, | | | |
| | MEMBER 6) (INWARD RECTIFIER K+ | | | |
| | CHANNEL KIR3.2) (KATP-2) (BIR1). | | | |
| 23218 | KCNJ9: (KCNJ9 OR GIRK3) G PROTEIN- | Q92806 | NM_004983 | |
| | ACTIVATED INWARD RECTIFIER | Q5JW75 | | |
| | POTASSIUM CHANNEL 3 (GIRK3) | | | |
| | (POTASSIUM CHANNEL, INWARDLY | | | |
| | RECTIFYING, SUBFAMILY J, MEMBER 9) | | | |
| | (INWARDLY RECTIFIER K+ CHANNEL | | | |
| | KIR3.3). | | | |
| 23248 | SOX2: (SOX2) TRANSCRIPTION FACTOR | P48431 Q14537 | NM_003106 | |
| | SOX-2. | | | |
| 23322 | CLDN1: (CLDN1 OR CLD1 OR SEMP1) | O95832 | NM_021101 | 0.71/20% |
| | CLAUDIN-1 (SENESCENCE-ASSOCIATED | | | |
| | EPITHELIAL MEMBRANE PROTEIN). | | | |
| 23325 | CLDN10: (CLDN10) CLAUDIN-10 (OSP LIKE | P78369 | NM_006984 | |
| | PROTEIN). | | NM_182848 | |
| 23361 | CLDN4: (CLDN4 OR CPETR1 OR CPER OR | O14493 | NM_001305 | 0.11/10% |
| | WBSCR8) CLAUDIN-4 (CLOSTRIDIUM | | | |
| | PERFRINGENS ENTEROTOXIN RECEPTOR) | | | |
| | (CPE-RECEPTOR) (CPE-R). | | | |
| 23364 | CLDN5: (CLDN5 OR TMVCF) CLAUDIN-5 | O00501 | NM_003277 | |
| | (TRANSMEMBRANE PROTEIN DELETED IN | Q53XW2 | | |
| | VCFS) (TMDVCF) | Q8WUW3 | | |
| 23367 | CLDN6: (CLDN6) CLAUDIN-6 (SKULLIN 2). | P56747 | NM_021195 | 0.09/40% |
| 23370 | CLDN7: (CLDN7) CLAUDIN-7. | O95471 | NM_001307 | |
| | | Q9BVN0 | | |
| | | Q6IPN3 | | |
| | | Q7Z4Y7 | | |
| 23433 | C3ORF4: (C3ORF4 OR PSEC0054 OR | Q9NY35 | NM_019895 | |
| | HSPC174) PROTEIN C3ORF4 (MEMBRANE | Q9Y4S9 | | |
| | PROTEIN GENX-3745) (CHROMOSOME 3 | Q9BUZ9 | | |
| | OPEN READING FRAME 4) | Q9NZZ5 | | |
| | (UNQ2511/PRO6000). | Q6UVX2 | | |
| | | Q502Y8 | | |
| 23565 | GJB4: (GJB4 OR CXN-30.3) GAP JUNCTION | Q9NTQ9 | NM_153212 | |
| | BETA-4 PROTEIN (CONNEXIN 30.3) | | | |
| | (CX30.3). | | | |
| 24432 | CRDBP: (IGF2BP1 OR CRDBP) MRNA- | Q9NZI8 | NM_006546 | |
| | BINDING PROTEIN CRDBP (CODING | | | |
| | REGION DETERMINANT-BINDING | | | |
| | PROTEIN) (B-ACTIN ZIPCODE BINDING | | | |
| | PROTEIN 1). | | | |
| 24438 | ELAVL4: (ELAVL4 OR HUD OR PNEM) | P26378 Q96J74 | NM_021952 | |
| | ELAV-LIKE PROTEIN 4 (PARANEOPLASTIC | | | |
| | ENCEPHALOMYELITIS ANTIGEN HUD) | | | |
| | (HU-ANTIGEN D). | | | |
| 24570 | MSI2_1: (MSI2H OR MSI2) RNA-BINDING | Q96DH6 | NM_138962 | |
| | PROTEIN MUSASHI HOMOLOG 2 | Q7Z6M7 | | |
| | (MUSASHI-2) (RNA-BINDING PROTEIN | Q8N9T4 | | |
| | MUSASHI2). | | | |
| 24627 | CDH15: (CDH15 OR CDH14 OR CDH3) | P55291 | NM_004933 | |
| | MUSCLE-CADHERIN PRECURSOR (M- | | | |
| | CADHERIN) (CADHERIN-15) (CADHERIN- | | | |
| | 14). | | | |
| 24645 | CDH4: (CDH4) CADHERIN-4 PRECURSOR | P55283 | NM_001794 | |
| | (RETINAL-CADHERIN) (R-CADHERIN) (R- | Q2M208 | | |
| | CAD) (BA489M19.1). | Q5VZ44 | | |
| | | Q9BZ05 | | |
| 24678 | DSG2: (DSG2) DESMOGLEIN 2 PRECURSOR | Q14126 | NM_001943 | |
| | (HDGC). | | | |
| 24938 | C20ORF1: (C20ORF1 OR C20ORF2 OR DIL2 | Q9ULW0 | NM_012112 | |
| | OR TPX2) RESTRICTED EXPRESSION | Q9UL00 | | |
| | PROLIFERATION ASSOCIATED PROTEIN | Q9Y2M1 | | |
| | 100 (P100) (DIFFERENTIALLY EXPRESSED | Q9UFN9 | | |
| | IN LUNG CELLS 2) (DIL-2) (TARGETING | Q9NRA3 | | |
| | PROTEIN FOR XKLP2) (C20ORF1 PROTEIN) | Q9H1R4 | | |
| | (C20ORF2 PROTEIN) (PROTEIN FLS353). | | | |
| 24947 | CLCN6_1: (CLCN6 OR KIAA0046) | P51797 P78521 | NM_001286 | |
| | CHLORIDE CHANNEL PROTEIN 6 (CLC-6). | O60818 Q99427 | | |
| | | Q99428 Q99429 | | |
| | | O60819 O60820 | | |
| | | O60821 P | | |
| 24965 | DPYSL3: (DPYSL3 OR ULIP OR DRP3 OR | Q14195 Q93012 | NM_001387 | 0.37/25% |
| | CRMP4) DIHYDROPYRIMIDINASE | | | |
| | RELATED PROTEIN-3 (DRP-3) (UNC-33- | | | |
| | LIKE PHOSPHOPROTEIN) (ULIP PROTEIN) | | | |
| | (COLLAPSIN RESPONSE MEDIATOR | | | |
| | PROTEIN 4) (CRMP-4). | | | |
| 25040 | PUM2: (PUM2 OR PUMH2 OR KIAA0235) | Q8WY43 | NM_015317 | |
| | PUMILIO HOMOLOG 2 (PUMILIO2) | Q8TB72 | | |
| | (PUMM2) (PUMILIO 2) (TRANSLATIONAL | Q9HAN2 | | |
| | REPRESSOR PUMILIO). | O00234 | | |
| | | Q53TV7 | | |
| 25052 | KITLG: (KITLG OR MGF OR SCF) KIT | P21583 Q16487 | NM_003994 | |
| | LIGAND PRECURSOR (C-KIT LIGAND) | Q9UQK7 | | |
| | (STEM CELL FACTOR) (SCF) (MAST CELL | | | |
| | GROWTH FACTOR) (MGF). | | | |
| 25213 | HCN1: (HCN1 OR BCNG1 OR HAC2) | O60741 | NM_021072 | |
| | POTASSIUM/SODIUM | | | |
| | HYPERPOLARIZATION-ACTIVATED | | | |
| | CYCLIC NUCLEOTIDE-GATED CHANNEL 1 | | | |
| | (BRAIN CYCLIC NUCLEOTIDE GATED | | | |
| | CHANNEL 1) (BCNG-1) | | | |
| | (HYPERPOLARIZATION-ACTIVATED | | | |
| | CATION CHANNEL 2) (HAC-2). | | | |
| 25222 | CACNA1G_1: (CACNA1G OR KIAA1123) | O94770 O43497 | NM_018896 | 1.02/26% |
| | VOLTAGE-DEPENDENT T-TYPE CALCIUM | O43498 | NM_198376 | |
| | CHANNEL ALPHA-1G SUBUNIT (NBR13) | Q9UHN9 | NM_198377 | |
| | (CAV3.1C). | Q9NYU4 | NM_198378 | |
| | | Q9NYU5 | NM_198379 | |
| | | Q9NYU6 | NM_198380 | |
| | | Q9NYU7 | NM_1 | |
| | | Q9NYU8 Q | | |
| 25225 | CACNA1H: (CACNA1H) VOLTAGE- | O95180 O95802 | NM_021098 | 1.41/4% |
| | DEPENDENT T-TYPE CALCIUM CHANNEL | Q96RZ9 | | |
| | ALPHA-1H SUBUNIT. | Q9NYY4 | | |
| | | Q8WWI6 | | |
| | | Q9NYY5 | | |
| | | Q96QI6 | | |
| 25279 | KCNH5_1: (KCNH5 OR EAG2) POTASSIUM | Q8NCM2 | NM_139318 | |
| | VOLTAGE-GATED CHANNEL SUBFAMILY | | NM_172375 | |
| | H MEMBER 5 (ETHER-A-GO-GO | | | |
| | POTASSIUM CHANNEL 2) (HEAG2). | | | |
| 25297 | HCN2: (HCN2 OR BCNG2) | O60742 O60743 | NM_001194 | |
| | HYPERPOLARIZATION-ACTIVATED, | O75267 | | |
| | CYCLIC NUCLEOTIDE-GATED CHANNEL 2 | Q9UBS2 | | |
| | (HAC1). | Q9UL51 | | |
| 25300 | HCN4: (HCN4 OR BCNG3) | Q9UMQ7 | NM_005477 | |
| | POTASSIUM/SODIUM | Q9Y3Q4 | | |
| | HYPERPOLARIZATION-ACTIVATED | | | |
| | CYCLIC NUCLEOTIDE-GATED CHANNEL 4 | | | |
| | (BRAIN CYCLIC NUCLEOTIDE GATED | | | |
| | CHANNEL 3) (BCNG-3). | | | |
| 25315 | KCNA1: (KCNA1) VOLTAGE-GATED | Q09470 | NM_000217 | |
| | POTASSIUM CHANNEL PROTEIN KV1.1 | | | |
| | (HUKI) (HBK1). | | | |
| 25321 | KCNA3: (KCNA3 OR HGK5) VOLTAGE- | P22001 | NM_002232 | |
| | GATED POTASSIUM CHANNEL PROTEIN | | | |
| | KV1.3 (HPCN3) (HGK5) (HUKIII) (HLK3) | | | |
| | (MK3) (RCK3) (KV3). | | | |
| 25324 | KCNA4: (KCNA4) VOLTAGE-GATED | P22459 | NM_002233 | |
| | POTASSIUM CHANNEL PROTEIN KV1.4 | | | |
| | (HK1) (HPCN2) (HBK4) (HUKII) (RCK4) | | | |
| | (RHK1) (RK4). | | | |
| 25333 | KCNA7: (KCNA7) POTASSIUM VOLTAGE- | Q9BYS4 | NM_031886 | |
| | GATED CHANNEL, SHAKER-RELATED | | | |
| | SUBFAMILY, MEMBER 7) (KCNC7). | | | |
| 25336 | KCNB1: (KCNB1) VOLTAGE-GATED | Q14721 Q14193 | NM_004975 | |
| | POTASSIUM CHANNEL PROTEIN KV2.1 | | | |
| | (DHK1) H-DRK1 K(+) CHANNEL | | | |
| | (DJ791K14.1) (POTASSIUM VOLTAGE- | | | |
| | GATEDCHANNEL, SHAB-RELATED | | | |
| | SUBFAMILY, MEMBER 1) (SHAB). | | | |
| 25339 | KCNB2: (KCNB2) VOLTAGE-GATED | Q92953 | NM_004770 | |
| | POTASSIUM CHANNEL PROTEIN KV2.2, | Q9BXD3 | | |
| | SHAB-RELATED SUBFAMILY, MEMBER 2 | | | |
| | (CDRK). | | | |
| 25342 | KCNC1: (KCNC1) VOLTAGE-GATED | P48547 | NM_004976 | |
| | POTASSIUM CHANNEL PROTEIN KV3.1 | | | |
| | (KV4) (NGK2). | | | |
| 25351 | KCND1: (KCND1) SHAL-TYPE POTASSIUM | Q9NSA2 | NM_004979 | |
| | CHANNEL (VOLTAGE-GATED POTASSIUM | O75671 | | |
| | CHANNEL KV4.1) (MSHAL). | | | |
| 25354 | KCND2: (KCND2 OR KIAA1044) VOLTAGE- | Q9NZV8 | NM_012281 | |
| | GATED POTASSIUM CHANNEL KV4.2 | O95012 O95021 | | |
| | (SHAL1) (RK5) POTASSIUM CHANNEL | Q9UN98 | | |
| | PROTEIN RK5. | Q9UNH9 | | |
| | | Q9UBY7 | | |
| 25360 | KCNH2: (KCNH2 OR HERG OR HERG1 OR | Q12809 | NM_000238 | |
| | ERG OR ERG1) POTASSIUM VOLTAGE- | Q9H3P0 | NM_172056 | |
| | GATED CHANNEL SUBFAMILY H MEMBER | O75680 O75418 | NM_172057 | |
| | 2 (ETHER-A-GO-GO RELATED GENE | Q9BT72 | | |
| | POTASSIUM CHANNEL 1) (H-ERG) (ERG1) | Q9BUT7 | | |
| | (ETHER-A-GO-GO RELATED PROTEIN 1) | | | |
| | (EAG RELATED PROTEIN 1) (EAG | | | |
| | HOMOLOG) (MERG) (MERG1) (R-E | | | |
| 25363 | KCNJ1: (KCNJ1 OR ROMK1) ATP- | Q6LD67 P48048 | NM_000220 | |
| | SENSITIVE INWARD RECTIFIER | | NM_153764 | |
| | POTASSIUM CHANNEL 1 (POTASSIUM | | NM_153765 | |
| | CHANNEL, INWARDLY RECTIFYING, | | NM_153766 | |
| | SUBFAMILY J, MEMBER 1) (ATP- | | NM_153767 | |
| | REGULATED POTASSIUM CHANNEL ROM- | | | |
| | K) (KIR1.1) ROM-K POTASSIUM CHANNEL | | | |
| | PROTEIN ISOFORM ROMK2 (KAB-1). | | | |
| 25366 | KCNJ10: (KCNJ10) ATP-SENSITIVE | Q8N4I7 P78508 | NM_002241 | |
| | INWARD RECTIFIER POTASSIUM | Q92808 | | |
| | CHANNEL 10 (POTASSIUM CHANNEL, | | | |
| | INWARDLY RECTIFYING, SUBFAMILY J, | | | |
| | MEMBER 10) (INWARD RECTIFIER K+ | | | |
| | CHANNEL KIR1.2) (ATP-DEPENDENT | | | |
| | INWARDLY RECTIFYING POTASSIUM | | | |
| | CHANNEL KIR4.1). | | | |
| 25372 | KCNJ15: (KCNJ15 OR KCNJ14) ATP- | Q96L28 Q99712 | NM_002243 | |
| | SENSITIVE INWARD RECTIFIER | Q99446 O00564 | NM_170736 | |
| | POTASSIUM CHANNEL 15 (POTASSIUM | | NM_170737 | |
| | CHANNEL, INWARDLY RECTIFYING, | | | |
| | SUBFAMILY J, MEMBER 15) (INWARD | | | |
| | RECTIFIER K+ CHANNEL KIR4.2) (KIR1.3). | | | |
| 25375 | KCNJ16: (KCNJ16) INWARD RECTIFIER | Q9NPI9 | NM_018658 | |
| | POTASSIUM CHANNEL 16 (POTASSIUM | | NM_170741 | |
| | CHANNEL, INWARDLY RECTIFYING, | | NM_170742 | |
| | SUBFAMILY J, MEMBER 16) (INWARD | | | |
| | RECTIFIER K+ CHANNEL KIR5.1) (BIR9). | | | |
| 25378 | KCNJ2: (KCNJ2 OR HIRK1) INWARD | P63252 P48049 | NM_000891 | |
| | RECTIFIER POTASSIUM CHANNEL 2 | O15110 | | |
| | (POTASSIUM CHANNEL, INWARDLY | | | |
| | RECTIFYING, SUBFAMILY J, MEMBER 2) | | | |
| | (INWARD RECTIFIER K+ CHANNEL KIR2.1) | | | |
| | (CARDIAC INWARD RECTIFIER | | | |
| | POTASSIUM CHANNEL) (IRK1) (RBL-IRK1). | | | |
| 25384 | KCNJ8: (KCNJ8) ATP-SENSITIVE INWARD | Q15842 O00657 | NM_004982 | |
| | RECTIFIER POTASSIUM CHANNEL 8 | | | |
| | (POTASSIUM CHANNEL, INWARDLY | | | |
| | RECTIFYING, SUBFAMILY J, MEMBER 8) | | | |
| | (INWARDLY RECTIFIER K+ CHANNEL | | | |
| | KIR6.1) (UKATP-1). | | | |
| 25387 | KCNJN1-KCNJ12: (KCNJN1) INWARD | Q15756 Q14500 | NM_021012 | |
| | RECTIFYING K+ CHANNEL NEGATIVE | O43401 | | |
| | REGULATOR KIR2.2V. (KCNJ12 OR IRK2) | Q8NG63 | | |
| | ATP-SENSITIVE INWARD RECTIFIER | | | |
| | POTASSIUM CHANNEL 12 (POTASSIUM | | | |
| | CHANNEL, INWARDLY RECTIFYING, | | | |
| | SUBFAMILY J, MEMBER 12) | | | |
| | (INWARDRECTIFIER K+ CHANNEL KIR2.2). | | | |
| 25390 | KCNK1: (KCNK1 OR TWIK1 OR HOHO1 OR | O00180 Q13307 | NM_002245 | 0.65/— % |
| | KCNO1) POTASSIUM CHANNEL | | | |
| | SUBFAMILY K MEMBER 1 (INWARD | | | |
| | RECTIFYING POTASSIUM CHANNEL | | | |
| | PROTEIN TWIK-1) (POTASSIUM CHANNEL | | | |
| | KCNO1) PUTATIVE POTASSIUM CHANNEL | | | |
| | TWIK. | | | |
| 25411 | KCNK2: (KCNK2 OR TREK1 OR TREK) | O95069 | NM_014217 | |
| | POTASSIUM CHANNEL SUBFAMILY K | Q9UNE3 | | |
| | MEMBER 2 (OUTWARD RECTIFYING | | | |
| | POTASSIUM CHANNEL PROTEIN TREK-1) | | | |
| | (TREK-1 K+ CHANNEL SUBUNIT) (TWO- | | | |
| | PORE POTASSIUM CHANNEL TPKC1) 2P | | | |
| | DOMAIN POTASSIUM CHANNEL KCNK2. | | | |
| 25414 | KCNK3: (KCNK3 OR TASK) POTASSIUM | O14649 | NM_002246 | |
| | CHANNEL SUBFAMILY K MEMBER 3 | | | |
| | (ACID-SENSITIVE POTASSIUM CHANNEL | | | |
| | PROTEIN TASK) (TWIK-RELATED ACID- | | | |
| | SENSITIVE K+ CHANNEL). | | | |
| 25417 | KCNK4: (KCNK4 OR TRAAK) POTASSIUM | Q9NYG8 | NM_016611 | 1.26/— % |
| | CHANNEL SUBFAMILY K MEMBER 4 | Q96T94 | NM_033310 | |
| | (TWIK-RELATED ARACHIDONIC ACID- | | NM_033311 | |
| | STIMULATED POTASSIUM CHANNEL | | | |
| | PROTEIN) (TRAAK) MECHANOSENSITIVE | | | |
| | TANDEM PORE POTASSIUM CHANNEL. | | | |
| 25420 | KCNK5: (KCNK5 OR TASK2) POTASSIUM | O95279 | NM_003740 | |
| | CHANNEL SUBFAMILY K MEMBER 5 | | | |
| | (ACID-SENSITIVE POTASSIUM CHANNEL | | | |
| | PROTEIN TASK-2) (TWIK-RELATED ACID- | | | |
| | SENSITIVE K+ CHANNEL 2). | | | |
| 25423 | KCNK7: (KCNK7) POTASSIUM CHANNEL | Q9Y2U2 | NM_005714 | |
| | SUBFAMILY K MEMBER 7 (KCNK8 OR | Q9Y2U4 | NM_033347 | |
| | KCNK6 OR DPKCH3 OR KNOT1) | Q9Y2U3 | NM_033348 | |
| | POTASSIUM CHANNEL SUBFAMILY K | | NM_033455 | |
| | MEMBER 8 (PUTATIVE POTASSIUM | | NM_033456 | |
| | CHANNEL DP3) (DOUBLE-PORE K+ | | | |
| | CHANNEL 3) (NEUROMUSCULAR TWO | | | |
| | DOMAIN POTASSIUM CHANNEL). | | | |
| 25441 | KCNQ4: (KCNQ4) POTASSIUM VOLTAGE- | P56696 O96025 | NM_004700 | |
| | GATED CHANNEL SUBFAMILY KQT | | NM_172163 | |
| | MEMBER 4 (VOLTAGE-GATED POTASSIUM | | | |
| | CHANNEL SUBUNIT KV7.4) (POTASSIUM | | | |
| | CHANNEL ALPHA SUBUNIT KVLQT4) | | | |
| | (KQT-LIKE 4). | | | |
| 25444 | KCNQ5: (KCNQ5) POTASSIUM VOLTAGE- | Q9NR82 | NM_019842 | |
| | GATED CHANNEL SUBFAMILY KQT | Q9NRN0 | | |
| | MEMBER 5 (VOLTAGE-GATED POTASSIUM | Q9NYA6 | | |
| | CHANNEL SUBUNIT KV7.5) (POTASSIUM | Q17RE1 | | |
| | CHANNEL ALPHA SUBUNIT KVLQT5) | Q5VVP3 | | |
| | (KQT-LIKE 5). | Q86W40 | | |
| 25447 | KCNS1: (KCNS1 OR KV9.1) DJ211D12.1 | Q96KK3 | NM_002251 | |
| | (POTASSIUM VOLTAGE-GATED CHANNEL, | O43652 | | |
| | DELAYED-RECTIFIER, SUBFAMILY S, | Q6DJU6 | | |
| | MEMBER 1) (DELAYED-RECTIFIER K+ | | | |
| | CHANNEL ALPHA SUBUNIT) (POTASSIUM | | | |
| | CHANNEL ALPHA SUBUNIT). | | | |
| 25450 | KCNS2: (KCNS2 OR KV9.2) POTASSIUM | Q9ULS6 | NM_020697 | |
| | CHANNEL ALPHA SUBUNIT (KIAA1144). | | | |
| 25459 | KCNH3: (KCNH3 OR KIAA1282) | Q9ULD8 | NM_012284 | |
| | POTASSIUM VOLTAGE-GATED CHANNEL | Q9UQ06 | | |
| | SUBFAMILY H MEMBER 3 (ETHER-A-GO- | | | |
| | GO-LIKE POTASSIUM CHANNEL 2) (ELK | | | |
| | CHANNEL 2) (ELK2) (BRAIN-SPECIFIC | | | |
| | EAG-LIKE CHANNEL 1) (BEC1). | | | |
| 25462 | KIAA1535: (KIAA1535) (HCN3 OR HAC3) | Q8N6W6 | NM_020897 | |
| | HYPERPOLARIZATION-ACTIVATED | Q9BWQ2 | | |
| | CATION CHANNEL, HAC3. | Q9P1Z3 | | |
| | | Q4VX12 | | |
| 25468 | KIR2.4: (KIR2.4 OR KCNJ14) INWARD | Q9UNX9 | NM_013348 | |
| | RECTIFIER POTASSIUM CHANNEL | | NM_170720 | |
| | (INWARDLY RECTIFYING POTASSIUM | | | |
| | CHANNEL KIR2.4). | | | |
| 25525 | SCN5A: (SCN5A) SODIUM CHANNEL | Q14524 | NM_000335 | |
| | PROTEIN, CARDIAC MUSCLE ALPHA- | | NM_198056 | |
| | SUBUNIT (HH1). | | | |
| 25580 | CLCN2: (CLCN2) CHLORIDE CHANNEL | Q8WU13 | NM_004366 | |
| | PROTEIN 2 (CLC-2). | P51788 O14864 | | |
| 25583 | CLCN4: (CLCN4) CHLORIDE CHANNEL | P51793 | NM_001830 | |
| | PROTEIN 4 (CLC-4) (CLCN4-2) PUTATIVE | Q9UBU1 | | |
| | CHLORIDE CHANNEL (SIMILAR TO MM | | | |
| | CLCN4-2). | | | |
| 25586 | CLCN5: (CLCN5 OR CLCK2) CHLORIDE | P51795 Q5JQD5 | NM_000084 | |
| | CHANNEL PROTEIN 5 (CLC-5). | | | |
| 25793 | MYOCD: (MYOCD OR MYCD OR SRFCP OR | Q8N7Q1 | NM_153604 | |
| | BSAC2) MYOCARDIN (SRJF CO-FACTOR | Q8IZQ8 | | |
| | PROTEIN) (BASIC SAP COILED-COIL | | | |
| | TRANSCRIPTION ACTIVATOR 2). | | | |
| 25908 | CTNND2: (CTNND2 OR NPRAP) CATENIN | Q9UQB3 | NM_001332 | |
| | DELTA-2 (DELTA-CATENIN) (NEURAL | O00379 Q13589 | | |
| | PLAKOPHILIN-RELATED ARM-REPEAT | Q9UM66 | | |
| | PROTEIN) (NPRAP) (NEUROJUNGIN) | O43840 O43206 | | |
| | (GT24). | O15390 | | |
| | | Q9UPM3 | | |
| 25932 | HIST1H2AC: (HIST1H2AC OR H2AFL) | Q93077 O00775 | NM_003512 | |
| | HISTONE H2A.L (H2A/L). | O00776 O00777 | | |
| | | O00778 Q540R1 | | |
| 25938 | HMGIC: (HMGA2 OR HMGIC) HIGH | P52926 | NM_003483 | 1.38/22% |
| | MOBILITY GROUP PROTEIN HMGI-C (HIGH | | | |
| | MOBILITY GROUP AT-HOOK 2). | | | |
| 25941 | PBXIP1: (PBXIP1 OR 4732463H20RIK) PRE- | Q9H8X6 | NM_020524 | |
| | B-CELL LEUKEMIA TRANSCRIPTION | Q9HA02 | | |
| | FACTOR INTERACTING PROTEIN 1 | Q96AQ6 | | |
| | (HEMATOPOIETIC PBX-INTERACTING | | | |
| | PROTEIN) (FLJ12435) (FLJ13157) (HPIP) | | | |
| | (HPBXIP). | | | |
| 26175 | GPC4: (GPC4) GLYPICAN-4 PRECURSOR (K- | Q96L43 O75487 | NM_001448 | |
| | GLYPICAN). | Q9UPD9 | | |
| | | Q9NU08 | | |
| | | Q9UJN1 | | |
| | | Q6ZMA6 | | |
| 26188 | KCNQ2: (KCNQ2) VOLTAGE-GATED | O43526 Q99454 | NM_004518 | |
| | POTASSIUM CHANNEL PROTEIN KQT- | O43796 O95845 | NM_172106 | |
| | LIKE 2 (NEUROBLASTOMA-SPECIFIC | O75580 Q96J59 | NM_172107 | |
| | POTASSIUM CHANNEL PROTEIN). | Q5VYT8 | NM_172108 | |
| | | | NM_172109 | |
| 26268 | VAPA: (VAPA OR VAP33) VESICLE- | Q9UBZ2 | NM_003574 | |
| | ASSOCIATED MEMBRANE PROTEIN- | Q9P0L0 O75453 | NM_194434 | |
| | ASSOCIATED PROTEIN A (VAMP- | Q5U0E7 | | |
| | ASSOCIATED PROTEIN A) (VAMP-A) (VAP- | | | |
| | A) (33 KDA VAMP-ASSOCIATED PROTEIN) | | | |
| | (VAP-33). | | | |
| 26313 | CACNB2: (CACNB2 OR CACNLB2 OR | O00304 Q08289 | NM_000724 | |
| | MYSB) DIHYDROPYRIDINE-SENSITIVE L- | Q96NZ4 | NM_201570 | |
| | TYPE, CALCIUM CHANNEL BETA-2 | Q96NZ3 | NM_201571 | |
| | SUBUNIT (CAB2) (VOLTAGE-DEPENDENT | Q96NZ5 | NM_201572 | |
| | CALCIUM CHANNEL BETA-2 SUBUNIT) | Q9Y340 | NM_201590 | |
| | (LAMBERT-EATON MYASTHENIC | Q9Y341 | NM_201593 | |
| | SYNDROME ANTIGEN B) (MYSB). | Q9HD32 | NM_2 | |
| | | Q9BWU2 Q | | |
| 26316 | CACNB4: (CACNB4 OR CACNLB4) | O00305 O60515 | NM_000726 | |
| | DIHYDROPYRIDINE-SENSITIVE L-TYPE, | Q96L40 | | |
| | CALCIUM CHANNEL BETA-4 SUBUNIT | | | |
| | (CAB4) (VOLTAGE-DEPENDENT CALCIUM | | | |
| | CHANNEL BETA-4 SUBUNIT). | | | |
| 26388 | FGF23: (FGF23 OR HYPF) FIBROBLAST | Q9GZV9 | NM_020638 | 1.30/— % |
| | GROWTH FACTOR-23 PRECURSOR (FGF- | Q4V758 | | |
| | 23) (TUMOR-DERIVED | | | |
| | HYPOPHOPHATEMIA INDUCING FACTOR). | | | |
| 26497 | KCNE1: (KCNE1) ISK SLOW VOLTAGE- | P15382 Q8N709 | NM_000219 | |
| | GATED POTASSIUM CHANNEL PROTEIN | Q91Z94 | | |
| | (MINIMAL POTASSIUM CHANNEL) (MINK). | | | |
| 26500 | KCNE2: (KCNE2) MINIMUM POTASSIUM | Q9Y6J6 | NM_172201 | |
| | ION CHANNEL-RELATED PEPTIDE 1 | | | |
| | (MIRP1) (MINK-RELATED PEPTIDE 1). | | | |
| 26503 | KCNE3: (KCNE3) MINIMUM POTASSIUM | Q9Y6H6 | NM_005472 | |
| | ION CHANNEL-RELATED PEPTIDE 2 | | | |
| | (MIRP2) (MINK-RELATED PEPTIDE 2). | | | |
| 26623 | SCN1B: (SCN1B) SODIUM CHANNEL BETA- | Q07699 | NM_001037 | |
| | 1 SUBUNIT PRECURSOR. | | NM_199037 | |
| 26660 | TGFBR3: (TGFBR3) TGF-BETA RECEPTOR | Q5T2T4 | NM_003243 | |
| | TYPE III PRECURSOR (TGFR-3) | Q5U731 | | |
| | (BETAGLYCAN). | Q9UGI2 | | |
| | | Q03167 | | |
| 26941 | MYH6: (MYH6 OR MYHCA) MYOSIN | P13533 Q13943 | NM_002471 | |
| | HEAVY CHAIN, CARDIAC MUSCLE ALPHA | Q14906 Q14907 | | |
| | ISOFORM (MYHC-ALPHA). | | | |
| 26951 | NME2: (NME2 OR NM23B) NUCLEOSIDE | P22392 | NM_001018136 | 1.48/13% |
| | DIPHOSPHATE KINASE B (EC 2.7.4.6) (NDK | | NM_001018137 | |
| | B) (NDP KINASE B) (P18). | | NM_001018138 | |
| | | | NM_001018139 | |
| | | | NM_002512 | |
| 26966 | PRPH: (PRPH) PERIPHERIN (PRPH1). | P41219 Q8N577 | NM_006262 | |
| 27068 | DERMO1: (TWIST2 OR DERMO1) TWIST | Q8WVJ9 | NM_057179 | |
| | RELATED PROTEIN 2 (DERMIS EXPRESSED | | | |
| | PROTEIN 1) (DERMO-1). | | | |
| 27246 | PTN: (PTN OR NEGF1 OR HBNF1) | P21246 | NM_002825 | 1.60/— % |
| | PLEIOTROPHIN PRECURSOR (PTN) | | | |
| | (HEPARIN-BINDING GROWTH- | | | |
| | ASSOCIATED MOLECULE) (HB-GAM) | | | |
| | (HEPARIN-BINDING GROWTH FACTOR 8) | | | |
| | (HBGF-8) (OSTEOBLAST SPECIFIC FACTOR | | | |
| | 1) (OSF-1) (HEPARIN-BINDING NEURITE | | | |
| | OUTGROWTH PROMOTING FACTOR 1) | | | |
| | (HBNF- | | | |
| 27251 | PTTG_HUMAN: ((PTTG1 OR EAP1 OR PTTG | O95211 O95997 | NM_004219 | 1.60/6% |
| | OR TUTR1) AND (PTTG2) AND (PTTG3)) | O95355 | NM_006607 | |
| | SECURIN (PITUITARY TUMOR- | Q9NZH4 | NR_002734 | |
| | TRANSFORMING PROTEIN 1) (TUMOR- | O95356 | | |
| | TRANSFORMING PROTEIN 1) (ESP1- | Q9NZH5 | | |
| | ASSOCIATED PROTEIN) (HPTTG) | Q9UNJ6 | | |
| | (PITUITARY TUMOR TRANSFORMING | | | |
| | GENE PROTEIN 2) (PITUITARY TUMOR- | | | |
| | TRANSFO | | | |
| 27255 | RPS24: (RPS24 OR RPS19) 40S RIBOSOMAL | P62847 P16632 | NM_001026 | 1.06/8% |
| | PROTEIN S24 (S19). | | NM_033022 | |
| 27501 | ALPL: (ALPL) ALKALINE PHOSPHATASE, | O75090 P05186 | NM_000478 | |
| | TISSUE-NONSPECIFIC ISOZYME | Q9UIL5 | | |
| | PRECURSOR (EC 3.1.3.1) (AP-TNAP) | Q8WU32 | | |
| | (LIVER/BONE/KIDNEY ISOZYME) | Q9UBK0 | | |
| | (TNSALP) (AKP2 OR AKP-2). | | | |
| 27579 | PTHLH: (PTHLH OR PTHRP) | Q15251 P12272 | NM_002820 | 0.31/0% |
| | PARATHYROID HORMONE-RELATED | | NM_198964 | |
| | PROTEIN PRECURSOR (PTH-RP) (PTHRP) | | NM_198965 | |
| | [CONTAINS: OSTEOSTATIN (PTHRP[107-139])] | | NM_198966 | |
| | PARATHYROID HORMONE-LIKE | | | |
| | HORMONE. | | | |
| 27728 | JADE1_1: (PHF17 OR JADE1) | Q96JL8 | NM_199320 | |
| | HYPOTHETICAL PROTEIN KIAA1807 (PHD | Q96SQ1 | | |
| | ZINC FINGER PROTEIN JADE-1) (FLJ14714) | Q9H692 Q6IE81 | | |
| | (FLJ22479) (FLJ45397) (JADE1L) (FLJ90505). | Q6ZSL7 | | |
| | | Q8NC41 | | |
| | | Q4W5D5 | | |
| 27741 | MAD2L1: (MAD2L1 OR MAD2 OR MAD2A) | Q13257 | NM_002358 | |
| | MITOTIC SPINDLE ASSEMBLY | | | |
| | CHECKPOINT PROTEIN MAD2A (MAD2- | | | |
| | LIKE 1). | | | |
| 27762 | SOX11: (SOX11 OR SOX-11) | P35716 | NM_003108 | |
| | TRANSCRIPTION FACTOR SOX-11. | | | |
| 27831 | CITED2: (CITED2 OR MRG1) CBP/P300- | Q99967 O95426 | NM_006079 | |
| | INTERACTING TRANSACTIVATOR 2 (MSG- | | | |
| | RELATED PROTEIN 1) (MRG1 PROTEIN) | | | |
| | (P35SRJ). | | | |
| 27864 | TNNT1: (TNNT1 OR TNT) TROPONIN T, | O95472 Q16061 | NM_003283 | |
| | SLOW SKELETAL MUSCLE ISOFORMS | P13805 | | |
| | (SLOW SKELETAL MUSCLE TROPONIN T). | | | |
| 27870 | ANGPT1: (ANGPT1 OR KIAA0003) | Q15389 | NM_001146 | |
| | ANGIOPOIETIN-1 PRECURSOR (ANG-1). | | NM_139290 | |
| 27873 | ANGPT2: (ANGPT2) ANGIOPOIETIN-2 | O15123 | NM_001147 | |
| | PRECURSOR (ANG-2). | Q9NRR7 | | |
| | | Q9P2Y7 | | |
| 27964 | ALPPL2_HUMAN: (ALPPL2 OR ALPPL) | Q16727 P10696 | NM_031313 | |
| | ALKALINE PHOSPHATASE, PLACENTAL- | Q96CM1 | | |
| | LIKE PRECURSOR (EC 3.1.3.1) (NAGAO | | | |
| | ISOZYME) (GERM-CELL ALKALINE | | | |
| | PHOSPHATASE) (PLAP-LIKE) (ALP-1). | | | |
| 27965 | ALPP_HUMAN: (ALPP OR PLAP) ALKALINE | P05187 P05188 | NM_001632 | 1.55/41% |
| | PHOSPHATASE, PLACENTAL TYPE 1 | P06861 | | |
| | PRECURSOR (EC 3.1.3.1) (PLAP-1) (REGAN | Q96DB7 | | |
| | ISOZYME) (ALKALINE PHOSPHATASE, | | | |
| | PLACENTAL TYPE 3 PRECURSOR). | | | |
| 28160 | SOX5: (SOX5 OR SOX-5) TRANSCRIPTION | Q8J017 Q8J018 | NM_006940 | |
| | FACTOR SOX-5. | Q8J019 Q8J020 | NM_152989 | |
| | | Q8N1D9 | NM_178010 | |
| | | Q8N7E0 | | |
| | | Q8TEA4 | | |
| | | Q86UK8 | | |
| | | P35711 | | |
| 28292 | CHI3L2_HUMAN: (CHI3L2) CHITINASE 3- | Q15782 Q15749 | NM_001025197 | |
| | LIKE PROTEIN 2 PRECURSOR (YKL-39) | Q15783 | NM_001025199 | |
| | (CHONDROCYTE PROTEIN 39). | | NM_004000 | |
| 28320 | KPNA2: (KPNA2 OR RCH1 OR SRP1) | P52292 | NM_002266 | |
| | IMPORTIN ALPHA-2 SUBUNIT | Q9BRU5 | | |
| | (KARYOPHERIN ALPHA-2 SUBUNIT) (SRP1- | | | |
| | ALPHA) (RAG COHORT PROTEIN 1). | | | |
| 28475 | LAPTM4B: (LAPTM4B OR LAPTM4BETA OR | Q9H060 | NM_018407 | 1.12/19% |
| | DKFZP586E1124) LYSOSOMAL- | Q86VI4 | | |
| | ASSOCIATED TRANSMEMBRANE PROTEIN | Q86VH8 | | |
| | 4 BETA (NT2RM1000066) (LC27) (INTEGRAL | Q7L909 | | |
| | MEMBRANE TRANSPORTER) | Q3ZCV5 | | |
| | (HYPOTHETICAL PROTEIN PSEC0001). | | | |
| 28604 | NPPA: (NPPA OR PND) ATRIAL | P01160 Q13766 | NM_006172 | |
| | NATRIURETIC FACTOR PRECURSOR (ANF) | | | |
| | (ATRIAL NATRIURETIC PEPTIDE) (ANP) | | | |
| | (PREPRONATRIODILATIN). | | | |
| 28658 | RPL24: (RPL24) 60S RIBOSOMAL PROTEIN | Q6IBS3 P83731 | NM_000986 | 1.32/13% |
| | L24 (L30). | | | |
| 28891 | DAB1: (DAB1) DISABLED HOMOLOG 1. | O75553 | NM_021080 | |
| | | Q9NYA8 | | |
| 28915 | OLIG1: (OLIG1) OLIGODENDROCYTE | Q7RTS0 | NM_138983 | |
| | TRANSCRIPTION FACTOR 1 (OLIGO1) | Q8TAK6 | | |
| | (OLIGODENDROCYTE-SPECIFIC BHLH | | | |
| | TRANSCRIPTION FACTOR 1). | | | |
| 28921 | RELN: (RELN OR RL) REELIN PRECURSOR | Q86UJ0 Q86UJ8 | NM_005045 | |
| | (EC 3.4.21.—) (REELER PROTEIN). | Q8NDV0 | NM_173054 | |
| | | P78509 | | |
| | | Q9UDQ2 | | |
| 28924 | ROBO1: (ROBO1 OR DUTT1) DUTT1 | Q7Z300 | NM_002941 | |
| | PROTEIN. | Q9BUS7 | NM_133631 | |
| | | Q9Y6N7 | | |
| 28937 | TUBB3_HUMAN: (TUBB3 OR TUBB4) | Q9BTZ0 | NM_006086 | 1.46/9% |
| | TUBULIN BETA-3 CHAIN (TUBULIN BETA- | Q13509 | | |
| | III) (TUBULIN BETA-4). | Q9BW10 | | |
| 29197 | LEFTY2_HUMAN: (LEFTY2 OR EBAF OR | O00292 O75611 | NM_003240 | 1.07/7% |
| | TGFB4 OR LEFTA OR LEFTYA) TLEFT- | Q8NBQ9 | | |
| | RIGHT DETERMINATION FACTOR 2 | | | |
| | PRECURSOR (PROTEIN LEFTY-2) (LEFT- | | | |
| | RIGHT DETERMINATION FACTOR A) | | | |
| | (PROTEIN LEFTY-A) (TRANSFORMING | | | |
| | GROWTH FACTOR BETA-4) (TGF-BETA-4) | | | |
| | (ENDOMETRIAL BLEEDING-ASSOCIAT | | | |
| 29198 | LEFTY1_HUMAN: (LEFTY1 OR LEFTB OR | O75610 | NM_020997 | |
| | LEFTYB) LEFT-RIGHT DETERMINATION | | | |
| | FACTOR 1 PRECURSOR (PROTEIN LEFTY- | | | |
| | 1) (LEFT-RIGHT DETERMINATION FACTOR | | | |
| | B) (PROTEIN LEFTY-B). | | | |
| 29221 | NCAM2: (NCAM2 OR NCAM21) NEURAL | O15394 | NM_004540 | 1.20/15% |
| | CELL ADHESION MOLECULE 2 | | | |
| | PRECURSOR (N-CAM 2). | | | |
| 29310 | SNAI2: (SNAI2 OR SLUG OR SLUGH) ZINC | O43623 | NM_003068 | 1.07/24% |
| | FINGER PROTEIN SLUG (NEURAL CREST | | | |
| | TRANSCRIPTION FACTOR SLUG) (SNAIL | | | |
| | HOMOLOG 2). | | | |
| 29322 | SST: (SST OR SMST) SOMATOSTATIN | P61278 P01166 | NM_001048 | |
| | PRECURSOR [CONTAINS: ANTRIN; | | | |
| | SOMATOSTATIN-28; SOMATOSTATIN-14]. | | | |
| 29328 | TH: (TH OR TYH) TYROSINE 3- | P07101 Q15585 | NM_000360 | |
| | MONOOXYGENASE (EC 1.14.16.2) | Q15588 Q15589 | NM_199292 | |
| | (TYROSINE 3-HYDROXYLASE) (TH). | | NM_199293 | |
| 29367 | NEUROG2: (NEUROG2 OR NGN2 OR ATH4 | Q9H2A3 | NM_024019 | 1.11/— % |
| | OR ATOH4 OR ATH4A) NEUROGENIN 2 | Q8N416 | | |
| | (ATONAL PROTEIN HOMOLOG 4) (HELIX- | | | |
| | LOOP-HELIX PROTEIN MATH-4A) | | | |
| | (MATH4A). | | | |
| 29371 | DLX1: (DLX1) HOMEOBOX PROTEIN DLX- | Q8IYB2 P56177 | NM_178120 | |
| | 1. | Q53ZU4 | | |
| 29475 | CEBPA_3: (CEBPA) CCAAT/ENHANCER | P78319 P49715 | NM_004364 | |
| | BINDING PROTEIN ALPHA (C/EBP ALPHA). | | | |
| 29909 | IGHA1-IGHA2_M_HUMAN: (IGHA1) IG | 184707 | — | |
| | ALPHA-1 CHAIN C REGION (IGHA2) (IG | | | |
| | ALPHA-2 CHAIN C REGION). | | | |
| 30025 | PROM1: (PROM1 OR PROML1 OR PROM OR | O43490 | NM_006017 | |
| | CD133 OR AC133) PROMININ 1 PRECURSOR | | | |
| | (PROMININ-LIKE PROTEIN 1) (ANTIGEN | | | |
| | AC133) (CD133 ANTIGEN). | | | |
| 30155 | L30: (L30) 60S RIBOSOMAL PROTEIN L30 | Q8N6S8 | NM_016304 | |
| | ISOLOG (MY024 PROTEIN) (RPL24) | Q9UHA3 | | |
| | (CHROMOSOME 15 OPEN READING FRAME | Q96HJ1 | | |
| | 15). | Q96C76 | | |
| | | Q96B04 | | |
| | | Q9BS42 | | |
| | | Q561V8 | | |
| 30231 | SNRPF: (SNRPF OR PBSCF) SMALL | Q15356 P62306 | NM_003095 | |
| | NUCLEAR RIBONUCLEOPROTEIN F | | | |
| | (SNRNP-F) (SM PROTEIN F) (SM-F) (SMF). | | | |
| 30327 | ACTC: (ACTC OR ACTC1) ACTIN, ALPHA | P68032 | NM_005159 | |
| | CARDIAC. | | | |
| 30331 | CFC1: (CFC1) CRYPTIC PROTEIN | Q9GZR3 | NM_032545 | |
| | PRECURSOR. | Q53T05 | | |
| 30334 | CHF1: (CHF1 OR HRT2 OR HEY2 OR HESR2) | Q9UBP5 | NM_012259 | |
| | BASIC HELIX-LOOP-HELIX FACTOR 1 | | | |
| | (BASIC-HELIX-LOOP-HELIX PROTEIN) | | | |
| | (HES-RELATED REPRESSOR PROTEIN 1 | | | |
| | HERP1) (HAIRY AND ENHANCER OF SPLIT | | | |
| | RELATED 2) (CARDIOVASCULAR BASIC | | | |
| | HELIX-LOOP-HELIXFACTOR 1, CHF1). | | | |
| 30346 | MMRN: (MMRN OR ECM) ENDOTHELIAL | Q13201 Q6P3T8 | NM_007351 | |
| | CELL MULTIMERIN PRECURSOR. | | | |
| 30350 | PODXL: (PODXL OR PCLP1 OR PCLP) | O00592 | NM_005397 | |
| | PODOCALYXIN-LIKE PROTEIN 1 | | | |
| | PRECURSOR. | | | |
| 30353 | ROBO4: (ROBO4 OR 1200012D01RIK) | Q8WZ75 | NM_019055 | |
| | MAGIC ROUNDABOUT (FLJ14946) | Q96JV6 | | |
| | (FLJ00236) (FLJ20798) (FLJ21542). | Q8TEG1 | | |
| | | Q9NWJ8 | | |
| | | Q9H718 | | |
| 30355 | TEAD1: (TEAD1 OR TEF1 OR TEF-1 OR | P28347 | NM_021961 | |
| | TCF13) TRANSCRIPTIONAL ENHANCER | | | |
| | FACTOR TEF-1 (TEA DOMAIN FAMILY | | | |
| | MEMBER 1) (TEAD-1) (PROTEIN GT-IIC) | | | |
| | (TRANSCRIPTION FACTOR 13) (NTEF-1). | | | |
| 30362 | TNNT2: (TNNT2) TROPONIN T, CARDIAC | P45379 O60214 | NM_000364 | |
| | MUSCLE ISOFORMS (TNTC). | Q99596 Q99597 | | |
| | | Q9UM96 | | |
| 30368 | ZFPM2: (ZFPM2 OR FOG2 OR FOG-2) FOG2 | Q9UNI5 | NM_012082 | |
| | TRANSCRIPTION FACTOR (FRIEND OF | Q9NPL7 | | |
| | GATA2) (B330005D23RIK). | Q8WW38 | | |
| | | Q9NPS4 | | |
| | | Q9NPQ0 | | |
| 30433 | ITGAX: (ITGAX OR CD11C) INTEGRIN | P20702 Q8IVA6 | NM_000887 | |
| | ALPHA-X PRECURSOR (LEUKOCYTE | | | |
| | ADHESION GLYCOPROTEIN P150,95 | | | |
| | ALPHA CHAIN) (LEUKOCYTE ADHESION | | | |
| | RECEPTOR P150,95) (CD11C) (LEU M5). | | | |
| 30439 | TGFB1: (TGFB1 OR TGFB) TRANSFORMING | P01137 | NM_000660 | 0.71/17% |
| | GROWTH FACTOR BETA 1 PRECURSOR | Q9UCG4 | | |
| | (TGF-BETA 1). | | | |
| 30442 | TGFB3: (TGFB3 OR TGF-B3) | P10600 | NM_003239 | 0.94/23% |
| | TRANSFORMING GROWTH FACTOR BETA | | | |
| | 3 PRECURSOR (TGF-BETA 3). | | | |
| 30459 | PF4-PF4V1_HUMAN: (SCYB4 OR PF4) | P02776 P10720 | NM_002619 | |
| | PLATELET FACTOR 4 PRECURSOR (PF-4) | | NM_002620 | |
| | (ONCOSTATIN A) (IROPLACT) (PF4V1 OR | | | |
| | SCYB4V1) (PLATELET FACTOR 4 VARIANT | | | |
| | PRECURSOR) (PF4VAR1) (PF4ALT) | | | |
| | (CXCL4). | | | |
| 30523 | CLF-1: (CLF-1) CYTOKINE-LIKE FACTOR-1 | O75462 | NM_004750 | |
| | PRECURSOR (SIMILAR TO CYTOKINE | Q9UHH5 | | |
| | RECEPTOR-LIKE FACTOR 1) (ZCYTOR5) | | | |
| | (CLASS I CYTOKINE RECEPTOR) (CRLF1 | | | |
| | OR CRLM3) (CYTOKINE RECEPTOR LIKE | | | |
| | MOLECULE 3 PRECURSOR). | | | |
| 30546 | FGF20: (FGF20) FIBROBLAST GROWTH | Q9NP95 | NM_019851 | |
| | FACTOR-20 (FGF-20). | | | |
| 30615 | SDF2: (SDF2) STROMAL CELL-DERIVED | Q99470 | NM_006923 | 0.89/8% |
| | FACTOR 2 PRECURSOR (SDF-2). | Q9BQ79 | | |
| 30624 | BMP11: (GDF11 OR BMP11) | O95390 | NM_005811 | 1.20/32% |
| | GROWTH/DIFFERENTIATION FACTOR 11 | Q9UID1 | | |
| | PRECURSOR (BONE MORPHOGENETIC | Q9UID2 | | |
| | PROTEIN 11). | | | |
| 30632 | FGF17: (FGF17) FIBROBLAST GROWTH | O60258 | NM_003867 | |
| | FACTOR-17 PRECURSOR (FGF-17). | | | |
| 30635 | FGF18: (FGF18) FIBROBLAST GROWTH | O76093 | NM_003862 | |
| | FACTOR-18 PRECURSOR (FGF-18). | Q6UWF1 | NM_033649 | |
| 30637 | FGF4: (FGF4 OR HST OR HSTF1 OR KS3) | P08620 | NM_002007 | |
| | FIBROBLAST GROWTH FACTOR-4 | | | |
| | PRECURSOR (FGF-4) (HEPARIN | | | |
| | SECRETORY TRANSFORMING PROTEIN) | | | |
| | (HST-1) (HST) (TRANSFORMING PROTEIN | | | |
| | KS3) (HBGF-4). | | | |
| 30671 | RARA1: (RARA OR NR1B1) RETINOIC ACID | P10276 P78456 | NM_000964 | |
| | RECEPTOR ALPHA (RAR-ALPHA). | Q13440 Q13441 | NM_001024809 | |
| | | Q96S41 | NM_001033603 | |
| | | Q9NQS0 | | |
| 30683 | FN1_REPEAT-1TO6: (FN1 OR FN) | P02751 O95609 | NM_002026 | 0.58/2% |
| | FIBRONECTIN PRECURSOR (FN) (COLD- | O95610 Q14312 | NM_212474 | |
| | INSOLUBLE GLOBULIN) (CIG). | Q14325 Q14326 | NM_212475 | |
| | | Q86T27 Q8IVI8 | NM_212476 | |
| | | Q96KP7 Q | NM_212478 | |
| | | | NM_212482 | |
| 30686 | FN1_REPEAT-A: (FN1 OR FN) | P02751 O95609 | NM_002026 | 0.24/2% |
| | FIBRONECTIN PRECURSOR (FN) (COLD- | O95610 Q14312 | NM_212474 | |
| | INSOLUBLE GLOBULIN) (CIG). | Q14325 Q14326 | NM_212475 | |
| | | Q86T27 Q8IVI8 | NM_212476 | |
| | | Q96KP7 Q | NM_212478 | |
| | | | NM_212482 | |
| 30689 | FN1_REPEAT-B: (FN1 OR FN) | P02751 O95609 | NM_002026 | 0.19/8% |
| | FIBRONECTIN PRECURSOR (FN) (COLD- | O95610 Q14312 | NM_212474 | |
| | INSOLUBLE GLOBULIN) (CIG). | Q14325 Q14326 | NM_212475 | |
| | | Q86T27 Q8IVI8 | NM_212476 | |
| | | Q96KP7 Q | NM_212478 | |
| | | | NM_212482 | |
| 30808 | CNTFR: (CNTFR) CILIARY NEUROTROPHIC | P26992 | NM_001842 | |
| | FACTOR RECEPTOR ALPHA PRECURSOR | | NM_147164 | |
| | (CNTFR ALPHA). | | | |
| 30815 | GATA5: (GATA5) TRANSCRIPTION | Q9BWX5 | NM_080473 | |
| | FACTOR GATA-5 (GATA BINDING | | | |
| | FACTOR-5). | | | |
| 30954 | ACRP: (ACRP OR CTNNAL1) ALPHA- | O76084 | NM_003798 | 1.44/11% |
| | CATENIN-LIKE PROTEIN. | Q9UBT7 | | |
| | | Q9Y401 | | |
| | | Q53FQ2 | | |
| | | Q5JTQ7 | | |
| | | Q5JTQ8 | | |
| 30957 | ADH4: (ADH4) ALCOHOL | P08319 | NM_000670 | |
| | DEHYDROGENASE CLASS II PI CHAIN | Q8TCD7 | | |
| | PRECURSOR (EC 1.1.1.1). | | | |
| 30963 | ARHGAP9: (ARHGAP9) RHO-GTPASE | Q96S74 | NM_032496 | |
| | ACTIVATING PROTEIN (FLJ35444) (RGL1) | Q96EZ2 | | |
| | (DKFZP667F149) (AU043488). | Q9BRR9 | | |
| | | Q8WYR0 | | |
| | | Q8NAF3 | | |
| | | Q8TCJ3 | | |
| 30969 | BUB1B: (BUB1B OR MAD3L OR BUBR1) | O60566 O60501 | NM_001211 | |
| | MITOTIC CHECKPOINT | O60627 O60758 | | |
| | SERINE/THREONINE-PROTEIN KINASE | O75389 | | |
| | BUB1 BETA (EC 2.7.1.—) (HBUBR1) | Q96KM4 | | |
| | (MAD3/BUB1-RELATED PROTEIN KINASE) | | | |
| | (MITOTIC CHECKPOINT KINASE MAD3L). | | | |
| 30972 | BUB3: (BUB3) MITOTIC CHECKPOINT | O43684 O43685 | NM_004725 | |
| | PROTEIN BUB3. | | | |
| 30975 | CA14: (CA14) CARBONIC ANHYDRASE XIV | Q9ULX7 | NM_012113 | |
| | PRECURSOR (EC 4.2.1.1) (CARBONATE | Q8NCF4 | | |
| | DEHYDRATASE XIV) (CA-XIV). | Q5TB24 | | |
| 30978 | CCCAP: (SDCCAG8 OR CCCAP) | Q86SQ7 | NM_006642 | |
| | CENTROSOMAL COLON CANCER | Q9P0F1 | | |
| | AUTOANTIGEN PROTEIN (HSPC085) (NY- | Q8N5F2 | | |
| | CO-8) (2700048G21RIK) (5730470G24RIK) | O60527 | | |
| | (SLINKY). | Q3ZCR6 | | |
| 30981 | CDO1: (CDO1) CYSTEINE DIOXYGENASE | Q16878 P78513 | NM_001801 | |
| | TYPE I (EC 1.13.11.20) (CDO) (CDO-I). | Q6FHZ8 | | |
| | | Q8TB64 | | |
| 30984 | CHI3L1: (CHI3L1) CHITINASE-3 LIKE | P36222 P30923 | NM_001276 | |
| | PROTEIN 1 PRECURSOR (CARTILAGE | Q8IVA4 | | |
| | GLYCOPROTEIN-39) (GP-39) (39 KDA | Q96HI7 | | |
| | SYNOVIAL PROTEIN) (YKL-40). | | | |
| 30987 | CPN2: (CPN2) CARBOXYPEPTIDASE N 83 | P22792 Q86SU4 | NM_001309 | 1.40/25% |
| | KDA CHAIN (CARBOXYPEPTIDASE N | Q8N5V4 | | |
| | REGULATORY SUBUNIT) | | | |
| | (CARBOXYPEPTIDASE N POLYPEPTIDE 2). | | | |
| 30990 | CRM1: (CRM1) CRM1 PROTEIN (XPO1) | O14980 Q99433 | NM_003400 | 1.04/14% |
| | (EXPORTIN 1) (EXPRESSED SEQUENCE | Q63HP8 | | |
| | AA420417) (NUCLEAR EXPORT FACTOR | Q68CP3 | | |
| | CRM1). | | | |
| 30993 | CRYL1: (CRYL1) LAMBDA-CRYSTALLIN | Q9Y2S2 | NM_015974 | 1.39/29% |
| | HOMOLOG. | Q7Z4Z9 | | |
| 30996 | CRYZ: (CRYZ) QUINONE | Q08257 Q53FT0 | NM_001889 | 0.88/26% |
| | OXIDOREDUCTASE (EC 1.6.5.5) | Q59EU7 | | |
| | (NADPH: QUINONE REDUCTASE) (ZETA- | Q6NSK9 | | |
| | CRYSTALLIN). | | | |
| 30999 | CTNNA2: (CTNNA2 OR CAPR) ALPHA-2 | P26232 | NM_004389 | |
| | CATENIN (ALPHA-CATENIN RELATED | Q4ZFW1 | | |
| | PROTEIN) (ALPHA N-CATENIN). | Q53R26 | | |
| | | Q53R33 | | |
| | | Q53T67 | | |
| | | Q53T71 | | |
| | | Q53TM8 | | |
| | | Q7Z3Y0 | | |
| 31002 | ZFP644: (ZFP644 OR DJ924G13.1 OR | Q9H582 | NM_016620 | 0.82/31% |
| | KIAA1221) ZINC FINGER PROTEIN 644 (BM- | Q9NZF0 | NM_032186 | |
| | 005) (FLJ10725) (FLJ13534) (FLJ13964) | Q9NVH8 | NM_201269 | |
| | (D5ERTD689E OR MKIAA1221) | Q9H8J8 | | |
| | (1110068L01RIK). | Q9H835 | | |
| | | Q8NEI6 | | |
| | | Q9ULJ9 | | |
| | | Q6BEP7 | | |
| | | Q6P446 Q | | |
| 31006 | DPPA5: (DPPA5) DEVELOPMENTAL | O95431 | — | |
| | PLURIPOTENCY ASSOCIATED 5 | | | |
| | (EMBRYONAL STEM CELL SPECIFIC GENE | | | |
| | 1) (2410024L16RIK) (LOC340168). | | | |
| 31008 | DTYMK: (DTYMK OR TYMK OR TMPK OR | P23919 | NM_012145 | |
| | CDC8) THYMIDYLATE KINASE (EC 2.7.4.9) | Q9BUX4 | | |
| | (DTMP KINASE). | Q6FGX1 | | |
| 31014 | EED: (EED) EMBRYONIC ECTODERM | O00149 O75530 | NM_003797 | |
| | DEVELOPMENT PROTEIN HOMOLOG | Q86VV2 | NM_152991 | |
| | (WAIT1). | Q9UNY7 | | |
| 31017 | EFNA2: (EFNA2 OR EPLG6 OR LERK6) | O43921 O76020 | NM_001405 | 1.48/8% |
| | EPHRIN-A2 PRECURSOR (EPH-RELATED | | | |
| | RECEPTOR TYROSINE KINASE LIGAND 6) | | | |
| | (LERK-6) (HEK7-LIGAND) (HEK7-L). | | | |
| 31020 | F11R: (F11R OR JAM1 OR JCAM) | Q9Y624 | NM_016946 | |
| | JUNCTIONAL ADHESION MOLECULE 1 | | NM_144501 | |
| | PRECURSOR (JAM) (PLATELET ADHESION | | NM_144502 | |
| | MOLECULE 1) (PAM-1) (PLATELET F11 | | NM_144503 | |
| | RECEPTOR) (CD321 ANTIGEN) | | NM_144504 | |
| | (UNQ264/PRO301). | | | |
| 31023 | FGA: (FGA) FIBRINOGEN ALPHA/ALPHA-E | P02671 | NM_000508 | 1.66/9% |
| | CHAIN PRECURSOR (HQ0582). | Q9BX62 | NM_021871 | |
| | | Q9UCH2 | | |
| 31026 | FGL1: (FGL1 OR HFREP1) FIBRINOGEN- | Q08830 | NM_004467 | |
| | LIKE PROTEIN 1 PRECURSOR | Q96KW6 | NM_147203 | |
| | (HEPATOCYTE-DERIVED FIBRINOGEN- | Q96QM6 | | |
| | RELATED PROTEIN 1) (HFREP-1) | Q4PJH9 | | |
| | (HEPASSOCIN) (HP-041). | | | |
| 31032 | FLJ10884: (DKFZP434B1629) | Q8NDA1 | NM_019079 | |
| | HYPOTHETICAL PROTEIN FLJ10884 | Q9NUV8 | | |
| | (FLJ11111) (ECAT11) (ES CELL | Q9NV78 | | |
| | ASSOCIATED TRANSCRIPT 11). | | | |
| 31035 | FLJ21190: HYPOTHETICAL PROTEIN | Q9H773 | NM_024096 | 1.57/12% |
| | FLJ21190 (CDA03) (RS21C6) (TDRG-TL1 OR | | | |
| | 2410015N17RIK) (RS21-C6). | | | |
| 31038 | FLJ21702: HYPOTHETICAL PROTEIN | Q9H6Y2 | NM_017706 | |
| | FLJ21702 (2410080P20RIK) (FLJ20195). | Q9NXK4 | | |
| 31041 | FLJ22362: (FNMP1) HYPOTHETICAL | Q9H6D8 | NM_022823 | |
| | PROTEIN FLJ22362 (FRCP1 OR | | | |
| | 2810430J06RIK). | | | |
| 31044 | FLJ25967: (FLJ25967) MI RELATED NOVEL | — | — | |
| | MRNA (FLJ45323) (FLJ38367) (FLJ42830) | | | |
| | (FLJ25887) (DKFZP564O163). | | | |
| 31047 | FBXL13: (FBXL13) F-BOX AND LEUCINE- | Q8N1P0 | NM_145032 | |
| | RICH REPEAT PROTEIN 13 (FLJ38068) | Q8WUG0 | | |
| | (4921539K22RIK) (MGC21636) (FLJ40218) | Q8N7Y4 | | |
| | (DKFZP434L2422). | Q8TCL2 | | |
| | | Q8NEE6 | | |
| | | Q8WUF9 | | |
| | | Q86UJ5 | | |
| | | Q6UVW7 | | |
| | | Q6UVW8 Q | | |
| 31050 | GGH: (GGH) GAMMA-GLUTAMYL | Q92820 | NM_003878 | 0.98/42% |
| | HYDROLASE PRECURSOR (EC 3.4.19.9) | | | |
| | 9430073I07). | | | |
| 31090 | KIAA1698: (KIAA1698) HYPOTHETICAL | Q9C0G5 | NM_030628 | |
| | PROTEIN KIAA1698 (1110055N21RIK). | Q8N6W5 | | |
| | | Q6P9B9 | | |
| 31093 | KS: (KS) KIDNEY-SPECIFIC PROTEIN | Q86YT1 | NM_182617 | |
| | (XENOBIOTIC/MEDIUM-CHAIN FATTY | O75202 | | |
| | ACID: COA LIGASE) (FLJ26434) (FLJ38720). | | | |
| 31096 | MAD1: (MAD1L1 OR MAD1) MITOTIC | Q9UNH0 | NM_03550 | 1.43/68% |
| | CHECKPOINT PROTEIN (MAD1 (MITOTIC | Q9Y6D9 | | |
| | ARREST DEFICIENT, YEAST, HOMOLOG)- | Q13312 | | |
| | LIKE 1) (TXBP181) (MAD1A) (MAD1B). | Q86UM4 | | |
| | | Q75MI0 | | |
| 31099 | MAD2L2: (MAD2L2 OR MAD2B OR REV7) | Q9UI95 | NM_006341 | |
| | MITOTIC SPINDLE ASSEMBLY | Q9UNA7 | | |
| | CHECKPOINT PROTEIN MAD2B (MAD2- | Q9Y6I6 | | |
| | LIKE 2) (HREV7) (2310033C13RIK). | Q5TGW7 | | |
| 31102 | MAT1A: (MAT1A OR MATA1 OR AMS1) S- | Q00266 | NM_000429 | |
| | ADENOSYLMETHIONINE SYNTHETASE | Q5QP09 | | |
| | ALPHA AND BETA FORMS (EC 2.5.1.6) | | | |
| | (METHIONINE ADENOSYLTRANSFERASE) | | | |
| | (ADOMET SYNTHETASE) (MAT-I/III). | | | |
| 31105 | MAWBP: (MAWBP) MAWD BINDING | P30039 | NM_022129 | |
| | PROTEIN (UNKNOWN PROTEIN 32 FROM | Q9HCC2 | | |
| | 2D-PAGE OF LIVER TISSUE) (PROBABLE | | | |
| | OXIDOREDUCTASE 0610038K03RIK) | | | |
| | (PROBABLE OXIDOREDUCTASE | | | |
| | 3110049J23RIK). | | | |
| 31108 | MGC16491: (4732473B16RIK OR AU045678) | Q96A09 | NM_052943 | |
| | HYPOTHETICAL PROTEIN (MGC16491). | | | |
| 31112 | NANOG: (NANOG) HOMEOBOX | Q9H9S0 | NM_024865 | |
| | TRANSCRIPTION FACTOR NANOG | Q8N7R0 | | |
| | (FLJ12581) (FLJ40451) (EMBRYONIC STEM | Q2TTG0 | | |
| | CELL SPECIFIC HOMEOBOX PROTEIN). | Q6JZS5 | | |
| 31114 | NOP5: (NOP5) NUCLEOLAR PROTEIN NOP5 | Q9Y2X3 | NM_015934 | |
| | (NUCLEOLAR PROTEIN 5) (NOP58) | Q9P036 | | |
| | (HSPC120) (NOL5) (SIK SIMILAR PROTEIN). | Q9UFN3 | | |
| | | Q6PK08 | | |
| 31117 | NUMB: (NUMB) NUMB PROTEIN | P49757 | NM_003744 | |
| | HOMOLOG (H-NUMB) (PROTEIN S171). | Q9UBG1 | | |
| | | Q9UEQ4 | | |
| | | Q9UKE8 | | |
| | | Q9UKE9 | | |
| | | Q9UKF0 | | |
| | | Q9UQJ4 | | |
| | | Q6NUQ7 | | |
| | | Q86SY1 Q | | |
| 31120 | PCPB: (PCPB OR CPB2 OR TAFI) PCPB | Q15114 Q96IY4 | NM_001872 | |
| | PROTEIN (CARBOXYPEPTIDASE B2 | Q9P2Y6 | NM_016413 | |
| | (PLASMA)) (CARBOXYPEPTIDASE B-LIKE | Q5T9K1 | | |
| | PROTEIN) (CPB2) (BA139H14.2) | Q5T9K2 | | |
| | (CARBOXYPEPTIDASE R) (THROMBIN- | | | |
| | ACTIVATABLE FIBRINOLYSIS INHIBITOR) | | | |
| | (1110032P04RIK PROTEIN) | | | |
| | (CARBOXYPEPTIDASE U). | | | |
| 31123 | PON1: (PON1 OR PON) SERUM | P27169 Q16052 | NM_000446 | 0.73/— % |
| | PARAOXONASE/ARYLESTERASE 1 (EC | | | |
| | 3.1.1.2) (EC 3.1.8.1) (PON 1) (SERUM | | | |
| | ARYLDIALKYLPHOSPHATASE 1) (A- | | | |
| | ESTERASE 1) (AROMATIC ESTERASE 1) (K- | | | |
| | 45). | | | |
| 31126 | PON3: (PON3) SERUM | Q15166 O75855 | NM_000940 | |
| | PARAOXONASE/ARYLESTERASE 3 (EC | O76060 Q6IRU9 | | |
| | 3.1.1.2) (EC 3.1.8.1) (PON 3) (SERUM | Q8IX97 | | |
| | ARYLDIALKYLPHOSPHATASE 3) (A- | Q9BZH9 | | |
| | ESTERASE 3) (AROMATIC ESTERASE 3). | | | |
| 31129 | PPP2R1B_1: (PPP2R1B) | P30154 O75620 | NM_002716 | |
| | SERINE/THREONINE PROTEIN | | | |
| | PHOSPHATASE 2A, 65 KDA REGULATORY | | | |
| | SUBUNIT A, BETA ISOFORM (PP2A, | | | |
| | SUBUNIT A, PR65-BETA ISOFORM) (PP2A, | | | |
| | SUBUNIT A, R1-BETA ISOFORM) | | | |
| | (TRANSCRIPT VARIANT 1). | | | |
| 31132 | PPP2R1B_2: (PPP2R1B) | P30154 O75620 | NM_181699 | |
| | SERINE/THREONINE PROTEIN | | | |
| | PHOSPHATASE 2A, 65 KDA REGULATORY | | | |
| | SUBUNIT A, BETA ISOFORM (PP2A, | | | |
| | SUBUNIT A, PR65-BETA ISOFORM) (PP2A, | | | |
| | SUBUNIT A, R1-BETA ISOFORM) | | | |
| | (TRANSCRIPT VARIANT 2). | | | |
| 31135 | PROX1: (PROX1) HOMEOBOX PROSPERO- | Q92786 | NM_002763 | |
| | LIKE PROTEIN PROX1 (PROX 1). | Q5SW76 | | |
| | | Q8TB91 | | |
| 31138 | REC1: (REC1 OR RAD1A OR HRAD1) CELL | O60671 O75572 | NM_002853 | |
| | CYCLE CHECKPOINT PROTEIN HRAD1 | Q9UEP1 | NM_133282 | |
| | (DNA REPAIR EXONUCLEASE) (RAD1 | O95304 | NM_133377 | |
| | (S. POMBE) HOMOLOG) (RAD1 HOMOLOG) | Q5KSM0 | | |
| | (S. POMBE). | Q5KSM1 | | |
| 31141 | RNASE4: (RNASE4 OR RNS4) | P34096 | NM_002937 | 1.06/12% |
| | RIBONUCLEASE 4 PRECURSOR (EC 3.1.27.—) | | | |
| | (RNASE 4). | | | |
| 31144 | RPL13A: (RPL13A) 60S RIBOSOMAL | P40429 | NM_012423 | 1.19/14% |
| | PROTEIN L13A (23 KDA HIGHLY BASIC | | | |
| | PROTEIN). | | | |
| 31147 | SALL2: (SALL2 OR SAL2 OR KIAA0360) | Q9Y467 | NM_005407 | |
| | SAL-LIKE PROTEIN 2 (ZINC FINGER | Q9Y4G1 | | |
| | PROTEIN SALL2) (HSAL2). | | | |
| 31153 | SNAI1: (SNAI1 OR SNAH) ZINC FINGER | O95863 Q9P113 | NM_005985 | |
| | PROTEIN SNAI1 (SNAIL PROTEIN | Q9UBP7 | | |
| | HOMOLOG) (SNA PROTEIN). | Q9UHH7 | | |
| 31156 | SOX1: (SOX1) SOX-1 PROTEIN. | O00570 | NM_005986 | |
| 31159 | SOX10: (SOX10) TRANSCRIPTION FACTOR | P56693 | NM_006941 | |
| | SOX-10. | | | |
| 31162 | SOX13: (SOX13) SOX-13 PROTEIN (TYPE 1 | Q9UN79 | NM_005686 | |
| | DIABETES AUTOANTIGEN ICA12) (ISLET | O95275 O95826 | | |
| | CELL ANTIGEN 12). | Q9UHW7 | | |
| 31168 | SOX3: (SOX3) TRANSCRIPTION FACTOR | P41225 P35714 | NM_005634 | 1.30/31% |
| | SOX-3. | Q9NP49 | | |
| 31171 | SOX6: (SOX6) TRANSCRIPTION FACTOR | P35712 | NM_033326 | |
| | SOX-6. | Q9BXQ3 | | |
| | | Q9BXQ4 | | |
| | | Q9BXQ5 | | |
| | | Q9H0I8 | | |
| 31174 | SSPN: (SSPN OR KRAG) SARCOSPAN (K- | Q14714 | NM_005086 | 0.87/74% |
| | RAS ONCOGENE-ASSOCIATED PROTEIN) | | | |
| | (KIRSTEN-RAS-ASSOCIATED PROTEIN). | | | |
| 31177 | TFPI: (TFPI OR TFPI1 OR LACI) TISSUE | P10646 O95103 | NM_006287 | 1.02/— % |
| | FACTOR PATHWAY INHIBITOR | | | |
| | PRECURSOR (TFPI) (LIPOPROTEIN- | | | |
| | ASSOCIATED COAGULATION INHIBITOR) | | | |
| | (LACI) (EXTRINSIC PATHWAY INHIBITOR) | | | |
| | (EPI). | | | |
| 31180 | TINF2: (TINF2 OR TIN2) TERF1- | Q9BSI4 | NM_012461 | |
| | INTERACTING NUCLEAR FACTOR 2 (TRF1- | Q9H904 | | |
| | INTERACTING NUCLEAR PROTEIN 2). | Q9UHC2 | | |
| 31183 | TM4SF4: (TM4SF4 OR ILTMP) | P48230 | NM_004617 | |
| | TRANSMEMBRANE 4 SUPERFAMILY, | | | |
| | MEMBER 4 (INTESTINE AND LIVER | | | |
| | TETRASPAN MEMBRANE PROTEIN) (IL- | | | |
| | TMP). | | | |
| 31186 | TREM1: (TREM1) TRIGGERING-RECEPTOR | Q86YU1 | NM_018643 | 1.40/13% |
| | TREM1. | Q9NP99 | | |
| | | Q53FL8 | | |
| | | Q5T2C9 | | |
| 31192 | ZFP42: (ZFP42 OR REX1 OR REX-1) ZINC | Q96MM3 | NM_174900 | |
| | FINGER PROTEIN 42 (ZFP-42) (REX-1 | Q8WXE2 | | |
| | PROTEIN) (REDUCED EXPRESSION-1 | | | |
| | PROTEIN). | | | |
| 31195 | ZFX: (ZFX) ZINC FINGER X- | P17010 O43668 | NM_003410 | 1.33/7% |
| | CHROMOSOMAL PROTEIN. | Q8WYJ8 | | |
| 31198 | ZNF206: (ZNF206 OR ZSCAN10) ZINC | Q96SZ4 | NM_032805 | 1.34/10% |
| | FINGER PROTEIN 206 (ZINC FINGER AND | | | |
| | SCAN DOMAIN-CONTAINING PROTEIN 10) | | | |
| | (FLJ14549). | | | |
| 31201 | SOX18: (SOX18) TRANSCRIPTION FACTOR | P35713 | NM_018419 | |
| | SOX-18. | Q9NPH8 | | |
| 31460 | KIAA0152_1: (KIAA0152) HYPOTHETICAL | Q14165 | NM_014730 | 1.39/5% |
| | PROTEIN KIAA0152 (2410014A08RIK). | | | |
| 31466 | KIAA0152_3: (KIAA0152) HYPOTHETICAL | Q14165 | NM_014730 | 0.98/39% |
| | PROTEIN KIAA0152 (2410014A08RIK). | | | |
| 32031 | TFRC_3PRIME: (TFRC) TRANSFERRIN | P02786 | NM_003234 | 0.70/6% |
| | RECEPTOR PROTEIN (TFR1) (TR) (TFR) | Q9UCN0 | | |
| | (TRFR) (CD71 ANTIGEN) (T9) (P90). | Q9UCU5 | | |
| | | Q9UDF9 | | |
| | | Q9UK21 | | |
| | | Q59G55 | | |
| 32034 | TFRC_5PRIME: (TFRC) TRANSFERRIN | P02786 | NM_003234 | 1.17/12% |
| | RECEPTOR PROTEIN (TFR1) (TR) (TFR) | Q9UCN0 | | |
| | (TRFR) (CD71 ANTIGEN) (T9) (P90). | Q9UCU5 | | |
| | | Q9UDF9 | | |
| | | Q9UK21 | | |
| | | Q59G55 | | |
| 32488 | NPM1: (NPM1 OR NPM) NUCLEOPHOSMIN | P06748 | NM_001004419 | |
| | (NPM) (NUCLEOLAR PHOSPHOPROTEIN | Q9UHP7 | NM_002520 | |
| | B23) (NUMATRIN) (NUCLEOLAR PROTEIN | P08693 Q12826 | NM_013269 | |
| | NO38). | Q13440 Q13441 | NM_199185 | |
| | | Q14115 | | |
| | | Q5EU94 | | |
| | | Q5EU95 Q | | |
| 32647 | TREM2: TRIGGERING RECEPTOR | Q8WYN6 | NM_018965 | 1.26/28% |
| | EXPRESSED ON MYELOID CELLS 2. | Q9NZC2 | | |
| | | Q8N5H8 | | |
| 32676 | ARL8: (ARL8) ADP-RIBOSYLATION | Q96KC2 | NM_178815 | |
| | FACTOR-LIKE PROTEIN 8. | | | |
| 32679 | BRIX: (BRIX) RIBOSOME BIOGENESIS | Q8TDN6 | NM_018321 | 1.61/8% |
| | PROTEIN BRIX. | Q8N453 | | |
| | | Q96DH1 | | |
| | | Q3ZTT4 | | |
| 32682 | C20ORF129: (C20ORF129) PROTEIN | Q9H4H8 | NM_030919 | |
| | C20ORF129. | Q96DF5 | | |
| | | Q96N89 | | |
| | | Q9BVM8 | | |
| | | Q5THR2 | | |
| 32685 | CYP26A1: (CYP26A1 OR CYP26) | O43174 | NM_000783 | |
| | CYTOCHROME P450 26 (EC 1.14.—.—) | | | |
| | (RETINOIC ACID-METABOLIZING | | | |
| | CYTOCHROME) (P450RAI) (HP450RAI) | | | |
| | (RETINOIC ACID 4-HYDROXYLASE). | | | |
| 32688 | EIF4A1: (EIF4A1 OR EIF4A OR DDX2A) | P04765 P60842 | NM_001416 | 1.21/12% |
| | EUKARYOTIC INITIATION FACTOR 4A-I | Q61516 Q5U018 | | |
| | (EIF4A-I)(EIF-4A-I). | | | |
| 32691 | IDH1: (IDH1 OR PICD) ISOCITRATE | O75874 Q93090 | NM_005896 | 0.72/9% |
| | DEHYDROGENASE CYTOPLASMIC (EC | Q9NTJ9 | | |
| | 1.1.1.42) (OXALOSUCCINATE | Q9UKW8 | | |
| | DECARBOXYLASE) (IDH) (NADP+- | | | |
| | SPECIFIC ICDH) (IDP). | | | |
| 32694 | IMPDH2: (IMPDH2 OR IMPD2) INOSINE-5′- | P12268 Q6LEF3 | NM_000884 | 1.53/10% |
| | MONOPHOSPHATE DEHYDROGENASE 2 | | | |
| | (EC 1.1.1.205) (IMP DEHYDROGENASE 2) | | | |
| | (IMPDH-II) (IMPD 2). | | | |
| 32700 | KIAA1573: (KIAA1573) HYPOTHETICAL | Q9HCJ9 | NM_020925 | |
| | PROTEIN KIAA1573 (B430218L07RIK) | Q7Z3P2 | | |
| | (DKFZP686L04115) (FLJ12509) (FLJ14194). | Q9H7W4 | | |
| | | Q9H9W3 | | |
| 32703 | KIF4A: (KIF4A OR KIF4) CHROMOSOME- | O95239 | NM_012310 | 1.62/26% |
| | ASSOCIATED KINESIN KIF4A | Q9NNY6 | | |
| | (CHROMOKINESIN). | Q9NY24 | | |
| | | Q9UMW3 | | |
| | | Q86TN3 | | |
| | | Q86XX7 | | |
| 32706 | LIN-28: (LIN28 OR LIN-28) HYPOTHETICAL | Q9H9Z2 | NM_024674 | 1.19/29% |
| | PROTEIN FLJ12457 (RNA-BINDING | | | |
| | PROTEIN LIN-28). | | | |
| 32709 | LRRN1: (LRRN1 OR NLRR-1) LEUCINE RICH | Q9P231 | NM_020873 | |
| | REPEAT PROTEIN 1, NEURONAL | Q8IYV5 | | |
| | (KIAA1497) (NLRR). | Q9H8V1 | | |
| | | Q6UXK5 | | |
| | | Q3LID5 | | |
| 32712 | MTHFD1: (MTHFD1 OR MTHFD OR MTHFC) | P11586 Q86VC9 | NM_005956 | |
| | C-1-TETRAHYDROFOLATE SYNTHASE, | Q9BVP5 | | |
| | CYTOPLASMIC (C1-THF SYNTHASE). | | | |
| 32715 | NBR2_HUMAN: (NBR2) NBR2 PROTEIN | O15453 | NM_005821 | |
| | (NEXT TO BRCA1 GENE 2 PROTEIN). | | | |
| 32716 | PPAT: (PPAT OR GPAT) | Q06203 | NM_002703 | |
| | AMIDOPHOSPHORIBOSYLTRANSFERASE | | | |
| | PRECURSOR (EC 2.4.2.14) (GLUTAMINE | | | |
| | PHOSPHORIBOSYLPYROPHOSPHATE | | | |
| | AMIDOTRANSFERASE) (ATASE) (GPAT). | | | |
| 32719 | RPL4: (RPL4 OR RPL1) 60S RIBOSOMAL | P36578 P39029 | NM_000968 | 1.37/15% |
| | PROTEIN L4 (L1). | Q969Z9 | | |
| | | Q4VBR0 | | |
| 32722 | SMS: (SMS) SPERMINE SYNTHASE (EC | P52788 O00544 | NM_004595 | 1.05/15% |
| | 2.5.1.22) (SPERMIDINE | Q9UQS1 | | |
| | AMINOPROPYLTRANSFERASE) (SPMSY). | | | |
| 32725 | ZNF117_HUMAN: (ZNF117) ZINC FINGER | Q03924 | NM_015852 | |
| | PROTEIN 117 (ZINC FINGER PROTEIN | | | |
| | HPF9). | | | |
| 32726 | ZNF257-ZNF92-ZNF43-ZNF273- | Q9Y2Q1 | NM_003423 | |
| | ZNF680_HUMAN: (ZNF257 OR BMZF4) ZINC | Q96HL5 | NM_007139 | |
| | FINGER PROTEIN 257 (BONE MARROW | Q8NB35 | NM_021148 | |
| | ZINC FINGER 4) (BMZF-4) (MGC12518) | Q8N492 P17038 | NM_033468 | |
| | (FLJ34299) (ZNF43 OR ZNF39 OR KOX27) | Q8NEM1 | NM_152626 | |
| | ZINC FINGER PROTEIN 43 (ZINC PROTEIN | P28160 | NM_178558 | |
| | HTF6) (ZINC FINGER PROTEIN KOX27) | Q96DG1 | | |
| | (ZNF273) (ZNF92) | Q14593 Q | | |

What is claimed is:

1. An amniotic fluid cell composition comprising:
pluripotent embryonic stem cells expressing DAZL; and
amniotic fluid.

2. The composition of claim 1, wherein said amniotic fluid, comprises 5-50% of said basal growth medium.

3. The composition of claim 1 wherein said stem cells may further express at least one marker selected from alkaline phosphatase; SSEA-3; SSEA-4; TRA-1-60; TRA-1-81; TRA2-54; c-kit; Oct-4 and oxytocin receptor.

4. The composition of claim 1 wherein said germline-like stem cells may further express at least one of HLA Class I, CD13, CD44, CD49b, and CD105.

5. The composition of claim 1 wherein said germline-like stem cells may further express at least one of POU5F1, TDGF, GABRB3, FGF4 and TERT.

6. The composition of claim 1 wherein said composition further comprises an agent selected from the group consisting of forskolin ([3R-(3a, 4αβ, 5B, 6B, 6aα, 10α, 10αβ, 10bα)]-5-(acetyloxy)-3-ethenyldodecahydro-6,10,10b-trihydroxy-3,4a,7,7,10a-pentamethyl-1H-naphtho[2,1-b]pyran-1-one), cholera toxin, isobutylmethylxanthine (IBMX), and dibutyrladenosine cyclic monophosphate (dbcAMP).

7. The composition of claim 1, wherein said growth factor is basic fibroblast growth factor (bFGF).

8. The composition of claim 7 wherein the concentration of said bFGF is in the range of about 1-10 ng/ml.

9. The composition of claim 1, wherein said basal growth medium comprises one or more of L-glutamine, essential amino acids, non-essential amino acids, antibiotics and combinations thereof.

10. A method for isolating germline-like stem cells from amniotic fluid, said method comprising:
(a) providing amniotic fluid cells on a substrate for a sufficient time to permit a portion of said amniotic fluid cells to adhere to said substrate;
(b) removing a non-adherent portion of said amniotic fluid cells;
(c) identifying from said non-adherent portion of amniotic fluid cells, those cells expressing at least one germline-like stem marker, said at least one germline-like stem marker being DAZL.

11. The method of claim 10, wherein said identifying step comprises performing flow cytometry analysis, immunocytochemical analysis, or RT-PCR or a combination thereof.

12. A cell line isolated by method of claim 10.

13. The method of claim 10 wherein said germline-like stem cells may further express at least one of alkaline phosphatase; SSEA-3; SSEA-4; TRA-1-60; TRA-1-81; TRA2-54; c-kit; Oct-4 and oxytocin receptor.

14. The method of claim 10 wherein said germline-like stem cells do not express SSEA-1.

15. The method of claim 10 wherein said germline-like stem cells may further express at least one of HLA Class I, CD13, CD44, CD49b, and CD105.

16. The method of claim 10 wherein said germline-like stem cells may further express at least one of POU5F1, TDGF, GABRB3, FGF4 and TERT.

17. An isolated amniotic fluid germline-like stem cell which is pluripotent and DAZL positive.

18. The isolated germline-like stem cell of claim 17 wherein said cell further expresses at least one of alkaline phosphatase; SSEA-3; SSEA-4; TRA-1-60; TRA-1-81; TRA2-54; c-kit; Oct-4 and oxytocin receptor.

19. The isolated germline-like stem cell as in claim 18 wherein said germline-like stem cells do not express SSEA-1.

20. The isolated germline-like stem cell as in claim 19 wherein said cell further expresses at least one of HLA Class I, CD13, CD44, CD49b, and CD105.

21. The isolated germline-like stem cell as in claim 17 wherein said cell further expresses at least one of POU5F1, TDGF, GABRB3, FGF4 and TERT.

22. A method of treating a disease in a human comprising administering the cell or a plurality of said cell of claim 17 into an individual in need thereof.

23. The method of claim 22 wherein said disease is selected from the group consisting of: infertility, cirrhosis of the liver, pancreatitis, diabetes, Parkinson's disease, spinal cord injury, stroke, burns, heart disease, certain types of cancer, osteoarthritis, rheumatoid arthritis, leukemia, lymphoma, genetic blood disorders, and Alzheimer's disease.

24. A culture medium for the growth of germline-like stem cells isolated from amniotic fluid, said culture medium comprising aminotic fluid and basal medium, said basal medium comprising: about 80% Dulbeco's modified Eagle's medium; and serum replacement medium.

25. A culture medium of claim 24, further comprising one of more of L-glutamine, nonessential amino acids, antibiotics, a growth factor and other agent.

26. The culture medium of claim 24, wherein said growth factor is basic fibroblast growth factor (bFGF).
